(19) United States
(12) Patent Application Publication
(10) Pub. No.: US 2022/0192984 A1
(43) Pub. Date: Jun. 23, 2022

(54) PHARMACEUTICAL COMPOSITIONS COMPRISING OTIC THERAPEUTIC AGENTS AND RELATED METHODS

(71) Applicant: Frequency Therapeutics, Inc., Lexington, MA (US)

(72) Inventors: Will MCLEAN, North Haven, CT (US); Rajesh MANCHANDA, Acton, MA (US); Snehal KHEDKAR, Lexington, MA (US); Richard A. STRONG, Jr., Chelmsford, MA (US); Ashley BANKS, Somerville, MA (US); Bradley TAIT, North Andover, MA (US); Christopher LOOSE, Winchester, MA (US)

(21) Appl. No.: 17/282,069

(22) PCT Filed: Oct. 2, 2019
(86) PCT No.: PCT/US2019/054235

§ 371 Date: Apr. 1, 2021

Related U.S. Application Data
(60) Provisional application No. 62/739,933, filed on Oct. 2, 2018.

(51) Int. Cl. A61K 9/16; A61K 9/00; A61K 45/06; A61K 9/19; A61P 27/16

(52) U.S. Cl. CPC A61K 9/1641; A61K 9/0046; A61P 27/16; A61K 9/19; A61K 45/06

(57) ABSTRACT

The present disclosure relates to pharmaceutical compositions (e.g., pre-lyophilized pharmaceutical compositions, lyophilized pharmaceutical compositions, and reconstituted solutions) comprising one or more otic therapeutic agents. The present disclosure also relates to methods of preparing the pharmaceutical compositions, and methods of using the pharmaceutical compositions for therapeutic purpose.

### RELATED APPLICATIONS

This application is a U.S. National Phase application, filed under U.S.C. 371, of International Application No. PCT/US2019/054235, filed Oct. 2, 2019, which claims priority to U.S. Provisional Patent Application Ser. No. 62/739,933, filed Oct. 2,2018; the contents of each of which are incorporated herein by reference in their entireties.

### BACKGROUND

Stem cells exhibit an extraordinary ability to generate multiple cell types in the body. Besides embryonic stem cells, tissue specific stem cells serve a critical role during development as well as in homeostasis and injury repair in the adult. Stem cells renew themselves through proliferation as well as generate tissue specific cell types through differentiation. The characteristics of different stem cells vary from tissue to tissue, and are determined by their intrinsic genetic and epigenetic status. However, the balance between self-renewal and differentiation of different stem cells are all stringently controlled. Uncontrolled self-renewal may lead to overgrowth of stem cells and possibly tumor formation, while uncontrolled differentiation may exhaust the stem cell pool, leading to an impaired ability to sustain tissue homeostasis. Thus, stem cells continuously sense their environment and appropriately respond with proliferation, differentiation or apoptosis. It would be desirable to drive regeneration by controlling the timing and extent of stem cell proliferation and differentiation. Controlling the proliferation with small molecules that are cleared over time would allow for control of the timing and extent of stem cell proliferation and differentiation. Remarkably, tissue stem cells from different tissues share a limited number of signaling pathways for the regulation of their self-renewal and differentiation, albeit in a very context dependent manner. Some of these pathways are the Wnt and GSK3-β pathways.

Lgr5 is expressed across a diverse range of tissues and has been identified as a biomarker of adult stem cells in a variety of tissues such as the gut epithelia (Barker et al. 2007), kidney, hair follicle, and stomach (Barker et al, 2010; Haegebarth & Clevers, 2009). For example, it was first published in 2011, that mammalian inner ear hair cells are derived from LGR5⁺ cells (Chai et al, 2011, Shi et al. 2012). Lgr5 is a known component of the Wnt/β-catenin pathway, which has been shown to play major roles in differentiation, proliferation, and inducing stem cell characteristics (Barker et al. 2007).

Permanent damage to the hair cells of the inner ear results in sensorineural hearing loss, leading to communication difficulties in a large percentage of the population. Hair cells are the receptor cells that transduce the acoustic stimulus. Regeneration of damaged hair cells would provide an avenue for the treatment of a condition that currently has no therapies other than prosthetic devices. Although hair cells do not regenerate in the mammalian cochlea, new hair cells in lower vertebrates are generated from epithelial cells, called supporting cells, that surround hair cells.

Thus, there remains a need for novel pharmaceutical compositions to protect auditory cells before injury and preserve/promote the function of existing cells after injury. There remains a need for for novel pharmaceutical compositions to regenerate cochlear supporting cells or hair cells after injury.

In addition to the above reasons for the need of novel pharmaceutical compositions to regenerate cochlea supporting cells or hair cells after injury, there remains a need to be able to provide the novel pharmaceutical compositions in a manner to efficiently facilitate their intended use. For example, manufacturing and storing the pharmaceutical compositions until required poses many challenges, such as those relating to stability of the pharmaceutically active ingredients. For example, gel formulations may pose particular challenges in relation to stability and a dry composition might not be readily reconstituted to form a gel formulation.

### SUMMARY

In some aspects, the present disclosure provides a lyophilized pharmaceutical composition comprising a gelling agent.

In some aspects, the present disclosure provides a gel pharmaceutical composition, for example a thermoreversible gel, comprising one or more otic therapeutic agents.

In some aspects, the lyophilized pharmaceutical compositions disclosed herein are reconstituted to form the gel pharmaceutical composition, for example a thermoreversible gel, disclosed herein.

In some aspects, the present disclosure provides, inter alia, a lyophilized pharmaceutical composition comprising one or more otic therapeutic agents and a gelling agent

In some aspects, the present disclosure provides a lyophilized pharmaceutical composition comprising about 50 to about 500 mg of poloxamer and about 50 to about 500 mg of a compound of formula (I), for example valproic acid or a pharmaceutically acceptable salt thereof.

In some aspects, the present disclosure provides a pharmaceutical composition comprising one or more otic therapeutic agents and a gelling agent. For example, a pharmaceutical composition may comprise purified poloxamer and an increased concentration of valproic acid or a pharmaceutically acceptable salt thereof while maintaining suitable gelling characteristics. In a further example, a pharmaceutical composition may comprise an increased concentration of valproic acid or a pharmaceutically acceptable salt thereof and CHIR99021 or a pharmaceutically acceptable salt thereof, wherein the increased concentration of valproic acid or a pharmaceutically acceptable salt thereof increases the level of CHR99021 or a pharmaceutically acceptable salt thereof in the inner ear.

In some aspects, the present disclosure provides comprising a gelling agent and a compound of formula (I):

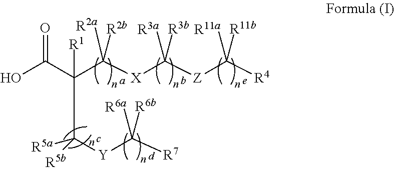

or a pharmaceutically acceptable salt thereof.

In some aspects, the present disclosure provides a pharmaceutical composition comprising a gelling agent, valproic acid or a pharmaceutically acceptable salt thereof at a concentration of greater than about 70 mg/ml, and one or more otic therapeutic agents.

In some aspects, the present disclosure provides a composition that is suitable for intratympanic injection.

In some aspects, the present disclosure provides a pharmaceutical composition comprising a poloxamer, wherein at least 85% by wt. % of the poloxamer has an average molecular weight of greater than about 7250 Da, and valproic acid or a pharmaceutically acceptable salt thereof is present at a concentration of greater than 70 mg/ml.

In some aspects, the present disclosure provides a pharmaceutical composition comprising a poloxamer, wherein less than 20% by wt. % of the poloxamer has an average molecular weight less about 7250 Da, and valproic acid or a pharmaceutically acceptable salt thereof at a concentration of greater than 70 mg/ml.

In some aspects, the present disclosure provides a method for preparing a pharmaceutical composition comprising the steps of (a) having an aqueous solution comprising a gelling agent; and (b) adding a solution of one or more otic therapeutic agents or a pharmaceutically acceptable salt thereof.

In some aspects, the present disclosure provides a lyophilized pharmaceutical composition comprising a gelling agent and one or more otic therapeutic agents, wherein the composition does not contain an additional bulking agent.

In some aspects, the present disclosure provides a lyophilized pharmaceutical composition comprising a poloxamer and one or more otic agents, wherein the composition does not contain an antioxidant.

In some aspects, the present disclosure provides a method of lyophilizing a pharmaceutical composition.

In some aspects, the present disclosure provides a method of reconstituting a lyophilized pharmaceutical composition.

In some aspects, the present disclosure provides a reconstituted pharmaceutical composition.

In some embodiments, the one or more otic therapeutic agents are one or more hearing loss treatment agents.

In some embodiments, the one or more otic therapeutic agents are modulators of one or more biological pathways and biological targets associated with hearing loss.

In some embodiments, the one or more otic therapeutic agents are hair cell regeneration agents and/or otoprotective agents.

In some embodiments, the one or more otic therapeutic agents are selected from the group consisting of the agents described in Tables 1-13, and pharmaceutical salts thereof.

In some embodiments, the one or more otic therapeutic agents are CHIR99021 or a pharmaceutical acceptable salt thereof, and valproic acid or a pharmaceutical acceptable salt thereof.

In some embodiments, the composition comprises CHIR99021 or a pharmaceutically acceptable salt thereof, valproic acid or a pharmaceutically acceptable salt thereof, and a gelling agent.

In some embodiments, the pharmaceutically acceptable salt of valproic acid is a sodium salt (e.g., sodium valproate).

In some embodiments, the gelling agent is a thermoreversible gelling agent (e.g., a poloxamer).

In some embodiments, the poloxamer is Poloxamer 407.

In some embodiments, the poloxamer is a purified poloxamer (e.g., purified Poloxamer 407).

In some aspects, the present disclosure provides a method of treating hearing loss, comprising administering to a subject in need thereof a pharmaceutically acceptable amount of a reconstituted solution, wherein the reconstituted solution is prepared by a reconstitution process using the lyophilized pharmaceutical composition of any one of the preceding claims.

In some aspects, the present disclosure provides a pharmaceutical composition, comprising:

i) CHIR99021 or a pharmaceutically acceptable salt thereof being present at a concentration ranging from 0.025 mg/ml to about 25 mg/ml;

ii) valproic acid or a pharmaceutically acceptable salt thereof being present at a concentration ranging from 0.5 mg/ml to about 500 mg/ml;

iii) poloxamer 407 being present at a concentration ranging from 1 wt % to about 25 wt %; and

iv) dimethyl sulfoxide (DMSO) being present at a concentration below 7.5 wt %.

In some aspects, the present disclosure provides a pharmaceutical composition, comprising:

i) CHIR99021 or a pharmaceutically acceptable salt thereof being present at a concentration ranging from 0.025 mg/ml to about 25 mg/ml;

ii) valproic acid or a pharmaceutically acceptable salt thereof being present at a concentration ranging from 1 mg/ml to about 500 mg/ml;

iii) poloxamer 407 being present at a concentration ranging from 1 wt % to about 25 wt %; and

iv) dimethyl sulfoxide (DMSO) being present at a concentration below 7.5 wt %.

In some aspects, the present disclosure provides a pharmaceutical composition, comprising:

i) CHIR99021 or a pharmaceutically acceptable salt thereof being present at a concentration ranging from 0.025 mg/ml to about 25 mg/ml;

ii) valproic acid or a pharmaceutically acceptable salt thereof being present at a concentration ranging from 0.5 mg/ml to about 500 mg/ml;

iii) poloxamer 407 being present at a concentration ranging from 1 wt % to about 25 wt %; and

iv) dimethyl sulfoxide (DMSO) being present at a concentration below 7.5 wt %.

In some aspects, the present disclosure provides a method of processing the pharmaceutical composition of the present disclosure to form a lyophilized pharmaceutical composition.

In some aspects, the present disclosure provides a lyophilized pharmaceutical composition being prepared by lyophilizing the pharmaceutical composition of the present disclosure.

In some aspects, the present disclosure provides a lyophilized pharmaceutical composition being prepared by the method of the present disclosure.

In some aspects, the present disclosure provides a reconstituted solution being prepared by adding a diluent to the lyophilized pharmaceutical composition of the present disclosure.

In some aspects, the present disclosure provides a reconstituted solution being prepared by adding a diluent to a lyophilized pharmaceutical composition which is prepared by lyophilizing the pharmaceutical composition of the present disclosure.

In some aspects, the present disclosure provides a reconstituted solution being prepared by adding a diluent to a lyophilized pharmaceutical composition which is prepared by the method of the present disclosure.

In some aspects, the present disclosure provides a reconstituted solution being prepared by adding a diluent to a lyophilized pharmaceutical composition, comprising one or more otic therapeutic agents and a gelling agent.

In some aspects, the present disclosure provides a method of facilitating the generation of a tissue and/or a cell, comprising delivering a pharmaceutically effective amount of the lyophilized pharmaceutical composition, the pharmaceutical composition, or the reconstituted solution of the present disclosure to the tissue and/or the cell.

In some aspects, the present disclosure provides a method of treating a subject who has, or is at risk of developing, a disease associated with absence or a lack of a tissue and/or a cell, comprising administering to the subject a pharmaceutically effective amount of the lyophilized pharmaceutical composition, the pharmaceutical composition, or the reconstituted solution of the present disclosure.

In some aspects, the present disclosure provides a method of increasing a population of vestibular cells in a vestibular tissue, comprising delivering a pharmaceutically effective amount of the lyophilized pharmaceutical composition, the pharmaceutical composition, or the reconstituted solution of the present disclosure.

In some aspects, the present disclosure provides a method of treating a subject who has, or is at risk of developing a vestibular condition, comprising administering to the subject a pharmaceutically effective amount of the lyophilized pharmaceutical composition of the lyophilized pharmaceutical composition, the pharmaceutical composition, or the reconstituted solution of the present disclosure.

In some aspects, the present disclosure provides a method of increasing a population of cochlear cells in a cochlear tissue, comprising delivering a pharmaceutically effective amount of the lyophilized pharmaceutical composition, the pharmaceutical composition, or the reconstituted solution of the present disclosure.

In some aspects, the present disclosure provides a method of treating a subject who has, or is at risk of developing a cochlear condition, comprising administering to the subject a pharmaceutically effective amount of the lyophilized pharmaceutical composition, the pharmaceutical composition, or the reconstituted solution of the present disclosure.

In some aspects, the present disclosure provides a method of increasing a population of cells found in the Organ of Corti, comprising delivering a pharmaceutically effective amount of the lyophilized pharmaceutical composition, the pharmaceutical composition, or the reconstituted solution of the present disclosure to the population.

In some aspects, the present disclosure provides a method of increasing a population of hair cells found in the Organ of Corti, comprising delivering a pharmaceutically effective amount of the lyophilized pharmaceutical composition, the pharmaceutical composition, or the reconstituted solution of the present disclosure to the population.

In some aspects, the present disclosure provides a method of increasing a population of inner hair cells found in the Organ of Corti, comprising delivering a pharmaceutically effective amount of the lyophilized pharmaceutical composition, the pharmaceutical composition, or the reconstituted solution of the present disclosure to the population.

In some aspects, the present disclosure provides a method of increasing a population of outer hair cells found in the Organ of Corti, comprising delivering a pharmaceutically effective amount of the lyophilized pharmaceutical composition, the pharmaceutical composition, or the reconstituted solution of the present disclosure to the population.

In some aspects, the present disclosure provides a method of increasing a population of neuronal cells found in the Organ of Corti, comprising delivering a pharmaceutically effective amount of the lyophilized pharmaceutical composition, the pharmaceutical composition, or the reconstituted solution of the present disclosure to the population.

In some aspects, the present disclosure provides a method of treating a subject who has, or is at risk of developing a hearing condition, comprising administering to the subject a pharmaceutically effective amount of the lyophilized pharmaceutical composition, the pharmaceutical composition, or the reconstituted solution of the present disclosure.

In some aspects, the present disclosure provides the lyophilized pharmaceutical composition, the pharmaceutical composition, or the reconstituted solution of the present disclosure, for use in facilitating the generation of a tissue and/or a cell.

In some aspects, the present disclosure provides the lyophilized pharmaceutical composition, the pharmaceutical composition, or the reconstituted solution of the present disclosure, for use in in treating a subject who has, or is at risk of developing, a disease associated with absence or a lack of a tissue and/or a cell.

In some aspects, the present disclosure provides the lyophilized pharmaceutical composition, the pharmaceutical composition, or the reconstituted solution of the present disclosure, for use in increasing a population of vestibular cells in a vestibular tissue.

In some aspects, the present disclosure provides the lyophilized pharmaceutical composition, the pharmaceutical composition, or the reconstituted solution of the present disclosure, for use in treating a subject who has, or is at risk of developing a vestibular condition.

In some aspects, the present disclosure provides the lyophilized pharmaceutical composition, the pharmaceutical composition, or the reconstituted solution of the present disclosure, for use in increasing a population of cochlear cells in a cochlear tissue.

In some aspects, the present disclosure provides the lyophilized pharmaceutical composition, the pharmaceutical composition, or the reconstituted solution of the present disclosure, for use in treating a subject who has, or is at risk of developing a cochlear condition.

In some aspects, the present disclosure provides the lyophilized pharmaceutical composition, the pharmaceutical composition, or the reconstituted solution of the present disclosure, for use in increasing a population of cells found in the Organ of Corti.

In some aspects, the present disclosure provides the lyophilized pharmaceutical composition, the pharmaceutical composition, or the reconstituted solution of the present disclosure, for use in increasing a population of hair cells found in the Organ of Corti.

In some aspects, the present disclosure provides the lyophilized pharmaceutical composition, the pharmaceutical composition, or the reconstituted solution of the present disclosure, for use in increasing a population of inner hair cells found in the Organ of Corti.

In some aspects, the present disclosure provides the lyophilized pharmaceutical composition, the pharmaceutical composition, or the reconstituted solution of the present disclosure, for use in increasing a population of outer hair cells found in the Organ of Corti.

In some aspects, the present disclosure provides the lyophilized pharmaceutical composition, the pharmaceutical composition, or the reconstituted solution of the present disclosure, for use in increasing a population of neuronal cells found in the Organ of Corti.

In some aspects, the present disclosure provides the lyophilized pharmaceutical composition, the pharmaceutical composition, or the reconstituted solution of the present disclosure, for use in treating a subject who has, or is at risk of developing a hearing condition.

In some aspects, the present disclosure provides for the use of the lyophilized pharmaceutical composition, the pharmaceutical composition, or the reconstituted solution of the present disclosure, in the manufacture of a medicament for facilitating the generation of a tissue and/or a cell.

In some aspects, the present disclosure provides for the use of the lyophilized pharmaceutical composition, the pharmaceutical composition, or the reconstituted solution of the present disclosure, in the manufacture of a medicament for in treating a subject who has, or is at risk of developing, a disease associated with absence or a lack of a tissue and/or a cell.

In some aspects, the present disclosure provides for the use of the lyophilized pharmaceutical composition, the pharmaceutical composition, or the reconstituted solution of the present disclosure, in the manufacture of a medicament for increasing a population of vestibular cells in a vestibular tissue.

In some aspects, the present disclosure provides for the use of the lyophilized pharmaceutical composition, the pharmaceutical composition, or the reconstituted solution of the present disclosure, in the manufacture of a medicament for treating a subject who has, or is at risk of developing a vestibular condition.

In some aspects, the present disclosure provides for the use of the lyophilized pharmaceutical composition, the pharmaceutical composition, or the reconstituted solution of the present disclosure, in the manufacture of a medicament for increasing a population of cochlear cells in a cochlear tissue.

In some aspects, the present disclosure provides for the use of the lyophilized pharmaceutical composition, the pharmaceutical composition, or the reconstituted solution of the present disclosure, in the manufacture of a medicament for treating a subject who has, or is at risk of developing a cochlear condition.

In some aspects, the present disclosure provides for the use of the lyophilized pharmaceutical composition, the pharmaceutical composition, or the reconstituted solution of the present disclosure, in the manufacture of a medicament for increasing a population of cells found in the Organ of Corti.

In some aspects, the present disclosure provides for the use of the lyophilized pharmaceutical composition, the pharmaceutical composition, or the reconstituted solution of the present disclosure, in the manufacture of a medicament for increasing a population of hair cells found in the Organ of Corti.

In some aspects, the present disclosure provides for the use of the lyophilized pharmaceutical composition, the pharmaceutical composition, or the reconstituted solution of the present disclosure, in the manufacture of a medicament for increasing a population of inner hair cells found in the Organ of Corti.

In some aspects, the present disclosure provides for the use of the lyophilized pharmaceutical composition, the pharmaceutical composition, or the reconstituted solution of the present disclosure, in the manufacture of a medicament for increasing a population of outer hair cells found in the Organ of Corti.

In some aspects, the present disclosure provides for the use of the lyophilized pharmaceutical composition, the pharmaceutical composition, or the reconstituted solution of the present disclosure, in the manufacture of a medicament for increasing a population of neuronal cells found in the Organ of Corti.

In some aspects, the present disclosure provides for the use of the lyophilized pharmaceutical composition, the pharmaceutical composition, or the reconstituted solution of the present disclosure, in the manufacture of a medicament for treating a subject who has, or is at risk of developing a hearing condition.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference for all purposes. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

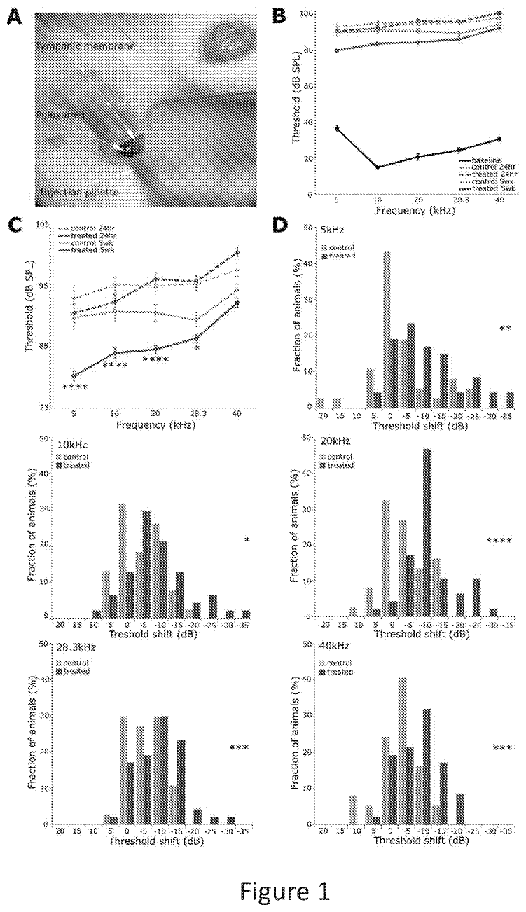

FIG. 1: Shows an analysis of auditory brainstem responses (ABR) for the treatment in a noise-damage model for induced hearing loss. Treatment with CHIR99021+VPA leads to hearing improvement in an in vivo noise damage model. (A) Image of injection procedure to transtympanically inject poloxamer into the middle ear of mice. (B) Animals designated to control and treated groups had elevated thresholds at 24 hrs and 5 wks after noise exposure compared to pre-exposure baseline. Control n=37 animals, treated n=−47 animals. (C) At 5 wks after injection, treated animals had significantly lower hearing thresholds relative to control animals for 4 of the 5 frequencies tested. (D) The distribution of individual hearing recoveries was analyzed. Values represent the change in dB needed to elicit an ABR response, with positive values representing further threshold increases (further hearing loss) and negative values representing threshold decreases (improved hearing). The faction of animals with a given ABR change from 24 hr to 5 wks are shown for each frequency tested. The treated group had a higher incidence of animals with hearing improvement and the greatest individual recoveries. Values are presented as means SE; *=p<0.05, **=p<0.01, ***=p<0.001, ****=p<0.0001.

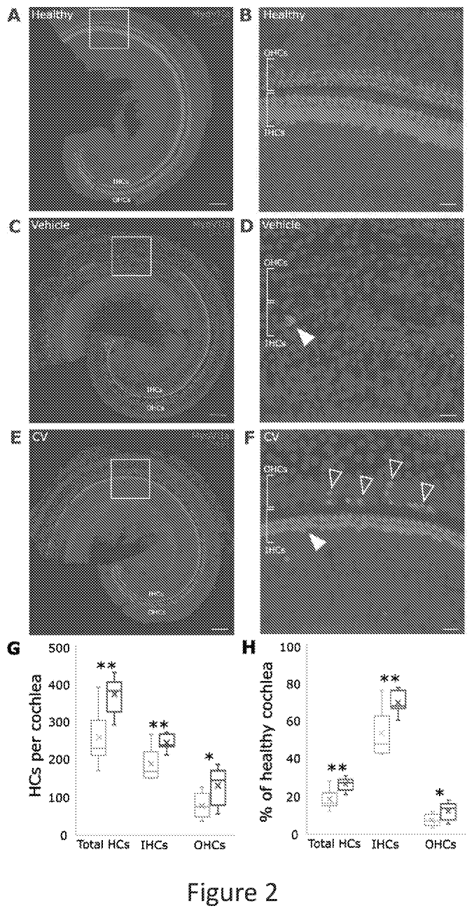

FIG. 2 shows an analysis of hair cell count for treatment in a noise-damage model for induced hearing loss. (A) Low magnification view of a healthy isolated cochlear section showing complete rows of inner hair cells (IHCs) and outer hair cells (OHCs). (B) High magnification view of the region highlighted in a) showing intact IHCs and OHCs in mid frequency regions. (C) Cochleae of vehicle injected animals show widespread hair cell loss throughout the cochlea (apex and mid region shown). (D) High magnification view of the region highlighted in (C) showing substantial absence of hair cells in mid frequency regions, where a single IHC can be seen in the field of view (solid arrow). (E) Cochleae of CV (CHIR99021 and NaVPA) treated animals show a greater overall population of hair cells compared to vehicle treated animals (apex and mid region shown). (F) High magnification view of the region highlighted in (E) showing a complete row of IHCs (solid arrow) and a population of OHCs (open arrow). (G) CV treated cochlea (blue) show significantly more total hair cells, IHCs, and OHCs relative to vehicle treated cochleae (grey). (H) The number of hair cells depicted as the percentage relative to an undamaged healthy cochlea. CV treated cochlea (blue) show significantly higher percentage of total hair cells, IHCs, and OHCs relative to vehicle treated cochleae (grey). Scale bais, 100 μm low magnification, 20 μm high magnification. Values are presented as box-whisker plots; n=7 animals per group, *=p<0.05, **=p<0.01.

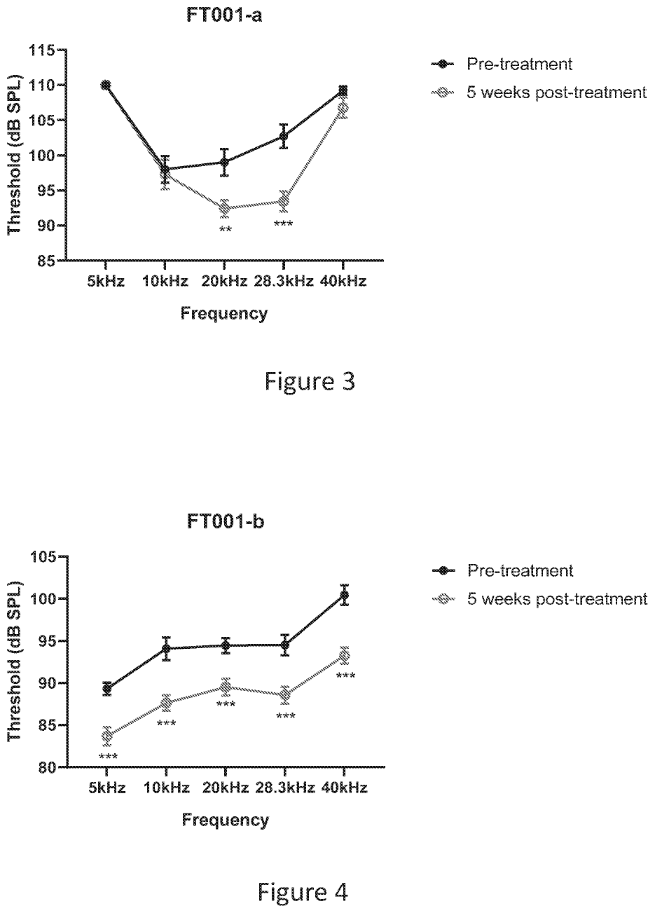

FIG. 3. Animal model data: significant improvement in thresholds seen at 20 kHz and 28.3 kHz.

FIG. 4. Animal model data: significant improvement in thresholds seen at all frequencies.

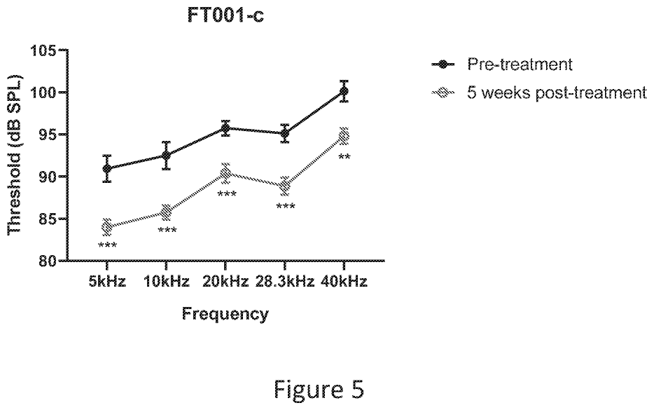

FIG. 5 Animal model data: significant improvement in thresholds seen at all frequencies.

FIG. 6: NaVPA logarithmic mean concentrations.

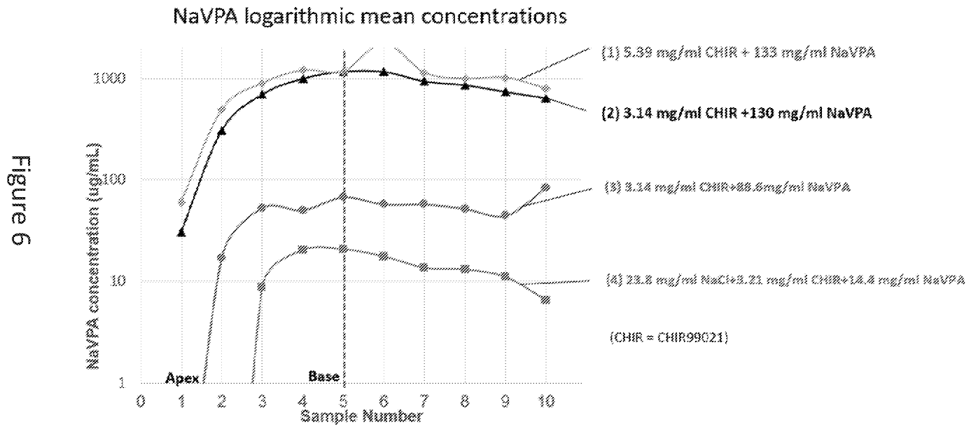

FIG. 7: CHIR99021 logarithmic mean concentrations.

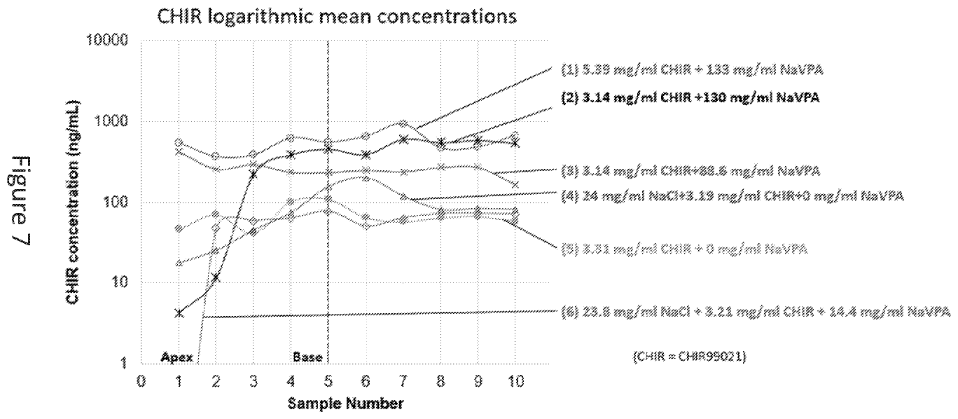

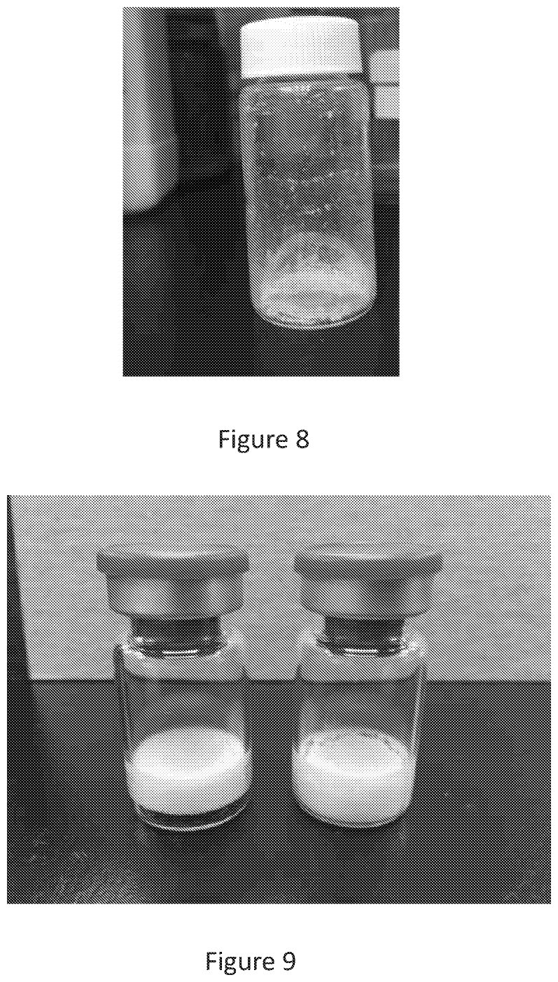

FIG. 8: Lyophilized test composition without use of an appropriate lyophilization cycle.

FIG. 9: Lyophilized test composition manufactured using the developed lyophilization cycle.

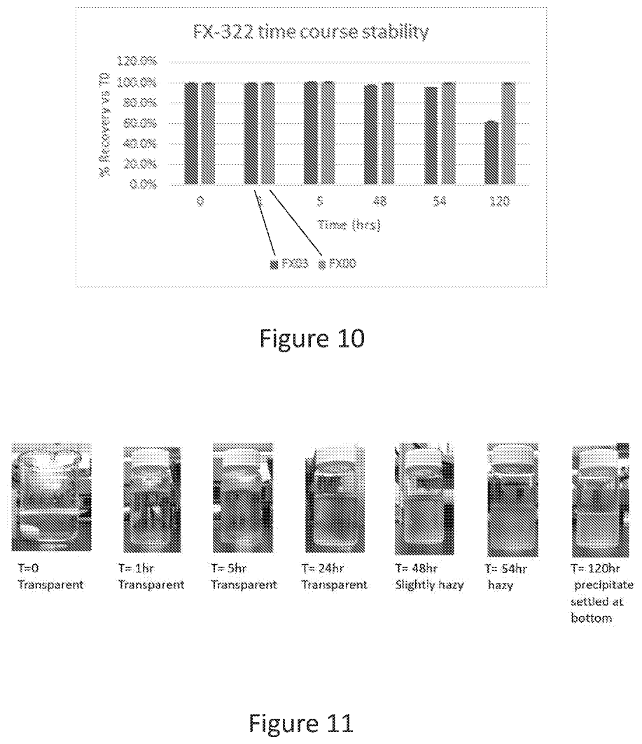

FIG. 10. Test composition time course stability.

FIG. 11. Solutions of the test composition after time, T.

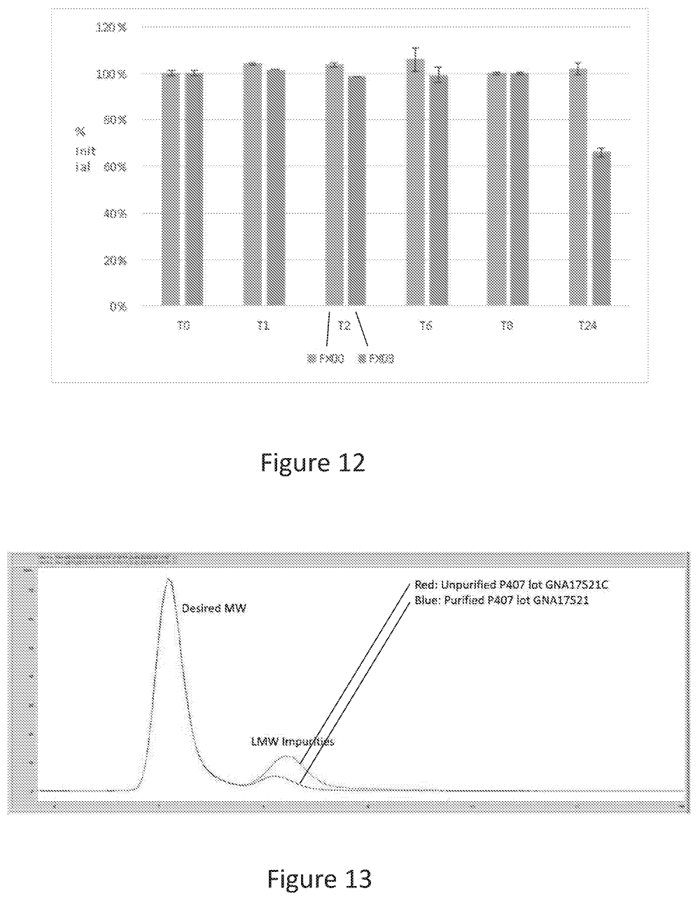

FIG. 12. Reconstituted NaVPA and CHIR99021 assay levels within refrigerated syringes.

FIG. 13: The chromatogram P407 Lot GNAC17521C before (red trace) and after purification (blue trace).

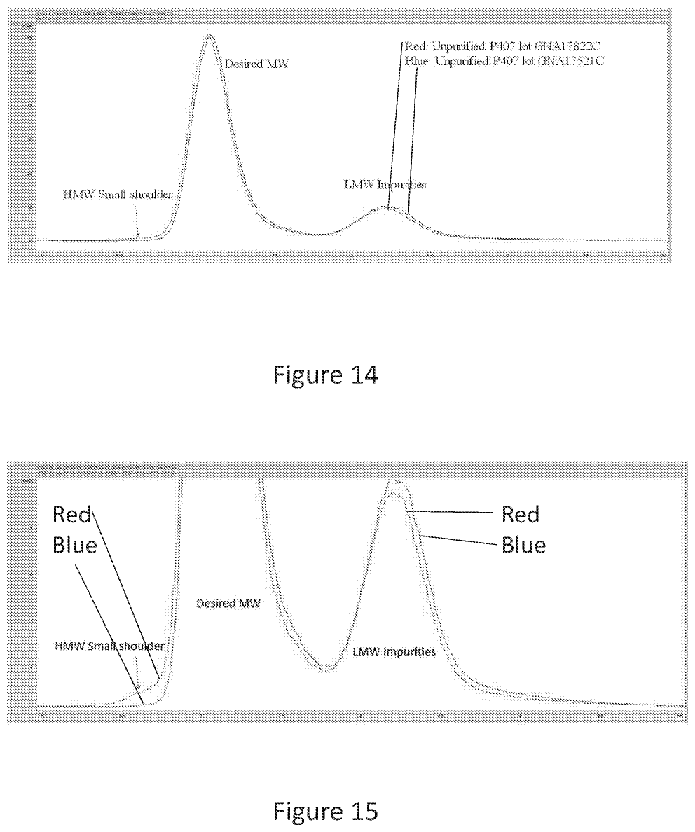

FIG. 14: High molecular weight (HWM) impurities correspond to a very small percentage by weight. Where present, high molecular weight impurities are observed as a small shoulder eluting before the desired MW peak. The chromatogram illustrates the HMW content for two lots of unpurified P407.

FIG. 15: A zoomed in portion of FIG. 12.

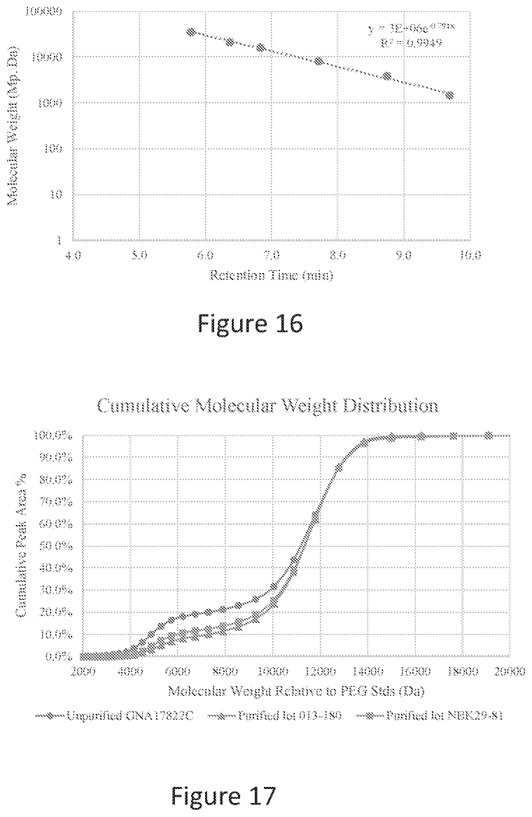

FIG. 16: Molecular weight calibration curve for PEG standards analyzed by SEC.

FIG. 17: Cumulative molecular weight distribution.

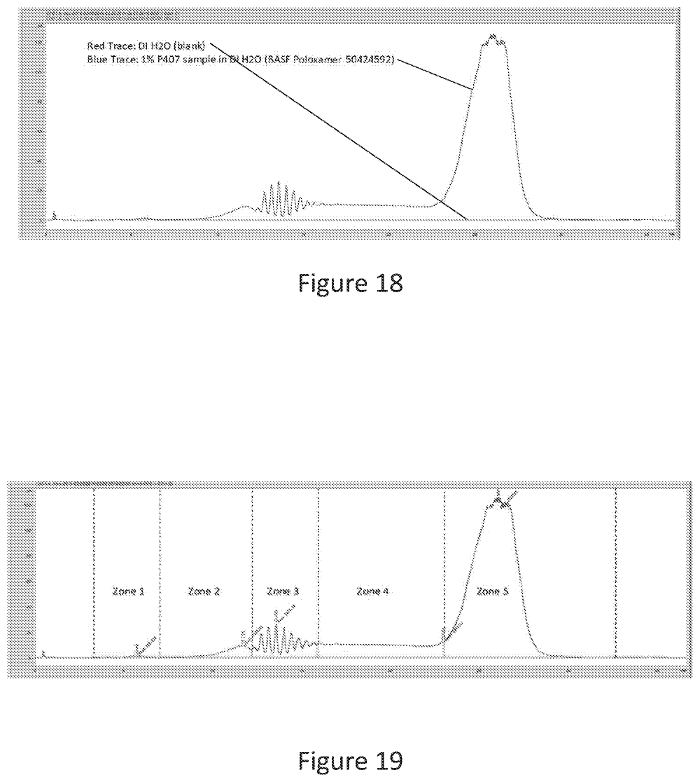

FIG. 18: A typical CAD chromatogram for a blank H₂O injection compared to a 1% P407 sample.

FIG. 19: RPLC-CAD chromatogram of P407 with impurities are divided into “zones” in the chromatogram.

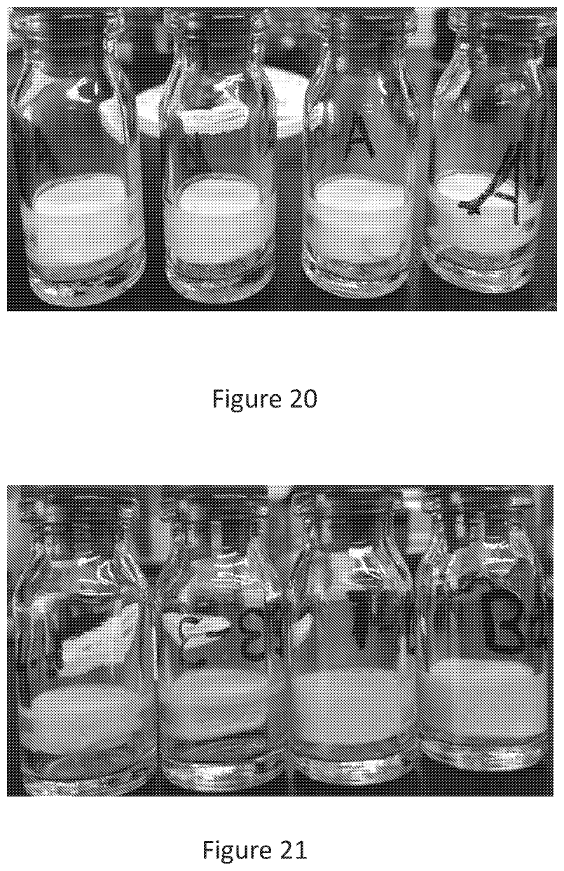

FIG. 20: lyophilized test composition A (entry 2, Table 35).

FIG. 21: lyophilized test composition B (entry 3, Table 35).

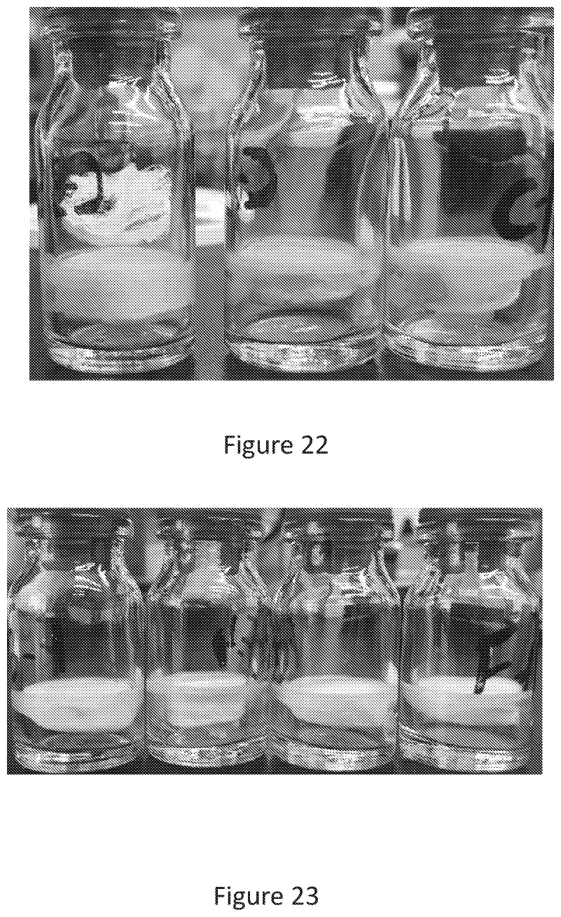

FIG. 22 lyophilized test composition C (entry 4, Table 35).

FIG. 23 lyophilized test composition D (ently 5, Table 35).

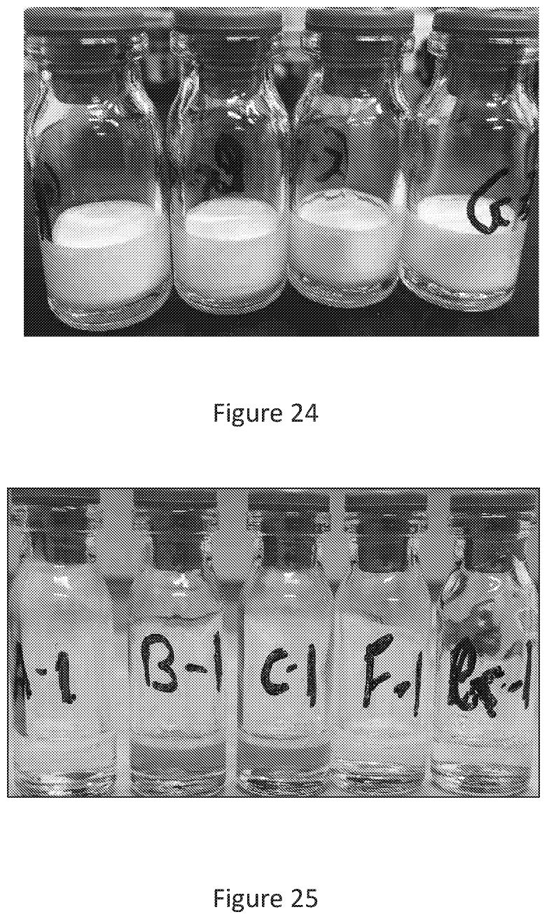

FIG. 24: lyophilized test composition E (ently 6, Table 35).

FIG. 25: reconstituted compositions A (A1), B (B-1), C (C-1), D (F-1), and E (G-1) from Table 35.

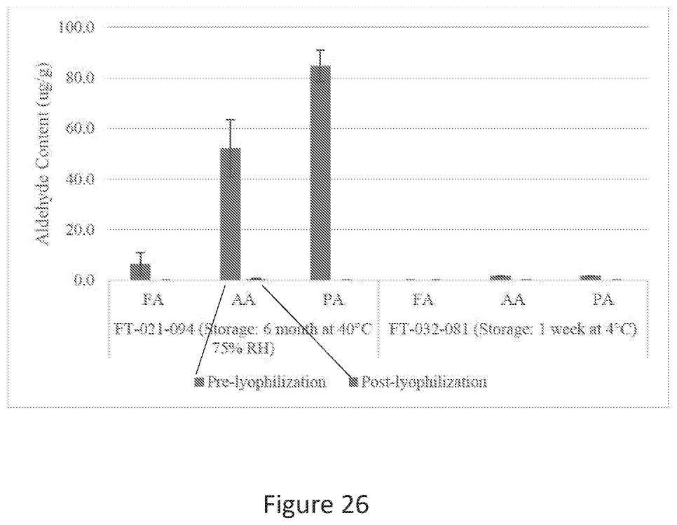

FIG. 26: Aldehyde content in liquid placebo before and after lyophilization.

### DETAILED DESCRIPTION

In some aspects, the present disclosure provides, inter alia, a lyophilized pharmaceutical composition comprising one or more otic therapeutic agents (e.g., CHIR99021 and sodium valproate) and a gelling agent (e.g., Poloxamer 407).

In some aspects, the present disclosure provides a lyophilized pharmaceutical composition comprising one or more otic therapeutic agents (e.g., CHIR99021 or a pharmaceutically acceptable salt thereof and sodium valproate or a pharmaceutically acceptable salt thereof) and a gelling agent (e.g., a poloxamer).

In some aspects, the present disclosure provides a lyophilized pharmaceutical composition comprising one or more otic therapeutic agents (e.g., LY2090314 or a pharmaceutically acceptable salt thereof and sodium valproate or a pharmaceutically acceptable salt thereof) and a gelling agent (e.g., a poloxamer).

In some aspects, the present disclosure provides a lyophilized pharmaceutical composition comprising a gelling agent (e.g., a poloxamer) and a compound of formula (I) (e.g., an HDAC inhibitor, such as valproic acid or a pharmaceutically acceptable salt thereof).

In some aspects, the present disclosure provides a pharmaceutical composition comprising one or more otic therapeutic agents (e.g., CHIR99021 or a pharmaceutically acceptable salt thereof, and valproic acid or a pharmaceutically acceptable salt thereof), wherein the increased concentration of one of the one or more otic therapeutic agents (e.g., valproic acid or a pharmaceutically acceptable salt thereof), increases the level of the other one or more otic therapeutic agents (e.g., CHIR99021 or a pharmaceutically acceptable salt thereof) in the inner ear.

In some aspects, the present disclosure provides a pharmaceutical composition comprising a gelling agent (e.g., a poloxamer) at a certain purity and one or more otic therapeutic agents (e.g., valproic acid or a pharmaceutically acceptable salt thereof) at a certain concentration.

In some aspects, the present disclosure provides a lyophilized pharmaceutical composition comprising one or more otic therapeutic agents (e.g., CHIR99021 or a pharmaceutically acceptable salt thereof and valproic acid or a pharmaceutically acceptable salt thereof) and a gelling agent (e.g., poloxamer), where the composition does not comprise an additional bulking agent.

In some aspects, the present disclosure provides a lyophilized pharmaceutical composition comprising one or more otic therapeutic agents (e.g., CHIR99021 or a pharmaceutically acceptable salt thereof and valproic acid or a pharmaceutically acceptable salt thereof) and a gelling agent (e.g. poloxamer), where the composition does not comprise an antioxidant.

In some aspects, the present disclosure provides a method of preparing the pharmaceutical composition of the present disclosure.

In some aspects, the present disclosure provides a method for preparing a pharmaceutical composition comprising the steps of (a) having a solution comprising a gelling agent (e.g. a poloxamer) and one or more otic therapeutic agents (e.g. valproic acid or a pharmaceutically acceptable salt thereof); and (b) adding a solution of one or more otic therapeutic agents (e.g. CHIR99021 or a pharmaceutically acceptable salt thereof).

In some aspects, the present disclosure provides a method for lyophilizing a pharmaceutical composition.

In some aspects, the present disclosure provides a pharmaceutical composition (e.g., a pre-lyophilized pharmaceutical composition) comprising one or more otic therapeutic agents (e.g., CHIR99021 and sodium valproate) and a gelling (e.g., Poloxamer 407 and other polyethylene oxide-polypropylene oxide block copolymers, including triblock polymers) or other thermoreversible (also called “thermosetting” gelling agents) such as polylactic acid (PLA)-polyethylene oxide block copolymers (including PEO-PLA-PEO triblock copolymers).

In some aspects, the present disclosure provides a method of processing the pharmaceutical composition of the present disclosure to form a lyophilized pharmaceutical composition (e.g., the pharmaceutical composition of the present disclosure).

In some aspects, the present disclosure provides a reconstituted solution comprising one or more otic therapeutic agents (e.g., CHIR99021 and sodium valproate) and a gelling (e.g., Poloxamer 407).

In some aspects, the present disclosure provides a lyophilized pharmaceutical composition comprising Poloxamer 407, CHIR99021 or a pharmaceutically acceptable salt thereof and valproic acid or a pharmaceutically acceptable salt thereof (e.g. sodium valproate).

In some aspects, the present disclosure provides a lyophilized pharmaceutical composition comprising Poloxamer 407, CHIR99021 or a pharmaceutically acceptable salt thereof and 2-hexyl-5-pentynoic acid or a pharmaceutically acceptable salt thereof (e.g. sodium 2-hexyl-5-pentynoic acid).

In some aspects, the present disclosure provides a lyophilized pharmaceutical composition comprising Poloxamer 407, CHIR99021 or a pharmaceutically acceptable salt thereof and linoleic acid or a pharmaceutically acceptable salt thereof (e.g. sodium lineolate).

In some aspects, the present disclosure provides a lyophilized pharmaceutical composition comprising Poloxamer 407, LY2090314 or a pharmaceutically acceptable salt thereof and valproic acid or a pharmaceutically acceptable salt thereof (e.g. sodium valproate).

In some aspects, the present disclosure provides a lyophilized pharmaceutical composition comprising Poloxamer 407, AZD1080 or a pharmaceutically acceptable salt thereof and valproic acid or a pharmaceutically acceptable salt thereof (e.g. sodium valproate).

In some aspects, the present disclosure provides a lyophilized pharmaceutical composition comprising Poloxamer 407, GSK3 XXII or a pharmaceutically acceptable salt thereof and valproic acid or a pharmaceutically acceptable salt thereof (e.g. sodium valproate).

In some aspects, the present disclosure provides a lyophilized pharmaceutical composition comprising Poloxamer 407, Compound I-7 or a pharmaceutically acceptable salt thereof and valproic acid or a pharmaceutically acceptable salt thereof (e.g. sodium valproate).

In some aspects, the present disclosure provides a lyophilized pharmaceutical composition comprising Poloxamer 407, Compound I-1 or a pharmaceutically acceptable salt thereof and valproic acid or a pharmaceutically acceptable salt thereof (e.g. sodium valproate).

In some aspects, the present disclosure provides a lyophilized pharmaceutical composition comprising Poloxamer 407, Compound I-3 or a pharmaceutically acceptable salt thereof and valproic acid or a pharmaceutically acceptable salt thereof (e.g. sodium valproate).

In some aspects, the present disclosure provides a lyophilized pharmaceutical composition comprising Poloxamer 407 and valproic acid or a pharmaceutically acceptable salt thereof (e.g. sodium valproate).

In some aspects, the present disclosure provides a pharmaceutical composition suitable for intratympanic injection comprising Poloxamer 407, valproic acid or a pharmaceutically acceptable salt thereof (e.g. sodium valproate) at a concentration of at least about 120 mg/ml, and CHIR99021 or a pharmaceutically acceptable salt thereof.

In some aspects, the present disclosure provides a pharmaceutical composition comprising at least 85 wt %.% Poloxamer 407 having an average molecular weight greater than about 7250, and valproic acid or a pharmaceutically acceptable salt thereof (e.g. sodium valproate) at a concentration of greater than 120 mg/ml, and CHIR99021 or a pharmaceutically acceptable salt thereof.

In some aspects, the present disclosure provides a lyophilized pharmaceutical composition comprising Poloxamer 407, valproic acid or a pharmaceutically acceptable salt thereof (e.g. sodium valproate), and CHIR99021 or a pharmaceutically acceptable salt thereof, wherein in the composition does not comprise an additional bulking agent.

In some aspects, the present disclosure provides a lyophilized pharmaceutical composition comprising Poloxamer 407, valproic acid or a pharmaceutically acceptable salt thereof (e.g. sodium valproate), and CHIR99021 or a pharmaceutically acceptable salt thereof, wherein in the composition does not comprise an antioxidant.

In some aspects, the present disclosure provides a method for preparing a pharmaceutical composition comprising the steps of (a) having an aqueous solution comprising Poloxamer 407 and valproic acid or a pharmaceutically acceptable salt thereof (e.g. sodium valproate); and (b) adding a solution comprising DMSO and CHIR99021 or a pharmaceutically acceptable salt thereof.

In some aspects, the present disclosure provides a method for lyophilizing a pharmaceutical composition comprising Poloxamer 407, valproic acid or a pharmaceutically acceptable salt thereof (e.g. sodium valproate), and CHIR99021 or a pharmaceutically acceptable salt thereof, wherein the method comprises:

(a) providing the pharmaceutical composition; (b) lyophilizing the composition by: (i) reducing the temperature in the lyophilizer to −45° C. at a rate of 0.5° C. per minute, and then holding it at −45° C. for 3 hours; (ii) applying a vacuum of 80 mTorr; (iii) increasing the temperature to −30° C. (at a rate of 0.5° C. per minute) and holding it at −30° C. for 15 hours under a vacuum of 80 mTorr; (iv) increasing the temperature to 15° C. (at a rate of 0.5° C. per minute); and/or (v) holding the temperature at 15° C. for 20 hours under a vacuum of 80 mTorr; and (c) obtaining a lyophilized pharmaceutical composition.

In some aspects, the present disclosure provides a method for lyophilizing a pharmaceutical composition comprising Poloxamer 407, valproic acid or a pharmaceutically acceptable salt thereof (e.g. sodium valproate), and CHIR99021 or a pharmaceutically acceptable salt thereof, wherein the method comprises:

(a) providing the pharmaceutical composition; (b) lyophilizing the composition by: (i) reducing the temperature in the lyophilizer to about −45° C. at a rate of about 0.5° C. per minute, and then holding it at about −45° C. for about 3 hours; (ii) applying a vacuum of about 80 mTorr; (iii) increasing the temperature to about −30° C. (at a rate of about 0.5° C. per minute) and holding it at about −30° C. for about 15 hours under a vacuum of about 80 mTorr; (iv) increasing the temperature to about 15° C. (at a rate of about 0.5° C. per minute); and/or (v) holding the temperature at about 15° C. for about 20 hours under a vacuum of about 80 mTorr; and (c) obtaining a lyophilized pharmaceutical composition.

Improved Reconstitution Time

Away to provide a pharmaceutical composition is in a dry or non-hydrated form, e.g. as a tablet, since this typically renders the pharmaceutically active ingredient(s) in the composition stable for a useful time period that may elapse between the composition being manufactured and to when composition is administered. The pharmaceutically active ingredient(s) is usually stable in the dry composition at varying conditions (temperature, humidity etc.) over the time period that it may be subjected to.

However, for a pharmaceutical composition that is administered as a solution or a gel, the time between manufacturing to administration poses significant challenges because the pharmaceutically active ingredient(s) in the composition may be not be stable in solution for extended periods of time, and start to degrade, thus creating a degradation problem. The inventors addressed this degradation problem by lyophilizing the pharmaceutical composition to improve stability, for example for a useful time period between manufacturing and administration.

The degradation problem can be further exacerbated when the components of the composition are slow to dissolve into the solution (i.e. have poor solubility). For example, with the extended time period time taken to dissolve the components in the solution, degradation can occur. In addition, components can precipitate out of the solution over periods of time. Lyophilization of the composition does not necessarily solve the degradation problem in this scenario where the component(s) also has poor solubility because the composition has two instances, one when the composition is being manufactured and another when the composition is being reconstituted, where the composition is in the form of a solution for an extended period of time, which can lead to degradation of the components. While the long period of time to manufacture the composition may be acceptable since this can be done in a controlled environment, the long period of time taken to reconstitute the lyophilized pharmaceutical composition is not always practical since this typically would occur immediately before the composition is administered in an environment that may vary and cannot be controlled, e.g. in a medical environment. Accordingly, there remains a need to be able to manufacture a lyophilized composition that is stable and reconstitutes on an acceptable time scale.

The present disclosure offers a solution to the problem described above. Surprisingly, it has been discovered that a lyophilized composition comprising a gelling agent and a salt of an organic acid reconstitutes (i.e. dissolves into solution) more quickly than the time taken to dissolve its constituent parts prior to lyophilization. This means that the composition can be manufactured, lyophilized to produce a stable composition, stored, and then reconstituted quickly prior to administration. It has also been shown that the components of the lyophilized composition are stable for extended periods of time, unlike the composition in solution form. Thus, the present disclosure provides compositions with improved reconstitution time, for example relative to its constituent parts prior to lyophilization. In one embodiment the present disclosure provides compositions with improved reconstitution time relative to its constituent parts without lyophilization (for example as non-lyophilized powders, crystals or other forms).

The solution to the problem will be illustrated by a non-limiting example. For example, a lyophilized composition comprising a poloxamer and valproic acid or a pharmaceutically acceptable salt thereof can be reconstituted about three times faster than a lyophilized poloxamer alone or powdered poloxamer (i.e. non-lyophilized poloxamer). This result is unexpected and enables the fast reconstitution of pharmaceutical compositions. The fast reconstitution time is especially useful where it is not practical to either freshly prepare the composition, or to wait for long periods time for the composition to reconstitute e.g. because this would lead to the degradation of components of the composition.

Increased Permeation of Otic Therapeutic Agents

Delivery of a pharmaceutical composition to the inner ear, in particular the cochlea, often relies on diffusion and/or permeation of the pharmaceutical composition into the cochlea (and in particular into the Organ of Corti). Increasing permeation into the cochlea and/or the Organ of Corti is therefore desirable, and it is also desirable to avoid decomposition of the composition, prior to this point, and/or the otic therapeutic agent(s) precipitating out of solution prior to delivery to the cochlea or Organ of Corti.

Accordingly, there is a need for pharmaceutical compositions in which the otic agent(s) diffuse and/or permeate into the cochlea (and Organ of Corti) more effectively.

The present invention offers a solution to the problem described above. Surprisingly, it has been discovered that a pharmaceutical composition comprising high concentrations of an organic acid as defined herein by Formula (I), for example valproic acid, or a pharmaceutically acceptable salt thereof, increases the levels of otic therapeutic agent(s) in the cochlea.

The solution to the problem will be illustrated by anon-limiting example. For example, a pharmaceutical composition comprising CH99021 or a pharmaceutically acceptable salt thereof and an increased amount of valproic acid or a pharmaceutically acceptable salt thereof, e.g. greater than 100 mg/mL, leads to a non-linear increase in the levels of CH99021 found in the cochlea after administration. For example, a ˜50% increase in the amount of valproic acid or a pharmaceutically acceptable salt thereof in the composition can result in far more than a 50% increase of CHIR99021 in the cochlea. The increase of CHIR99021 in the cochlea can be in region of 4-14 fold. Additionally, the increased concentration of valproic acid or a pharmaceutically acceptable salt thereof in the composition can increase the concentration of valproic acid or a pharmaceutically acceptable salt thereof in cochlea by at least an order of magnitude. This result is unexpected and enables the improved delivery of a pharmaceutically active agent(s) to a part of the ear that is difficult to target and difficult to access.

Purified Poloxamer

In some instances, the present invention describes a pharmaceutical composition in the form of a solution, which comprises a poloxamer. The poloxamer, when dissolved in the composition at a certain concentration, may impart various properties to the composition, such as a certain viscosity and/or a certain gelation temperature. In some instances, the present invention requires a pharmaceutical composition with a viscosity to form an immobile gel when heated to about body temperature.

The inclusion of a further component(s) at particular concentration(s) in the composition may perturb the composition's viscosity and/or gelation in a manner such that the ability to form an immobile gel when heated to about body temperature is diminished (for example where the gel is a thermoreversible gel). Therefore, there may be an upper limit of the concentration(s) of the further component(s), e.g. therapeutic component(s), that can be tolerated by the composition while retaining physical properties that are suitable for use. Accordingly, there is a need to provide a pharmaceutical composition with an increased amount of a further component(s), e.g. therapeutic component(s), while maintaining gelling characteristics in order to manufacture pharmaceutical compositions.

The present invention offers a solution to the problem described above. Surprisingly, it has been discovered that purifying a poloxamer prior to manufacture of a pharmaceutical composition enables an increased concentration of the other component(s) to be tolerated while maintaining the composition's gelling characteristics. For example, the composition comprising purified poloxamer can tolerate increased concentrations of ionic components, such as salts of organic acids. The increased concentration of component(s) allowed by purifying the poloxamer can allow increased concentrations of therapeutic components to be achieved without adversely affecting other properties of the composition. The purified poloxamer can be prepared or characterized by any of the methods and/or measures set out herein, in any combination, including those disclosed in the numbered embodiments and examples.

The solution to the problem will be illustrated by anon-limiting example. For example, a pharmaceutical composition comprising Poloxamer 407 will have a certain gelation temperature. In some instances, the composition desirably forms a gel at about body temperature. However, other components in the composition can perturb the temperature that the composition forms a gel. For a particular composition comprising Poloxamer 407, where Poloxamer 407 has not been purified, a concentration of about 80 mg/mL of sodium valproate can be achieved. At concentrations higher than 80 mg/mL, the gelation temperature may be perturbed and the composition's desirable characteristics, such as gelation temperature, diminish. Unexpectedly, for a pharmaceutical composition comprising purified Poloxamer 407, a concentration of greater than about 80 mg/mL of sodium valproate can be achieved, while the desirable gelation temperature is maintained.

As gel compositions are often not suitable for storage or distribution, the gel compositions may be lyophilized as set out herein. Those lyophilized compositions will therefore have higher concentrations of further component(s), such as therapeutic components, than would otherwise be possible (e.g. with unpurified poloxamer) while retaining favorable gel properties when reconstituted. For example, where the gel contains a given amount of water, the lyophilized composition made from that gel provides a number of benefits. For example, such a lyophilized composition can be reconstituted, for example with the same or similar given amount of water, to provide the compositions disclosed herein that retain their gel properties despite the increased levels of further component(s).

Therefore, one aspect of the present invention is a composition comprising a poloxamer having an increased amount of VPA, or pharmaceutically acceptable salt thereof, as disclosed herein. In such embodiments, one approach to achieve the increased level of VPA, or pharmaceutically acceptable salts thereof, is to purify the poloxamer as disclosed herein. In these aspects the composition may, for example, be lyophilized or reconstituted with water.

No Additional Bulking Agent

An additional bulking agent, such as a polysaccharide, is typically added to a pharmaceutical composition prior to lyophilization in order to help control the morphology of the lyophilized composition. The additional bulking agent, such as a polysaccharide, can be added to a composition before it is lyophilized to impart improved characteristics to the lyophilized product. For example, the characteristics may be the improved morphology of the lyophilized product, in the form of a cake. It is also advantageous if the lyophilized cake is porous, has a large volume, and/or is a fluffy cake. Balanced with the need to provide a suitable lyophilized pharmaceutical composition, there is a need to provide a pharmaceutical composition with minimal components since the compositions are administered to subjects in need thereof.

The present invention offers a solution to the problem described above. Surprisingly, it has been discovered that a lyophilized composition of the present invention can be successfully lyophilized even when the composition does not comprise an additional bulking agent.

No Antioxidant

Many pharmaceutical compositions comprise an antioxidant to increase the stability of the composition over an extended period of time. Typically, an antioxidant is required where the composition contains, or degrades over time to produce, a reactive species that may react further with other components, thereby affecting the stability the composition. A species in a composition that contains an aldehyde functional group can be a reactive species, for example reacting through undesired redox pathways, which may cause degradation of the other components. Hence, the inclusion of an antioxidant may increase stability of the composition by inhibiting the redox pathways. Balanced with the need to provide a stable pharmaceutical composition, there is a need to provide a pharmaceutical composition with minimal components since the compositions are administered to subjects in need thereof.

The present invention offers a solution to the problem described above. Surprisingly, it has been discovered that a lyophilized composition of the present disclosure, that comprises a poloxamer, is stable when the composition does not comprise an antioxidant even though the poloxamer component can degrade to produce aldehydes.

The solution to the problem will be illustrated by a non-limiting example. For example, compositions of the present disclosure comprise a poloxamer, which may degrade to produce aldehydes. Unexpectedly, when lyophilizing compositions of the present disclosure, it was found that lyophilization removed substantially all of the aldehydes from the composition and/or resulted in a composition that does not produce further aldehydes once lyophilized. This result means that an antioxidant not required in the composition.

Order of Addition of Ingredients

A pharmaceutical composition that is suitable for administration as a solution or a gel typically comprises an aqueous component, such as water. This poses a problem for many pharmaceutically acceptable agents since they can be sparingly soluble in aqueous solutions. Furthermore, the actives can take extended periods of time to dissolve, precipitate out of solution and/or be unstable in solution. Accordingly, there remains a need to provide further methods of making a pharmaceutical composition as an aqueous solution in less time while maintaining the integrity of the components.

The present disclosure offers a solution to the problem described above. Surprisingly, it has been discovered that adding a pharmaceutically acceptable active(s) in the form of a concentrated solution of a polar aprotic solvent to an aqueous component results in a pharmaceutical composition where the pharmaceutically acceptable agent(s) has been solubilized in the aqueous solution. Crucially, the time taken to form the composition is reduced in comparison to alternative orders of addition, and the time that any potentially unstable components are in solution is minimized.

The solution to the problem will be illustrated by a non-limiting example. For example, CHIR99021 may exhibit low solubility in aqueous solutions and manufacturing is especially problematic where large quantities of an aqueous solution and long durations of time are required to dissolve CHIR99021 or its salts. However, pre-dissolving CHIR99021 in a polar aprotic solvent and adding that solution to the aqueous component of the composition successfully solvates CHIR99021 in an aqueous system. This result is unexpected since it occurs on a relatively short timescale, does not lead to precipitation of CHIR99021, is amenable to scale up, and is reproducible. This result is useful since it allows the formation of previously inaccessible compositions.

Lyophilization Method

Lyophilizing a pharmaceutical composition to produce an acceptable form of the lyophilized product, such as a porous cake, may be challenging. Many factors affect the outcome of the method, and the factors are amenable to a wide range of variation. For example, temperature, rate of temperature change, pressure, and duration at various temperatures and/or pressures all require careful consideration. Thus, obtaining a suitable lyophilized product from a method is no small endeavor and there remains a need to provide more lyophilization methods.

The present disclosure offers a solution to the problem described above. Surprisingly, it has been discovered that a particular method gives a suitable lyophilized composition in the form of a lyophilized cake. For example, the lyophilization method of the present disclosure is particularly advantageous because it is requires mild conditions, achievable on commercial lyophilizers, which results in a lyophilized product with good characteristics, e.g. the product cake is porous.

### Otic Therapeutic Agents

As used herein, the term “otic therapeutic agent” refers to an agent capable of treating or preventing a disease associated with the ear (e.g., Meniere's disease, hearing loss, a disease of the vestibular system, vertigo, ear inflammation, or ear infection) or a condition associated with (e.g., resulting into or resulting from) the disease.

In some embodiments, the otic therapeutic agent is a hearing loss treatment agent.

As used herein, the term “hearing loss treatment agent” refers to an agent capable for treating or preventing hearing loss or a condition associated with (e.g., causing or developing into or resulting from) hearing loss.

In some embodiments, the one or more otic therapeutic agents are one or more hearing loss treatment agents.

In some embodiments, the one or more otic therapeutic agents (e.g., hearing loss treatment agents) are modulators of one or more biological pathways and/or biological targets associated with hearing loss. Each of the modulators may independently be an agonist (e.g., activator) or antagonist (e.g., inhibitor) of one or more biological pathways and/or biological targets. In some embodiments, one or more of the modulators are agents that increase or activate the activity of one or more biological pathways and/or biological targets. In some embodiments, one or more of the modulators are agents that decrease or eliminate the activity of one or more biological pathways and/or biological targets.

In some embodiments, the one or more otic therapeutic agents (e.g., hearing loss treatment agents) are selected from the group consisting of Wnt pathway agonists, histone deacetylase (HDAC) inhibitors, Dkk1 inhibitors, Axin inhibitors, SFRP1 inhibitors, bone morphogenetic protein (BMP) inhibitors, beta-catenin agonists, CyclinD1 activators, REST corepressor 1 (CoREST) inhibitors, NOTCH agonists, TGF-beta inhibitors, cAMP response element binding protein (CREB) activators, cyclin-dependent kinase (CDK) activators, CDK inhibitors, PI3K-AKT activators, PI3K-AKT inhibitors, PTEN inhibitors, ATOH1 agonists, ATOH1 antagonists, POU4F3 agonists, POU4F3 antagonists, GFI1 agonists, GFI1 antagonists, ERK/MAPK agonists, ERK/MAPK antagonists, FGF agonists, FGF antagonists, γ-aminobutync acids (GABAs), voltage-gated Na+ channel antagonists, inositol, PKC agonists, PKC antagonists, FOXO inhibitors, FOXO agonists, Kv3 channel antagonists, p27Kip1 inhibitors, IL-1β, N-Methyl-D-aspartate (NMDA) receptor antagonists, NADPH quinone oxidoreductase 1, gamma secretase inhibitors, gamma secretase activators, NK1 receptor antagonist, NK1 receptor agonist, AMPA receptor agonist, AMPA receptor antagonist, Toll-Like Receptor (TLR) agonist, Toll-Like Receptor (TLR) antagonist, histamine H4 receptor agonist, H4 receptor antagonist, 5-HT3 receptor agonist, 5-HT3 receptor antagonist, Oct4 activators, Sox2 activators, Sox17 inducers, Klf4 inducers, cMyc activators, Sonic Hedgehog agonists, Sonic Hedgehog antagonists, Epidermal Growth Factor (EGF), Insulin Like Growth Factor (IGF), vascular endothelial growth factor (VEGF), endothelial nitric oxide synthase (eNOS), prostaglandin E (PGF), Brain-derived neurotrophic factor (BDNF), SMAD inhibitors, Sall4 inducers, Gata4 inducers, Gata6 inducers, proteasome inhibitors, retinoic acid receptor agonists, mTOR inhibitors, mTOR activators, Ascorbic acid, 2-phospho-1-ascorbic acid, KDM inhibitors, TTNPB, neurotrophin 3, DNA-modifying enzymes, LSD-1 inhibitors, Nicotinomide, Sirtuin, Histone methyl transferase inhibitors, Histone demethylase inhibitors, Histone Lysine Methyltransferase inhibitors, DNMT inhibitors, p53 inhibitors, p21 inhibitors, AMPK activators, Hippo activators, Hippo inhibitors, YAP/TAZ inhibitors, Mst1/2 inhibitors, CK1 activators, CK1 inhibitors, Noggin, R-spondin 1, BET activators, Sirt1 activators, Sirt1 inhibitors, Sirt2 activators, Sirt2 inhibitors, Sirt3 activators, Sirt3 inhibitors, JMJD3 inhibitors, DMNT inhibitors, Stat3 inhibitors, LSD1 inhibitors, active prostaglandins, cAMP activators, Oxidative phosphorylation uncouplers, arginine methyltransferase inhibitors, ALK4 inhibitors, Peroxisome proliferator-activated receptor gamma activators, EGFR inhibitors, SHH inhibitors, VitD activators, DOT1L inhibitors, Thyroid hormones, E box-dependent transcriptional activators, and protein degradation inhibitors.

In some embodiments, the one or more otic therapeutic agents (e.g., hearing loss treatment agents) are hair cell regeneration agents and/or otoprotective agents.

In some embodiments, the one or more otic therapeutic agents (e.g., hearing loss treatment agents) are selected from the group consisting of the agents described in Tables 1-13, and pharmaceutical salts thereof

TABLE 1

| | | |
|---|---|---|
| AZD1080 | Sodium p-aminosalicylate | GP-HL1 |
| GSK XXII | Oxyphenbutazone | QP-HL-3 |
| LY2090314 | Deoxycholic acid | SENS-111 |
| DMH1 | Metoprine | R-azasetron besylate |
| Sodium Butyrate | Dasatinib | SPI-1005 |
| Sodium Phenylbutyrate | Terreic Acid | Alpha lipoic acid |
| Vo-ohpic | PGE2 | Ancrod |
| SF1670 | dmPGE2 | Zonisamide |
| Rapamycin | GW9662 | 2-phospho-1-ascorbic acid |
| AICAR | BML-284 | Vitamin C |
| Foxy-5 | Chloroquine | Oct4 |
| EPZ004777 | YH249 | Sox2 |
| SGC0944 | Carbamazepine | Klf4 |
| Anandamide | Lamotrigine | cMyc |
| Simvastatin | Portion of Jag-1 residue 188-04 | BF844 |
| Pravastatin | N-methylhemeanthidine chloride | EGF |
| Amino-bisphosphonates | Tubastatin A | Brain-derived neurotrophic factor (BDNF) |
| A-83-01 | Trichostatin A | rosiglitazone |
| 616452 | Panobinostat | BIX 01294 |
| SB431542 | MS-275 | 5-azacytidine |
| XMU-MP-1 | Apicidin | Reversine |
| WAY-262611 | Febuxostat | Purmorphamine |
| Purpurogalin | Phenyl butyrate | SAG |
| Exifone | MiR-182-5p | Hh-Ag1.5 |
| Gallic acid | AUT1 | LDE225 |
| RG108 | AUT3 | SMER28 |
| WAY 316606 | AUT00063 | PGD2 |
| HLY78 | AM-101 | Metformin |
| Lycorine | OAC2 | S1P |
| D 4476 | AM-111 | 1-AA |
| PF-4800567 | LY3056480 | UM17116 |
| (R)-roscovitine | DzNep | 16-dimethyl |
| PF-670464 | PD0325901 | Forskolin |
| PF-5006739 | PS48 | QS11 |
| TA 01 | Fructose 2 | BIO |
| (R)-DRF053 | 6-bisphosphonate | Cyclopamine |
| TG003 | IM-12 | Neuropathiazol |
| IC261 | Casin | Pluripotin |
| (S)-CR8 | LY294002 | Y-27632 |
| Riluzole | Dorsomorphin | SKL2001 |
| YM298198 | LDN1193189 | AS1842856 |
| JNJ16259685 | Quercetin | Neurotrophin 3 |
| JNJ10198409 | Peptide P13 | AS 8351 |
| LY 456236 | EPI-743 | SJ403 |
| Flunarizine | Vincerinone | TC-E 5002 |
| DAPT | EPI-589 | Tranylcypromine |
| LY411575 | Sodium Thiosulfate | SU16F |
| MDL 28170 | RO4929097 | GSK2879552 |
| SC1 | Concanavalin A | GSK-LSD1 |
| SU5402 | Ciprofloxacin | CBB1007 |
| Selumetinib (AZD6244) | Dexamethasone | 3TFA |
| Methotrexate | Betamethasone | SP-2509 |
| Pyrimethamine | ORC-13661 | Noggin |
| Trimetrexate | Betahistine | R-spondin 1 |
| Nolatrexed | HPN-07 | Valproic acid |
| Raltitrexed | NHPN-1010 | |
| 6-Mercaptopurine | Gacyclidine | |
| Retinoic acid | BDNF | |
| TTNPB | N-acetylcysteine | |
| Azathioprine | PF-04958242 | |

TABLE 2

| | |
|---|---|
| DZNep | AS8351 |
| SC-1 | TC-E 5002 |
| CBB1003 | DNP |
| SP-2509 | PS48 |
| AS8351 | SC-1 |
| TC-E 5002 | LY-364947 |
| NSC 636839 | XMU-MP-1 |
| AZD5438 | IBS008738 |
| Prostaglandin E2 | Ethacridine |
| Forskolin | Metformin |
| DNP | SGC0946 |
| RSC133 | EPZ004777 |
| LY-364947 | OAC2 |
| AMI-5 | Robotinikin |
| AMI-1 | XMU-MP-1 |
| Compound B4 (TGFb-RI) | Metformin |
| PS48 | SGC0946 |
| 5-aza-2′-deoxycytidine | EPZ004777 |
| Rosiglitazone | OAC2 |
| A-83-01 | Robotinikin |
| TTNPB | Cyclopamine |

{2-Methyl-4-{{4-methyl-2-[4-(trifluoromethyl)phenyl]-
5-thiazolyl}methylthio}phenoxy}-acetic acid

| | |
|---|---|
| Gefitinib | 3,3,5 Triiodo-L-thyronine |
| Cyclopamine | IBS008738 |
| Calcitriol | Ethacridine |
| Prostaglandin E2 | CAY10591 |
| Compound B4 (TGFb-RI) | SRT1720 |
| AMI-1 | CAY10602 |
| AMI-5 | EX-527 |
| Forskolin | AGK2 |
| AZD5438 | Tenovin-6 |
| 5-aza-2′-deoxycytidine | Sirtinol |
| RSC133 | (+)-JQ1 |
| DZNep | GSK-J4 |

TABLE 3

| Compound | Target |
|---|---|
| CHIR-98023 | GSK-3β |
| CHIR-99021 | GSK-3β |
| CHIR-99030 | GSK-3β |
| Hymenialdisine | GSK-3β |
| debromohymeialdisine | GSK-3β |
| dibromocantherelline | GSK-3β |
| Meridianine A | GSK-3β |
| alsterpaullone | GSK-3β |
| cazapaullone | GSK-3β |
| Aloisine A | GSK-3β |
| NSC 693868 | GSK-3β |
| (1H-Pyrazolo[3,4-b]quinoxalin-3-amine) | |
| Indirubin-3′-oxime | GSK-3β |
| (Indirubin-3′-monoxime; 3-[1,3-Dihydro- | |
| 3-(hydroxyimino)-2H-indol-2-ylidene]- | |
| 1,3-dihydro-2H-indol-2-one) | |
| A 1070722 | GSK-3β |
| (1-(7-Methoxyquinolin-4-yl)-3-[6- | |
| (trifluoromethyl)pyridin-2-yl]urea) | |
| L803 | GSK-3β |
| L803-mts | GSK-3β |
| TDZD8 | GSK-3β |
| NP00111 | GSK-3β |
| HMK-32 | GSK-3β |
| Manzamine A | GSK-3β |
| Palinurin | GSK-3β |
| Tricantin | GSK-3β |
| IM-12 | GSK-3β |
| (3-(4-Fluorophenylethylamino)-1-methyl- | |
| 4-(2-methyl-1H-indol-3-yl)-1H-pyrrole- | |
| 2,5-dione) | |
| NP031112 | GSK-3β |
| NP00111 | GSK-3β |
| NP031115 | GSK-3β |
| VP 2.51 | GSK-3β |
| VP2.54 | GSK-3β |
| VP 3.16 | GSK-3β |
| VP 3.35 | GSK-3β |
| HLY78 | Axin |
| (4-Ethyl-5,6-Dihydro-5-methyl- | |
| [1,3]dioxolo[4,5-j]phenanthridine, 4- | |
| Ethyl-5-methyl-5,6-dihydro- | |
| [1,3]dioxolo[4,5-j]phenanthridine) | |
| WAY-262611 | Dickkopf-1 (DKK1) |
| ((1-(4-(Naphthalen-2-yl)pyrimidin-2- | |
| yl)piperidin-4-yl)methanamine)) | |
| BHQ880 | DKK1 |
| NCI8642 | DKK1 |
| gallocyanine dyes | DKK1 |
| Compounds 3-8 | secreted frizzled-related |
| (Moore et al., J. Med. Chem., 2009; | protein 1 (sFRP-1) |
| 52: 105) | |
| WAY-316606 | sFRP-1 |

TABLE 4

| Compound | Target |
|---|---|
| A01 | SMAD1/5/8 |
| (Cao et al., Scientific Reports, 2014; | |
| 4: 4965) | |
| A17 | SMAD1/5/8 |
| (Cao et al., Scientific Reports, 2014; | |
| 4: 4965) | |

TABLE 5

| Compound | Target |
|---|---|
| Cerivastatin | p27Kip1 |
| (Baycol; Lipobay) | |
| Alsterpaullone 2-cyanoethyl | p27Kip1 |
| SJ403 | p27Kip1 |

TABLE 6

| Compound | Target |
|---|---|
| Compound A (See FIG. 7) | Atoh1 |
| Compound B (See FIG. 7) | Atoh1 |
| Compound C (See FIG. 7) | Atoh1 |
| 1-Azakenpaullone | Atoh1 |
| (Pyrido[3′,2′: 2,3]azepino[4,5-b]indol- | |
| 6(5H)-one,9-bromo-7,12-dihydro-) | |
| 2-(N)-benzyl ellipticene | Atoh1 |

TABLE 7

| Compound | Target |
|---|---|
| Delta/Serrate/Lag-2 peptide | Notch receptor |

TABLE 8

| Compound | Target |
|---|---|
| Vorinostat | HDAC class I (HDAC1, 2, |
| (rINN; suberanilohydroxamic acid; suberoylanilide | 3, and 8) and HDAC class II |
| hydroxamic acid; SAHA | (IIa: HDAC4, 5, 7, and 9; |
| (suberoyl + anilide + hydroxamic acid abbreviated); N- | IIb: 6 and 10) |
| Hydroxy-N′-phenyloctanediamide; Zolinza ®) | |
| Trichostatin A | HDAC class I (HDAC1, 2, |
| (TSA; (2E,4E,6R)-7-(4-(Dimethylamino)phenyl)-N- | 3, and 8) and HDAC class II |
| hydroxy-4,6-dimethyl-7-oxo-2,4-heptadienamide) | (IIa: HDAC4, 5, 7, and 9; |
| | IIb: 6 and 10) |
| belinostat | HDAC |
| (PXD101; Beleodaq) | |
| Valproic acid | HDAC |
| (VPA; sodium valproate; Sodium 2-propylpentanoate) | |
| FK 228 | HDAC class I (HDAC1, 2, |
| (Depsipeptide; FR 901228; Romidepsin; Cyclo[(2Z)- | 3, and 8), HDAC4, and |
| 2-amino-2-butenoyl-L-valyl-(3S,4E)-3-hydroxy-7- | HDAC6 |
| mercapto-4-heptenoyl-D-valyl-D-cysteinyl], cyclic (3- | |
| 5) disulfide) | |
| Sodium butyrate | HDAC |
| (Butanoic acid sodium salt; NaB) | |
| LMK 235 | HDAC4 and HDAC5 |
| (N-[[6-(Hydroxyamino)-6-oxohexyl]oxy]-3,5- | |
| dimethylbenzamide) | |
| Scriptaid | HDAC |
| (N-Hydroxy-1,3-dioxo-1H-benz[de]isoquinoline- | |
| 2(3H)-hexanamide) | |
| M 344 | HDAC |
| (4-(Diethylamino)-N-[7-(hydroxyamino)-7- | |
| oxoheptyl]benzamide) | |
| SBHA | HDAC1 and HDAC3 |
| (N,N′-Dihydroxyoctanediamide; suberic | |
| bishydroxamate) | |
| CBHA | HDAC1 and HDAC3 |
| (m-carboxycinnamic acid bishydroxamide) | |
| HMBA | HDAC |
| (hexamethylene bisacetamide). | |
| Tubacin | HDAC6 |
| (N-[4-[(2R,4R,6S)-4-[[(4,5-Diphenyl-2- | |
| oxazolyl)thio]methyl]-6-[4-(hydroxymethyl)phenyl]- | |
| 1,3-dioxan-2-yl]phenyl]-N′-hydroxyoctanediamide) | |
| Sodium 4-phenylbutyrate | HDAC |
| (4-PB; sodium pheylbutyrate; 4-Phenylbutyric acid, | |
| sodium salt; 4-phenylbutyrate) | |
| MC 1568 | HDAC class IIa (HDAC4, 5, |
| (3-[5-(3-(3-Fluorophenyl)-3-oxopropen-1-yl)-1- | 7, and 9) |
| methyl-1H-pyrrol-2-yl]-N-hydroxy-2-propenamide) | |
| Compound 9 | HDAC class IIa (HDAC4, 5, |
| (Mai et al., J. Med. Chem., 2005; 48: 3344) | 7, and 9) |
| Compound 24 | HDAC class IIa (HDAC4, 5, |
| (Mai et al., J. Med. Chem., 2005; 48: 3344) | 7, and 9) |
| TC-H 106 | HDAC class I (HDAC1, 2, |
| (N1-(2-Aminophenyl)-N7-(4- | 3, and 8) |
| methylphenyl)heptanediamide; Pimelic | |
| Diphenylamide 106) | |
| Pyroxamide | HDAC1 |
| (N-Hydroxy-N′-3-pyridinyloctanediamide) | |
| NCH 51 | HDAC |
| (PTACH; 2-Methylpropanethioic acid S-[7-oxo-7-[(4- | |
| phenyl-2-thiazolyl)amino]heptyl] ester) | |
| NCH 31 | HDAC |
| PCI 34051 | HDAC8 |
| (N-Hydroxy-1-[(4-methoxyphenyl)methyl]-1H-indole- | |
| 6-carboxamide) | |
| thiophene benzamide | HDAC1 and HDAC2 |
| KD 5170 | HDAC class I (HDAC1, 2, |
| (S-[2-[6-[[[4-[3-(Dimethylamino)propoxy] | 3, and 8) and HDAC class II |
| phenyl]sulfonyl]amino]-3-pyridinyl]-2- | (IIa: HDAC4, 5, 7, and 9; |
| oxoethyl]ethanethioc acid ester) | IIb: 6 and 10) |
| TCS HDAC6 20b | HDAC6 |
| (2-Methylpropanethioic acid-S-[(6S)-6-[[(1,1- | |
| dimethylethoxy)carbonyl]amino]-7-oxo-7- | |
| (tricyclo[3.3.1.1^{3,7}]dec-1-ylamino)heptyl] ester) | |
| NSC 3852 | HDAC |
| (5-Nitroso-8-quinolinol) | |
| NSC69603 | HDAC |
| NSC86371 | HDAC |
| NSC305819 | HDAC |
| CI 994 | HDAC class I |
| (N-acetyldinaline; Acetyldinaline; 4- | |
| (Acetylamino)-N-(2-aminophenyl)benzamide) | |
| LAQ824 | HDAC class I |
| LBH589 | pan-HDAC |
| (panobinostat; Farydak) | |
| MS275 | HDAC1-3 |
| (SNDX-275; entinostat) | |
| MGCD0103 | HDAC1-8 and 11 |
| (mocetinostat) | |
| UF 010 | HDAC1-3 |
| (4-Bromo-N′-butylbenzohydrazide) | |
| Cpd60 | HDAC1-3 |
| Romidepsin | HDAC1 and HDAC2 |
| MS-27-275 | HDAC |
| NaBu | HDAC |
| (n-butyrate) | |
| trapoxin | HDAC |
| Apicidin | HDAC |
| (Cyclo[(2S)-2-Amino-8-oxodecanoyl-1-methoxy-L- | |
| tryptophyl-L-isoleucyl-(2R)-2-piperidinecarbonyl]) | |
| depudesin | HDAC |
| EX 527 | SIRT1 |
| (6-Chloro-2,3,4,9-tetrahydro-1H-carbazole-1- | |
| carboxamide) | |
| AGK 2 | SIRT2 |
| (2-Cyano-3-[[5-(2,5-dichlorophenyl)-2-furanyl]-N-5- | |
| quinolinyl-2-propenamide) | |
| AK 7 | SIRT2 |
| (N-(3-Bromophenyl)-3-[(hexahydro-1H-azepin-1- | |
| yl)sulfonyl]benzamide) | |
| SirReal2 | SIRT2 |
| (2-[(4,6-Dimethyl-2-pyrimidinyl)thio]-N-[5-(1- | |
| naphthalenylmethyl)-2-thiazolyl]acetamide) | |
| Salermide | SIRT1 and SIRT2 |
| (N-[3-[[(2-Hydroxy-1-naphthalenyl) | |
| methylene]amino]phenyl]-α-methylbenzeneacetamide) | |
| Splitomicin | Sir2p (yeast form of SIRT1) |
| (1,2-Dihydro-3H-naphtho[2,1-b]pyran-3-one) | |

TABLE 9

| Compound | Target |
|---|---|
| MG132 | proteasome |
| (Z-LLL-al, Z-Leu-Leu-Leu-CHO; N- | |
| [(Phenylmethoxy)carbonyl]-L-leucyl-N-[(1S)-1- | |
| formyl-3-methylbutyl]-L-leucinamide) | |
| MG262 | proteasome |
| (Z-Leu-Leu-Leu-B(OH)2) | |
| MG115 | proteasome |
| (Z-Leu-Leu-Nva-CHO) | |
| Z-Leu-Leu-Phe-CHO | proteasome |
| (Z-LLF-CHO) | |
| N-Acetyl-leucyl-leucyl-norleucinal | proteasome |
| (Ac-Leu-Leu-Nle-CHO) | |
| N-acetyl-leucyl-leucyl-methional | proteasome |
| (Ac-Leu-Leu-Met-CHO) | |
| N-benzyloxycarbonyl-isoleucyl-γ-t-butyl-glutamyl- | proteasome |
| alanyl-leucinal | |
| (Z-Ile-Glu(OtBu)-Ala-Leu-CHO) | |
| N-benzyloxycarbonyl-leucyl-leucyl-leucinal | proteasome |
| (Z-Leu-Leu-Leu-CHO), | |
| N-benzyloxycarbonyl-leucyl-leucyl-tyrosyl α-keto | proteasome |
| aldehyde | |
| (Z-Leu-Leu-Tyr-COCHO) | |
| N-benzyloxycarbonyl-leucyl-leucyl-phenylalanal | proteasome |
| (Z-Leu-Leu-Phe-CHO) | |
| N-benzyloxycarbonyl-leucyl-leucyl-leucyl boronic | proteasome |
| acid | |
| (Z-Leu-Leu-Leu-B(OH)₂) | |
| Bortezomib | proteasome |
| (PS-341; Velcade; Neomib; Bortecad) | |
| Lactacystin | proteasome |
| ((2R,3S,4R)-3-Hydroxy-2-[(1S)-1-hydroxy-2- | |
| methylpropyl]-4-methyl-5-oxo-2-pyrrolidinecarboxy- | |
| N-acetyl-L-cysteine thioester) | |
| Disulfiram | proteasome |
| (Antabuse and Antabus) | |
| Epigallocatechin-3-gallate | proteasome |
| (Epigallocatechin gallate; EGCG) | |
| Salinosporamide A | proteasome |
| Carfilzomib | proteasome |
| (Kyprolis)_{—} | |
| epoxomicin | proteasome |
| Ixazomib | proteasome |
| (Ninlaro; MLN2238) | |
| ixazomib citrate | proteasome |
| (MLN9708) | |
| PS-341 | proteasome |
| VLX1500 | proteasome |
| (b-AP15) | |
| clasto-Lactacystin beta Lactone | proteasome |
| Gliotoxin | proteasome |
| (Aspergillin; (3R,5aS,6S,10aR)-2,3,5a,6-Tetrahydro-6- | |
| hydroxy-3-(hydroxymethyl)-2-methyl-10H-3,10a- | |
| epidithiopyrazino[1,2-a]indole-1,4-dione) | |
| AM 114 | proteasome |
| (3,5-Bis-[benzylidene-4-boronic acid]-1- | |
| methylpiperidin-4-one) | |
| PSI | proteasome |
| (N-[(Phenylmethoxy)carbonyl]-L-isoleucyl-L-α- | |
| glutamyl-tert-butyl ester-N-[(1S)-1-formyl-3- | |
| methylbutyl]-L-alaninamide) | |
| Oprozomib | proteasome |
| (ONX 0912) | |
| Delanzomib | proteasome |
| (CEP-18770) | |
| BI8622 | Huwe1 (E3 ubiquitin ligase) |
| BI8626 | Huwe1 (E3 ubiquitin ligase) |

TABLE 10

| Compound | Target |
|---|---|
| MLN4929 | Akt |
| (Pevonedistat) | |
| API-2 | Akt |
| (Triciribine; NSC 154020; TCN; 1,5-Dihydro-5- | |
| methyl-1-β-D-ribofuranosyl-1,4,5,6,8- | |
| pentaazaacenaphthylen-3-amine; Akt/protein kinase B | |
| signaling inhibitor-2) | |
| API-1 | Akt |
| (4-Amino-5,8-dihydro-5-oxo-8-β-D-ribofuranosyl- | |
| pyrido[2,3-d]pyrimidine-6-carboxamide) | |
| GSK 690693 | Akt |
| (4-[2-(4-Amino-1,2,5-oxadiazol-3-yl)-1-ethyl-7-[(3S)- | |
| 3-piperidinylmethoxy)-1H-imidazo[4,5-c]pyridin-4- | |
| yl]-2-methyl-3-butyn-2-ol) | |
| 10-DEBC hydrochloride | Akt |
| (10-[4′-(N,N-Diethylamino)butyl]-2- | |
| chlorophenoxazine hydrochloride) | |
| FPA124 | Akt |
| (Dichloro[(2Z)-2-[(4-oxo-4H-1-benzopyran-3- | |
| yl)methylene] | |
| hydrazinecarbothioamide copper complex) | |
| SC66 | Akt |
| ((2E,6E)-2,6-Bis(4- | |
| pyridinylmethylene)cyclohexanone) | |
| LY 294002 hydrochloride | PI3K |
| (2-(4-Morpholinyl)-8-phenyl-4H-1-benzopyran-4-one | |
| hydrochloride) | |
| wortmannin | PI3K |
| PI 103 | PI3K |
| Quercetin | PI3K and PKC |
| (2-(3,4-Dihydroxyphenyl)-3,5,7-trihydroxy-4H-1- | |
| benzopyran-4-one) | |
| PHT 427 | Akt and PDK1 |
| (4-Dodecyl-N-1,3,4-thiadiazol-2-yl- | |
| benzenesulfonamide) | |
| GSK 2334470 | PDK1 |
| ((3S,6R)-1-[6-(3-Amino-1H-indazol-6-yl)-2- | |
| (methylamino)-4-pyrimidinyl]-N-cyclohexyl-6- | |
| methyl-3-piperidinecarboxamide) | |
| Fisetin | PI3K, Akt |
| (2-(3,4-Dihydroxyphenyl)-3,7-dihydroxy-4H-1- | |
| benzopyran-4-one) | |
| OSU 03012 | Akt and PDK1 |
| (2-Amino-N-[4-[5-(2-phenanthrenyl)-3- | |
| (trifluoromethyl)-1H-pyrazol-1-yl]phenyl]acetamide) | |
| PIT 1 | Akt |
| (N-[[(3-Chloro-2-hydroxy-5-nitrophenyl) | |
| amino]thioxomethyl]benzamide) | |

TABLE 11

| Compound | Target |
|---|---|
| AC102 | CREB |
| (6-fluoro-9-methyl-β-carboline; 6F9MβC | |

TABLE 12

| Compound | Target |
|---|---|
| LY411575 | γ-secretase |
| (LSN-411575; Compound 5; benzeneacetamide; N- | |
| [(1s)-2-[[(7s)-6,7-dihydro-5-methyl-6-oxo-5H- | |
| dibenz[b,d]azepin-7-yl]amino]-1-methyl-2-oxoethyl]- | |
| 3,5-difluoro-α-hyroxy-(αS)-); N2-[(2S)-2-(3,5- | |
| Difluorophenyl)-2-hydroxyethanoyl]-N1-[(7S)-5- | |
| methyl-6-oxo-6,7-dihydro-5H-dibenzo[b,d]azepin-7- | |
| yl]-L-alaninamide) | |
| L-685458 | γ-secretase |
| ((5S)-(tert-Butoxycarbonylamino)-6-phenyl-(4R)- | |
| hydroxy-(2R)-benzylhexanoyl)-L-leucy-L- | |
| phenylalaninamide; LY-685458; GSI-X) | |
| DBZ | γ-secretase |
| (Dibenzazepine; YO-01027; GSI-XX, deshydroxy LY- | |
| 411575; N-[(1S)-2-[[(7S)-6,7-Dihydro-5-methyl-6-oxo- | |
| 5H-dibenz[b,d]azepin-7-yl]amino]-1-methyl-2- | |
| oxoethyl]-3,5-difluorobenzeneacetamide) | |
| MRK560 | γ-secretase |
| (N-[cis-4-[(4-Chlorophenyl)sulfonyl]-4-(2,5- | |
| difluorophenyl)cyclohexyl]-1,1,1- | |
| trifluoromethanesulfonamide) | |
| MRK-003 | γ-secretase |
| MK-0752 | γ-secretase |
| Compound W | γ-secretase |
| (CW; 3,5-Bis(4-nitrophenoxy)benzoic acid) | |
| (Okochi et al., J. Biol. Chem., 2006; 281: 7890; Ford et | |
| al., J Neurosci Meth., 2008; 168: 465-474) | |
| Compound E | γ-secretase |
| (GSI-XXI) | |
| (Olsauskas-Kuprys et al., Onco Targets Ther., 2013; | |
| 6: 943) | |
| BMS 2289948 | γ-secretase |
| (4-chloro-N-(2,5-difluorophenyl)-N-((1R)-{4-fluoro-2- | |
| [3-(1H-imidazol-1- | |
| yl)propyl]phenyl}ethyl)benzenesulfonamide | |
| hydrochloride) | |
| BMS-433796 | γ-secretase |
| ((S)-2-((S)-2-(3,5-difluorophenyl)-2- | |
| hydroxyacetamido)-N-((S,Z)-3-methyl-4-oxo-4,5- | |
| dihydro-3H-benzo[d][1,2]diazepin-5-yl)propanamide) | |
| IN973 | γ-secretase |
| Flurbiprofen | γ-secretase |
| bi((R)-Flurbiprofen; tarenflurbil; Flurizan; (R)-2- | |
| Fluoro-α-methyl[1,1′-biphenyl]-4-acetic acid) | |
| JLK2, JLK4, JLK6, JLK7 | γ-secretase |
| (7-Amino-4-chloro-3-methoxy-1H-2-benzopyran) | |
| Begacestat | γ-secretase |
| (GSI-953; 5-Chloro-N-[(1S)-3,3,3-trifluoro-1- | |
| (hydroxymethyl)-2-(trifluoromethyl)propyl]-2- | |
| thiophenesulfonamide) | |
| DFK167 | γ-secretase |
| PF-0308414 | γ-secretase |

TABLE 13

| Compound | Target |
|---|---|
| TTNBP | RAR |
| (RO 13-7410, arotinoid acid, AGN 191183) | |
| ATRA | RAR |
| 9-cis RA | RAR |
| CD271 | RAR |
| (6-(4-Methoxy-3-tricyclo[3. 3. 1. 1 3,7]dec-1-ylphenyl)-2- | |
| naphthalenecarboxylic acid) | |
| CD336 | RAR |
| CD-394 | RAR |
| CD437 | RAR |
| (6-3-(1-adamantyl)-4-hydroXyphenyl)-2 naphthanoic acid) | |
| (6-(4-Hydroxy-3-tricyclo[3. 3. 1. 13,7]dec-1-ylphenyl)-2- | |
| naphthalenecarboxylic acid) | |
| CD666 | RAR |
| ((E)-4-(1-hydroXy-1-(5,6,7,8-tetrahydro-5,5,8,8 tetramethyl- | |
| 2-naphthyl)-2-propenyl)benzoic acid) | |
| CD1530 (4-(6-Hydroxy-7-tricyclo[3. 3. 1. 13,7]dec-1-yl-2- | RAR |
| naphthalenyl)benzoic acid) | |
| CD2019 | RAR |
| (6-(3-(1-methylcycloheXyl)-4-methoXyphenyl)-2 naphthanoic | |
| acid) | |
| CD2247 | RAR |
| CD2081 | RAR |
| CD2314 | RAR |
| CD2325 | RAR |
| (4-[(E)-2-(3-(1-adamantyl)-4-hydroXyphenyl)-1 | |
| propenyl]benZoic acid) | |
| CD2425 | RAR |
| CD2503 | RAR |
| CD2665 | RAR |
| BMS-270394 (enantiomer of BMS-189961) | RAR |
| (3-Fluoro-4-[(R)-2-hydroxy-2-(5,5,8,8-tetramethyl-5,6,7,8- | |
| tetrahydro-naphthalen-2-yl)-acetylaminol-benzoic acid) | |
| BMS-189961 | RAR |
| (3-Fluoro-4-[2-hydroxy-2-(5,5,8,8-tetramethyl-5,6,7,8- | |
| tetrahydro-naphthalen-2-yl)-acetylamino]-benzoic acid) | |
| 6-[3-(adamantan-1-yl)-4-(prop-2- | RAR |
| ynyloxy)phenyl]naphthalene-2-carboxylic acid | |
| 5-[(E)-3-oxo-3-(5,5,8,8-tetrahydronaphthalene-2- | RAR |
| yl)propenyl]thiophene-2-carboxylic acid | |
| Palovarotene | RAR |
| (4[(1E)-2-[5,6,7,8-Tetrahydro-5,5,8,8-tetramethyl-3-(1H- | |
| pyrazol-1-ylmethyl)-2-naphthalenyl]-ethenyl]-benzoic acid; | |
| R667; CLM-001, RG667) | |
| CH-55 | RAR |
| (4-[(E)-3-(3,5-Di-tert-butyl-phenyl)-3-oxo-propenyl]-benzoic | |
| acid) | |
| Docosahexaenoic acid | RXR |
| (DHA; (4Z,7Z,10Z,13Z,16Z,19Z)-4,7,10,13,16,19- | |
| Docosahexaenoic acid) | |
| CD 3254 | RXR |
| (3-[4-Hydroxy-3-(5,6,7,8-tetrahydro-3,5,5,8,8-pentamethyl-2- | |
| naphthalenyl)phenyl]-2-propenoic acid) | |
| 9 cis-RA | RXR |
| 3-cis-retinoic acid | RXR |
| (Accutane; isotretinoin; 13-cis-Retinoic acid) | |
| LG 100754 | RXR |
| ((2E,4E,6Z)-3-Methyl-7-(5,6,7,8-tetrahydro-5,5,8,8- | |
| tetramethyl-3-propoxy-3-naphthalenyl)-2,4,6-octatrienoic | |
| acid) | |
| SR 11237 | RXR |
| (BMS 649; 4-[2-(5,6,7,8-Tetrahydro-5,5,8,8-tetramethyl-2- | |
| naphthalenyl)-1,3-dioxolan-2-yl]-benzoic acid) | |
| Fluorobexarotene | RXR |
| (2-Fluoro-4-[1-(5-,6,7,8-tetrahydro-3,5,5,8,8-pentamethyl-2- | |
| naphthalenyl)ethenyl]benzoic acid) | |
| LGD1069 | RXR |
| (4-[1-(3,5,5,8,8-pentamethyl-5,6,7,8-tetrahydro-2- | |
| naphthyl)ethenyl]benzoic acid) | |
| LG100268 | RXR |
| (6-[1-(3,5,5,8,8-pentamethyl-5,6,7,8-tetrahydronaphthalen-2- | |
| yl)cyclopropyl]nicotinic acid) | |
| LG100754 | RXR |
| (2E,4E,6Z)-3-Methyl-7-(5,6,7,8-tetrahydro-5,5,8,8- | |
| tetramethyl-3-propoxy-2-naphthalenyl)-2,4,6-Octatrienoic | |
| acid) | |
| Compounds 1-11 | RXR |
| (Wagner et al., J. Med. Chem., 2009; 52: 5950) | |
| HX 630 | RXR |
| (4-(7,8,9,10-Tetrahydro-7,7,10,10- | |
| tetramethylbenzo[b]naphtho[2,3-f][1,4]thiazepin-12-yl- | |
| benzoic acid) | |
| HX 640 | RXR |
| HX 600 | RXR |
| TZ335 | RXR |
| Adapalene | RXR |
| (6-(4-Methoxy-3-tricyclo[3.3.1.1^{3,7}]dec-1-ylphenyl)-2- | |
| naphthalenecarboxylic acid, 6-[3-(1-Adamantyl)-4- | |
| methoxyphenyl]-2-naphthoic acid; CD-271; Differin) | |
| Bexarotene | RXR |
| (4-[1-(5,6,7,8-Tetrahydro-3,5,5,8,8-pentamethyl-2- | |
| naphthalenyl)ethenyl]benzoic acid, LGD-1069; SR-11247; | |
| targretin; TRG) | |
| Retinoic acid | RXR |
| (ATRA; Tretinoin; Vitamin A acid; all-trans-Retinoic acid) | |
| 4-[N-methanesulfonyl-N-(5,5,8,8-tetramethyl-5,6,7,8- | RXR |
| tetrahydro-2-naphthyl)amino]benzoic acid | |
| 6-[N-ethyl-N-(3-isopropoxy-4- | RXR |
| isopropylphenyl)amino]nicotinic acid (NEt-3IP) | |
| 6-[N-ethyl-N-(3-isobutoxy-4-isopropylphenyl)amino]nicotinic | RXR |
| acid (NEt-3IB | |
| PA024 | RXR |
| AGN 194204 | RXR |
| CNX-013-B2 | RXR |
| UAB30 | RXR |
| IRX4204 | RXR |

### Hair Cell Regeneration Agents

The one or more otic therapeutic agents in any embodiment disclosed could be one or more of the following hair cell regeneration agents.

A hair cell regeneration agent is an agent that promotes regeneration of hair cells. A single agent may be used as a hair cell regeneration agent or a combination of agents may provide the hair cell regenerative function. Thus, in some embodiments, the hair cell regeneration agent is a single agent. In other embodiments the hair cell regeneration agent is a combination of agents. In certain such embodiments, the combination of agents may be compounded together in a single composition. In other embodiments, the combination of agents may be provided to a patient separately.

A hair cell regeneration agent may promote regeneration of hair cells by stimulating transdifferentiation of supporting cells within the sensory epithelium of cochlea into replacement hair cells. Alternatively, or additionally, a hair cell regeneration agent may activate a proliferative response in the sensory epithelium of the cochlea, thereby providing a new population of cells that can subsequently differentiate into supporting cells.

In some embodiments, the hair cell regeneration agent stimulates proliferation of cochlear supporting cells in which proliferation is stimulated expresses Lgr5 (Leucine-rich repeat-containing G-protein coupled receptor 5). However the hair cell regeneration agent may also stimulate proliferation of supporting cells with little or no Lgr5 expression. In some embodiments, the hair cell regeneration agent produces an expanded population of cochlea cells. In some embodiments, the expanded cells are enriched for Lgr5 expression (i.e. a greater percentage of the expanded cell population express Lgr5 compared to the starting cell population).

Lgr5 is a member of GPCR class A receptor proteins that is expressed across a diverse range of tissues such as in the muscle, placenta, spinal cord and brain, and particularly as a biomarker of adult stem cells in certain tissues. Lgr5+ stem cells are the precursors for sensory hair cells that are present in the cochlea. Increasing the population of Lgr5+ cochlear cells is therefore beneficial because it increases the population of precursor cells which may differentiate into sensory hair cells.

In some embodiments, the hair cell regeneration agent is a Wnt agonist and an epigenetic modulator. Any Wnt agonist and epigenetic modulator described herein may be used.

In some embodiments, the hair cell regeneration agent is a Wnt agonist and two or more epigenetic modulator. Any Wnt agonist and epigenetic modulator described herein may be used.

In some embodiments, the hair cell regeneration agent is a Wnt agonist alone. A Wnt agonist may be used alone in line with any of the treatments disclosed herein that relate to Wnt agonists and/or epigenetic modulators in which both the Wnt agonist and epigenetic modulator are administered to the patient. In these embodiments, the epigenetic modulator is not included. Any Wnt agonist described herein may be used. In certain such embodiments, the hair cell regeneration agents is a GSK3 inhibitor. Any GSK3 inhibitor described herein may be used.

In some embodiments, the hair cell regeneration agent is gamma secretase inhibitor. Suitable gamma secretase inhibitors are described in WO 2018007331 A1; WO 2018111926 A2; WO 2018065340 A1; WO 2018060300 A1; WO 2018011164 A1; WO 2018087018 A1; WO 2018001918 A1; WO 2018118791 A2; WO 2018118782 A2 and WO 2014045156 A1, each of which is incorporated by reference. Any gamma secretase inhibitor described herein may be used.

In some embodiments, the hair cell regeneration agent is an Atoh1 activator. Suitable Atoh1 activators are described in US 2016003011115 A1; WO 2018172997 A1; WO 2016022776 A2; WO 2014145205 A2 and WO 2009100438 A2, each of which is incorporated by reference.

In some embodiments, the hair cell regeneration agent is a Notch inhibitor. Suitable Notch inhibitors are described in WO2017007702-A1; WO2016056999-A1; WO2014039781A1; WO2014047369A1; WO2014047372A1; WO2014047390A1; WO2014047391A1; WO2014047397A1; WO2014047392A1; WO2014047370A1; WO2014047374A1; WO2013093885A1; WO2013178821A1 and WO2013016081A1, each of which is incorporated by reference.

In some embodiments, the hair cell regeneration agent is a Wnt agonist and a Notch inhibitor. Any Wnt agonist and Notch inhibitor may be used as described herein. In certain such embodiments the Wnt agonist is a GSK3 inhibitor. Any GSK3 inhibitor described herein may be used.

In some embodiments, the hair cell regeneration agent is a Wnt agonist and a gamma secretase inhibitor. Any Wnt agonist and gamma secretase inhibitor may be used as described herein. In certain such embodiments, the Wnt agonist is a GSK inhibitor. Any GSK3 inhibitor described herein may be used.

### WNT Agonists

The one or more otic therapeutic agents in any embodiment disclosed could be one or more of the following WNT agonists.

Provided in one aspect is a Wnt agonist and/or an epigenetic modulator for use in treating sensorineural hearing loss in a human patient, wherein said Wnt agonist and said epigenetic modulator are administered to a human patient. Also provided is a method of treating sensorineural hearing loss in a human patient comprising administering to the patient a Wnt agonist and an epigenetic modulator. A Wnt agonist and/or an epigenetic modulator may be used for treating a patient as described elsewhere herein.

A Wnt agonist refers to an agent that increases the expression, levels, and/or activity of a Wnt gene, protein, or signaling pathway (e.g. TCF/LEF, Frizzled receptor family, Wif1, Lef1, Axing, β-catenin) in a cell, for example, a cochlear cell. A Wnt agonist includes a GSK3 inhibitor, such as a GSK3-α or a GSK3-β inhibitor. In some embodiments the Wnt agonist is a GSK inhibitor that inhibits both GSK3-α and GSK3-β.

The TCF/LEF family is a group of transcription factors that bind to DNA through a high mobility group domain, and which are involved in the Wnt signaling pathway where they recruit the coactivator β-catenin to enhancer elements of targeted genes. Frizzled is a family of G protein-coupled receptor proteins that serves as receptors in the Wnt signaling pathway. Frizzled receptors inhibit intracellular β-catenin degradation and activate TCF/LEF-mediated transcription.

In some embodiments, the Wnt agonist increases Wnt signaling in a cochlear cell by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) relative to a control, for example relative to a baseline level of activity.

In some embodiments, the Wnt agonist increases TCF/LEF-mediated transcription in a cochlear cell, for example, by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) relative to a control, for example relative to a baseline level of activity.

In some embodiments, the Wnt agonist binds and activates a Frizzled receptor family member, for example, by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) relative to a control, for example relative to a baseline level of activity.

In some embodiments, the Wnt agonist inhibits GSK3 for example, by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) relative to a control, for example relative to a baseline level of activity.

In some embodiments, the Wnt agonist preferentially upregulates Jag-1, Deltex-1 or Hif-1 more than the Wnt agonist upregulates Hes or Hey. In some embodiments, the Wnt agonist increases the expression of Jag-1, Deltex-1 and/or Hif-1 10%, 25%, 50%, 75%, 100%, 125%, 150%, 175%, 200%, 250% or more than it increases the expression or activity of Hes and Hey.

Exemplary agents having activity as a Wnt agonist are provided in Table 14 and 15 below, including pharmaceutically-acceptable salts thereof.

TABLE 14

| | | GSK-3 | | GSK-3 | | Lgr5+ | | Perilymph | | Formul. Conc. | |
|---|---|---|---|---|---|---|---|---|---|---|---|
| Agent | CAS | alpha | | beta | | Assay | | Conc. | | Intraymp | |
| CHIR99021 | 252917-06-9 | 4.4 | nM | 6.6 | nM | 2-6 | uM | 2-6 | uM | 4 | mM |
| AZD 1080 | 612487-72-6 | 6.9 | nM | 31 | nM | 1-5 | uM | 1-5 | uM | 1-5 | mM |
| GSK XXII | 1195901-31-5 | 2.3 | nM | 2.0 | nM | 0.2-1 | uM | 0.2-1 | uM | 0.2-1 | mM |
| LY2090314 | 603288-22-8 | 2.1 | nM | 0.9 | nM | 5-20 | nM | 5-20 | nM | 5-20 | uM |

TABLE 15

| Class | Agent | CAS |
|---|---|---|
| WNT | | |
| ARFGAP1 | QS 11 | 944328-88-5 |
| ARFGAP1 | WASP-1, ZINC00087877 | 352328-82-6 |
| Axin | Cpd1 | 1357473-75-6 |
| Axin | Cpd2 | 1228659-47-9 |
| Axin | HLY78 | 854847-61-3 |
| Axin | SKL2001 | 909089-13-0 |
| beta-catenin | DCA | 56-47-3 |
| Disrupts the Axin | Compound 2 | 1360540-82-4 |
| Complex | | |
| Disrupts the Axin | Compound 71 | 1622429-71-3 |
| Complex | | |
| Disrupts the Axin | ISX 9 | 832115-62-5 |
| Complex | | |
| DKK1 inhibitor | WAY-262611 | 1123231-07-1 |
| MEK | Radicicol | 12772-57-5 |
| MEK | Selumetinib (AZD6244) | 606143-52-6 |
| PP2A | IQ 1 | 331001-62-8 |
| sFRP-1 inhibitor | (Dimethylamino)propyl)-2-ethyl-5- | 915754-88-0 |
| | (phenylsulfonyl)benzenesulfonamide | |
| sFRP-1 inhibitor | Cyclosporine A (CsA) | 59865-13-3 |
| sFRP-1 inhibitor | Cyclosporine analogs | |
| sFRP-1 inhibitor | PSC833 (Valspodar) | 121584-18-7 |
| sFRP-1 inhibitor | WAY 316606 | 915759-45-4 |
| Target Undetermined | Diketones | WO 2016029021 A1; |
| | | WO 2012024404 A1 |
| Target Undetermined | Diketones | 1622429-56-4 |
| Target Undetermined | Diketones | 1360540-88-0 |
| Target Undetermined | Diketones | 1360540-89-1 |
| Target Undetermined | Diketones | 1622429-79-1 |
| Target Undetermined | Diketones | 1622429-75-7 |
| Target Undetermined | Diketones | 1622429-74-6 |
| Target Undetermined | Diketones | 1622430-76-5 |
| Target Undetermined | Diketones | 1622430-31-2 |
| Target Undetermined | Diketones | 1622430-52-7 |
| Target Undetermined | Diketones | 1622429-67-7 |
| Target Undetermined | Diketones | 1622429-65-5 |
| Target Undetermined | Diketones | 1622429-69-9 |
| van-Gogh-like receptor | Compound 109 | 1314885-81-8 |
| proteins (Vangl) | | |
| Wnt Ligand | Wnt-1 | Protein |
| Wnt Ligand | Wnt-10a | Protein |
| Wnt Ligand | Wnt-10b/12 | Protein |
| Wnt Ligand | Wnt-11 | Protein |
| Wnt Ligand | Wnt-16 | Protein |
| Wnt Ligand | Wnt-2/Irp (Int-I-related protein) | Protein |
| Wnt Ligand | Wnt-2b/13 | Protein |
| Wnt Ligand | Wnt-3/Int-4 | Protein |
| Wnt Ligand | Wnt-3a | Protein |
| Wnt Ligand | Wnt-4 | Protein |
| Wnt Ligand | Wnt-5a | Protein |
| Wnt Ligand | Wnt-5b | Protein |
| Wnt Ligand | Wnt-6 | Protein |
| Wnt Ligand | Wnt-7a | Protein |
| Wnt Ligand | Wnt-7b | Protein |
| Wnt Ligand | Wnt-8a/8d | Protein |
| Wnt Ligand | Wnt-8b | Protein |
| Wnt Ligand | Wnt-9a/14 | Protein |
| Wnt Ligand | Wnt-9b/14b/15 | Protein |
| Wnt Related Protein | Norrin | Protein |
| Wnt Related Protein | R-Spondin 1/2/3/4 | Protein |
| Wnt-3a/Dkk-1 | BML-284 | 853220-52-7 |
| Wnt-3a/Dkk-1 | Compound 1 | 1084833-94-2 |
| Wnt-3a/Dkk-1 | Compound 25 | 1084834-05-8 |
| GSK3 alpha | | |
| CREB knockdown | 666-15 | 1433286-70-4 |
| Isonicotinamides | Compound 29 | 1772823-37-6 |
| Isonicotinamides | Compound 33 | 1772823-64-9 |
| Isonicotinamides | Compound 39 | 1772824-10-8 |
| Maleimide | I5 | 264217-24-5 |
| Maleimide | Tivantinib | 905854-02-6 |
| Organometallic | Compound (R)-DW12 | 1047684-07-0 |
| Organometallic | Compound 3 | 1498285-39-4 |
| | | 1498285-48-5 |
| Organometallic | Compound lambda-OS1 | 1291104-51-2 |
| | | 1292843-11-8 |
| Oxadiazoles | Compound 14d | 1374671-64-3 |
| Oxadiazoles | Compound 15b | 1374671-66-5 |
| Oxadiazoles | Compound 27 | 1820758-44-8 |
| Oxindole | AZD1080 | 612487-72-6 |
| Pyrazole | AT 7519 | 844442-38-2 |
| Pyrazole | Compound 4a | 1627557-91-8 |
| Pyrazole | Compound 4t | 1627558-10-4 |
| Pyrazole | Compound 4z | 1627558-16-0 |
| Pyrazole | GSK-3b XXII | 1195901-31-5 |
| Pyrazolopyridazines | Compound 18 | 405223-20-3 |
| Pyrazolopyridazines | Compound 19 | 405223-71-4 |
| Pyrazolopyridines | Compound 14 | 583038-63-5 |
| Pyrazolopyridines | Compound 23 | 583038-76-0 |
| Pyrazolopyridines | Pyrazolopyridine 34 | 583039-27-4 |
| Pyrazolo- | BRD1172 | 1597438-86-2 |
| tetrahydroquinolinone | | |
| Pyrazolo- | BRD1652 | 1597438-93-1 |
| tetrahydroquinolinone | | |
| Pyrazolo- | BRD4003 chiral | 1597439-60-5 |
| tetrahydroquinolinone | | |
| Pyrazolo- | BRD4003 chiral | 1597439-59-2 |
| tetrahydroquinolinone | | |
| Pyrazolo- | Compound 11 | 1597439-12-7 |
| tetrahydroquinolinone | | |
| Pyrazolo- | Compound 16 | 1597440-17-9 |
| tetrahydroquinolinone | | |
| Pyrazolo- | Compound 8 | 1597439-01-4 |
| tetrahydroquinolinone | | |
| Pyrazolo- | Compound 9 | 1597439-02-5 |
| tetrahydroquinolinone | | |
| Triazolpyrimidine | Compound 90 | 91322-11-1 |
| Triazolpyrimidine | Compound 92 | 1043429-30-6 |
| Urea | AR-A014418 | 487021-52-3 |
| GSK3-beta | | |
| Acid | Bikinin | 188011-69-0 |
| Acid | Valproic Acid, Sodium Salt | 99-66-1 |
| Aloisines | Aloisine A | 496864-16-5 |
| Aloisines | Aloisine B | 496864-14-3 |
| Aloisines | TWS119 | 1507095-58-0 |
| Aminopyrimidine | CHIR98014 (CT98014) | 252935-94-7 |
| Aminopyrimidine | CHIR98023 (CT98023) | 252904-84-0 |
| Aminopyrimidine | CHIR98024 (CT98024) | 556813-39-9 |
| Aminopyrimidine | CHIR99021 (CT99021) | 252917-06-9 |
| Aminopyrimidine | CT20026 | 403808-63-9 |
| Aminopyrimidinyl | CGP60474 | 164658-13-3 |
| Aminopyrimidinyl | GSK-3β Inhibitor XVIII | 1139875-74-3 |
| Azaindolylmaleimide | Compound 29 | 436866-61-4 |
| Azaindolylmaleimide | Compound 46 | 682807-74-5 |
| Bisindolylmaleimide | Bisindolylmaleimide X HCl | 131848-97-0 |
| Bisindolylmaleimide | Compound 5a | 436866-54-5 |
| Bisindolylmaleimide | Enzastaurin (LY317615) | 170364-57-5 |
| Bisindolylmaleimide | GF109203x | 176504-36-2 |
| Bisindolylmaleimide | Ro318220 | 125314-64-9 |
| Dihydropyridine | ML320 | 1597438-84-0 |
| Flavone | Flavopiridol | 146426-40-6 |
| Furanosesquiterpenes | Palinurin | 254901-27-4 |
| Furanosesquiterpenes | Tricantin | 853885-55-9 |
| Furopyrimidine | Compound 100 | 744255-19-4 |
| Halomethylketones | Compound 17 | 62673-69-2 |
| Halomethylketones | GSK-3β Inhibitor VI | 62673-69-2 |
| Halomethylketones | GSK-3β Inhibitor VII | 99-73-0 |
| Hymenidin | Hymenidin | 107019-95-4 |
| Indirubins | 5-Iodo-indirubin-3′-monoxime | 331467-03-9 |
| Indirubins | 6-Bromoindirubin-3-acetoxime | 667463-85-6 |
| Indirubins | GSK-3 Inhibitor IX | 667463-62-9 |
| Indirubins | GSK-3 Inhibitor X | 740841-15-0 |
| Indirubins | Indirubin | 479AM |
| Indirubins | Indirubin-3′-monoxime | 160807-49-8 |
| Indirubins | Indirubin-5-sulfonic acid sodium salt | 331467-05-1 |
| Inorganic atom | Beryllium | |
| Inorganic atom | Lithium Chloride | |
| Inorganic atom | Tungstate | |
| Inorganic atom | Zinc | |
| Isoindolone | Staurosporine | 62996-74-1 |
| Isonicotinamides | Compound 29 | 1772823-37-6 |
| Isonicotinamides | Compound 33 | 1772823-64-9 |
| Isonicotinamides | Compound 39 | 1772824-10-8 |
| Maleimide | 3F8 | 159109-11-2 |
| Maleimide | 603281-31-8 | 603281-31-8 |
| Maleimide | BIP-135 | 941575-71-9 |
| Maleimide | Compound 34 | 396091-16-0 |
| Maleimide | CP21R7 | 125314-13-8 |
| Maleimide | GSK-3 inhibitor 1 | 603272-51-1 |
| Maleimide | GSK-3β Inhibitor XI | 626604-39-5 |
| Maleimide | I5 | 264217-24-5 |
| Maleimide | IM-12 | 1129669-05-1 |
| Maleimide | Isogranulatimide | 244148-46-7 |
| Maleimide | KT 5720 | 108068-98-0 |
| Maleimide | LY2090314 | 603288-22-8 |
| Maleimide | SB-216763 | 280744-09-4 |
| Maleimide | SB-415286 (SB-41528) | 264218-23-7 |
| Maleimide | TCS 21311 | 1260181-14-3 |
| Maleimide | Tivantinib | 905854-02-6 |
| Manzamines | Manzamine A | 104196-68-1 |
| Miscellaneous | AZD2858 (AR28) | 486424-20-8 |
| Miscellaneous | CID 755673 | 521937-07-5 |
| Miscellaneous | Dibromocantharelline | 101481-34-9 |
| Miscellaneous | TCS 2002 | 1005201-24-0 |
| Organometallic | (RRu)-HB1229 | |
| Organometallic | (RRu)-NP549 | |
| Organometallic | Compound (R)-DW12 | 1047684-07-0 |
| Organometallic | Compound 3 | 1498285-39-4, |
| | | 1498285-48-5 |
| Organometallic | Compound lambda-OS1 | 1291104-51-2, |
| | | 1292843-11-8 |
| Organometallic | DW12 | 861251-33-4 |
| Organometallic | HB12 | 800384-87-6 |
| Organometallic | NP309 | 937810-13-4 |
| Oxadiazol | Compound 14d | 1374671-64-3 |
| Oxadiazol | Compound 15b | 1374671-66-5 |
| Oxadiazol | Compound 20x | 1005201-80-8 |
| Oxadiazol | GSK-3 Inhibitor II | 478482-75-6 |
| Oxadiazol | GSK3 Inhibitor, 2 | 1377154-01-2 |
| Oxadiazol | TC-G24 | 1257256-44-2 |
| Oxindole | AZD1080 | 612487-72-6 |
| Oxindole | SU9516 | 77090-84-1 |
| Patent | CN 101341138 B | |
| Patent | CN 1319968 C | |
| Patent | CP-70949 | |
| Patent | CT118637 | |
| Patent | EP 1739087 A1 | |
| Patent | EP 1961748 A2 | |
| Patent | EP 2765188 A1 | |
| Patent | GI179186X | |
| Patent | GW784752X | |
| Patent | GW784775X | |
| Patent | US 20070088080 A1 | |
| Patent | US 20100292205 A1 | |
| Patent | U.S. Pat. No. 7,514,445 B2 | |
| Patent | U.S. Pat. No. 8,071,591 B2 | |
| Patent | U.S. Pat. No. 8,207,216 B2 | |
| Patent | U.S. Pat. No. 8,686,042 B2 | |
| Patent | U.S. Pat. No. 8,771,754 B2 | |
| Patent | WO 2001085685 A1 | |
| Patent | WO 2003037891 A1 | |
| Patent | WO 2006018633 A1 | |
| Patent | WO 2007102770 A1 | |
| Patent | WO 2008077138 A1 | |
| Patent | WO 2007106537 A2 | |
| Patent | WO 2009017453 A1 | |
| Patent | WO 2010075551 A1 | |
| Patent | WO 2010104205 A1 | |
| Patent | WO 2011089416 A1 | |
| Patent | WO 2013124413 A1 | |
| Patent | WO 2014003098 A1 | |
| Patent | WO 2014013255 A1 | |
| Patent | WO 2014050779 A1 | |
| Patent | WO 2014059383 A1 | |
| Patent | WO 2014083132 A1 | |
| Patent | WO2006100490A1/EP 1863904 A1 | |
| Patent | WO2009017455 A1 | |
| Paullone | Cmpd 17b | 408532-42-3 |
| Paullone | Kenpaullone | 142273-20-9 |
| Paullones | Alsterpaullone | 237430-03-4 |
| Paullones | Alsterpaullone CN Ethyl | 852529-97-0 |
| Paullones | Azakenpaullone | 676596-65-9 |
| Paullones | Cazpaullone | 914088-64-5 |
| Peptide | FRATtide | |
| Peptide | L803 | |
| Peptides | L803-mts | |
| Publication | 705701 | |
| Publication | 708244 | |
| Publication | 709125 | |
| Publication | AR79 | |
| Publication | AZ13282107 | No Structure |
| Publication | AZ13282107 | |
| Publication | CEP-16805 | No Structure |
| Publication | CG-301338 | No Structure |
| Publication | CT73911 | |
| Publication | LY2064827 | |
| Publication | NP-103 | No Structure |
| Publication | SAR 502250 | No Structure |
| Publication | SAR 502250 (Sanofi) | 1073653-58-3 |
| Publication | XD-4241 | No Structure |
| Pyrazole | AT 7519 | 844442-38-2 |
| Pyrazole | Compound 4a | 1627557-91-8 |
| Pyrazole | Compound 4t | 1627558-10-4 |
| Pyrazole | Compound 4z | 1627558-16-0 |
| Pyrazole | GSK-3 Inhibitor XXII | 1195901-31-5 |
| Pyrazolone | GSK-3beta Inhibitor XXVI | 871843-09-3 |
| Pyrazolopyridazines | Compound 18 | 405223-20-3 |
| Pyrazolopyridazines | Compound 19 | 405223-71-4 |
| Pyrazolopyridine | Pyrazolopyridine 18 | 405221-39-8 |
| Pyrazolopyridine | Pyrazolopyridine 34 | 583039-27-4 |
| Pyrazolopyridine | Pyrazolopyridine 9 | 923029-74-7 |
| Pyrazolopyridines | Compound 14 | 583038-63-5 |
| Pyrazolopyridines | Compound 14 | 583038-63-5 |
| Pyrazolopyridines | Compound 23 | 583038-76-0 |
| Pyrazoloquinoxaline | NSC 693868 (Compound 1) | 40254-90-8 |
| Pyrazoloquinoxaline | NSC 693868 (Compound 1) | 40254-90-8 |
| Pyridinone | Compound 150 | 1282042-18-5 |
| Pyrrolopyridinyl | Compound 12 | 2025388-10-5 |
| Pyrrolopyridinyl | Compound 27 | 2025388-25-2 |
| Pyrroloazepine | Hymenialdisine | 82005-12-7 |
| Quinazolin | GSK-3 Inhibitor XIII | 404828-08-6 |
| Quinolinecarb | VP0.7 | 331963-23-6 |
| Quinoline | | 1132813M6-7 |
| Quinoline | | 1132812-98-6 |
| Quinoline | | 950727-66-9 |
| Quinoline | | 950727-04-5 |
| Quinoline | | 1132812-98-6 |
| Thiadiazolidindiones | GSK-3β Inhibitor I | 327036-89-5 |
| Thiadiazolidindiones | NP031112 (Tideglusib) | 865854-05-3 |
| Thiadiazolidindiones | NP031115 | 1400575-57-6 |
| Triazolpyrimidine | Compound 90 | 91322-11-1 |
| Triazolpyrimidine | Compound 92 | 1043429-30-6 |
| Urea | GSK-3β Inh. VIII AR-A014418 | 487021-52-3 |
| Urea | A-1070722 | 1384424-80-9 |

In some embodiments, an agent having activity as a Wnt agonist is a GSK3 inhibitor. Preferably, the GSK3 inhibitor is AZD1080, GSK3 inhibitor XXII, CHIR99021 or LY2090314. In a preferred embodiment, the Wnt agonist is CHIR99021. In other preferred embodiments, the Wnt agonist and/or GSK3 inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione. (Formula A.)

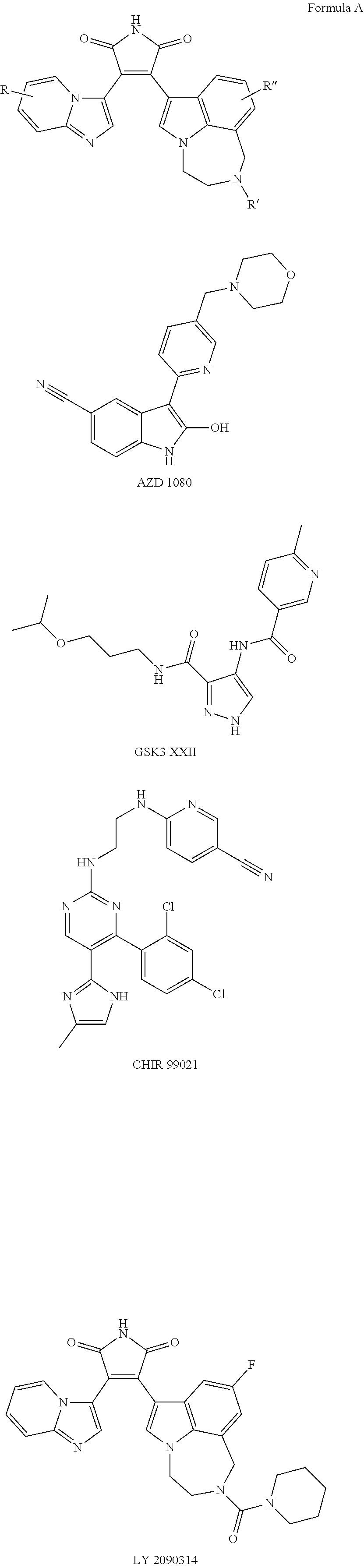

The Wnt agonist can be any selected from WO 2018/125746, which is hereby incorporated by reference. In some embodiments, the Wnt agonist can be the compound as defined in claim 1 of WO 2018/125746. In some embodiments, the Wnt agonist can be the compound as defined in claim 12 of WO 2018/125746.

Exemplary substituted 3-Imidazo[1,2-a]pyridin-3-yl-4-(1,2,3,4-tetrahydro-[1,4]diazepino-[6,7,1-hi]indol-7-yl)pyrrole-2,5-diones include: 3-(imidazo[1,2-a]pyridin-3-yl)-4-(2-(piperidine-1-carbonyl)-9-(trifluoromethyl)-1,2,3,4-tetrahydro-[1,4]diazepino[6,7,1-hi]indol-7-yl)-1H-pyrrole-2,5-dione; 7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indole-9-carbonitrile; 3-(9-ethynyl-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-amino-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 1-(9-fluoro-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-2-carbonyl)piperidine-4-carbaldehyde; 3-(9-fluoro-2-(4-(hydroxymethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(8-oxa-3-azabicyclo[3.2.1]octane-3-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(benzo[d]isoxazol-3-yl)-4-(9-fluoro-2-(piperidine-1-carbonyl)-1,2,3,4-tetrahydro-[1,4]diazepino[6,7,1-hi]indol-7-yl)-1H-pyrrole-2,5-dione; N-(7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indol-9-yl)acetamide; 3-(9-(difluoromethyl)-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(3,3-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-((1R,4R)-2,5-diazabicyclo[2.2.1]heptane-2-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 2-(8-oxa-3-azabicyclo[3.2.1]octane-3-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 2-(3,3-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 2-(4,4-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 3-(2-(4,4-difluoropipendine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(8-oxa-3-azabicyclo[3.2.1]octane-3-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(aminomethyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(hydroxymethyl)piperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 2-(4-(hydroxymethyl)piperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 3-(9-fluoro-2-(3,3,4,4,5,5-hexafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(3,3,5,5-tetrafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(2,2,6,6-tetrafluoromorpholine-4-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoro-3-hydroxypipendine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(difluoro(hydroxy)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(6,6-difluoro-1,4-oxazepane-4-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-([1,2,4]triazolo[4,3-a]pyridin-3-yl)-4-(9-fluoro-2-(piperidine-1-carbonyl)-1,2,3,4-tetrahydro-[1,4]diazepino[6,7,1-hi]indol-7-yl)-1H-pyrrole-2,5-dione; 3-(9-fluoro-2-(piperidine-1-carbonyl-d10)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(piperidine-1-carbonyl)-1,2,3,4-tetrahydro-[1,4]diazepino[6,7,1-hi]indol-7-yl-3,3,4,4-d4)-4-(imidazo[1,2-a]pyridin-3-yl)-1H-pyrrole-2,5-dione; 3-(9-fluoro-2-(4-(2,2,2-trifluoro-1-hydroxyethyl)piperidine-1-carbonyl)-1,2,3,4-tetrahydro-diazepino[6,7,1-hi]indol-7-yl)-4-(imidazo[1,2-a]pyridin-3-yl)-1H-pyrrole-2,5-dione; 3-(9-fluoro-2-(4-((methylamino)methyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-((dimethylamino)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-aminopiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(4-(methylamino)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(dimethylamino)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 9-fluoro-7-(4-(imidazo[1,2-a]pyridin-3-yl)-2,5-dioxo-2,5-dihydro-1H-pyrrol-3-yl)-N-(piperidin-4-ylmethyl)-3,4-dihydro-[1,4]diazepino[6,7,1-hi]indole-2(1H)-carboxamide; 9-fluoro-7-(4-(imidazo[1,2-a]pyridin-3-yl)-2,5-dioxo-2,5-dihydro-1H-pyrrol-3-yl)-N-methyl-N-(piperidin-4-ylmethyl)-3,4-dihydro-[1,4]diazepino[6,7,1-hi]indole-2(1H)-carboxamide; 9-fluoro-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide; 3-(9-fluoro-2-((1R,4R)-5-methyl-2,5-diazabicyclo[2.2.1]heptane-2-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(2-methyl-2,8-diazaspiro[4.5]decane-8-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(8-methyl-2,8-diazaspiro[4.5]decane-2-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(imidazo[1,2-a]pyridin-3-yl)-4-(2-(2,2,6,6-tetrafluoromorpholine-4-carbonyl)-9-(trifluoromethyl)-1,2,3,4-tetrahydro-[1,4]diazepino[6,7,1-hi]indol-7-yl)-1H-pyrrole-2,5-dione; 3-(2-(6,6-difluoro-1,4-oxazepane-4-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 2-(4-(dimethylamino)piperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 9-cyano-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide; 7-(4-(imidazo[1,2-a]pyridin-3-yl)-2,5-dioxo-2,5-dihydro-1H-pyrrol-3-yl)-2-(8-methyl-2,8-diazaspiro[4.5]decane-2-carbonyl)-1,2,3,4-tetrahydro-[1,4]diazepino[6,7,1-hi]indole-9-carbonitrile; 3-(8,9-difluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; or 3-(9-fluoro-2-(piperdine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione (LY20900314).

Preferably, the substituted 3-Imidazo[1,2-a]pyridin-3-yl-4-(1,2,3,4-tetrahydro-[1,4]diazepino-[6,7,1-hi]indol-7-yl)pyrrole-2,5-dione is: 3-(imidazo[1,2-a]pyridin-3-yl)-4-(2-(piperidine-1-carbonyl)-9-(trifluoromethyl)-1,2,3,4-tetrahydro-[1,4]diazepino[6,7,1-hi]indol-7-yl)-1H-pyrrole-2,5-dione; 7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indole-9-carbonitrile; 3-(9-ethynyl-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(4-(hydroxymethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(8-oxa-3-azabicyclo[3.2.1]octane-3-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-(difluoromethyl)-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(3,3-difluoropipendine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 2-(4,4-difluoropipendine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 3-(2-(8-oxa-3-azabicyclo[3.2.1]octane-3-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(hydroxymethyl)piperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(3,3,4,4,5,5-hexafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(3,3,5,5-tetrafluoropipendine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(2,2,6,6-tetrafluoromorpholine-4-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoro-3-hydroxypipendine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(difluoro(hydroxy)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(6,6-difluoro-1,4-oxazepane-4-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(piperidine-1-carbonyl-d10)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(piperidine-1-carbonyl)-1,2,3,4-tetrahydro-[1,4]diazepino[6,7,1-hi]indol-7-yl-3,3,4,4-d4)-4-(imidazo[1,2-a]pyridin-3-yl)-1H-pyrrole-2,5-dione; 3-(9-fluoro-2-(4-(2,2,2-trifluoro-1-hydroxyethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-((dimethylamino)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(dimethylamino)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 9-fluoro-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide; 3-(imidazo[1,2-a]pyridin-3-yl)-4-(2-(2,2,6,6-tetrafluoromorpholine-4-carbonyl)-9-(trifluoromethyl)-1,2,3,4-tetrahydro-[1,4]diazepino[6,7,1-hi]indol-7-yl)-1H-pyrrole-2,5-dione; 3-(2-(6,6-difluoro-1,4-oxazepane-4-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(8,9-difluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; or 3-(9-fluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione. (LY2090314).

Most preferably, the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione. (LY2090314).

The structures of the substituted 3-Imidazo[1,2-a]pyridin-3-yl-4-(1,2,3,4-tetrahydro-[1,4]diazepino-[6,7,1-hi]indol-7-yl)pyrrole-2,5-diones are shown below in Table 16.

TABLE 16

| | | |
|---|---|---|
| Compound I-1 | 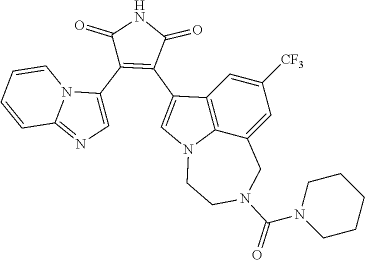 [SMILES: O=C1NC(=O)C(c2cnc3ccccn23)=C1c1cn2c3c(cc(C(F)(F)F)cc13)CN(C(=O)N1CCCCC1)CC2] | 3-(imidazo[1,2-a]pyridin-3-yl)-4-(2-(piperidine-1- carbonyl)-9-(trifluoromethyl)-1,2,3,4-tetrahydro- [1,4]diazepino[6,7,1-hi]indol-7-yl)-1H-pyrrole- 2,5-dione |
| Compound I-2 | 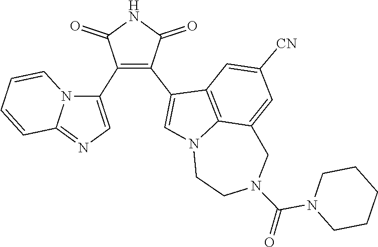 [SMILES: N#Cc1cc2c3c(c1)c(C1=C(c4cnc5ccccn45)C(=O)NC1=O)cn3CCN(C(=O)N1CCCCC1)C2] | 7-(4-(imidazo[1,2-a]pyridin-3-yl)-2,5-dioxo-2,5- dihydro-1H-pyrrol-3-yl)-2-(piperidine-1-carbonyl)- 1,2,3,4-tetrahydro-[1,4]diazepino[6,7,1-hi]indole-9- carbonitrile |
| Compound I-3 | 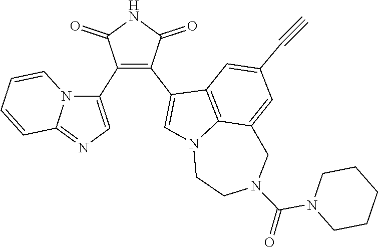 [SMILES: C#Cc1cc2c3c(c1)c(C1=C(c4cnc5ccccn45)C(=O)NC1=O)cn3CCN(C(=O)N1CCCCC1)C2] | 3-(9-ethynyl-2-(piperidine-1-carbonyl)-1,2,3,4- tetrahydro-[1,4]diazepino[6,7,1-hi]indol-7-yl)-4- (imidazo[1,2-a]pyridin-3-yl)-1H-pyrrole-2,5-dione |
| Compound I-4 | 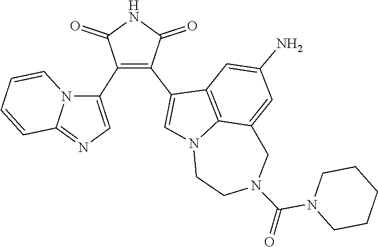 [SMILES: Nc1cc2c3c(c1)c(C1=C(c4cnc5ccccn45)C(=O)NC1=O)cn3CCN(C(=O)N1CCCCC1)C2] | 3-(9-amino-2-(piperidine-1-carbonyl)-1,2,3,4- tetrahydro-[1,4]diazepino[6,7,1-hi]indol-7-yl)-4- (imidazo[1,2-a]pyridin-3-yl)-1H-pyrrole-2,5-dione |
| Compound I-5 | 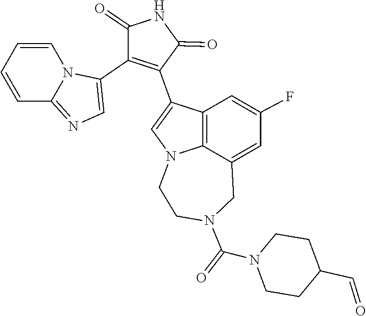 [SMILES: O=CC1CCN(C(=O)N2CCn3cc(C4=C(c5cnc6ccccn56)C(=O)NC4=O)c4cc(F)cc(c43)C2)CC1] | 1-(9-fluoro-7-(4-(imidazo[1,2-a]pyridin-3-yl)-2,5- dioxo-2,5-dihydro-1H-pyrrol-3-yl)-1,2,3,4- tetrahydro-[1,4]diazepino[6,7,1-hi]indole-2- carbonyl)piperidine-4-carbaldehyde |
| Compound I-6 | 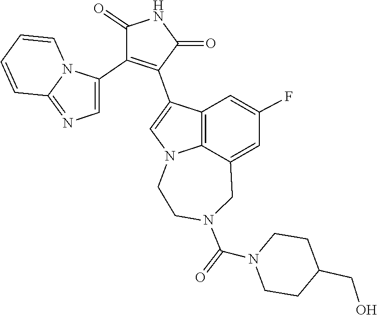 [SMILES: O=C1NC(=O)C(c2cnc3ccccn23)=C1c1cn2c3c(cc(F)cc13)CN(C(=O)N1CCC(CO)CC1)CC2] | 3-(9-fluoro-2-(4-(hydroxymethyl)piperidine-1- carbonyl)-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-7 | 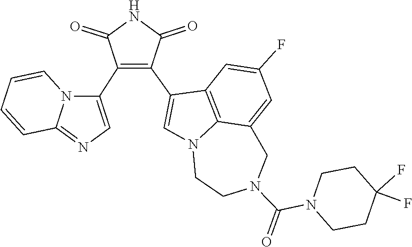 [SMILES: O=C1NC(=O)C(c2cnc3ccccn23)=C1c1cn2c3c(cc(F)cc13)CN(C(=O)N1CCC(F)(F)CC1)CC2] | 3-(2-(4,4-difluoropiperidine-1-carbonyl)-9-fluoro- 1,2,3,4-tetrahydro-[1,4]diazepino[6,7,1-hi]indol-7- yl)-4-(imidazo[1,2-a]pyridin-3-yl)-1H-pyrrole-2,5- dione |
| Compound I-8 | 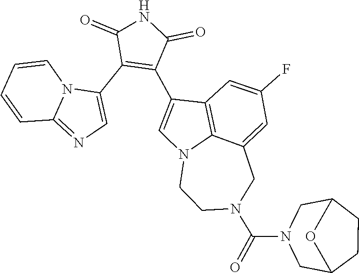 [SMILES: O=C1NC(=O)C(c2cnc3ccccn23)=C1c1cn2c3c(cc(F)cc13)CN(C(=O)N1CC3CCC(C1)O3)CC2] | 3-(2-(8-oxa-3-azabicyclo[3.2.1]octane-3-carbonyl)- 9-fluoro-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-9 | 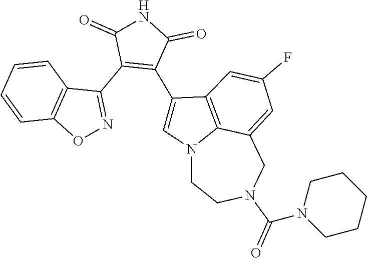 [SMILES: O=C1NC(=O)C(c2cn3c4c(cc(F)cc24)CN(C(=O)N2CCCCC2)CC3)=C1c1noc2ccccc12] | 3-(benzo[d]isoxazol-3-yl)-4-(9-fluoro-2-(piperidine- 1-carbonyl)-1,2,3,4-tetrahydro- [1,4]diazepino[6,7,1-hi]indol-7-yl)-1H-pyrrole- 2,5-dione |
| Compound I-10 | 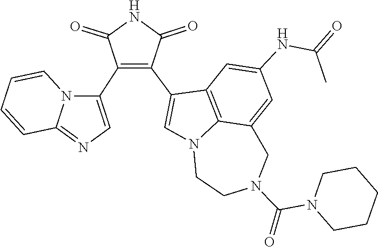 [SMILES: CC(=O)Nc1cc2c3c(c1)c(C1=C(c4cnc5ccccn45)C(=O)NC1=O)cn3CCN(C(=O)N1CCCCC1)C2] | N-(7-(4-(imidazo[1,2-a]pyridin-3-yl)-2,5-dioxo-2,5- dihydro-1H-pyrrol-3-yl)-2-(piperidine-1-carbonyl)- 1,2,3,4-tetrahydro-[1,4]diazepino[6,7,1-hi]indol-9- yl)acetamide |
| Compound I-11 | 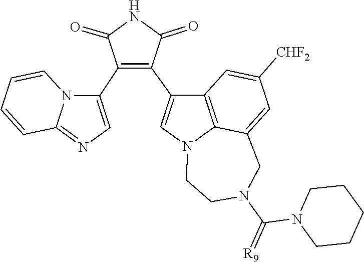 | 3-(9-(difluoromethyl)-2-(piperidine-1-carbonyl)- 1,2,3,4-tetrahydro-[1,4]diazepino[6,7,1-hi]indol-7- yl)-4-(imidazo[1,2-a]pyridin-3-yl)-1H-pyrrole-2,5- dione |
| Compound I-12 | 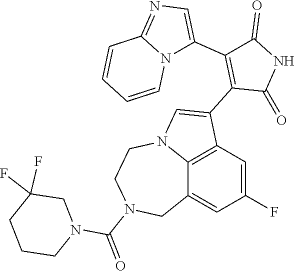 [SMILES: O=C1NC(=O)C(c2cnc3ccccn23)=C1c1cn2c3c(cc(F)cc13)CN(C(=O)N1CCCC(F)(F)C1)CC2] | 3-(2-(3,3-difluoropiperidine-1-carbonyl)-9-fluoro- 1,2,3,4-tetrahydro-[1,4]diazepino[6,7,1-hi]indol-7- yl)-4-(imidazo[1,2-a]pyridin-3-yl)-1H-pyrrole-2,5- dione |
| Compound I-13 | 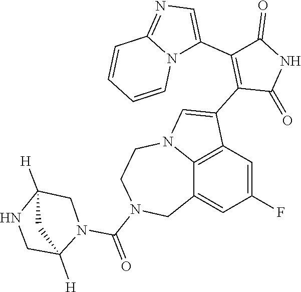 [SMILES: O=C1NC(=O)C(c2cnc3ccccn23)=C1c1cn2c3c(cc(F)cc13)CN(C(=O)N1C[C@H]3C[C@@H]1CN3)CC2] | 3-(2-((1R,4R)-2,5-diazabicyclo[2.2.1]heptane-2- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-14 | 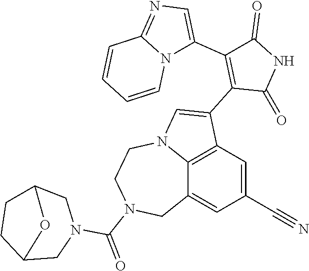 [SMILES: N#Cc1cc2c3c(c1)c(C1=C(c4cnc5ccccn45)C(=O)NC1=O)cn3CCN(C(=O)N1CC3CCC(C1)O3)C2] | 2-(8-oxa-3-azabicyclo[3.2.1]octane-3-carbonyl)-7-(4- (imidazo[1,2-a]pyridin-3-yl)-2,5-dioxo-2,5-dihydro- 1H-pyrrol-3-yl)-1,2,3,4-tetrahydro- [1,4]diazepino[6,7,1-hi]indole-9-carbonitrile |
| Compound I-15 | 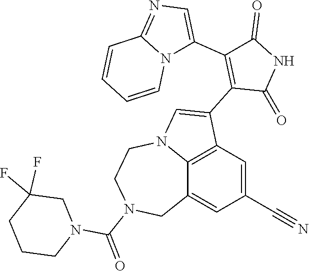 [SMILES: N#Cc1cc2c3c(c1)c(C1=C(c4cnc5ccccn45)C(=O)NC1=O)cn3CCN(C(=O)N1CCCC(F)(F)C1)C2] | 2-(3,3-difluoropiperidine-1-carbonyl)-7-(4- (imidazo[1,2-a]pyridin-3-yl)-2,5-dioxo-2,5- dihydro-1H-pyrrol-3-yl)-1,2,3,4-tetrahydro- [1,4]diazepino[6,7,1-hi]indole-9-carbonitrile |
| Compound I-16 | 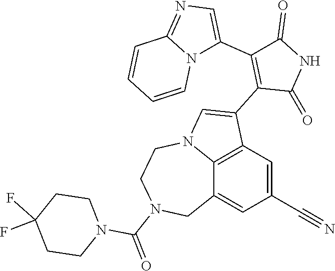 [SMILES: N#Cc1cc2c3c(c1)c(C1=C(c4cnc5ccccn45)C(=O)NC1=O)cn3CCN(C(=O)N1CCC(F)(F)CC1)C2] | 2-(4,4-difluoropiperidine-1-carbonyl)-7-(4- (imidazo[1,2-a]pyridin-3-yl)-2,5-dioxo-2,5- dihydro-1H-pyrrol-3-yl)-1,2,3,4-tetrahydro- [1,4]diazepino[6,7,1-hi]indole-9-carbonitrile |
| Compound I-17 | 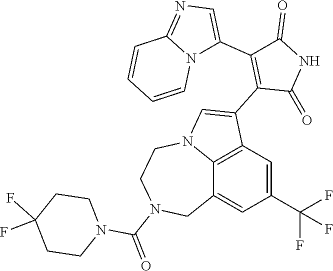 [SMILES: O=C1NC(=O)C(c2cnc3ccccn23)=C1c1cn2c3c(cc(C(F)(F)F)cc13)CN(C(=O)N1CCC(F)(F)CC1)CC2] | 3-(2-(4,4-difluoropiperidine-1-carbonyl)-9- (trifluoromethyl)-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-18 | 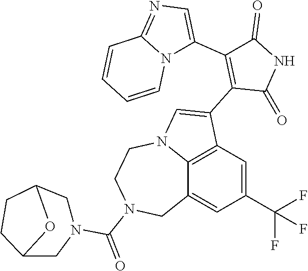 [SMILES: O=C1NC(=O)C(c2cnc3ccccn23)=C1c1cn2c3c(cc(C(F)(F)F)cc13)CN(C(=O)N1CC3CCC(C1)O3)CC2] | 3-(2-(8-oxa-3-azabicyclo[3.2.1]octane-3-carbonyl)- 9-(trifluoromethyl)-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-19 | 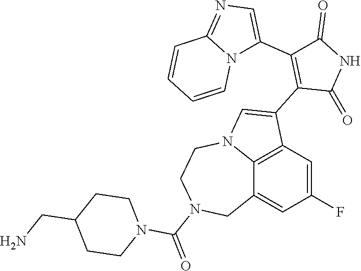 [SMILES: NCC1CCN(C(=O)N2CCn3cc(C4=C(c5cnc6ccccn56)C(=O)NC4=O)c4cc(F)cc(c43)C2)CC1] | 3-(2-(4-(aminomethyl)piperidine-1-carbonyl)-9- fluoro-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-20 | 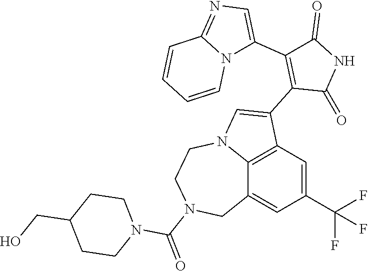 [SMILES: O=C1NC(=O)C(c2cnc3ccccn23)=C1c1cn2c3c(cc(C(F)(F)F)cc13)CN(C(=O)N1CCC(CO)CC1)CC2] | 3-(2-(4-(hydroxymethyl)piperidine-1-carbonyl)-9- (trifluoromethyl)-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-21 | 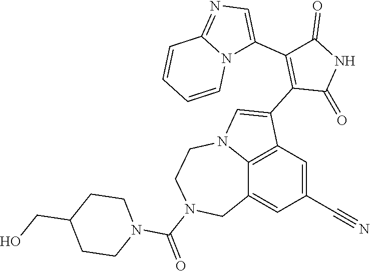 [SMILES: N#Cc1cc2c3c(c1)c(C1=C(c4cnc5ccccn45)C(=O)NC1=O)cn3CCN(C(=O)N1CCC(CO)CC1)C2] | 2-(4-(hydroxymethyl)piperidine-1-carbonyl)-7-(4- (imidazo[1,2-a]pyridin-3-yl)-2,5-dioxo-2,5-dihydro- 1H-pyrrol-3-yl)-1,2,3,4-tetrahydro- [1,4]diazepino[6,7,1-hi]indole-9-carbonitrile |
| Compound I-22 | 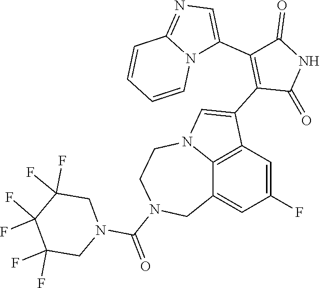 [SMILES: O=C1NC(=O)C(c2cnc3ccccn23)=C1c1cn2c3c(cc(F)cc13)CN(C(=O)N1CC(F)(F)C(F)(F)C(F)(F)C1)CC2] | 3-(9-fluoro-2-(3,3,4,4,5,5-hexafluoropiperidine-1- carbonyl)-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-23 | 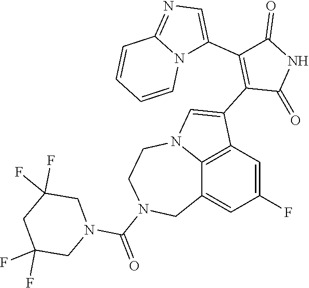 [SMILES: O=C1NC(=O)C(c2cnc3ccccn23)=C1c1cn2c3c(cc(F)cc13)CN(C(=O)N1CC(F)(F)CC(F)(F)C1)CC2] | 3-(9-fluoro-2-(3,3,5,5-tetrafluoropiperidine-1- carbonyl)-1,2,3,4-tetrahydro- [1,4]diazepino[6,7,1-hi]indol-7-yl)-4- (imidazo[1,2-a]pyridin-3-yl)-1H-pyrrole-2,5-dione |
| 1Compound I-24 | 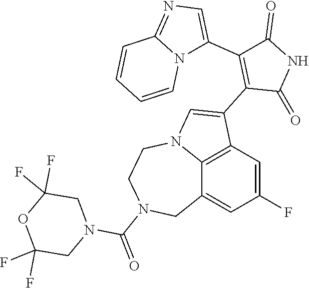 [SMILES: O=C1NC(=O)C(c2cnc3ccccn23)=C1c1cn2c3c(cc(F)cc13)CN(C(=O)N1CC(F)(F)OC(F)(F)C1)CC2] | 3-(9-fluoro-2-(2,2,6,6-tetrafluoromorpholine-4- carbonyl)-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-25 | 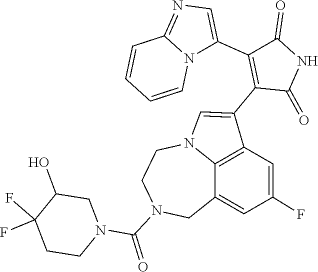 [SMILES: O=C1NC(=O)C(c2cnc3ccccn23)=C1c1cn2c3c(cc(F)cc13)CN(C(=O)N1CCC(F)(F)C(O)C1)CC2] | 3-(2-(4,4-difluoro-3-hydroxypiperidine-1-carbonyl)- 9-fluoro-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-26 | 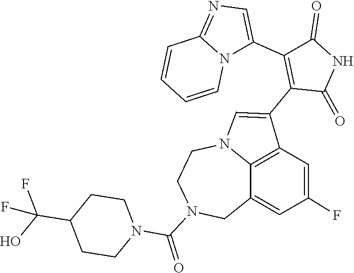 [SMILES: O=C1NC(=O)C(c2cnc3ccccn23)=C1c1cn2c3c(cc(F)cc13)CN(C(=O)N1CCC(C(O)(F)F)CC1)CC2] | 3-(2-(4-(difluoro(hydroxy)methyl)piperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-27 | 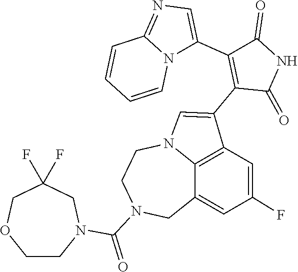 [SMILES: O=C1NC(=O)C(c2cnc3ccccn23)=C1c1cn2c3c(cc(F)cc13)CN(C(=O)N1CCOCC(F)(F)C1)CC2] | 3-(2-(6,6-difluoro-1,4-oxazepane-4-carbonyl)-9- fluoro-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-28 | 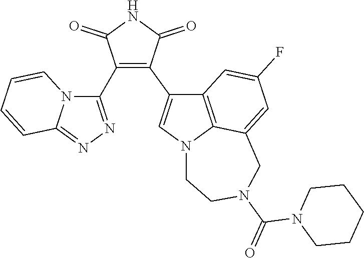 [SMILES: O=C1NC(=O)C(c2nnc3ccccn23)=C1c1cn2c3c(cc(F)cc13)CN(C(=O)N1CCCCC1)CC2] | 3-([1,2,4]triazolo[4,3-a]pyridin-3-yl)-4-(9-fluoro-2- (piperidine-1-carbonyl)-1,2,3,4-tetrahydro- [1,4]diazepino[6,7,1-hi]indol-7-yl)-1H-pyrrole- 2,5-dione |
| Compound I-29 | 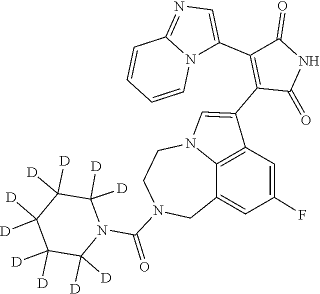 [SMILES: [2H]C1([2H])N(C(=O)N2CCn3cc(C4=C(c5cnc6ccccn56)C(=O)NC4=O)c4cc(F)cc(c43)C2)C([2H])([2H])C([2H])([2H])C([2H])([2H])C1([2H])[2H]] | 3-(9-fluoro-2-(piperidine-1-carbonyl-d10)-1,2,3,4- tetrahydro-[1,4]diazepino[6,7,1-hi]indol-7-yl)-4- (imidazo[1,2-a]pyridin-3-yl)-1H-pyrrole-2,5-dione |
| Compound I-30 | 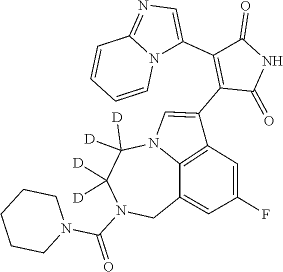 [SMILES: [2H]C1([2H])N(C(=O)N2CCCCC2)Cc2cc(F)cc3c(C4=C(c5cnc6ccccn56)C(=O)NC4=O)cn(c23)C1([2H])[2H]] | 3-(9-fluoro-2-(piperidine-1-carbonyl)-1,2,3,4- tetrahydro-[1,4]diazepino[6,7,1-hi]indol-7-yl- 3,3,4,4-d4)-4-(imidazo[1,2-a]pyridin-3-yl)-1H- pyrrole-2,5-dione |
| Compound I-31 | 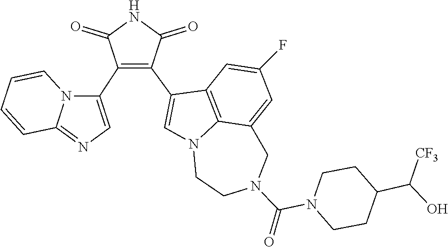 [SMILES: O=C1NC(=O)C(c2cnc3ccccn23)=C1c1cn2c3c(cc(F)cc13)CN(C(=O)N1CCC(C(O)C(F)(F)F)CC1)CC2] | 3-(9-fluoro-2-(4-(2,2,2-trifluoro-1- hydroxyethyl)piperidine-1-carbonyl)-1,2,3,4- tetrahydro-[1,4]diazepino[6,7,1-hi]indol-7-yl)-4- (imidazo[1,2-a]pyridin-3-yl)-1H-pyrrole-2,5-dione |
| Compound I-32 | 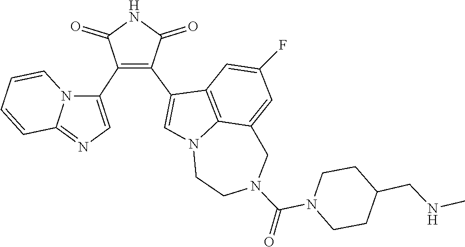 [SMILES: CNCC1CCN(C(=O)N2CCn3cc(C4=C(c5cnc6ccccn56)C(=O)NC4=O)c4cc(F)cc(c43)C2)CC1] | 3-(9-fluoro-2-(4-((methylamino)methyl)piperidine- 1-carbonyl)-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-33 | 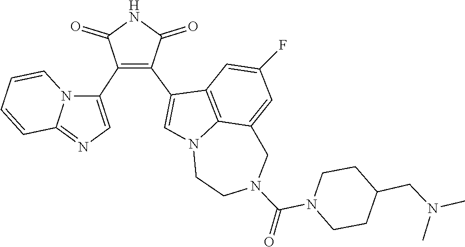 [SMILES: CN(C)CC1CCN(C(=O)N2CCn3cc(C4=C(c5cnc6ccccn56)C(=O)NC4=O)c4cc(F)cc(c43)C2)CC1] | 3-(2-(4-((dimethylamino)methyl)piperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-34 | 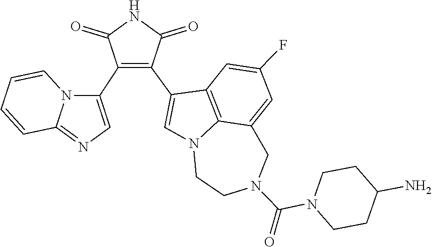 [SMILES: NC1CCN(C(=O)N2CCn3cc(C4=C(c5cnc6ccccn56)C(=O)NC4=O)c4cc(F)cc(c43)C2)CC1] | 3-(2-(4-aminopiperidine-1-carbonyl)-9-fluoro- 1,2,3,4-tetrahydro-[1,4]diazepino[6,7,1-hi]indol-7- yl)-4-(imidazo[1,2-a]pyridin-3-yl)-1H-pyrrole- 2,5-dione |
| Compound I-35 | 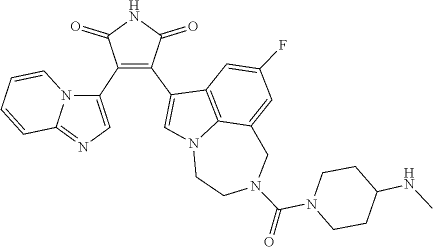 [SMILES: CNC1CCN(C(=O)N2CCn3cc(C4=C(c5cnc6ccccn56)C(=O)NC4=O)c4cc(F)cc(c43)C2)CC1] | 3-(9-fluoro-2-(4-(methylamino)piperidine-1- carbonyl)-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-36 | 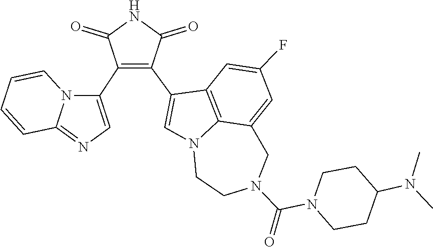 [SMILES: CN(C)C1CCN(C(=O)N2CCn3cc(C4=C(c5cnc6ccccn56)C(=O)NC4=O)c4cc(F)cc(c43)C2)CC1] | 3-(2-(4-(dimethylamino)piperidine-1-carbonyl)-9- fluoro-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-37 | 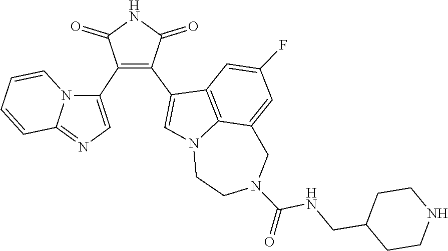 [SMILES: O=C1NC(=O)C(c2cnc3ccccn23)=C1c1cn2c3c(cc(F)cc13)CN(C(=O)NCC1CCNCC1)CC2] | 9-fluoro-7-(4-(imidazo[1,2-a]pyridin-3-yl)-2,5- dioxo-2,5-dihydro-1H-pyrrol-3-yl)-N-(piperidin-4- ylmethyl)-3,4-dihydro- [1,4]diazepino[6,7,1-hi]indole-2(1H)-carboxamide |
| Compound I-38 | 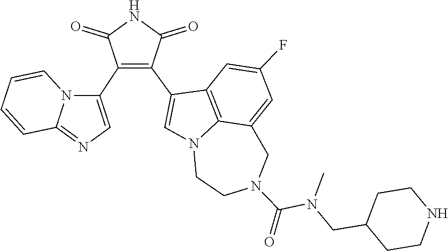 [SMILES: CN(CC1CCNCC1)C(=O)N1CCn2cc(C3=C(c4cnc5ccccn45)C(=O)NC3=O)c3cc(F)cc(c32)C1] | 9-fluoro-7-(4-(imidazo[1,2-a]pyridin-3-yl)-2,5- dioxo-2,5-dihydro-1H-pyrrol-3-yl)-N-methyl-N- (piperidin-4-ylmethyl)-3,4-dihydro- [1,4]diazepino[6,7,1-hi]indole-2(1H)-carboxamide |
| Compound I-39 | 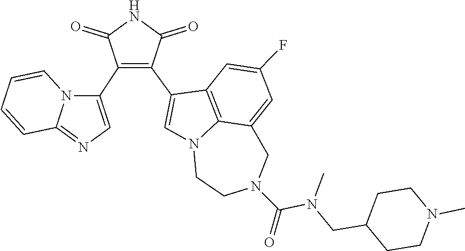 [SMILES: CN1CCC(CN(C)C(=O)N2CCn3cc(C4=C(c5cnc6ccccn56)C(=O)NC4=O)c4cc(F)cc(c43)C2)CC1] | 9-fluoro-7-(4-(imidazo[1,2-a]pyridin-3-yl)-2,5- dioxo-2,5-dihydro-1H-pyrrol-3-yl)-N-methyl-N- ((1-methylpiperidin-4-yl)methyl)-3,4-dihydro- [1,4]diazepino[6,7,1-hi]indole-2(1H)-carboxamide |
| Compound I-40 | 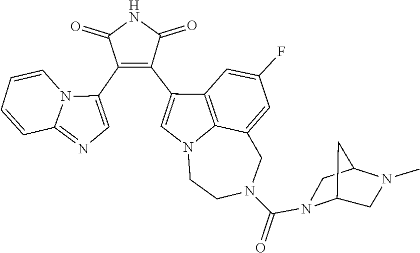 [SMILES: CN1CC2CC1CN2C(=O)N1CCn2cc(C3=C(c4cnc5ccccn45)C(=O)NC3=O)c3cc(F)cc(c32)C1] | 3-(9-fluoro-2-((1R,4R)-5-methyl-2,5- diazabicyclo[2.2.1]heptane-2-carbonyl)-1,2,3,4- tetrahydro-[1,4]diazepino[6,7,1-hi]indol-7-yl)-4- (imidazo[1,2-a]pyridin-3-yl)-1H-pyrrole-2,5-dione |
| Compound I-41 | 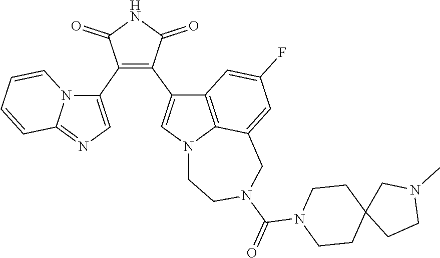 [SMILES: CN1CCC2(CCN(C(=O)N3CCn4cc(C5=C(c6cnc7ccccn67)C(=O)NC5=O)c5cc(F)cc(c54)C3)CC2)C1] | 3-(9-fluoro-2-(2-methyl-2,8-diazaspiro[4.5]decane- 8-carbonyl)-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-42 | 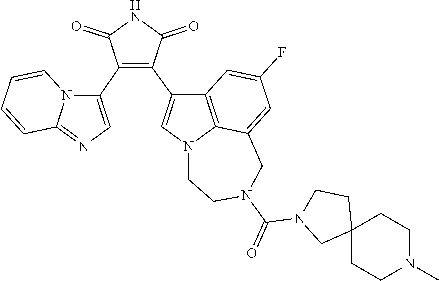 [SMILES: CN1CCC2(CC1)CCN(C(=O)N1CCn3cc(C4=C(c5cnc6ccccn56)C(=O)NC4=O)c4cc(F)cc(c43)C1)C2] | 3-(9-fluoro-2-(8-methyl-2,8-diazaspiro[4.5]decane- 2-carbonyl)-1,2,3,4-tetrahydro-[1,4]diazepino[6,7,1- hi]indol-7-yl)-4-(imidazo[1,2-a]pyridin-3-yl)-1H- pyrrole-2,5-dione |
| Compound I-43 | 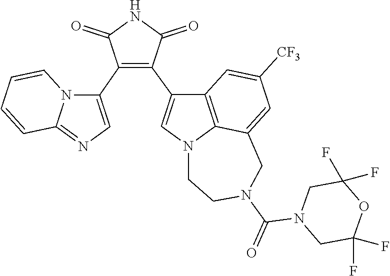 [SMILES: O=C1NC(=O)C(c2cnc3ccccn23)=C1c1cn2c3c(cc(C(F)(F)F)cc13)CN(C(=O)N1CC(F)(F)OC(F)(F)C1)CC2] | 3-(imidazo[1,2-a]pyridin-3-yl)-4-(2-(2,2,6,6- tetrafluoromorpholine-4-carbonyl)-9- (trifluoromethyl)-1,2,3,4-tetrahydro- [1,4]diazepino[6,7,1-hi]indol-7-yl)-1H-pyrrole- 2,5-dione |
| Compound I-44 | 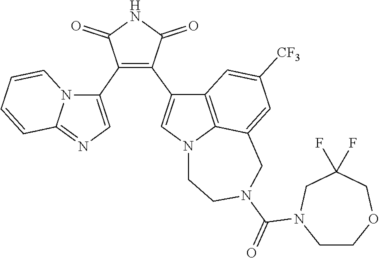 [SMILES: O=C1NC(=O)C(c2cnc3ccccn23)=C1c1cn2c3c(cc(C(F)(F)F)cc13)CN(C(=O)N1CCOCC(F)(F)C1)CC2] | 3-(2-(6,6-difluoro-1,4-oxazepane-4-carbonyl)-9- (trifluoromethyl)-1,2,3,4-tetrahydro- [1,4]diazepino[6,7,1-hi]indol-7-yl)-4- (imidazo[1,2-a]pyridin-3-yl)-1H-pyrrole-2,5-dione |
| Compound I-45 | 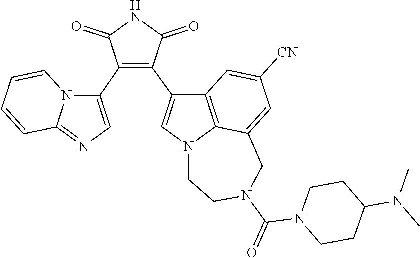 [SMILES: CN(C)C1CCN(C(=O)N2CCn3cc(C4=C(c5cnc6ccccn56)C(=O)NC4=O)c4cc(C#N)cc(c43)C2)CC1] | 2-(4-(dimethylamino)piperidine-1-carbonyl)-7-(4- (imidazo[1,2-a]pyridin-3-yl)-2,5-dioxo-2,5- dihydro-1H-pyrrol-3-yl)-1,2,3,4-tetrahydro- [1,4]diazepino[6,7,1-hi]indole-9-carbonitrile |
| Compound I-46 | 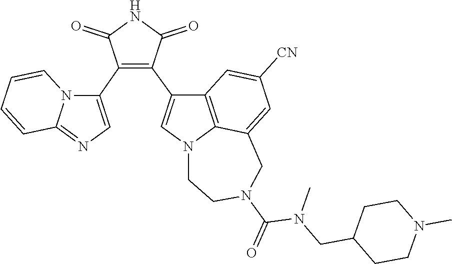 [SMILES: CN1CCC(CN(C)C(=O)N2CCn3cc(C4=C(c5cnc6ccccn56)C(=O)NC4=O)c4cc(C#N)cc(c43)C2)CC1] | 9-cyano-7-(4-(imidazo[1,2-a]pyridin-3-yl)-2,5- dioxo-2,5-dihydro-1H-pyrrol-3-yl)-N-methyl-N- ((1-methylpiperidin-4-yl)methyl)-3,4-dihydro- [1,4]diazepino[6,7,1-hi]indole-2(1H)-carboxamide |
| Compound I-47 | 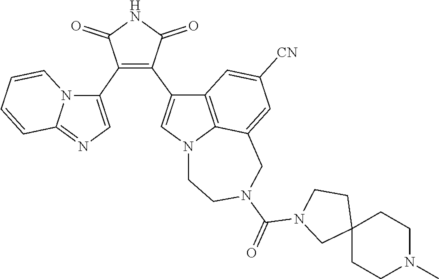 [SMILES: CN1CCC2(CC1)CCN(C(=O)N1CCn3cc(C4=C(c5cnc6ccccn56)C(=O)NC4=O)c4cc(C#N)cc(c43)C1)C2] | 7-(4-(imidazo[1,2-a]pyridin-3-yl)-2,5-dioxo-2,5- dihydro-1H-pyrrol-3-yl)-2-(8-methyl-2,8- diazaspiro[4.5]decane-2-carbonyl)-1,2,3,4- tetrahydro-[1,4]diazepino[6,7,1-hi]indole-9- carbonitrile |
| Compound I-48 | 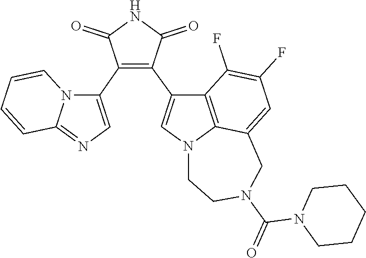 [SMILES: O=C1NC(=O)C(c2cnc3ccccn23)=C1c1cn2c3c(cc(F)c(F)c13)CN(C(=O)N1CCCCC1)CC2] | 3-(8,9-difluoro-2-(piperidine-1-carbonyl)-1,2,3,4- tetrahydro-[1,4]diazepino[6,7,1-hi]indol-7-yl)-4- (imidazo[1,2-a]pyridin-3-yl)-1H-pyrrole-2,5-dione |

In other embodiments, the Wnt agonist and/or GSK3 inhibitor are as described in WO 2018/125746, US 20180214458 and U.S. Ser. No. 62/608,663 the contents of which are each incorporated by reference in their entireties for all purposes.

### Epigenetic Modulators

The one or more otic therapeutic agents in any embodiment disclosed could be one or more of the following epigenetic modulators.

Epigenetic modulators included epigenetic modifiers, mediators and modulators. Epigenetic modifiers are genes whose products modify the epigenome directly through DNA methylation, the post-translational modification of chromatin or the alteration of the structure of chromatin. The epigenetic mediators, are often the target of epigenetic modification, although they are rarely mutated themselves. The epigenetic mediators largely overlap with the genes involved in stem cell reprogramming and their role in cancer followed directly from the discovery of their reprogramming role. Epigenetic mediators are those genes whose products are the targets of the epigenetic modifiers. Epigenetic modulators are the genes lying upstream of the modifiers and mediators in signaling and metabolic pathways

In some embodiments, an agent having activity as an epigenetic modulator is an HDAC inhibitor, a LSD-1 inhibitor, an EZH2 inhibitor, a DOT1L inhibitor, and a KDM inhibitor.

### HDAC Inhibitors

The one or more otic therapeutic agents in any embodiment disclosed could be one or more of the following HDAC inhibitors.

Histone deacetylases (HDAC) are a class of enzymes that remove acetyl groups (O═C—CH3) from an E-N-acetyl lysine amino acid on a histone, allowing the histones to wrap the DNA more tightly. This is important because DNA is wrapped around histones, and DNA expression is regulated by acetylation and de-acetylation.

HDACs are classified in four classes depending on sequence homology to the yeast original enzymes and domain organization. The HDAC classes include HDACI, HDAC IIA, HDAC IIB, HDAC III and HDAC IV.

Histone deacetylase (HDAC) inhibitors (HDACi, HDIs) are chemical compounds that inhibit histone deacetylases.

Thus, “HDAC inhibitor” refers to an agent capable of the decreasing the expression or enzymatic activity of HDAC. For example treatment with an HDAC inhibitor results in a decrease in histone deacetylation of a target gene in a cell.

In certain embodiments, the HDAC inhibitor decreases the expression or enzymatic activity of HDAC by at least 5, 10, 20, 30, 40, 50, 60, 70, 80, 90, or 100% relative to a control, for example relative to a baseline level of activity.

In certain embodiments, the HDAC inhibitor decreases histone deacetylation of a target gene by at least 5, 10, 20, 30, 40, 50, 60, 70, 80, 90, or 100% relative to a control, for example relative to a baseline level of activity.

In some embodiments, the HDAC inhibitor increases expression or activity of a target gene by at least 5, 10, 20, 30, 40, 50, 60, 70, 80, 90, or 100% relative to a control, for example relative to a baseline level of activity.

In some embodiments, the HDAC inhibitor decreases expression or enzymatic activity of HDAC by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the HDAC inhibitor decreases histone deacetylation of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the HDAC inhibitor increases expression or activity of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

TABLE 4

| | | Chemo- | Mechanism | Class | HDAC | | Lgr5+ | Perilymph | Formulation |
|---|---|---|---|---|---|---|---|---|---|
| Agent | CAS | type | HDAC Inhib | selectivity | Potenc | | Assay | Conc | Conc |
| Sodium | 1069-66-5 | Acid | 1, 2, 3, 8 | Class I | 39-161 | uM | 100 uM- | 100 uM- | 100 mM- |
| Valproate | | | | | | | 4 mM | 4 mM | 4000 mM |
| 2-hexyl-4- | 96017-59-3 | Acid | 1, 2, 3, 8 | Class I | 13 | uM | 100 uM- | 100 uM- | 100 mM- |
| pentynoic | | | | | | | 4 mM | 4 mM | 4000 mM |
| acid | | | | | | | | | |
| Na | 1716-12-7 | Acid | 1, 2, 3, 8 | Class I > | 9-16 | uM | 100 uM- | 100 uM- | 100 mM- |
| phenylbutyrate | | | | Class IIb | | | 4 mM | 4 mM | 4000 mM |

In various embodiments, the treatments disclosed herein include use an HDAC inhibitor. Exemplary HDAC inhibitors are provided in Table 17.

TABLE 17

| Class | Agent | CAS |
|---|---|---|
| Aliphatic Acid | Butyrate | 107-92-6 |
| Aliphatic Acid | Phenyl butyrate | 1821-12-1 |
| Aliphatic Acid | Valproic Acid | 99-66-1 |
| Aliphatic Acid Ester | AN-9 | 122110-53-6 |
| Amine | 932718-22-4 | 932718-22-4 |
| Benzamide | 4SC-202 | 1186222-89-8 |
| Benzamide | BML-210 | 537034-17-6 |
| Benzamide | Chidamide | 743438-44-0 |
| Benzamide | Entinostat (MS-275) | 209783-80-2 |
| Benzamide | HDAC Inhibitor IV | 537034-15-4 |
| Benzamide | Mocetinostat (MGCD0103) | 726169-73-9 |
| Benzamide | NKL 22 | 537034-15-4 |
| Benzamide | RGFP109 | 1215493-56-3 |
| Benzamide | RGFP136 | 1215493-97-2 |
| Benzamide | RGFP966 | 1357389-11-7 |
| Benzamide | Tacedinaline | 112522-64-2 |
| Benzamide | TC-H 106, HDAC Inhibitor VII | 937039-45-7 |
| Cyclic peptide | Apicidin | 183506-66-3 |
| Cyclic peptide | Dihydrochlamydocin | 52574-64-8 |
| Cyclic peptide | HC Toxin | 83209-65-8 |
| Cyclic peptide | Romidepsin | 128517-07-7 |
| Cyclic Peptide | Thailandepsin A | 1269219-30-8 |
| Cyclic peptide | Trapoxin A | 133155-89-2 |
| Epoxide | (−)-Depudecin | 139508-73-9 |
| Epoxide | Parthenolide | 20554-84-1 |
| Hydroxamate | (S)-HDAC-42 | 935881-37-1 |
| Hydroxamate | 4-(dimethylamino)-N-[6- | 193551-00-7 |
| | (hydroxyamino)-6-oxohexyl]- | |
| | benzamide | |
| Hydroxamate | 4-iodo-SAHA | 1219807-87-0 |
| Hydroxamate | 4SC-201 (Resminostat) | 864814-88-0 |
| Hydroxamate | ACY1215 | 1316214-52-4 |
| Hydroxamate | APHA Compound 8 | 676599-90-9 |
| Hydroxamate | BRD9757 | 1423058-85-8 |
| Hydroxamate | Bufexamac | 2438-72-4 |
| Hydroxamate | Butyrylhydroxamic acid | 4312-91-8 |
| Hydroxamate | CAY10603 | 1045792-66-2 |
| Hydroxamate | CBHA | 174664-65-4 |
| Hydroxamate | CG200745 | 936221-33-9 |
| Hydroxamate | CHR-3996 | 1256448-47-1 |
| Hydroxamate | CUDC-101 | 1012054-59-9 |
| Hydroxamate | Droxinostat | 99873-43-5 |
| Hydroxamate | HDAC Inhibitor II | 174664-65-4 |
| Hydroxamate | HDAC Inhibitor VI | 926908-04-5 |
| Hydroxamate | HDAC Inhibitor XXIV | 854779-95-6 |
| Hydroxamate | HDAC6 Inhibitor III | 1450618A9-1 |
| Hydroxamate | HDAC-IN-1 | 1239610A4-6 |
| Hydroxamate | HNHA | 926908-04-5 |
| Hydroxamate | HPOB | 1429651-50-2 |
| Hydroxamate | ITF2357 | 497833-27-9 |
| Hydroxamate | ITF2357 (Givinostat) | 497833-27-9 |
| Hydroxamate | LAQ-824 | 591207-53-3 |
| Hydroxamate | LBH-589 (panobinostat) | 404950-80-7 |
| Hydroxamate | LMK235 | 1418033-25-6 |
| Hydroxamate | M 344 | 251456-60-7 |
| Hydroxamate | MC 1568 | 852475-26-4 |
| Hydroxamate | Nexturastat A | 1403783-31-2 |
| Hydroxamate | NSC 57457 | 6953-61-3 |
| Hydroxamate | Oxamflatin | 151720-43-3 |
| Hydroxamate | PCI-24781 (Abexinostat) | 783355-60-2 |
| Hydroxamate | PCI-34051 | 950762-95-5 |
| Hydroxamate | PDX-101 (belinostat) | 866323-14-0 |
| Hydroxamate | Pyroxamide | 382180-17-8 |
| Hydroxamate | SAHA (Zolinza, vorinostat) | 149647-78-9 |
| Hydroxamate | SB939 (Pracinostat) | 929016-96-6 |
| Hydroxamate | SBHA | 38937-66-5 |
| Hydroxamate | Scriptaid | 287383-59-9 |
| Hydroxamate | Tefinostat (CHR-2845) | 914382-60-8 |
| Hydroxamate | Trichostatin A (TSA) | 58880-19-6 |
| Hydroxamate | Tubacin | 537049-40-4 |
| Hydroxamate | Tubastatin A | 1252003-15-8 |
| Hydroxamate | VAHA | 106132-78-9 |
| Ketone | Compound 43 | 891259-76-0 |
| Ketone - a- | 436150-82-2 | 436150-82-2 |
| ketoamides | | |
| Ketone - CF3 | Compound 27 | 946499-86-1 |
| Ketone - CF3 | Compound 6e | 946500-31-8 |
| Ketone - CF3 | Compound 6H | 946500-39-6 |
| Non classical | Tasquinimod | 254964-60-8 |
| Non classical | TMP269 | 1314890-29-3 |
| Polyketide | Ratjadone A | 163564-92-9 |
| Silylalcohol | 1587636-32-5 | 1587636-32-5 |
| Sulphonamide | 1587636-33-6 | 1587636-33-6 |
| Sulphonamide | 329967-25-1 | 329967-25-1 |
| Sulphonyl Urea | 960130-17-0 | 960130-17-0 |
| Thioester | HDAC Inhibitor XXII | 848354-66-5 |
| Thioester | KD 5170 | 940943-37-3 |
| Thioester | PTACH | 848354-66-5 |
| Thioester | TCS HDAC6 20b | 956154-63-5 |
| Thioketone | SIRT1/2 Inhibitor VII | 143034-06-4 |
| Thiol | 1368806-68-1 | 1368806-68-1 |
| Thiol | 1428536-05-3 | 1428536-05-3 |
| Thiol | 827036-76-0 | 827036-76-0 |
| Thiol | 828920-13-4 | 828920-13-4 |
| Thiol | 908860-21-9 | 908860-21-9 |
| Tropones | 1411673-95-4 | 1411673-95-4 |
| Tropones | 46189-88-2 | 46189-88-2 |

In some embodiments the HDAC inhibitor is a class I HDAC inhibitor. In these embodiments, the class I HDAC inhibitor may be a short chain carboxylic acid. In a preferred embodiment, the HDAC inhibitor is valproic acid (VPA), 2-hexyl-4-pentynoic acid, or Na phenylbutyrate. More preferably, the HDAC inhibitor is valproic acid (VPA). In certain such embodiments, the HDAC inhibitor is sodium valproate.

As used herein the terms “valproic acid” and “WA” are used interchangably to refer to the same compound. Moreover, as used herein the terms “valproic acid” and “WA” also refer any pharmaceutically acceptable salts thereof.

### LSD1 Inhibitors

The one or more otic therapeutic agents in any embodiment disclosed herein could be one or more of the following LSD1 inhibitors.

LSD1 mediated H3K4 demethylation can result in a repressive chromatin environment that silences gene expression. LSD1 has been shown to play a role in development in various contexts. LSD1 can interact with pluripotency factors in human embryonic stem cells and is important for decommissioning enhancers in stem cell differentiation. Beyond embryonic settings, LSD1 is also critical for hematopoietic differentiation. LSD1 is overexpressed in multiple cancer types and recent studies suggest inhibition of LSD1 reactivates the all-trans retinoic acid receptor pathway in acute myeloid leukemia (AML). These studies implicate LSD1 as a key regulator of the epigenome that modulates gene expression through post-translational modification of histones and through its presence in transcriptional complexes.

Thus, a “LSD1 inhibitor” refers to an agent capable of decreasing the expression or enzymatic activity of LSD1. For example a LSD1 inhibitor results in a decrease in H3K4 demethylation of a target gene in a cell, for instance, in a cochlear cell or a vestibular. cell

In certain embodiments, a LSD1 inhibitor decreases the expression or enzymatic activity of LSD1 by at least 5, 10, 20, 30, 40, 50, 60, 70, 80, 90, or 100% relative to a control, for example relative to a baseline level of activity.

In certain embodiments, a LSD1 inhibitor decreases H3K4 demethylation by at least 5, 10, 20, 30, 40, 50, 60, 70, 80, 90, or 100% relative to a control, for example relative to a baseline level of activity.

In some instances, a LSD1 inhibitor decreases H3K4 demethylation by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, or 1000-fold or more relative to a control, for example relative to a baseline level of activity.

In some instances, a LSD1 inhibitor modulates (i.e., increases or decreases) expression or activity of a target gene by at least 5, 10, 20, 30, 40, 50, 60, 70, 80, 90, or 100% relative to a control, for example relative to a baseline level of activity.

In some instances, a LSD1 inhibitor modulates (i.e., increases or decreases) expression or enzymatic activity of LSD1 by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some instances the LSD1 inhibitor is reversible. In other instances the LSD1 inhibitor is irreversible.

Exemplary agents having activity as a LSD1 inhibitor are provided in Table 18 below, including pharmaceutically-acceptable salts thereof

TABLE 18

| | | | | | | | | Select | | |
|---|---|---|---|---|---|---|---|---|---|---|
| | | pKi or | | Reversible or | | Select | | MAOs | | Literat. |
| Agent | CAS | IC50 | | Irreversible | Chemo-type | KDM1b | | A and B | | Cell |
| GSK-2879552 | 1401966-69-5 | 1.7 uM | | Irreversible | Cyproylamine | | | 20 | uM | EC50 = |
| | | (0.11 uM) | | | | | | | | 2-240 nM |
| GSK-LSD1 | 1431368-48-7 | 16 | nM | Irreversible | Cyproylamine | >1000X | | >1000X | | |
| Phenelzine | 51-71-8 | 5.6 | uM | Irreversible | Hydrazine | | | MAO | | 900 nM |
| sulfate | | | | | | | | inhibitor | | in Cell |
| | | | | | | | | 900 nM | | |
| | | | | | | | | in Cell | | |
| TCP | 155-09-9 | 11-477 | uM | Irreversible | Cyproylamine | 186 | uM | 1 | uM, | |
| (Tranylcypromine) | | | | | | | | | | |

| CC-90011 | 2179319-65-2 | | | Reversible | Likely | | | | | |
|---|---|---|---|---|---|---|---|---|---|---|
| | | | | | pyrimidinyl | | | | | |
| GCG-11047 | 308145-19-9 | | | Reversible | Polyamine | | | | | |
| (PG-11047) | | | | | | | | | | |
| IMG-7289 | 2229826-41-7 | | | Irreversible | Likely | | | | | |
| | | | | | Cyproylamine | | | | | |
| INCB059872 | 1802909-49-4 | | | Ineversible | Likely | | | | | |
| | | | | | Cyproylamine | | | | | |
| ORY-1001 | 1431326-61-2 | <20 | nM | Irreversible | Cyproylamine | >100 | uM | >100 uM | 0.5-3 | nM |
| (RG6016, | | | | | | | | >100 uM | | |
| RO7051790, | | | | | | | | | | |
| Iadademstat) | | | | | | | | | | |

| ORY-2001 | 1357362-02-7 | | | Irreversible | Cyproylamine | | |
|---|---|---|---|---|---|---|---|
| (Vafidemstat) | | | | | | | |
| Osimertinib | 1421373-65-0 | 3.98 | uM | Reversible | Pyrimidinyl | 43 | nM |
| (AZD9291) | | | | | | | |

| SP-2577 | 1423715-37-0 | | | Irreversible | Hydrazone | | | | |
|---|---|---|---|---|---|---|---|---|---|
| (Seclidemstat) | | | | | | | | | |
| | 1821307-10-1 | | | | | | | | |
| TCP Trans | 3721-28-6 | 284 | uM | Irreversible | Cyproylamine | 137 | uM | B:4.4 | uM |
| Chiral | | | | | | | | | |
| TCP Trans | 3721-26-4 | 168 | uM | Irreversible | Cyproylamine | 127 | uM | B:89 | uM |
| Chiral | | | | | | | | | |

| TCP Cis | 13531-35-6 | | | Irreversible | Cyproylamine | 11 uM | | | |
|---|---|---|---|---|---|---|---|---|---|
| | | | | | | 19 uM | | | |
| TCP Cis Chiral | 69684-88-4 | | | Irreversible | Cyproylamine | | | | |
| TCP Cis Chiral | 69684-89-5 | | | Irreversible | Cyproylamine | | | | |
| RN-1 | 1781835-13-9 | 70 | nM | Irreversible | Cyproylamine | 0.51 uM | | | |
| | | | | | | 2.78 uM | | | |
| Compound 1 | 1221595-26-1 | 10 | nM | Irreversible | Cyproylamine | | | | |
| Compound 45 | 1667721-01-8 | 9 | nM | Irreversible | Cyproylamine | 15 uM | | | |
| | | | | | | >40 uM | | | |
| RN-7 | 1352345-82-4 | 31 | nM | Irreversible | Cyproylamine | | | | |
| Compound 5A | 1613476-09-7 | 12 | nM | Irreversible | Cyproylamine | | | | |
| Compound 2 | 1235863-51-0 | 67 | nM | Irreversible | Cyproylamine | >37 | uM | | |
| Compound 43 | 1784703-61-2 | 610 | nM | Irreversible | Cyproylamine | | | | |
| Compound 12f | 1802319-25-0 | 86 | nM | Irreversible | Cyproylamine | 460 nM, | | | |
| | | | | | | >70 uM | | | |
| T-3775440 | 1422620-34-5 | 2.1 | nM | Irreversible | Cyproylamine | 110 uM, | | | |
| | | | | | | 17 uM | | | |
| OG-L002 | 1357299-45-6 | 20 | nM | Irreversible | Cyproylamine | | | | |
| S2101 | 1239262-36-2 | 990 | nM | Irreversible | Cyproylamine | | | | |
| NCL-1 | 1196119-03-5 | 1.6 | uM | Irreversible | Cyproylamine | | | | |
| Compound 9A | 2095849-74-2 | 1.2 | uM | Irreversible | Cyproylamine | | | | |
| Compound 191 | 2173543-81-0 | 0.97 | uM | Irreversible | Cyproylamine | | | | |
| NCD-25 | 1456972-46-5 | 480 | nM | Irreversible | Cyproylamine | | | | |
| NCD-38 | 2078047-42-2 | 590 | nM | Irreversible | Cyproylamine | | | | |
| Compound 14A | 2247939-53-1 | 2.2 | nM | Irreversible | Cyproylamine | | | | |
| Compound 15A | 2247939-55-3 | 70 | nM | Irreversible | Cyproylamine | | | | |
| Compound 15B | 2247939-56-4 | 11 | nM | Irreversible | Cyproylamine | | | | |
| Compound 4 | 2226461-60-3 | 43 | nM | Irreversible | Cyproylamine | | | 3.8 | uM |
| Pargyline | 555-57-7 | 1000 | uM | Irreversible | Amino-propyne | | | | |
| Peptide | 945548-35-6 | | | Irreversible | Amino-propyne | | | | |
| Bizine | 1591932-50-1 | 59 | nM | Irreversible | Hydrazine | | | | |
| Compound 5a | 1990536-90-7 | 1.4 | nM | Reversible | Hydrazone | | | | |
| Compound 5n | 1990537-03-5 | 1.7 | nM | Reversible | Hydrazone | | | | |
| SP-2509 | 1423715-09-6 | 13 | nM | Reversible | Hydrazone | >300 | uM | 350-650 | nM |
| (HCI-2509) | | | | | | | | | |

| LSD1-IN-32 | 2137044-49-4 | 83 | nM | Reversible | Amide | | 670 | nM |
|---|---|---|---|---|---|---|---|---|
| LSD1-IN-11p | 2101951-67-9 | 20-80 | nM | Reversible | Pyrazole | | 0.52 | uM |
| Resveratrol | 501-36-0 | 15 | uM | Reversible | Resveratrol | | | |
| Hydroxylamine | 2035912-55-9 | 121 | nM | Reversible | Resveratrol | | | |
| Compound 8c | 2170023-28-4 | 283 | nM | Reversible | Resveratrol | | 5 to 9 | |
| | | | | | | | μM | |
| CBB-1007 | 1379573-92-8 | 2.1 | μM | Reversible | Polyamine | | IC50 < | |
| | | | | | | | 5 μM | |
| Namoline | 342795-11-3 | 51 | μM | Reversible | Benzopyran-4- | | | |
| | | | | | one | | | |
| GSK-354 | 1841508-96-0 | 29-80 | nM | Reversible | Diphpyridine | A > 50 uM | 1.3 | μM |
| | | | | | | B = 19 uM | | |
| GSK-690 | 2101305-84-2 | 37 | nM | Reversible | Diphpyridine | | | |
| E11 | 1239589-91-3 | 243 | nM | Reversible | 2,4- | | | |
| | | | | | Quinazolinediamine | | | |
| MC2694 | 1435055-66-5 | 1 | uM | Reversible | 2,4- | | | |
| | | | | | Quinazolinediamine | | | |
| Alpha-mangostin | 11/1/6147 | 2.8 | uM | Reversible | mangostin | | | |
| Compound 12 A | 1923750-07-5 | 0.41 | uM | Reversible | Barbituate | | | |
| Compound 4 | 126118-57-8 | 6.4 | uM | Reversible | Purine-2,6-dione | | | |
| Compound 10d | 2226997-31-3 | 4 | uM | Reversible | Carboxamide | | | |
| Compound 90 | 1884266-15-2 | 162 | nM | Reversible | Carboxamide | | | |
| Compound 46 | 1884266-36-7 | 8 | nM | Reversible | Carboxamide | | 1-4 | uM |
| Compound 49 | 1884266-49-2 | 7 | nM | Reversible | Carboxamide | | 1-4 | uM |
| Compound 50 | 1884266-48-1 | 8 | nM | Reversible | Carboxamide | | 1-4 | uM |

| Polymyxin B | 1404-26-8 | 157 | nM | Reversible | Polymyxin B | | |
|---|---|---|---|---|---|---|---|
| Polymyxin E | 1066-17-7 | 193 | nM | Reversible | Polymyxin E | | |
| Baicalin | 21967-41-9 | 3.0 | uM | Reversible | Baicalin | | |
| Compound 16Q | 1612870-90-2 | 9.5 | uM | Reversible | Benzenesulfonamide | >500 | uM |
| LSD1 inhibitor | 1853269-07-4 | 1 | nM | Reversible | Imidazole | | |
| 24 | | | | | | | |
| geranylgeranoic | 35750-48-2 | 120 | uM | Reversible | Geranyl | | |
| acid | | | | | | | |
| Geranylgeraniol | 24034-73-9 | 80 | uM | Reversible | Geranyl | | |
| Thiocarbamate | 1430852-56-4 | 390 | nM | Reversible | Thiocarbamate | >1250 | uM |
| Thiourea | 1637373-61-5 | 650 | nM | Reversible | Thiourea | >1250 | uM |

| Thiourea | 2035417-23-1 | 154 | nM | Reversible | Thiourea | | | |
|---|---|---|---|---|---|---|---|---|
| Thienopyrrole | 1206028-57-0 | 2.9 | uM | Reversible | Thienopyrrole | >100 | uM | >100 uM, |
| | | | | | | | | 57 uM |
| Thienopyrrole | 1884266-15-2 | 162 | nM | Reversible | Thienopyrrole | | | |
| Thienopyrrole | 1884266-48-1 | 7.8 | nM | Reversible | Thienopyrrole | 13 | uM | 41 uM, |
| | | | | | | | | 100 uM |

| | | | | | |
|---|---|---|---|---|---|
| 4SC-202 | 910462-43-0 | 1-10 | uM | Reversible | o-aminoph |
| ORY-3001 | 2179325-30-3 | | | | |
| JL1037 | | | | | |
| FLI-06 | 313967-18-9 | 92 | nM | Inhibits | Dihydropyridine |
| | | | | expression of | |
| | | | | LSD1 | |
| Rhodium | | 40 | nM | | Rhodium |
| Complex 1 | | | | | |

| | | | | | | Formul. | | Human | |
|---|---|---|---|---|---|---|---|---|---|
| | | Lgr5+ | | Perilymph | | Conc. | | Plasma | Human |
| Agent | CAS | Assay | | Conc. | | Intraymp. | | Conc | Dosage |
| GSK-2879552 | 1401966-69-5 | 40 nM- | | 40 nM- | | 40 uM to | | 1-100 nM | 1 or 2 |
| | | 30 uM | | 30 μM | | 30 mM | | | mg QD |
| | | | | | | | | | PO |
| GSK-LSD1 | 143136848-7 | 4 nM- | | 4 nM- | | 4 uM to | | 1-100 nM | 10-100 |
| | | 50 uM | | 50 uM | | 50 mM | | | mg PO |
| Phenelzine | 51-71-8 | | | 0.1-10 | uM | 0.1-10 | mM | Cmax 10 | 15-90 |
| sulfate | | | | | | | | to 60 | mg/day |
| | | | | | | | | ng/mL | PO |
| | | | | | | | | (73-440 | |
| | | | | | | | | nM) | |
| TCP | 155-09-9 | 0.1-20 | uM | 0.1-20 | uM | 0.1-20 | mM | Cmax | 15-150 |
| (Tranylcypromine) | | | | | | | | 30-200 | mg/day |
| | | | | | | | | ng/ml | PO |
| | | | | | | | | (225- | |
| | | | | | | | | 1500 nM) | |

| CC-90011 | 2179319-65-2 | | | | |
|---|---|---|---|---|---|
| GCG-11047 | 308145-19-9 | | | | |
| (PG-11047) | | | | | |
| IMG-7289 | 2229826-41-7 | | Cmax 63 | 80 mg | |
| | | | ng/ml | QD PO | |
| INCB059872 | 1802909-49-4 | | | | |
| ORY-1001 | 1431326-61-2 | Activity | | Up to | |
| (RG6016, | | | | 2 mg | |
| RO7051790, | | | | | |
| Iadademstat) | | | | | |
| ORY-2001 | 1357362-02-7 | | | | |
| (Vafidemstat) | | | | | |
| Osimertinib | 1421373-65-0 | | | 10-80 | mg |
| (AZD9291) | | | | | |
| SP-2577 | 1423715-37-0 | | | | |
| (Seclidemstat) | | | | | |
| | 1821307-10-1 | | | | |
| TCP Trans | 3721-28-6 | | | | |
| Chiral | | | | | |
| TCP Trans | 3721-26-4 | | | | |
| Chiral | | | | | |
| TCP Cis | 13531-35-6 | | | | |
| TCP Cis Chiral | 69684-88-4 | | | | |
| TCP Cis Chiral | 69684-89-5 | | | | |
| RN-1 | 1781835-13-9 | FHZ-455 | | | |
| Compound 1 | 1221595-26-1 | | | | |
| Compound 45 | 1667721-01-8 | | | | |
| RN-7 | 1352345-82-4 | | | | |
| Compound 5A | 1613476-09-7 | | | | |
| Compound 2 | 1235863-51-0 | | | | |
| Compound 43 | 1784703-61-2 | | | | |
| Compound 12f | 1802319-25-0 | | | | |
| T-3775440 | 1422620-34-5 | | | | |
| OG-L002 | 1357299-45-6 | | | | |
| S2101 | 1239262-36-2 | | | | |
| NCL-1 | 1196119-03-5 | | | | |
| Compound 9A | 2095849-74-2 | | | | |
| Compound 191 | 2173543-81-0 | | | | |
| NCD-25 | 1456972-46-5 | | | | |
| NCD-38 | 2078047-42-2 | | | | |
| Compound 14A | 2247939-53-1 | | | | |
| Compound 15A | 2247939-55-3 | | | | |
| Compound 15B | 2247939-56-4 | | | | |
| Compound 4 | 2226461-60-3 | | | | |
| Pargyline | 555-57-7 | | | | |
| Peptide | 945548-35-6 | | | | |
| Bizine | 1591932-50-1 | FHZ-457 | | | |
| Compound 5a | 1990536-90-7 | | | | |
| Compound 5n | 1990537-03-5 | | | | |
| SP-2509 | 1423715-09-6 | Activity | | | |
| (HCI-2509) | | | | | |
| LSD1-IN-32 | 2137044-49-4 | | | | |
| LSD1-IN-11p | 2101951-67-9 | | | | |
| Resveratrol | 501-36-0 | | | | |
| Hydroxylamine | 2035912-55-9 | | | | |
| Compound 8c | 2170023-28-4 | | | | |
| CBB-1007 | 1379573-92-8 | Activity | | | |
| Namoline | 342795-11-3 | | | | |
| GSK-354 | 1841508-96-0 | | | | |
| GSK-690 | 2101305-84-2 | | | | |
| E11 | 1239589-91-3 | | | | |
| MC2694 | 1435055-66-5 | | | | |
| Alpha-mangostin | 11/1/6147 | | | | |
| Compound 12 A | 1923750-07-5 | | | | |
| Compound 4 | 126118-57-8 | | | | |
| Compound 10d | 2226997-31-3 | | | | |
| Compound 90 | 1884266-15-2 | | | | |
| Compound 46 | 1884266-36-7 | | | | |
| Compound 49 | 1884266-49-2 | | | | |
| Compound 50 | 1884266-48-1 | | | | |
| Polymyxin B | 1404-26-8 | | | | |
| Polymyxin E | 1066-17-7 | | | | |
| Baicalin | 21967-41-9 | | | | |
| Compound 16Q | 1612870-90-2 | | | | |
| LSD1 inhibitor | 1853269-07-4 | | | | |
| 24 | | | | | |
| geranylgeranoic | 35750-48-2 | | | | |
| acid | | | | | |
| Geranylgeraniol | 24034-73-9 | | | | |
| Thiocarbamate | 1430852-56-4 | | | | |
| Thiourea | 1637373-61-5 | | | | |
| Thiourea | 2035417-23-1 | | | | |
| Thienopyrrole | 1206028-57-0 | | | | |
| Thienopyrrole | 1884266-15-2 | | | | |
| Thienopyrrole | 1884266-48-1 | | | | |
| 4SC-202 | 910462-43-0 | | | 25400 | mg/Day |

| | |
|---|---|
| ORY-3001 | 2179325-30-3 |
| JL1037 | |
| FLI-06 | 313967-18-9 |
| Rhodium | |
| Complex 1 | |

In some embodiments, an agent having activity as a LSD1 inhibitor is GSK-2879552, GSK-LSD1, Osimertinib (AZD9291), Phenelzine sulfate, Tranylcypromine (TCP), ORY-1001, Seclidemstat (SP-2577), Vafidemstat (ORY-2001), CC-90011, IMG-7289 or, INCB059872. Preferably, the LSD1 inhibitor is GSK-2879552, GSK-LSD1, Phenelzine sulfate or Tranylcypromine (TCP).

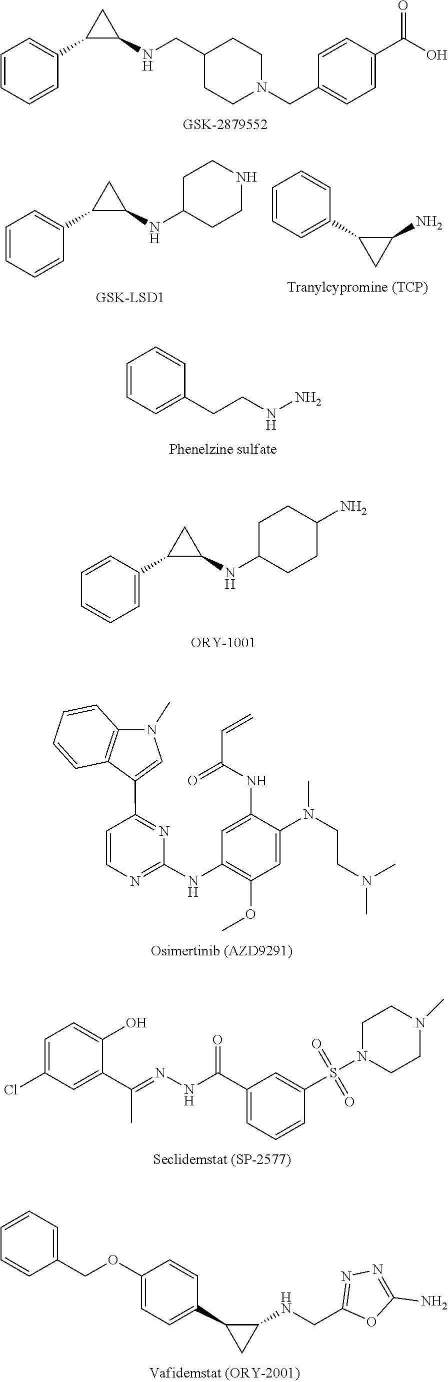

More preferably, the LSD1 inhibitor is GSK-2879552, GSK-LSD-1, or Tranylcypromine (TCP).

### EZH2 Inhibitors

The one or more otic therapeutic agents in any embodiment disclosed herein could be one or more of the following EZH2 inhibitors.

Enhancer of zeste homolog 2 (EZH2) is a histone-lysine N-methyltransferase enzyme encoded by an EZH2 gene, that participates in histone methylation and, ultimately, transcriptional repression. EZH2 catalyzes the addition of methyl groups to histone H3 at lysine 27, by using the cofactor S-adenosyl-L-methionine. Methylation activity of EZH2 facilitates heterochromatin formation thereby silences gene function. Remodeling of chromosomal heterochromatin by EZH2 is also required during cell mitosis.

EZH2 is the functional enzymatic component of the Polycomb Repressive Complex 2 (PRC2), which is responsible for healthy embryonic development through the epigenetic maintenance of genes responsible for regulating development and differentiation. EZH2 is responsible for the methylation activity of PRC2, and the complex also contains proteins required for optimal function (EED, SUZ12, JARID2, AEBP2, RbAp46/48, and PCL).

EZH2 inhibitors are chemical compounds that inhibit histone-lysine N-methyltransferase enzyme encoded by EZH2 gene

Thus, “EZH2 inhibitor” refers to an agent capable of the decreasing the expression or enzymatic activity of EZH2. For example, an EZH2 inhibitor results in a decrease in histone methylation of a target gene in a cell.

In certain embodiments, the EZH2 inhibitor decreases the expression or enzymatic activity of EZH2 by at least 5, 10, 20, 30, 40, 50, 60, 70, 80, 90, or 100% relative to a control, for example relative to a baseline level of activity.

In certain embodiments, the EZH2 inhibitor decreases histone methylation of a target gene by at least 5, 10, 20, 30, 40, 50, 60, 70, 80, 90, or 100% relative to a control, for example relative to a baseline level of activity.

In some embodiments, the EZH2 inhibitor increases expression or activity of a target gene by at least 5, 10, 20, 30, 40, 50, 60, 70, 80, 90, or 100% relative to a control, for example relative to a baseline level of activity.

In some embodiments, the EZH2 inhibitor decreases expression or enzymatic activity of EZH2 by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the EZH2 inhibitor decreases histone methylation of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the EZH2 inhibitor increases expression or activity of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

Exemplary EZH2 inhibitors are provided in Table 19.

TABLE 19

| | | pKi | | | | Select | | Lit | | |
|---|---|---|---|---|---|---|---|---|---|---|
| | | or | | Enzymatic/ | | vs | | Cell | | Lgr5+ |
| Agent | CAS | IC50 | | Non-enzymatic | Chemo-type | EZH-1 | | Poten | | Assay |
| PF-06821497 | 1844849-10-0 | <1 | nM | enzymatic | 2-Pyridone | 70 | nM | 4-6 | nM | |
| CPI-1205 | 1621862-70-1 | 2.2 | nM | enzymatic | 2-Pyridone | 24x | | 32 | nM | |
| Valemetostat | 1809336-39-7 | 2.5 | nM | enzymatic | 2-Pyridone | 8.4 | nM | 25-250 | nM | |
| (DS-3201b, | | | | | | | | | | |
| (R)-OR-S2) | | | | | | | | | | |

| | | | | | | | | |
|---|---|---|---|---|---|---|---|---|
| Tazemetostat | 1403254-99-8 | 2.5 | nM | enzymatic | 2-Pyridone | 35x | 0.37-1.1 | uM |
| (EPZ-6438) | | | | | | | | |
| El1 | 1418308-27-6 | 13 | nM | enzymatic | 2-Pyridone | 90x | 5 | uM |

| | | | | | | | |
|---|---|---|---|---|---|---|---|
| CPI-169 | | 0.24 | nM | enzymatic | 2-Pyridone | 6 | nM |
| (R)-OR-S1 | 1809336-19-3 | 10 | nM | enzymatic | 2-Pyridone | 7.4 | nM |

| A-395 | 2089148-72-9 | 0.3 | nM | EED | Amino | 90 | nM |
|---|---|---|---|---|---|---|---|
| | | | | Inhibit | pyrrolidines | | |
| Astemizole | 68844-77-9 | 94 | uM | EED | Benzimidazole | | |
| | | | | Inhibit | | | |
| Compound 19 | 2079895-22-8 | 1.3 | uM | EED | Imidazole | 1.9 | uM |
| | | | | Inhibit | | | |

| Compound 22 | 1802175-07-0 | 2 | nM | enzymatic | 2-Pyridone | | | | |
|---|---|---|---|---|---|---|---|---|---|
| Compound 24 | 1659298-29-9 | 40 | nM | enzymatic | 2-Pyridone | | | | |
| Compound 34 | 2055347-72-1 | 29 | nM | enzymatic | 2-Pyridone | >100x | | | |
| Compound 41 | 2055347-94-7 | 11 | nM | enzymatic | 2-Pyridone | >100x | | | |
| CPI-0169 | 1450655-76-1 | 0.24 | nM | enzymatic | 2-Pyridone | 6 | nM | | |
| CPI-0169 | 1802175-07-0 | 0.24 | nM | enzymatic | 2-Pyridone | 6 | nM | 1.1 | uM |

| CPI-0209 | | | | | | | |
|---|---|---|---|---|---|---|---|
| CPI-360 | 1802175-06-9 | 0.5 | nM | enzymatic | 2-Pyridone | ~50 | nM |

| EBI-2511 | 2098546-05-3 | 4 | nM | enzymatic | 2-Pyridone | | | | |
|---|---|---|---|---|---|---|---|---|---|
| EED162 | 1010897-73-0 | 30 | nM | EED | Triazo | | 80 | nM | |
| | | | | Inhibit | | | | | |
| EED226 | 2083627-02-3 | | | EED | Triazo | | | | Activity |
| | | | | Inhibit | | | | | |
| EPZ-005687 | 1396772-26-1 | 24 | nM | enzymatic | 2-Pyridone | 50X | | | |
| EPZ-011989 | 1598383-40-4 | <3 | nM | enzymatic | 2-Pyridone | | 94 | nM | |
| GSK126 | 1346574-57-9 | <3 | nM | enzymatic | 2-Pyridone | 150x | | | |
| GSK343 | 1346704-33-3 | 1.2 | nM | enzymatic | 2-Pyridone | 60x | 174 | nM | |
| GSK503 | 1346572-63-1 | <10 | nM | enzymatic | 2-Pyridone | | | | |
| GSK926 | 1346704-13-9 | 7.9 | nM | enzymatic | 2-Pyridone | | 324 | nM | |
| MAK683 | 1951408-58-4 | | | EED | Triazo | | | | |
| (EED162) | (likely patent) | | | inhibitor | | | | | |
| SHR2554 | 2098545-98-1 | | | enzymatic | 2-Pyridone | | | | |
| SKLB1049 | 1826865-42-2 | 7.2 | nM | enzymatic | 2-Pyridone | | 12 | uM | |
| ZLD1039 | 1826865-46-6 | <15 | nM | enzymatic | 2-Pyridone | | | | |
| ZLD1122 | 1826865-51-3 | <15 | nM | enzymatic | 2-Pyridone | | | | |
| | 1404094-15-0 | 74 | nM | enzymatic | 2-Pyridone | | 2510 | nM | |
| | 1404094-16-1 | 14 | nM | enzymatic | 2-Pyridone | | 1995 | nM | |
| DZNep | 102052-95-9 | | | SAH- | SAH derived | | 1 | uM | Activity |
| | | | | hydrolase | | | | | |
| | | | | inhibitor | | | | | |
| Cmpd 44 | 1378002-93-7 | 32 | nM | SAM | Benzamide | | 9 | uM | |
| | | | | Comp | | | | | |
| Compound 27 | 1676100-59-6 | 270 | nM | SAH- | SAH derived | | | | |
| | | | | hydrolase | | | | | |
| | | | | inhibitor | | | | | |
| Sinefungin | 58944-73-3 | 20 | nM | SAH- | SAH derived | | 33 | nM | |
| | | | | hydrolase | | | | | |
| | | | | inhibitor | | | | | |
| Tanshindiol B | 97465-70-8 | 520 | nM | enzymatic | Tanshindiols | | | | |
| Tanshindiol C | 97465-71-9 | 550 | nM | enzymatic | Tanshindiols | | | | |
| UNCI 999 | 1431612-23-5 | 10 | nM | enzymatic | 2-Pyridone | 10x | 124 | nM | Activity |

| | | | |
|---|---|---|---|
| (−)- | 989-51-5 | enzymatic | a,b-unsat |
| Epigallocatechin- | | | |
| 3-gallate | | | |
| (EGCG) | | | |
| Curcumin | 458-37-7 | enzymatic | a,b-unsat |
| MC1945 | 169903-68-8 | enzymatic | a,b-unsat |
| MC1947 | 949090-12-4 | Non-enzymatic | |
| MC1948 | 949090-20-4 | Non-enzymatic | |
| SAH-EZH2 | | Non-enzymatic | reactive |
| Sulfoiaphane | 4478-93-7 | EED | Stapled |
| | | Inhibit | Peptide |

| | | | | Formul. | | | | |
|---|---|---|---|---|---|---|---|---|
| | | Perilymph | | Conc. | | Human In | | Human |
| Agent | CAS | Conc | | Intraymp | | Vivo Conc | | Dosage |
| PF-06821497 | 1844849-10-0 | 1-100 | nM | 1-100 | uM | 5-50 | nM | 75 mg to |
| | | | | | | | | 625 mg BID |
| | | | | | | | | PO |
| CPI-1205 | 1621862-70-1 | 10-1000 | nM | 10-1000 | uM | 25-250 | nM | 800 mg BID |
| | | | | | | | | and |
| | | | | | | | | subsequently |
| | | | | | | | | TID-PO |
| Valemetostat | 1809336-39-7 | 10-1000 | nM | 10-1000 | uM | 25-250 | nM | PO starting |
| (DS-3201b, | | | | | | | | dose of 100 |
| (R)-OR-S2) | | | | | | | | mg QD with |
| | | | | | | | | dose escal |
| | | | | | | | | dep on tox |
| Tazemetostat | 1403254-99-8 | 0.37-1.1 | uM | 0.1-10 | mM | 100-800 ng/ml | | PO 100 BID |
| (EPZ-6438) | | | | | | (200-1600 nM) | | to 800 mg |
| | | | | | | | | BID. |
| El1 | 1418308-27-6 | 1-10 | uM | 1-10 | mM | 1-10 | uM | (100 to 1000/ |
| | | | | | | | | day mg PO) |
| CPI-169 | | 1-10 | uM | 1-10 | mM | 1-10 | uM | 100 to 1000/ |
| | | | | | | | | day mg PO |

| | | |
|---|---|---|
| (R)-OR-S1 | 1809336-19-3 | IV 50 mg- |
| | | poor oral |
| | | bio. |
| A-395 | 2089148-72-9 | 50 mg and |
| | | 200 mg PO |
| Astemizole | 68844-77-9 | |
| Compound 19 | 2079895-22-8 | |
| Compound 22 | 1802175-07-0 | |
| Compound 24 | 1659298-29-9 | |
| Compound 34 | 2055347-72-1 | |
| Compound 41 | 2055347-94-7 | |
| CPI-0169 | 1450655-76-1 | |
| CPI-0169 | 1802175-07-0 | |
| CPI-0209 | | |
| CPI-360 | 1802175-06-9 | |
| EBI-2511 | 2098546-05-3 | |
| EED162 | 1010897-73-0 | |
| EED226 | 2083627-02-3 | |
| EPZ-005687 | 1396772-26-1 | |
| EPZ-011989 | 1598383-40-4 | |
| GSK126 | 1346574-57-9 | |
| GSK343 | 1346704-33-3 | |
| GSK503 | 1346572-63-1 | |
| GSK926 | 1346704-13-9 | |
| MAK683 | 1951408-58-4 | |
| (EED162) | (likely patent) | |
| SHR2554 | 2098545-98-1 | |
| SKLB1049 | 1826865-42-2 | |
| ZLD1039 | 1826865-46-6 | |
| ZLD1122 | 1826865-51-3 | |
| | 1404094-15-0 | |
| | 1404094-16-1 | |
| DZNep | 102052-95-9 | |
| Cmpd 44 | 1378002-93-7 | |
| Compound 27 | 1676100-59-6 | |
| Sinefungin | 58944-73-3 | |
| Tanshindiol B | 97465-70-8 | |
| Tanshindiol C | 97465-71-9 | |
| UNCI 999 | 1431612-23-5 | |
| (−)- | 989-51-5 | |
| Epigallocatechin- | | |
| 3-gallate | | |
| (EGCG) | | |
| Curcumin | 458-37-7 | |
| MC1945 | 169903-68-8 | |
| MC1947 | 949090-12-4 | |
| MC1948 | 949090-20-4 | |
| SAH-EZH2 | | |
| Sulfoiaphane | 4478-93-7 | |

In some embodiments the EZH2 inhibitor is PF-06821497, CPI-120, Valemetostat, Tazemetostat or Ell.

### DOT1L Inhibitors

The one or more otic therapeutic agents in any embodiment disclosed could be one or more of the following DOT1L inhibitors.

DOT1-like (Disruptor of telomeric silencing 1-like), histone H3K79 methyltransferase (S. cerevisiae), also known as DOT1L, is a protein found in humans, as well as other eukaryotes. The methylation of histone H3 lysine 79 (H3K79) by DOT1L which is a conserved epigenetic mark in many eukaryotic epigenomes, increases progressively along the aging process.

DOT1L inhibitors are chemical compounds that inhibits histone H3K79 methyltransferase

Thus, “DOT1L inhibitor” refers to an agent capable of the decreasing the expression or enzymatic activity of DOT1L. For example, an EZH2 inhibitor results in a decrease in histone methylation of a target gene in a cell.

In certain embodiments, the DOT1L inhibitor decreases the expression or enzymatic activity of DOT1L by at least 5, 10, 20, 30, 40, 50, 60, 70, 80, 90, or 100% relative to a control, for example relative to a baseline level of activity.

In certain embodiments, the DOT1L inhibitor decreases histone methylation of a target gene by at least 5, 10, 20, 30, 40, 50, 60, 70, 80, 90, or 100% relative to a control, for example relative to a baseline level of activity.

In some embodiments, the DOT1L inhibitor increases expression or activity of a target gene by at least 5, 10, 20, 30, 40, 50, 60, 70, 80, 90, or 100% relative to a control, for example relative to a baseline level of activity.

In some embodiments, the DOT1L inhibitor decreases expression or enzymatic activity of DOT1L by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the DOT1L inhibitor decreases histone methylation of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the DOT1L inhibitor increases expression or activity of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

Exemplary DOT1L inhibitors are provided in Table 20.

TABLE 20

| | | pKi | | | | | | | | | Formulation | | Human | |
|---|---|---|---|---|---|---|---|---|---|---|---|---|---|---|
| | | or | | | Lit | | Lgr5+ | | Perilymph | | Conc. | | In Vivo | Human |
| Agent | CAS | IC50 | | Chemo-type | Cell | | Assay | | Conc | | Intratypm | | Conc | Dosage |
| EPZ004777 | 1338466-77-5 | 0.3 | nM | Adenosine | 11 | nM | 0.6-45 | uM | 0.6-45 | uM | 0.1-45 | mM | 0.1-45 uM | 10-100 |
| | | | | | | | | | | | | | | mg/m2 |
| | | | | | | | | | | | | | | per day |
| | | | | | | | | | | | | | | IV |
| Pinometostat | 1380288-87-8 | 0.08 | nM | Adenosine | 2.7 | nM | | | 0.1-10 | uM | 0.1-10 | mM | Total | 54-90 |
| (EPZ-5676) | | | | | | | | | | | | | plasma | mg/m2 |
| | | | | | | | | | | | | | Css | per day |
| | | | | | | | | | | | | | 800- | by |
| | | | | | | | | | | | | | 1600 | continuous |
| | | | | | | | | | | | | | ng/mL | IV, |
| | | | | | | | | | | | | | (1.42- | Potential |
| | | | | | | | | | | | | | 2.94 | for SC |
| | | | | | | | | | | | | | uM) | dosing |
| | | | | | | | | | | | | | (1-10 | |
| | | | | | | | | | | | | | uM) | |
| SGC0946 | 1561178-17-3 | 0.3 | nM | Adenosine | 10 | nM | 0.6-5 | uM | 0.6-5 | uM | 0.6-5 | mM | 0.1-5 uM | 10-100 |
| | | | | | | | | | | | | | | mg/m2 |
| | | | | | | | | | | | | | | per day |
| | | | | | | | | | | | | | | IV |

| Bromo- | 1428254-21-0 | 77 | nM | Adenosine | | |
|---|---|---|---|---|---|---|
| deaza- | | | | | | |
| SAH | | | | | | |
| CN SAH | 1985669-27-9 | 13 | nM | Adenosine | | |
| Compound | 1645266-99-4 | 29 | nM | Adenosine | | |
| 10 | | | | | | |
| Compound | 1940206-71-2 | 0.4 | nM | Aminopyrimidine | | |
| 13 | | | | | | |
| Compound | 2088518-50-5 | <1 | nM | pyrrolopyrimidine | | |
| 7 | | | | | | |
| Compound | 1940224-84-9 | 14 | nM | Acetylene | | |
| 8 | | | | | | |
| EPZ002696 | 1381760-94-6 | 13 | nM | Adenosine | | |
| EPZ004450 | 1380315-97-8 | 4 | nM | Adenosine | | |
| SAH | 979-92-0 | 600 | nM | Adenosine | | |
| SYC-522 | 1381761-52-9 | 0.76 | nM | Adenosine | 6 | uM |
| SYC-687 | 1440509-94-3 | 1.1 | nM | Non-Ribose | 200 | nM |
| | 1440510-03-1, | 1.1 | nM | Adenosine | 200 | nM |
| | 1440509-94-3 | | | | | |

| Compound | | | | Peptides |
|---|---|---|---|---|
| 21 | | | | |
| Compound | | | | Peptides |
| 28 | | | | |
| Compound | 167558-34-1 | 8.3 | uM | triazolothiadiazol |
| 6 | | | | |
| Compound | | | | pyrimidylaminoquinoline |
| 8H | | | | |
| | 1163729-79-0 | 1.5 | uM | pyrimidine |

In some embodiments the DOT1L inhibitor is EPZ004777, Pinometostat or SGC0946.

### KDM Inhibitors

The one or more otic therapeutic agents in any embodiment disclosed could be one or more of the following KDM inhibitors.

About 30 JmjC domain-containing proteins have been identified as lysine demethylases in the human genome. Based on histone lysine sites and demethylation states, the JmjC domain-containing protein family is divided into six subfamilies: KDM2, KDM3, KDM4, KDM5, KDM6 and PHF. The JmjC domain-containing proteins belong to the Fe(II) and 2-oxoglutarate (2-OG)-dependent dioxygenases, which demethylate a variety of targets, including histones (H3K4, H3K9, H3K27, H3K36 as well as H1K26) and non-histone proteins. Unlike the LSD family, the Jmj C-domain-containing histone demethylases (JHDMs) are able to erase all three kinds of histone lysine-methylation states since the JHDMs do not require protonated nitrogen for demethylation.

The KDM2 (also named FBXL) subfamily includes two members: KDM2A and KDM2B. KDM4 gene family, first identified in silico, consists of six members, including KDM4A, KDM4B, KDM4C, KDM4D, KDM4E and KDM4F. The KDM5 subfamily contains four enzymes: KDM5A, KDM5B, KDM5C and KDM5D, which specifically remove methyl marks from H3K4me2/3. In the human genome, the KDM6 subfamily is comprised of KDM6A, KDM6B and UTY, which share a well-conserved JmjC histone catalytic domain.

KDM inhibitors are chemical compounds that inhibits lysine demethylases.

Thus, “KDM inhibitor” refers to an agent capable of the decreasing the expression or enzymatic activity of KDM. For example, an KDM inhibitor results in a decrease in histone demethylation of a target gene in a cell.

In certain embodiments, the KDM inhibitor decreases the expression or enzymatic activity of KDM by at least 5, 10, 20, 30, 40, 50, 60, 70, 80, 90, or 100% relative to a control, for example relative to a baseline level of activity.

In certain embodiments, the KDM inhibitor decreases histone demethylation of a target gene by at least 5, 10, 20, 30, 40, 50, 60, 70, 80, 90, or 100% relative to a control, for example relative to a baseline level of activity.

In some embodiments, the KDM inhibitor increases expression or activity of a target gene by at least 5, 10, 20, 30, 40, 50, 60, 70, 80, 90, or 100% relative to a control, for example relative to a baseline level of activity.

In some embodiments, the KDM inhibitor decreases expression or enzymatic activity of KDM by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the KDM inhibitor decreases histone demethylation of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the KDM inhibitor increases expression or activity of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity. Exemplary KDM inhibitors are provide in Table 21.

TABLE 21

| | | | Covalent | Select | Select | | Select | Select | | Select | | Select | | Select |
|---|---|---|---|---|---|---|---|---|---|---|---|---|---|---|
| Agent | CAS | Chemo-type | or not | KDM 1 | KDM 2 | | KDM 3 | KDM 4 | | KDM 5 | | KDM 6 | | KDM 7 |
| AS 8351 | 796-42-9 | Hydrazone | | | | | | | | | | | | |
| TC-E5002 | 1453071-47-0 | Hydioamate | No | | 6.8 | uM | | 83 | uM | 55 | uM | >100 | uM | 200, |
| | | | | | | | | | | | | | | 1200 |
| | | | | | | | | | | | | | | nM |

| EPT- | | | | 20-50 | | <1 | nM | 3K |
|---|---|---|---|---|---|---|---|---|
| 103182 | | | | | | | | |
| Compound | 1844064-06-7 | pyrimidin-4-one | No | 102/31 | nM | 23 | nM | |
| 54k | | | | | | | | |

| | | | | | | | | | |
|---|---|---|---|---|---|---|---|---|---|
| Cmpd 1 | 1516899-38-9 | Cyproamine | Yes | 220 | nM | 190 | nM | 220 | nM |
| | | isonicotinic acid | | | | | | | |

| Cmpd 105 | 1613514-89-8 | Isonicotinic acid | No | | | <100 | nM |
|---|---|---|---|---|---|---|---|
| Compound | 1461602-86-7 | Isonicotinic acid | No | <100 | nM | <100 | nM |
| 34 | | | | | | | |
| Compound | 1628332-52-4 | pyridopyrimidinone | No | | | <100 | nM |
| 41 | | | | | | | |
| compound | 1628210-26-3 | cyanopyrazole | No | | | 15 | nM |
| 48 | | | | | | | |
| Compound | 1993438-65-5 | naphthyridones | No | | | 206 | nM |
| 18 | | | | | | | |
| Compound | 1613410-75-5 | pyrazolylpyridines | No | | | 10 | nM |
| 33 | | | | | | | |
| Compound | 1905482-57-6 | Amide | No | | | | |
| 48 | | | | | | | |
| Compound | 1905482-57-6 | Pyrazole | No | | | | |
| 48 | | | | | | | |
| Compound | 1905481-35-7 | Pyrazole | No | | | | |
| 49 | | | | | | | |
| Compound | 1905481-36-8 | Pyrazole | No | | | | |
| 50 | | | | | | | |
| Compound | 2169272-46-0 | 1-H-Indole | No | | | 50 | nM |
| 6 | | | | | | | |
| Compound | 1807514-47-1 | Triazole | No | | | 65 | nM |
| R-35 | | | | | | | |
| CPI-455 | 1628208-23-0 | cyanopyrazole | No | 41 | nM | 13, 2 | nM |

| CPI-4203 | 1628214-07-2 | cyanopyrazol | No | 1.1 | uM | | |
|---|---|---|---|---|---|---|---|
| E67-2 | 1364914-62-4 | Quinazoline | No | | | | |
| GSK467 | 1628332-52-4 | Pyrazole | No | 14 | nM | | |
| GSK-J1 | 1373422-53-7 | Acid | No | | | | |
| GSK-J4 | 1373423-53-0 | Ethyl Ester | No | 170 | nM | 28 | nM |

| KDM5- | 1596embodi- | Pyridine | No | | | | |
|---|---|---|---|---|---|---|---|
| C49 | ment-16-1 | | | | | | |
| KDM5- | 1596348-32-1 | Pyridine | No | | | | |
| C50 | | | | | | | |
| KDOAM25 | 2230731-99-2 | Amide | No | | | | |
| N11 | 1613515-45-9 | isonicotinic | No | | 65 | nM | |
| | 1807514-47-1 | Amide | No | | 90 | nM | |
| | 1844064-07-8 | Pyridopyrimidinone | No | | 45 | nM | |
| Compound | | Rh Complex | | | | | |
| 1 | | | | | | | |
| Compound | 1498996-89-6 | Hydrazine | | | X | | |
| 15e | | | | | | | |
| Daminozide | 1596-84-5 | Hydrazine | | X | | | X |
| JIB-04 | 99596-05-9 | Hydrazine | | | 220 | nM | |

| Methylstat | 1310877-95-2 | Unsatamide | | | |
|---|---|---|---|---|---|
| Compound | 2098902-68-0 | cyanopyrazole | No | | |
| 10r | | | | | |
| N71 | | | Yes | X | |
| NSC | | | | 410 | nM |
| 6369819 | | | | | |

| | | Select | Lit | | Lgr5+ | | Perilymph | | Formulation | | Human In | | Human |
|---|---|---|---|---|---|---|---|---|---|---|---|---|---|
| Agent | CAS | KDM 8 | Cell | | Assay | | Conc | | Conc | | Vivo Conc | | Dosage |
| AS 8351 | 796-42-9 | | | | 1-3 | uM | 1-3 | uM | 1-3 | mM | 1-3 | uM | 100- |
| | | | | | | | | | | | | | 2000 |
| | | | | | | | | | | | | | mg/day |
| TC-E5002 | 1453071-47-0 | | 1640 | uM | 0.12-10 | uM | 0.12-10 | uM | 0.12-10 | mM | 0.12-10 | uM | 100- |
| | | | | | | | | | | | | | 1000 |
| | | | | | | | | | | | | | mg/day |
| EPT- | | | 1.8 | nM | 1-100 | nM | 1-100 | nM | 1-100 | uM | 5-50 | nM | 10 mg |
| 103182 | | | | | | | | | | | | | to 1000 |
| | | | | | | | | | | | | | mg/day |

| Compound | 1844064-06-7 | | |
|---|---|---|---|
| 54k | | | |
| Cmpd 1 | 1516899-38-9 | | |
| Cmpd 105 | 1613514-89-8 | 0.1-1 | uM |
| Compound | 1461602-86-7 | | |
| 34 | | | |
| Compound | 1628332-524 | | |
| 41 | | | |
| compound | 1628210-26-3 | 340 | nM |
| 48 | | | |
| Compound | 1993438-65-5 | >10 | uM |
| 18 | | | |
| Compound | 1613410-75-5 | ~1 | uM |
| 33 | | | |
| Compound | 1905482-57-6 | 1-10 | uM |
| 48 | | | |
| Compound | 1905482-57-6 | | |
| 48 | | | |
| Compound | 1905481-35-7 | | |
| 49 | | | |
| Compound | 1905481-36-8 | | |
| 50 | | | |
| Compound | 2169272-46-0 | | |
| 6 | | | |
| Compound | 1807514-47-1 | 1.5 | uM |
| R-35 | | | |
| CPI-455 | 1628208-23-0 | 90 | nM |
| CPI-4203 | 1628214-07-2 | | |
| E67-2 | 1364914-62-4 | | |
| GSK467 | 1628332-52-4 | | |
| GSK-J1 | 1373422-53-7 | 50 | uM |

| | | |
|---|---|---|
| GSK-J4 | 1373423-53-0 | |
| KDM5- | 1596embodi- | |
| C49 | ment-16-1 | |
| KDM5- | 1596348-32-1 | |
| C50 | | |
| KDOAM25 | 2230731-99-2 | |
| N11 | 1613515-45-9 | 1600 |
| | 1807514-47-1 | |
| | 1844064-07-8 | 960 |
| Compound | | |
| 1 | | |
| Compound | 1498996-89-6 | |
| 15e | | |
| Daminozide | 1596-84-5 | |
| JIB-04 | 99596-05-9 | |
| Methylstat | 1310877-95-2 | |
| Compound | 2098902-68-0 | |
| 10r | | |
| N71 | | |
| NSC | | |
| 6369819 | | |

In some embodiments the KDM inhibitor is AS 8351 or TC-E 5002.

### TAZ Activators

The one or more otic therapeutic agents in any embodiment disclosed herein could be one or more of the following TAZ activators.

TAZ motif (also called WWTR1) a transcriptional coactivator with a PDZ-binding was identified as a 14-3-3-binding protein. It is similar to Yes-associated protein 1 (YAP1) in its molecular structure, which consists of an N-terminal TEAD binding domain, one or two WW domains, and a transcriptional activation domain.

TAZ is phosphorylated at four sites by large tumor suppressor kinase 1 (LATS1) and LATS2, which are core kinases of the Hippo pathway. Phosphorylated TAZ is trapped by 14-3-3, is recruited from the nucleus to the cytoplasm, and undergoes protein degradation. In this way, the Hippo pathway negatively regulates TAZ.

In addition to the Hippo pathway, TAZ is regulated by cell junction proteins such as ZO-1, ZO-2, and angiomotin. Recent studies have revealed that TAZ is under the control of the actin cytoskeleton and the mechanical stretch. Moreover, Wnt signaling stabilizes. Conversely, cytoplasmic TAZ binds-catenin and Dishevelled (DVL) and inhibits-catenin nuclear localization and DVL phosphorylation to negatively regulate the Wnt pathway.

TAZ activators are chemical compounds that stabilize and increase unphosphorylated TAZ levels.

Thus, “TAZ activator” refers to an agent capable of the increasing the stability or activity of TAZ. For example, an TAZ activator results in a decrease in TAZ phosphorylation and/or TAZ protein degradation.

In certain embodiments, the TAZ activator increases the stability or activity of TAZ by at least 5, 10, 20, 30, 40, 50, 60, 70, 80, 90, or 100% relative to a control, for example relative to a baseline level of activity.

In certain embodiments, the TAZ activator increases the expression of a target gene by at least 5, 10, 20, 30, 40, 50, 60, 70, 80, 90, or 100% relative to a control, for example relative to a baseline level of activity.

In some embodiments, the TAZ activator increases the stability or activity of TAZ by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, increases the expression of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

Exemplary TAZ Activators are provide in Table 22.

TABLE 22

| | | Chemo- | | | | Lgr5+ | | Perilymph | | Formulation | | Human In | Human |
|---|---|---|---|---|---|---|---|---|---|---|---|---|---|
| Agent | CAS | type | Mechanism | Lit Cell | | Assay | | Conc | | Conc | | Vivo Conc | Dosage |
| IBS008738 | 371128-48-2 | Hydrazone | TAZ Activ. | | | 1.1-30 | uM | 1.1-30 | uM | 1.1-30 | mM | | 25-500 mg |
| TM-25659 | 260553-97-7 | AT II | TAZ Activ. | 10-100 | uM | 10-100 | uM | 10-100 | uM | 10-100 | mM | | 25-500 mg |
| TT10 | 2230640-94-3 | Thiazole | TAZ Activ. | 1 | uM | 1-10 | uM | 1-10 | uM | 1-10 | mM | | 25-500 mg |

| IBS003031 | 381177-81-7 | Acridine | YAP Activ. | | |
|---|---|---|---|---|---|
| TAZ12 | 371128-48-2 | Thiazole | TAZ Activ. | | |
| TM-53 | 1257247-76-9 | AT II | TAZ Activ. | 10-100 | uM |
| TM-54 | 1257247-77-0 | AT II | TAZ Activ. | 10-100 | uM |

| | | | |
|---|---|---|---|
| (−)-epicatechin | 1257-08-5 | Nat Prod | |
| gallate | | | |
| Ethacridine | 1837-57-6 | Acridine | Activity |
| Ethacridine | 1837-57-6 | Acridine | Activity |
| kaempferol | 520-18-3 | Nat Prod | |
| KR 62980 | 867187-61-9 | N-Oxide | |
| phorbaketal A | 1196507-03-5 | Nat Prod | |

In some embodiments the TAZ activator is IBS008738, TM-25659 or TT10.

In some embodiments the agents are a gamma— secretase inhibitor, a Taz activator, a Notch inhibitor, or an ErbB3/HER3 inhibitor.

### Gamma Secretase Inhibitors

The one or more otic therapeutic agents in any embodiment disclosed herein could be one or more of the following gamma secretase inhibitors.

Gamma secretase is an internal protease that cleaves within the membrane-spanning domain of its substrate proteins, including amyloid precursor protein (APP) and Notch.

Sequential cleavages of the APP by β- and γ-secretases generates Aβ. First, APP is proteolytically processed by β-secretase (BACE1) and generates a 12 kDa C-terminal stub of APP (C99); second, C99 is cleaved by γ-secretase to yield two major species of Aβ ending at residue 40 (A1340) or 42 (A1342).

Gamma secretase inhibitors may target γ-secretase and reduce Aβ production.

Exemplary gamma secretase inhibitors are provided in Table 23

TABLE 23

| | | Chemo- | | | | |
|---|---|---|---|---|---|---|
| Agent | CAS | type | Lit cell conc | | Human Dosage | |
| Semagacestat LY | 425386-60-3 | Amide | Aβ38, Aβ40, and Aβ42 with | | 60 mg-140 mg | |
| 450139 | | | IC50 = 12.0, 12.1, 10.9 nM, | | | |
| Begacestat/GSI-953 | 769169-27-9 | Sulphonamide | Lowers Aβ42, Aβ40 (EC50 = | | 10 and 50-mg | |
| | | | 12.4, 14.8 nM in cells | | | |
| Avagacestat/BMS- | 1146699-66-2 | Sulphonamide | IC50 = 0.27 and 0.30 nM for | | 25 to 125 | mg |
| 708163 | | | Aβ42 and Aβ40, | | | |
| EVP-0962 | | | | | 10, 50, 100 or | |
| | | | | | 200 mg | |
| Crenigacestat | 1421438-814 | Amide | IC50 of ~1 nM in most of the | | 2.5 mg-100 mg | |
| LY 3039478 | | | tumor cell lines | | | |
| (JSMD194) | | | | | | |
| MK-0572 | 471905-41-6 | Acid | SH-SY5Y cells with an IC50 value | | | |
| | | | of 5 nM | | | |
| NIC5-15 | | | | | 800-2000 | mg |
| NGP 555 | 1304630-27-0 | Heterocycle | 10 | nM | 100 mg, 200 mg, | |
| | | | | | or 400 mg | |
| Nirogacestat PF | 1290543-63-3 | Amide | (IC50 values are 1.2 and 6.2 nM in | | 150 | mg |
| 03084014 | | | whole cell and cell-free assays | | | |
| PF-06648671 | 1587727-31-8 | Amide | | | 300 | mg |
| RO4929097 | 847925-91-1 | Amide | | | 20 mg, 30 mg, 45 mg, | |
| | | | | | 90 mg or 140 mg | |
| BMS-905024 | 1401066-79-2 | Amide | | | | |
| BMS-932481 | 1263871-36-8 | Heterocycle | IC50 at 6.6 to reduce Aβ42 | | | |
| BMS-986133 | | | IC50 3.5 nM to reduce Aβ42 | | | |
| BMS 299897 | 290315-45-6 | Sulphonamide | Inhibits Aβ40 and Aβ42 in | | | |
| | | | vitro (IC50 7.4 and 7.9 nM | | | |
| BPN-15606 | 191498949-3 | Heterocycle | IC50 of 7 nM and 17 nM to reduce | | | |
| | | | Aβ42 and Aβ40 cells | | | |
| Carprofen | 53716-49-7 | Acid | 76 | uM | | |
| CHF5022 | 749269-77-0 | Acid | | | | |
| CHF5074 | 749269-83-8 | Acid | reduces Aβ42 and Aβ40 secretion, | | | |
| | | | IC50 3.6, 18.4 μM | | | |
| Compound E | 209986-174 | Amide | | | | |
| Compound W | 173550-33-9 | Acid | neuronal culture (IC50 115, 200 nM for | | | |
| | | | total Aβ, Aβ42 | | | |
| DAPT | 208255-80-5 | Amide | neuronal culture (IC50 115, 200 nM for | | | |
| | | | total Aβ, Aβ42 | | | |
| DBZ | 209984-56-5 | Amide | | | | |
| E-2012 | 870843-42-8 | Unsaturamide | | | | |
| EVP-A | | | IC50 reduction of Aβ40 and Aβ42 | | | |
| | | | 0.24 μM and 0.14 μM, | | | |
| EVP-B | | | IC50 reduction of Aβ40 and Aβ42 | | | |
| | | | 0.24 μM and 0.14 μM, | | | |
| EVP- | 1447811-26-8 | Acid | | | | |
| 0015962 | | | | | | |
| Flurizan | 51543-40-9 | Acid | | | | |
| GSI-136 | 443989-01-3 | Sulphonamide | | | | |
| Indomethacin | 53-86-1 | Acid | 25-50 | uM | | |
| JLK 6 | 62252-26-0 | Aniline | 30 | μM | | |
| JNJ- | 1146594-87-7 | Acid | 0.18-0.2 | μM, | | |
| 40418677 | | | | | | |
| L-685, 458 | 292632-98-5 | Peptide | 48-67 | nM | | |
| LY 411575 | 209984-57-6 | Amide | | | | |
| Deshydroxy LY- | 209984-56-5 | Amide | | | | |
| 411575 | | | | | | |
| MDL 28170 | 88191-84-8 | Amide | | | | |
| MRK 560 | 677772-84-8 | Sulphonamide | 0.65 | nM | | |
| MW167 | | | | | | |
| NMK-T-057 | | | | | | |
| Suldinac sulfide | 2004-67-4 | Acid | 34 | uM | | |

### Notch Inhibitors

The one or more otic therapeutic agents in any embodiment disclosed could be one or more of the following Notch inhibitors.

Exemplary Notch inhibitors are provided in Table 24

TABLE 24

| | CAS |
|---|---|
| Agent | |
| 3H4MB | 1958071-88-9 |
| BMS-871 | 15894631-89-9 |
| EDD3 | 25279-15-6 |
| ELN-46719 | 1576239-16-1 |
| FLI-06 | 313967-28-9 |
| IMR-1 | 310456-65-6 |
| JLK6 | 62252-26-0 |
| TAPI-1 | 171235-71-5 |
| Natural Products | |
| Honokiol | |
| epigallocatechin-3-gallate (EGCG) | |
| 3,5-bis(2,4-difluorobenzylidene)4-piperidone | |
| (DiFiD) | |
| curcumin | |
| 3,3′-diindolylmethane (DIM) | |
| resveratrol | |
| Antibodies | |
| MEDI0639 | |

### ERBB3/HER3 Inhibitors

The one or more otic therapeutic agents in any embodiment disclosed could be one or more of the following ErbB3/HER3 inhibitors.

Exemplary ErbB3/HER3 inhibitors are provided in Table 25.

TABLE 25

| | | | | | |
|---|---|---|---|---|---|
| | | HER 3 pKi or | | | |
| Agent | CAS | IC50 | HER1 | HER2 | HER4 |

| | | | | | | | |
|---|---|---|---|---|---|---|---|
| Bosutinib/SKI-606 | 380843-75-4 | 0.77 | nM | 2500 | nM | 26 | nM |
| Dasatinib/KIN001-5 | 302962-49-8 | 18 | nM | 1400 | nM | 55 | nM |

| | | | | | | | |
|---|---|---|---|---|---|---|---|
| Sapitinib/AZD8931/ | 848942-61-0 | 4 | nM | 4 | nM | 3 | nM |

| Vandetanib | | 180 | nM | 2600 | nM | 480 | nM |
|---|---|---|---|---|---|---|---|
| WS3 | 1421227-52-2 | 74 | nM | | | | |
| WS6 | 1421227-53-3 | 280 | nM | | | | |
| Afatinib | 850140-72-6 | | | 14 | nM | | |
| Erlotinib | 183321-74-6 | 1100 | nM | 2900 | nM | 230 | nM |
| Gefitinib | 184475-35-2 | 790 | nM | 3500 | nM | 410 | nM |
| KIN001-51 | | | | | | | |
| KIN001-111 | | | | | | | |
| Lapatinib | 231277-92-2 | 5500 | nM | 7 | nM | 54 | nM |

| Neratinib | 698387-09-6 | | | 59 | nM | | |
|---|---|---|---|---|---|---|---|
| poziotinib | 1092364-38-9 | 3 | nM | 5 | nM | 23 | nM |

| TX2-121-1 | 1603845-42-6 | | |
|---|---|---|---|
| WS1 | 936099-44-4 | 3.8 | uM |

AV-203
Duligotuzumab
Elgemtumab LJM716/
GSK2849330
KTN3379/CDX-3379
Lumretuzumab RG7116
Patritumab/U3-1287
Seribantumab/MM-121
U3-1402
MEHD7945A/Duligotumab
MCLA-128
MM-111
MM-141/Istiratumab

In some embodiments the ErbB3/HER3 inhibitors is WS3 or WS6.

In some embodiments, at least one hearing loss treatment agent is CHIR99021:

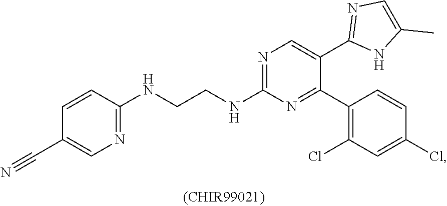 [SMILES: Cc1cnc(-c2cnc(NCCNc3ccc(C#N)cn3)nc2-c2ccc(Cl)cc2Cl)[nH]1]

or a pharmaceutical acceptable salt thereof.

Pharmaceutically acceptable salts include, for example salts formed by reacting any of the weakly basic active agents described herein, such as CHIR99021, with a pharmaceutically acceptable acid known in the art. A non-limiting list of suitable acid salts include hydrochloride, hydrobromide, citrate, malate, mesylate, phosphate, tartrate, hydrochloride, tosylate, glucuronate, ethanesulfonate, fumarate, sulfate, napthalene-2-sulfonate, ascorbate, oxalate, napthalene-1,5-disulfonate, malonate, aminosalicylate, benzenesulfonate, isethionate, genistate, 1-hydroxy-2-napthoate, dichloroacetate, cyclamate, and ethane-1,2-disulfonate.

In some embodiments, the composition of the present disclosure may comprise a compound of formula (I) or a pharmaceutically acceptable salt thereof. In some embodiments, the compound of formula (I) may also be an otic therapeutic agent. In some embodiments, wherein the compound of formula (I) is an otic therapeutic agent, it may be included in compositions of the present disclosure that comprise one or more otic therapeutic agents. In some embodiments, the compound of formula (I) may also be a hearing loss treatment agent. In some embodiments, the compound of formula (I) may be an HDAC inhibitor. In some embodiments, the compound of formula (I) or a pharmaceutically acceptable salt thereof is included in lyophilized pharmaceutical compositions of the present disclosure. In some embodiments, the compound of formula (I) or a pharmaceutically acceptable salt thereof is included in reconstituted pharmaceutical compositions of the present disclosure.

A compounds of formula (I), or a pharmaceutically acceptable salt has the following structure:

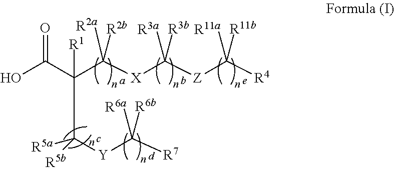

wherein:
R¹ is selected from H, alkyl, alkoxy, halo, cycloalkyl, alkenyl, alkynyl, carbocyclyl, and aryl;
R^{2a} is independently selected from H, alkyl, alkoxy, halo, cycloalkyl, alkenyl, alkynyl, carbocyclyl, and aryl;
R^{2b} is independently selected from H, alkyl, alkoxy, halo, cycloalkyl, alkenyl, alkynyl, carbocyclyl, and aryl;
X is selected from

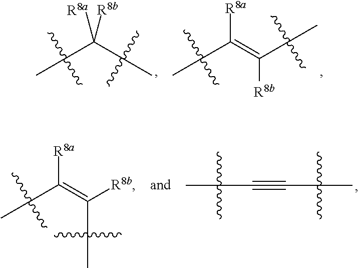

or is not present;
R^{3a} is independently selected from H, alkyl, alkoxy, halo, cycloalkyl, alkenyl, alkynyl, carbocyclyl, and aryl;
R^{3b} is independently selected from H, alkyl, alkoxy, halo, cycloalkyl, alkenyl, alkynyl, carbocyclyl, and aryl;
R⁴ is selected from H, alkyl, alkoxy, halo, cycloalkyl, alkenyl, alkynyl, carbocyclyl, and aryl;
R₅ₐ is independently selected from H, alkyl, alkoxy, halo, cycloalkyl, alkenyl, alkynyl, carbocyclyl, and aryl;
R^{5b} is independently selected from H, alkyl, alkoxy, halo, cycloalkyl, alkenyl, alkynyl, carbocyclyl, and aryl;
Y is selected from

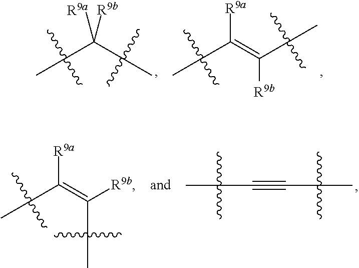

or is not present;
R^{6a} is selected from H, alkyl, alkoxy, halo, cycloalkyl, alkenyl, alkynyl, carbocyclyl, and aryl;
R^{6b} is selected from H, alkyl, alkoxy, halo, cycloalkyl, alkenyl, alkynyl, carbocyclyl, and aryl;
each R⁷ is independently selected from H, alkyl, alkoxy, halo, cycloalkyl, alkenyl, alkynyl, carbocyclyl, and aryl;
R^{8a} is independently selected from H, alkyl, alkoxy, halo, cycloalkyl, alkenyl, alkynyl, carbocyclyl, and aryl;
R^{8b} is independently selected from H, alkyl, alkoxy, halo, cycloalkyl, alkenyl, alkynyl, carbocyclyl, and aryl;
Z is selected from

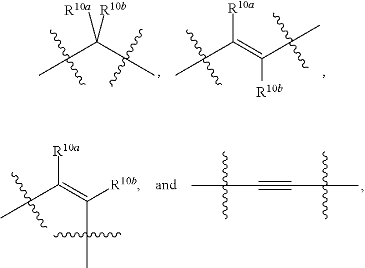

or is not present;
R^{10a} is independently selected from H, alkyl, alkoxy, halo, cycloalkyl, alkenyl, alkynyl, carbocyclyl, and aryl;
R^{10b} is independently selected from H, alkyl, alkoxy, halo, cycloalkyl, alkenyl, alkynyl, carbocyclyl, and aryl;
R^{11a} is selected from H, alkyl, alkoxy, halo, cycloalkyl, alkenyl, alkynyl, carbocyclyl, and aryl;
R^{11b} is selected from H, alkyl, alkoxy, halo, cycloalkyl, alkenyl, alkynyl, carbocyclyl, and aryl;
n^{a} is selected from 0, 1, 2, 3, 4, 5, 6, 7, and 8;
n^{b} is selected from 0, 1, 2, 3, and 4;
n^{c} is selected from 0, 1, and 2;
n^{d} is selected from 0, 1, and 2; and;
n^{e} is selected from 0, 1, 2, 3, 4, 5, and 6.

In some embodiments, R¹ is H. In some embodiments, R¹ is alkyl. In some embodiments, R¹ is alkoxy. In some embodiments, R¹ is halo. In some embodiments, R¹ is cycloalkyl. In some embodiments, R¹ is alkenyl. In some embodiments, R¹ is alkynyl. In some embodiments, R¹ is carbocyclyl. In some embodiments, R¹ is aryl.

In some embodiments, R^{2a} is H. In some embodiments, R^{2a} is alkyl. In some embodiments, R^{2a} is alkoxy. In some embodiments, R^{2a} is halo. In some embodiments, R^{2a} is cycloalkyl. In some embodiments, R^{2a} is alkenyl. In some embodiments, R^{2a} is alkenyl. In some embodiments, R^{2a} is carbocyclyl. In some embodiments, Ra is aryl. In some embodiments, R^{2a} is H. In some embodiments, R^{2a} is alkyl. In some embodiments, R^{2b} is alkoxy. In some embodiments, R^{2b} is halo. In some embodiments, R^{2b} is cycloalkyl. In some embodiments, R^{2b} is alkenyl. In some embodiments, R^{2b} is alkynyl. In some embodiments, R³ is carbocyclyl. In some embodiments, Rb is aryl.

In some embodiments, R^{3a} is H. In some embodiments, R^{3a} is alkyl. In some embodiments, R^{3a} is alkoxy. In some embodiments, R^{3a} is halo. In some embodiments, R^{3a} is cycloalkyl. In some embodiments, R^{3a} is alkenyl. In some embodiments, R^{3a} is alkynyl. In some embodiments, R^{3a} is carbocyclyl. In some embodiments, R^{3a} is aryl. In some embodiments, R^{3b} is H. In some embodiments, R^{3b} is alkyl. In some embodiments, R^{3b} is alkoxy. In some embodiments, R^{3b} is halo. In some embodiments, R³ is cycloalkyl. In some embodiments, R³ is alkenyl. In some embodiments, R^{3b} is alkynyl. In some embodiments, R^{3a} is carbocyclyl. In some embodiments, R^{3b} is aryl.

In some embodiments, R⁴ is H. In some embodiments, R⁴ is alkyl. In some embodiments, R⁴ is alkoxy. In some embodiments, R⁴ is halo. In some embodiments, R⁴ is cycloalkyl. In some embodiments, R⁴ is alkenyl. In some embodiments, R⁴ is alkynyl. In some embodiments, R⁴ is carbocyclyl.

In some embodiments, R⁴ is aryl.

In some embodiments, R^{5a} is H. In some embodiments, R^{5a} is alkyl. In some embodiments, R is alkoxy. In some embodiments, R^{5a} is halo. In some embodiments, R^{5a} is cycloalkyl. In some embodiments, R^{5a} is alkenyl. In some embodiments, R^{5a} is alkynyl. In some embodiments, R^{5a} is carbocyclyl. In some embodiments, R^{5a} is aryl.

In some embodiments, R^{5b} is H. In some embodiments, R^{5b} is alkyl. In some embodiments, R^{5b} is alkoxy. In some embodiments, R^{5b} is halo. In some embodiments, R^{5b} is cycloalkyl. In some embodiments, R^{5b} is alkenyl. In some embodiments, R^{5b} is alkynyl. In some embodiments, R^{5a} is carbocyclyl. In some embodiments, R^{5b} is aryl.

In some embodiments, R^{6a} is H. In some embodiments, R^{6a} is alkyl. In some embodiments, R^{6a} is alkoxy. In some embodiments, R^{6a} is halo. In some embodiments, R^{6a} is cycloalkyl. In some embodiments, R^{6a} is alkenyl. In some embodiments, R^{6a} alkynyl. In some embodiments, Reis carbocyclyl. In some embodiments, R^{6a} is aryl.

In some embodiments, R^{6b} is H. In some embodiments, R^{6b} is alkyl. In some embodiments, R^{6b} is alkoxy. In some embodiments, R^{6b} is halo. In some embodiments, R^{6b} is cycloalkyl. In some embodiments, R^{6b} is alkenyl. In some embodiments, R^{6b} is alkynyl. In some embodiments, R^{6b} is carbocyclyl. In some embodiments, R^{6b} is aryl.

In some embodiments, R⁷ is H. In some embodiments, R⁷ is alkyl. In some embodiments, R⁷ is alkoxy. In some embodiments, R⁷ is halo. In some embodiments, R⁷ is cycloalkyl. In some embodiments, R⁷ is alkenyl. In some embodiments, R⁷ is alkynyl. In some embodiments, R⁷ is carbocyclyl. In some embodiments, R⁷ is aryl.

In some embodiments, X is

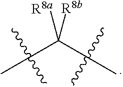

In some embodiments, X is

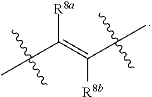

In some embodiments, X is

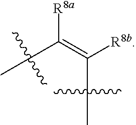

In some embodiments, X is

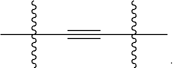 [SMILES: *C#C*]

In some embodiments, X is not present.

In some embodiments, Y is

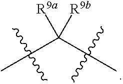

In some embodiments, Y is

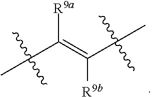

In some embodiments, Y is

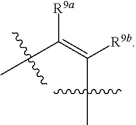

In some embodiments, Y is

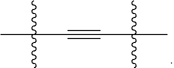 [SMILES: *C#C*]

In some embodiments, Y is not present.

In some embodiments, R^{8a} is H. In some embodiments, R^{8a} is alkyl. In some embodiments, R^{8a} is alkoxy. In some embodiments, R^{8a} is halo. In some embodiments, R^{8a} is cycloalkyl. In some embodiments, R^{8a} is alkenyl. In some embodiments, R^{8a} is alkynyl. In some embodiments, R^{8a} is carbocyclyl. In some embodiments, R^{8a} is aryl.

In some embodiments, R^{8b} is H. In some embodiments, R^{8b} is alkyl. In some embodiments, R^{8b} is alkoxy. In some embodiments, R^{8b} is halo. In some embodiments, R^{8b} is cycloalkyl. In some embodiments, R^{8b} is alkenyl. In some embodiments, R^{8b} is alkynyl. In some embodiments, R^{8b} is carbocyclyl. In some embodiments, R^{8b} is aryl.

In some embodiments, R^{9a} is H. In some embodiments, R^{8b} is alkyl. In some embodiments, R is alkoxy. In some embodiments, R^{9a} is halo. In some embodiments, R⁹ is cycloalkyl. In some embodiments, R^{9a} is alkenyl. In some embodiments, R^{9a} is alkynyl. In some embodiments, R^{9a} is carbocyclyl. In some embodiments, R^{9a} is aryl.

In some embodiments, R^{9b} is H. In some embodiments, R^{9b} is alkyl. In some embodiments, R^{9b} is alkoxy. In some embodiments, R^{9b} is halo. In some embodiments, R^{9b} is cycloalkyl. In some embodiments, R^{9b} is alkenyl. In some embodiments, R^{9b} is alkynyl. In some embodiments, Re is carbocyclyl. In some embodiments, R^{9b} is aryl.

In some embodiments, Z is

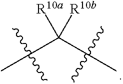

In some embodiments, Z is

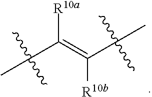

In some embodiments, Z is

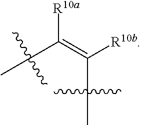

In some embodiments, Z is

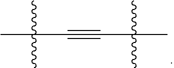 [SMILES: *C#C*]

In some embodiments, Z is not present.

In some embodiments, R^{10a} is H. In some embodiments, R^{10a} is alkyl. In some embodiments, R^{10a} is alkoxy. In some embodiments, R^{10a} is halo. In some embodiments, R^{10a} is cycloalkyl. In some embodiments, R^{10a} is alkenyl. In some embodiments, R^{10a} is alkynyl. In some embodiments, R^{10a} is carbocyclyl. In some embodiments, R^{10a} is aryl.

In some embodiments, R^{10b} is H. In some embodiments, R^{10b} is alkyl. In some embodiments, R^{10b} is alkoxy. In some embodiments, R^{10b} is halo. In some embodiments, R^{10b} is cycloalkyl. In some embodiments, R^{10b} is alkenyl. In some embodiments, R^{10b} is alkynyl. In some embodiments, R^{10b} is carbocyclyl. In some embodiments, R^{10b} is aryl.

In some embodiments, R^{11a} is H In some embodiments, R^{11b} is alkyl. In some embodiments, R^{11a} is alkoxy. In some embodiments, R^{11a} is halo. In some embodiments, R^{11a} is cycloalkyl. In some embodiments, R^{11a} is alkenyl. In some embodiments, R^{11a} is alkynyl. In some embodiments, R^{11a} is carbocyclyl. In some embodiments, R^{11a} is aryl.

In some embodiments, R^{11b} is H. In some embodiments, R^{11b} is alkyl. In some embodiments, R^{11b} is alkoxy. In some embodiments, R^{11b} is halo. In some embodiments, R^{11b} is cycloalkyl. In some embodiments, R^{11b} is alkenyl. In some embodiments, R^{11b} is alkynyl. In some embodiments, R^{11b} is carbocyclyl. In some embodiments, R^{11b} is aryl.

In some embodiments, n^{a} is 0. In some embodiments, n^{a} is 1. In some embodiments, n^{a} is 2. In some embodiments, n^{a} is 3. In some embodiments, n^{a} is 4. In some embodiments, n^{a} is 5. In some embodiments, n^{a} is 6. In some embodiments, n^{a} is 7. In some embodiments, n^{a} is 8.

In some embodiments, n^{b} is 0. In some embodiments, n^{b} is 1. In some embodiments, n^{b} is 2. In some embodiments, n^{b} is 3. In some embodiments, n^{b} is 4.

In some embodiments, n^{c} is 0. In some embodiments, n^{c} is 1. In some embodiments, n^{c} is 2.

In some embodiments, n^{d} is 0. In some embodiments, n^{d} is 1. In some embodiments, n^{d} is 2.

In some embodiments, n^{e} is 0. In some embodiments, n^{e} is 1. In some embodiments, n^{e} is 2. In some embodiments, n^{e} is 3. In some embodiments, n^{e} is 4. In some embodiments, n^{e} is 5. In some embodiments, n^{e} is 6.

In some embodiments, R¹ is Me. In some embodiments, R^{2a} is Me. In some embodiments, R^{2b} is Me. In some embodiments, R^{3a} is Me. In some embodiments, R^{3b} is Me. In some embodiments, R⁴ is Me. In some embodiments, R^{5a} is Me. In some embodiments, R^{5b} is Me. In some embodiments, R^{6a} is Me. In some embodiments, R^{6b} is Me. In some embodiments, R⁷ is Me. In some embodiments, R^{8a} is Me. In some embodiments, R^{8b} is Me. In some embodiments, R^{9a} is Me. In some embodiments, R^{9b} is Me. In some embodiments, R^{10a} is Me. In some embodiments, R^{10b} is Me. In some embodiments, R^{11a} is Me. In some embodiments, R^{11b} is Me.

In some embodiments, R¹ is F. In some embodiments, R^{2a} is F. In some embodiments, R^{2b} is F. In some embodiments, R^{3a} is F. In some embodiments, R^{3b} is F. In some embodiments, R⁴ is F. In some embodiments, R^{5a} is F. In some embodiments, R^{5b} is F. In some embodiments, R^{6a} is F. In some embodiments, R^{6b} is F. In some embodiments, R⁷ is F. In some embodiments, R^{8a} is F. In some embodiments, R^{8b} is F. In some embodiments, R^{9a} is F. In some embodiments, R^{9b} is F. In some embodiments, R^{10a} is F. In some embodiments, R^{10b} is F. In some embodiments, R^{11a} is F. In some embodiments, R^{11b} is F.

In some embodiments, R¹ is alkyl. In some embodiments, R^{2a} is alkyl. In some embodiments, R^{2b} is alkyl. In some embodiments, R^{3a} is alkyl. In some embodiments, R^{3b} is alkyl. In some embodiments, R⁴ is alkyl. In some embodiments, R^{5a} is alkyl. In some embodiments, R^{5b} is alkyl. In some embodiments, R^{6a} is alkyl. In some embodiments, R^{6b} is alkyl. In some embodiments, R⁷ is alkyl. In some embodiments, R^{8a} is alkyl. In some embodiments, R^{8b} is alkyl. In some embodiments, R^{9a} is alkyl. In some embodiments, R^{9b} is alkyl. In some embodiments, R^{10a} is alkyl. In some embodiments, R^{10b} is alkyl. In some embodiments, R^{11a} is alkyl. In some embodiments, R^{11b} is alkyl.

In some embodiments, alkyl is methyl. In some embodiments, alkyl is ethyl. In some embodiments, alkyl is n-propyl. In some embodiments, alkyl is iso-propyl. In some embodiments, alkyl is n-butyl. In some embodiments, alkyl is sec-butyl. In some embodiments, alkyl is iso-butyl. In some embodiments, alkyl is tert-butyl.

In some embodiments, alkoxy is methoxy. In some embodiments, alkoxy is ethoxy. In some embodiments, alkoxy is n-propoxy. In some embodiments, alkoxy is iso-propoxy. In some embodiments, alkoxy is n-butoxy. In some embodiments, alkoxy is sec-butoxy. In some embodiments, alkoxy is iso-butoxy. In some embodiments, alkoxy is tert-butoxy.

In some embodiments, halo is F. In some embodiments, halo is Cl. In some embodiments, halo is Br. In some embodiments, halo is I.

In some embodiments, cycloalkyl is cyclopropyl. In some embodiments, cycloalkyl is cyclobutyl. In some embodiments, cycloalkyl is cyclopentyl. In some embodiments, cycloalkyl is cyclohexyl.

In some embodiments, aryl is phenyl. In some embodiments, aryl is tolyl. In some embodiments, aryl is xylyl.

In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with methyl. In some embodiments, one of R¹, R^{2a}, R^{2b}, R^{3a}, R^{3b}, R⁴, R^{5a}, R^{5b}, R^{6a}, R^{6b}, R⁷, R₈ₐ, R^{8b}, R^{9a}, R^{9b}, R^{10a}, R^{10b}, R^{11a}, and R^{11b}, is further substituted with ethyl. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with n-propyl. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with iso-propyl. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with n-butyl.

In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with sec-butyl. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with iso-butyl. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with tert-butyl.

In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with methoxy. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with ethoxy. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with n-propoxy. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with iso-propoxy. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with n-butoxy. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with sec-butoxy. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with iso-butoxy. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with tert-butoxy.

In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with F. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with Cl. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with Br. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with I.

In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with cycloalkyl. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with alkenyl. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with alkynyl. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with carbocyclyl. In some embodiments, one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with aryl.

In some embodiments, the compound of formula (I) is valproic acid or a pharmaceutically acceptable salt thereof.

In some embodiments, the compound of formula (I) is 2-(prop-2-yn-1-yl)-octanoic acid or a pharmaceutically acceptable salt thereof.

In some embodiments, the compound of formula (I) is linoleic acid or a pharmaceutically acceptable salt thereof.

In some embodiments, the compound of formula (I) is phenylbutyric acid or a pharmaceutically acceptable salt thereof.

In some embodiments, at least one hearing loss treatment agent is valproic acid:

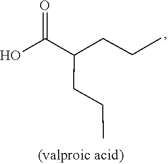

or a pharmaceutical acceptable salt thereof (e.g., sodium valproate). A non-limiting list of other suitable valproate salts includes potassium valproate, lithium valproate, etc. A further non-limiting list of other suitable of valproate salts includes sodium valproate, valproate semisodium, magnesium divalproate (magnesium valproate), calcium divalproate (calcium valproate). Valproic acid is also referred to as WA. Sodium valproate is also referred to as NaVPA.

In some embodiments, at least one hearing loss treatment agent is CHIR99021 or a pharmaceutical acceptable salt thereof, and at least one hearing loss treatment agent is valproic acid or a pharmaceutical acceptable salt thereof (e.g., sodium valproate).

In some embodiments, the one or more otic therapeutic agents (e.g., hearing loss treatment agents) are CHIR99021 or a pharmaceutical acceptable salt thereof, and valproic acid or a pharmaceutical acceptable salt thereof (e.g., sodium valproate).

In some embodiments, the pharmaceutically acceptable salt of valproic acid is a sodium valproate.

In some embodiments, the one or more otic therapeutic agents (e.g., hearing loss treatment agents) are CHIR99021 and sodium valproate.

In some embodiments the at least one otic therapeutic agent is LY2090314 or a pharmaceutically acceptable salt thereof.

In some embodiments, at least one hearing loss treatment agent is LY2090314 or a pharmaceutical acceptable salt thereof.

In some embodiments, at least one hearing loss treatment agent is LY2090314 or a pharmaceutical acceptable salt thereof, and at least one hearing loss treatment agent is valproic acid or a pharmaceutical acceptable salt thereof (e.g., sodium valproate).

In some embodiments, the one or more otic therapeutic agents (e.g., hearing loss treatment agents) are LY2090314 and sodium valproate.

### Gelling Agents

As used herein, the term “gelling agent” refers to an agent capable of imparting a gel-like or thickening quality to the pharmaceutical composition or reconstituted solution of the present disclosure upon being subjected to a gelling condition (e.g., a particular temperature or temperature range, the presence of an ion, a pH value or range, or a concentration of gelling agent that causes the gelling agent to undergoing a change or transition from low viscosity to high viscosity, or the reverse). In some embodiments, the gelling condition is a particular temperature (e.g., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.). In some embodiments, the gelling condition is a particular temperature range (e.g., about 26° C. or higher, about 27° C. or higher, about 28° C. or higher, about 29° C. or higher, about 30° C. or higher, about 31° C. or higher, about 32° C. or higher, about 33° C. or higher, about 34° C. or higher, about 35° C. or higher, about 36° C. or higher, about 37° C. or higher, about 38° C. or higher, about 39° C. or higher, or about 40° C. or higher). In some embodiments, the gelling agent provides a viscosity of between about 1,000 and 10,000,000 centipoise, between about 5,000 and 5,000,000 centipoise, or between about 100,000 and 4,000,000 centipoise, to the pharmaceutical composition or reconstituted solution of the present disclosure. In some embodiments, the gelling agent provides a viscosity of between about 50,000 and 2,000,000 centipoise to the pharmaceutical composition or reconstituted solution of the present disclosure.

In some embodiments, prior to gelling (e.g., at ambient temperature (e.g., between about 20° C. and about 26° C.)), the gelling agent provides a viscosity of less than about 100,000 centipoise, less than about 50,000 centipoise, 20,000 centipoise, less than about 10,000 centipoise, less than about 8,000 centipoise, less than about 7,000 centipoise, less than about 6,000 centipoise, less than about 5,000 centipoise, less than about 4,000 centipoise, less than about 3,000 centipoise, less than about 2,000 centipoise, or less than about 1,000 centipoise to the pharmaceutical composition or reconstituted solution of the present disclosure.

In some embodiments, upon gelling (e.g., at the temperature of a human body (e.g., between about 35° C. to about 39° C., between about 36° C. to about 38° C., or at about 37° C.)), the gelling agent provides a viscosity of greater than about 1,000 centipoise, greater than about 5,000 centipoise, greater than about 10,000 centipoise, greater than about 20,000 centipoise, greater than about 50,000 centipoise, greater than about 60,000 centipoise, greater than about 70,000 centipoise, greater than about 80,000 centipoise, greater than about 90,000 centipoise, or greater than about 100,000 centipoise.

In some embodiments, upon gelling (e.g., at the temperature of a human body (e.g., between about 36° C. to about 39° C., or at about 37° C.)), the viscosity of the pharmaceutical composition or reconstituted solution of the present disclosure, as measured in units of centipoise, being about 2 fold or greater, about 5 fold or greater, about 10 fold or greater, about 20 fold or greater, about 50 fold or greater, about 60 fold or greater, about 7 fold or greater, about 80 fold or greater, about 90 fold or greater, about 100 fold or greater as compared to the viscosity of the pharmaceutical composition or reconstituted solution prior to gelling (e.g., at ambient temperature (e.g., at about 25° C.)).

It is understood that the gelling condition (e.g., gelling temperature) of the pharmaceutical composition or reconstituted solution of the present disclosure may be measured with a variety of techniques in the art. In some embodiment, the gelling temperature is determined using a commercially available rheomoeter having a parallel plate geometry (e.g., with plate distance ranging from 0.5 mm to 1.0 mm). In some embodiments, the analysis is performed over a continuous temperature range (e.g., 15° C. to 40° C.) at a constant rate (e.g., 2 to 3° C./min) and a deformation frequency of 0.74 Hz to 1 Hz. The gelation temperature is determined at the temperature whereby the shear storage modulus (G′) and the shear loss modulus (G″) are equal.

In some embodiments, the gelling agent comprises acacia, alginic acid, bentonite, poly(acrylic acid) (Carbomer), carboxymethyl cellulose, ethylcellulose, gelatin, hydroxyethyl cellulose, hydroxypropyl cellulose, magnesium aluminum silicate (Veegum), methylcellulose, poloxamer, hyaluronic acid sodium, polylacticglycolic acid sodium, chitosan, polyvinyl alcohol, sodium alginate, tragacanth, xanthan gum, or any combination thereof. In some embodiment, the gelling agent comprises poloxamer. In some embodiments, the gelling agent comprises hyaluronic acid. In some embodiments, the gelling agent is hyaluronic acid. In some embodiments the hyaluronic has a MW average of between 7.0×10\hat{ }5 Daltons and 8.5 10\hat{ }5 Daltons. In some embodiments the hyaluronic has a MW average of 8.23×10\hat{ }5 Daltons. In some embodiments, the hyaluronic acid is ‘HA1M’ provided by Lifecore Bio. In some embodiments the hyaluronic acid is a 0.5-5% aq. solution. In some embodiments the hyaluronic acid is a 1-3% aq. solution. In some embodiments, the hyaluronic acid has an average MW of 8.23×10\hat{ }5 Daltons and is prepared as a 1-3% aq. solution.

In some embodiments, the gelling agent comprises acacia. In some embodiments, the gelling agent comprises alginic acid. In some embodiments, the gelling agent comprises bentonite. In some embodiments, the gelling agent comprises poly(acrylic acid) (Carbomer). In some embodiments, the gelling agent comprises carboxymethyl cellulose. In some embodiments, the gelling agent comprises ethylcellulose. In some embodiments, the gelling agent comprises gelatin. In some embodiments, the gelling agent comprises hydroxyethyl cellulose. In some embodiments, the gelling agent comprises hydroxypropyl cellulose. In some embodiments, the gelling agent comprises magnesium aluminum silicate (Veegum). In some embodiments, the gelling agent comprises methylcellulose. In some embodiments, the gelling agent comprises poloxamer. In some embodiments, the gelling agent comprises hyaluronic acid sodium. In some embodiments, the gelling agent comprises hyaluronic acid. In some embodiments, the gelling agent comprises polylacticglycolic acid sodium. In some embodiments, the gelling agent comprises chitosan. In some embodiments, the gelling agent comprises polyvinyl alcohol. In some embodiments, the gelling agent comprises sodium alginate. In some embodiments, the gelling agent comprises tragacanth. In some embodiments, the gelling agent comprises xanthan gum. In some embodiments, the gelling agent comprises a cellulosic derivative (e.g., carboxymethylcellulose sodium, powdered cellulose, hydroxymethyl cellulose, hydroxypropyl cellulose, hydroxypropyl methylcellulose, and/or methylcellulose).

In some embodiments, the gelling agent is a thermoreversible gelling agent.

As used herein, the term “thermoreversible” refers to a capability of being reversible by the application of heat. The “thermoreversible gelling agent” refers to an agent capable of reversibly imparting a gel-like or thickening quality to the pharmaceutical composition or reconstituted solution of the present disclosure upon application of heat

In some embodiments, the thermoreversible gelling agent comprises a poloxamer.

In some embodiments, poloxamer forms a thermoreversible gel. For example, with the application of heat to a solution of a poloxamer, the viscosity of the solution increases. The viscosity of the solution can increase to the extent that the solution forms a gel. In some embodiments, the solution of poloxamer forms a gel at about body temperature (37° C.). In some embodiments, the solution of poloxamer forms an immobile gel at about body temperature. In preferred such embodiments, the solution of poloxamer is a composition comprising further components, such as one or more otic therapeutic agents and/or valproic acid or a pharmaceutically acceptable salt thereof.

In certain embodiments it can be useful for a thermoreversible gelling agent disclosed herein to be a gel when at body temperature but a liquid when below body temperature. For example, it may be a liquid in order for it to be injected into the ear (for example the middle ear). Thermoreversible gelling agents are known in the art, for example those polymers that reversibly impart a gel-like or thickening quality upon application of heat disclosed in Shalaby et al. Water-Soluble Polymers, ACS Symposium Series, American Chemical Societry, 1991 (Chapter 33). Those include those polymers that have those properties are also disclosed in Molyneaux, P. “Water-Soluble Polymers: Properties and Behavior”, CRC Press, Vol. I, p. 58, Vol. II, p. 86, New York, 1982; Prasad, K. N., Luong, T. T., Florence, A. T., Paris, J., Vaution, C., Seiller, M and Puisieux, F., J. Colloid Interface Sci., 69, 225(1979); A. V. Kabanov et al./Journal of Controlled Release 82 (2002) 189 −212; Peppas and Khare, Advanced Drug Delivery Reviews, 11 (1993) 1-35; U.S. Pat. Nos. 6,316,011B1; 4,474,751; 4,478,822; 6,346,272 and 4,188,373. Any thermoreversible gelling agent disclosed in these references, and in particular those that are a gel when at body temperature but a liquid when below body temperature, can be used as a gelling agent in all aspects and options disclosed herein.

It is understood that the gelling agent (e.g., the thermoreversible gelling agent) may also be a bulking agent of the pharmaceutical composition or reconstituted solution of the present disclosure. In some embodiments, a poloxamer (e.g., poloxamer 407) is the gelling agent and/or the bulking agent of the pharmaceutical composition or reconstituted solution of the present disclosure. Poloxomers are a general class of commercially available and pharmaceutically acceptable triblock copolymers of polyethylene oxide-polypropylene oxide-polyethylene oxide which exhibit relatively low viscosity at low temperatures (e.g., room termpature or below) but much high viscosities at elevated temperatures (e.g., body temperatures of approximately 37° C.) whereby compositions containing such thermoreversible gelling agents effectively solidify in place. Other thermoreversible gelling agents such as polyethylene oxide-polylactic acid— polyethylene oxide polymers are also suitable in various embodiments of the present invention.

Poloxamers are a general class of commercially available triblock copolymers that in certain embodiments can be used as the gelling agent More specifically, such poloxamers can comprise a central hydrophobic chain of polyoxypropylene (poly(propylene oxide) or PPO) flanked by two hydrophilic chains of polyoxyethylene (poly(ethylene oxide) or PEG). This forms an A-B-A structure, shown below:

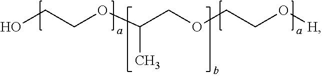

where a=2-130 and b=15-70.

In some embodiments, a is 10-120. In some embodiments, a is 20-120. In some embodiments, a is 30-120. In some embodiments, a is 40-120. In some embodiments, a is 50-120. In some embodiments, a is 60-120. In some embodiments, a is 70-120. In some embodiments, a is 80-120. In some embodiments, a is 90-120. In some embodiments, a is 100-120. In some embodiments, a is 110-120. In some embodiments, a is 10-110. In some embodiments, a is 20-110. In some embodiments, a is 30-110. In some embodiments, a is 40-110. In some embodiments, a is 50-110. In some embodiments, a is 60-110. In some embodiments, a is 70-110. In some embodiments, a is 80-110. In some embodiments, a is 90-110. In some embodiments, a is 100-110. In some embodiments, a is 10-100. In some embodiments, a is 20-100. In some embodiments, a is 30-100. In some embodiments, a is 40-100. In some embodiments, a is 50-100. In some embodiments, a is 60-100. In some embodiments, a is 70-100. In some embodiments, a is 80-100. In some embodiments, a is 90-100. In some embodiments, a is 95-105. In some embodiments, a is 95-115. In some embodiments, a is 85-105. In some embodiments, a is 85-115. In some embodiments, b is 25-70. In some embodiments, b is 35-70. In some embodiments, b is 45-70. In some embodiments, b is 55-70. In some embodiments, b is 60-70. In some embodiments, b is 65-70. In some embodiments, b is 56+/−10%, and each a is 101+/−10%. In some embodiments, b is 61+/−15%, and each a is 101+/−10%. In some embodiments, b is 70+/−20%, and each a is 101+/−20%. In some embodiments, b is 56+/−10%, and each a is 100+/−10%. In some embodiments, b is 61+/−15%, and each a is 100+/−10%. In some embodiments, b is 70+/−20%, and each a is 100+/−10%.

In certain embodiments, Poloxamers are also known by the tradenames of Synperonics, Pluronics, and Kolliphor. For the generic term poloxamer, these copolymers are commonly named with the letter P (for poloxamer) followed by three digits: the first two digits multiplied by 100 give the approximate molecular mass of the polyoxypropylene core, and the last digit multiplied by 10 gives the percentage polyoxyethylene content (e.g. P407=poloxamer with a polyoxypropylene molecular mass of 4000 g/mol and a 70% polyoxyethylene content). For the Pluronic and Synperonic tradenames, coding of these copolymers starts with a letter to define its physical form at room temperature (L=liquid, P=paste, F=flake (solid)) followed by two or three digits. The first digit (two digits in a three-digit number) in the numerical designation, multiplied by 300, indicates the approximate molecular weight of the hydrophobe; and the last digit×10 gives the percentage polyoxyethylene content (e.g., L61 indicates a polyoxypropylene molecular mass of 1800 g/mol and a 10% polyoxyethylene content). In the example given, poloxamer 181 (P181)=Pluronic L61 and Synperonic PE/L 61.

For Poloxamer 407 (P407), the approximate lengths of the two PEG blocks is about 100 repeat units while the approximate length of the propylene glycol block is about 56-67 repeat units (where about is +/−10%). P407 is also known by the BASF trade name Pluronic F127 or by the Croda trade name Synperonic PE/F 127.

Poloxamers can also be composed of a central hydrophilic chain of polyoxyethylene (poly(ethylene oxide) or PEG) flanked by two hydrophobic chains of polyoxypropylene (poly(propylene oxide)). This forms an analogous B-A-B structure. Other PPO-PEG block copolymers exist, such as those that comprise four PPO-PEO chains, which extend outward from an amine-terminated central chain (e.g. N—CH₂—CH₂—N), and in certain embodiments the disclosed compositions can comprise one or more of such four block polymers. (either in addition to or instead of the poloxamers otherwise disclosed herein).

In some embodiments, the poloxamer (e.g., poloxamer 407) is the gelling agent and the bulking agent of the pharmaceutical composition or reconstituted solution of the present disclosure. In some embodiments, the presence of the poloxamer (e.g., poloxamer 407) in the pharmaceutical composition (e.g., the lyophilized pharmaceutical composition) alleviates the need for any other excipient (e.g., additional bulking agent). Such alleviation may provide one or more advantages to the pharmaceutical composition (e.g., enhanced stability and/or reduced reconstitution time).

In some embodiments, the pharmaceutical composition of the present disclosure does not comprise an additional bulking agent.

In some embodiments, the lyophilized pharmaceutical composition of the present disclosure does not comprise an additional bulking agent.

In some embodiments, the reconstituted lyophilized pharmaceutical composition of the present disclosure does not comprise an additional bulking agent.

Several parameters may be used to characterize the poloxamers that feature in the compositions of the present disclosure, such as the percentage PEO in the polymer and/or average molecular weight and/or levels of purity. It will be appreciated that these parameters may be combinable and any number of different parameters may be used to described the poloxamer.

In some embodiments, the poloxamer is purified. In some embodiments, the poloxamer is not purified. In some embodiments, the poloxamer (e.g., Poloxamer 407) has an average molecular weight of about 7.25 KDa or greater, about 9 kDa or greater, about 9.2 kDa or greater, about 9.4 kDa or greater, about 9.6 kDa or greater, about 9.8 kDa or greater, about 10 kDa or greater, about 10.2 kDa or greater, about 10.4 kDa or greater, about 10.6 kDa or greater, about 10.8 kDa or greater, about 11 kDa or greater, about 11.2 kDa or greater, about 11.4 kDa or greater, about 11.6 kDa or greater, about 11.8 kDa or greater, about 12 kDa or greater, or about 12.1 kDa or greater. In some embodiments, the poloxamer comprises at least 50% polyethylene oxide by molecular mass. In some embodiments, the poloxamer comprises at least 55% polyethylene oxide by molecular mass. In some embodiments, the poloxamer comprises at least 60% polyethylene oxide by molecular mass. In some embodiments, the poloxamer comprises at least 65% polyethylene oxide by molecular mass. In some embodiments, the poloxamer comprises at least 66% polyethylene oxide by molecular mass. In some embodiments, the poloxamer comprises at least 67% polyethylene oxide by molecular mass. In some embodiments, the poloxamer comprises at least 68% polyethylene oxide by molecular mass. In some embodiments, the poloxamer comprises at least 69% polyethylene oxide by molecular mass. In some embodiments, the poloxamer comprises at least 70% polyethylene oxide by molecular mass. In some embodiments, the poloxamer comprises 60-80% polyethylene oxide by molecular mass. In some embodiments, the poloxamer comprises 65-75% polyethylene oxide by molecular mass.

In some embodiments, the poloxamer has an average molecular weight of about 7250 to about 17350 Daltons. In some embodiments, the poloxamer has an average molecular weight of about 8000 to about 17000 Daltons. In some embodiments, the poloxamer has an average molecular weight of about 8000 to about 16000 Daltons. In some embodiments, the poloxamer has an average molecular weight of about 9000 to about 16000 Daltons. In some embodiments, the poloxamer has an average molecular weight of about 9000 to about 15000 Daltons. In some embodiments, the poloxamer has an average molecular weight of about 9800 to about 14600 Daltons. In some embodiments, the poloxamer has an average molecular weight of about 10000 to about 14000 Daltons. In some embodiments, the poloxamer has an average molecular weight of about 10500 to about 14000 Daltons. In some embodiments, the poloxamer has an average molecular weight of about 10500 to about 13500 Daltons. In some embodiments, the poloxamer has an average molecular weight of about 11000 to about 14000 Daltons. In some embodiments, the poloxamer has an average molecular weight of about 11000 to about 13500 Daltons. In some embodiments, the poloxamer has an average molecular weight of about 11500 to about 14000 Daltons. In some embodiments, the poloxamer has an average molecular weight of about 11500 to about 13000 Daltons. In some embodiments, the poloxamer has an average molecular weight of about 12000 to about 14000 Daltons. In some embodiments, the poloxamer has an average molecular weight of about 12000 to about 13000 Daltons. In some embodiments, the poloxamer has an average molecular weight of about 10500 to about 12500 Daltons. In some embodiments, the poloxamer has an average molecular weight of about 10500 to about 11500 Daltons. In some embodiments, the poloxamer has an average molecular weight of about 11500 to about 12500 Daltons.

In some embodiments, at least 85% by wt. of the poloxamer has an average molecular weight of about 7250 to about 17350 Da. In some embodiments, at least 86% by weight of the poloxamer has an average molecular weight of about 7250 to about 17350 Da. In some embodiments, at least 87% by weight of the poloxamer has an average molecular weight of about 7250 to about 17350 Da. In some embodiments, at least 88% by weight of the poloxamer has an average molecular weight of about 7250 to about 17350 Da. In some embodiments, at least 89% by weight of the poloxamer has an average molecular weight of about 7250 to about 17350 Da. In some embodiments, at least 90% by weight of the poloxamer has an average molecular weight of about 7250 to about 17350 Da. In some embodiments, at least 91% by weight of the poloxamer has an average molecular weight of about 7250 to about 17350 Da. In some embodiments, at least 92% by weight of the poloxamer has an average molecular weight of about 7250 to about 17350 Da. In some embodiments, at least 86% by weight of the poloxamer has an average molecular weight of greater than about 7250 Da. In some embodiments, at least 87% by weight of the poloxamer has an average molecular weight of greater than about 7250 Da. In some embodiments, at least 88% by weight of the poloxamer has an average molecular weight of greater than about 7250 Da. In some embodiments, at least 89% by weight of the poloxamer has an average molecular weight of greater than about 7250 Da. In some embodiments, at least 90% by weight of the poloxamer has an average molecular weight of greater than about 7250 Da. In some embodiments, at least 91% by weight of the poloxamer has an average molecular weight of greater than about 7250 Da. Related to the embodiments in this paragraph, the poloxamer may have the following properties. In some embodiments, the poloxamer has a peak molecular weight of about 12,000 to about 12,500 Da. In some embodiments, the poloxamer has a number average molecular weight of about 11,500 to about 12,000 Da. In some embodiments, the poloxamer has a weight average molecular weight of about 11,750 to about 12,250 Da. In some embodiments, the poloxamer has a polydispersity index of about 1.02.

In some embodiments, less than 19% by weight of the poloxamer has an average molecular weight less about 7250 Da. In some embodiments, less than 18% by weight of the poloxamer has an average molecular weight less about 7250 Da. In some embodiments, less than 17% by weight of the poloxamer has an average molecular weight less about 7250 Da. In some embodiments, less than 16% by weight of the poloxamer has an average molecular weight less about 7250 Da. In some embodiments, less than 15% by weight of the poloxamer has an average molecular weight less about 7250 Da. In some embodiments, less than 14% by weight of the poloxamer has an average molecular weight less about 7250 Da. In some embodiments, less than 13% by weight of the poloxamer has an average molecular weight less about 7250 Da. In some embodiments, less than 12% by weight of the poloxamer has an average molecular weight less about 7250 Da. In some embodiments, less than 11% by weight of the poloxamer has an average molecular weight less about 7250 Da. In some embodiments, less than 10% by weight of the poloxamer has an average molecular weight less about 7250 Da. In some embodiments, less than 9% by weight of the poloxamer has an average molecular weight less about 7250 Da. Related to the embodiments in this paragraph, the poloxamer may have the following properties. In some embodiments, the poloxamer has a peak molecular weight of about 5,000 to about 5,500 Da. In some embodiments, the poloxamer has a number average molecular weight of about 5,000 to about 5,500 Da. In some embodiments, the poloxamer has a weight average molecular weight of about 5,000 to about 5,500 Da. In some embodiments, the poloxamer has a polydispersity index of about 1.02.

In some embodiments, the entire poloxamer distribution has a number average molecular weight of about 10,800 to about 11,200 Da. In some embodiments, the poloxamer distribution has a weight average molecular weight of about 11,500 to about 11,700 Da. In some embodiments, the poloxamer distribution is from 0 to about 16,600 Da. In some embodiments, the poloxamer has a polydispersity index of about less than 1.07.

In some embodiments, the poloxamer is selected from the group consisting of Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, and Poloxamer 407.

In some embodiments, the poloxamer is Poloxamer 188 or Poloxamer 407.

In some embodiments, the poloxamer is Poloxamer 407.

In some embodiments, the poloxamer comprises Poloxamer 407. In some embodiments, the Poloxamer 407 is at least 10% by weight of the poloxamer. In some embodiments, the Poloxamer 407 is at least 20% by weight of the poloxamer. In some embodiments, the Poloxamer 407 is at least 30% by weight of the poloxamer. In some embodiments, the Poloxamer 407 is at least 40% by weight of the poloxamer. In some embodiments, the Poloxamer 407 is at least 50% by weight of the poloxamer. In some embodiments, the Poloxamer 407 is at least 60% by weight of the poloxamer. In some embodiments, the Poloxamer 407 is at least 70% by weight of the poloxamer. In some embodiments, the Poloxamer 407 is at least 75% by weight of the poloxamer. In some embodiments, the Poloxamer 407 is at least 80% by weight of the poloxamer. In some embodiments, the Poloxamer 407 is at least 90% by weight of the poloxamer. In some embodiments, the poloxamer is Poloxamer 407.

In some embodiments, the poloxamer is purified Poloxamer 407.

In some embodiments, the poloxamer is a purified poloxamer (e.g., purified Poloxamer 407). In such embodiments, the solubility of the otic agent(s) may be usefully increased.

In some embodiments, the purified poloxamer (e.g., purified Poloxamer 407) has an average molecular weight of about 9 kDa or greater, about 9.2 kDa or greater, about 9.4 kDa or greater, about 9.6 kDa or greater, about 9.8 kDa or greater, about 10 kDa or greater, about 10.2 kDa or greater, about 10.4 kDa or greater, about 10.6 kDa or greater, about 10.8 kDa or greater, about 11 kDa or greater, about 11.2 kDa or greater, about 11.4 kDa or greater, about 11.6 kDa or greater, about 11.8 kDa or greater, about 12 kDa or greater, or about 12.1 kDa or greater.

In some embodiments, the purified poloxamer (e.g., purified Poloxamer 407) has a reduced level of polymer chains with molecular weight below 9 kDa as compared to the unpurified poloxamer (e.g., unpurified Poloxamer 407). In some embodiments, the polymer chains with molecular weight below 7250 Da may be regarded as impurities.

In some embodiments, the purified poloxamer (e.g., purified Poloxamer 407) has about 99% or less, about 98% or less, about 95% or less, about 90% or less, about 80% or less, about 70% or less, about 60% or less, about 50% or less, about 40% or less, about 30% or less, about 20% or less, or about 10% or less of polymer chains with molecular weight below 9 kDa as compared to the unpurified poloxamer (e.g., unpurified Poloxamer 407).

In some embodiments, the purified poloxamer (e.g., purified Poloxamer 407) contains less than about 15% by weight of polymer having a molecular weight below about 9 kDa (e.g., PEO homopolymer or PEO-PPO copolymer), for example less than about 15%, less than about 14%, less than about 13%, less than about 12%, less than about 11%, less than about 10%, less than about 5%, less than about 4%, less than about 3%, less than about 2%, less than about 1%, less than about 0.9%, less than about 0.8%, less than about 0.7%, less than about 0.6%, less than about 0.5%, less than about 0.4%, less than about 0.3%, less than about 0.2%, or less than about 0.1%, (by weight) of polymer with a molecular weight below about 9 kDa, inclusive of all ranges between any of these values.

In some embodiments, the purified poloxamer (e.g., purified Poloxamer 407) is prepared by liquid-liquid extraction or size exclusion chromatography.

General guidelines on purifying polymers are available, e.g., in U.S. Pat. No. 6,977,045, Fakhari et al. (Heliyon 3: e00390 (2017)), and PCT Application Publication No. WO/2017/108457, each of which is incorporated herein by reference. The liquid-liquid extraction procedure involves the fractionation of the poloxamer (e.g., Poloxamer 407) between two aqueous phases containing with different salt concentration. In some embodiments, one or more inpurities preferentially partition into the aqueous phase with high salt concentration, and the purified poloxamer (e.g., Poloxamer 407) remains in the aqueous phase with low salt concentration. The size exclusion chromatography provides separation based on hydrodynamic radius. The fractions containing purified poloxamer (e.g., Poloxamer 407) with the desired molecular weight range are collected.

In some embodiments, about 10% or more, about 20% or more, about 30% or more, about 40% or more, about 50% or more, about 60% or more, about 70% or more, about 80% or more, about 90% or more, about 95% or more, about 98% or more, or about 99% or more of the one or more impurities having molecular weights below 9 kDa are removed from the poloxamer (e.g., Poloxamer 407) during the purification.

In some embodiments, about 10% or more, about 20% or more, about 30% or more, about 40% or more, about 50% or more, about 60% or more, about 70% or more, about 80% or more, about 90% or more, about 95% or more, about 98% or more, or about 99% or more of the one or more diblock copolymers (e.g., PEO-PPO), single block polymers (e.g., PEO), and/or aldehydes are removed from the poloxamer (e.g., Poloxamer 407) during the purification.

In some embodiments, about 10% by weight or more, about 20% by weight or more, about 30% by weight or more, about 40% by weight or more, about 50% by weight or more, about 60% by weight or more, about 70% by weight or more, about 80% by weight or more, about 90% by weight or more, about 95% by weight or more, about 98% by weight or more, or about 99% by weight or more of the one or more diblock copolymers (e.g., PEO-PPO), single block polymers (homopolymers) (e.g., PEO), and/or aldehydes are removed from the poloxamer (e.g., Poloxamer 407) during the purification.

### Other Aspects of the Lyophilized Pharmaceutical Compositions

In some embodiments, the lyophilized pharmaceutical composition is in the form of a lyophilized cake.

In some embodiments, lyophilization of the pharmaceutical composition of the present disclosure may substantially remove all volatile components from the composition. For example, water may be substantially removed by lyophilization. For example, DMSO may be substantially removed by lyophilization. In some embodiments, the lyophilized composition is substantially free from water and/or DMSO. In some embodiments, the lyophilized composition contains less than about 5% by weight of water and/or DMSO. In some embodiments, the lyophilized composition contains less than about 4% by weight of water and/or DMSO. In some embodiments, the lyophilized composition contains less than about 3% by weight of water and/or DMSO. In some embodiments, the lyophilized composition contains less than about 2% by weight of water and/or DMSO. In some embodiments, the lyophilized composition contains less than about 1% by weight of water and/or DMSO.

In some embodiments, the lyophilized pharmaceutical composition has a higher stability to oxygen and/or light as compared to a comparable pharmaceutical composition comprising one or more solvents.

In general, where a composition with a property is compared to a composition with or without a feature to demonstrate that property, the comparative composition is an otherwise identical composition. This applies throughout the disclosure. For example, the paragraph above can be read as: the lyophilized pharmaceutical composition has a higher stability to oxygen and/or light, as compared to an otherwise identical pharmaceutical composition comprising one or more solvents.

In some embodiments, the lyophilized composition comprises at least about 1% by weight of CHIR99021 or a pharmaceutically acceptable salt thereof. In some embodiments, the lyophilized composition comprises about 1% by weight to about 2% by weight of CHIR99021. In some embodiments, the lyophilized composition comprises at least about 30% by weight of valproic acid or a pharmaceutically acceptable salt thereof. In some embodiments, the lyophilized composition comprises at least about 40% by weight of valproic acid or a pharmaceutically acceptable salt thereof. In some embodiments, the lyophilized composition comprises about 30% by weight to about 50% by weight of valproic acid or a pharmaceutically acceptable salt thereof. In some embodiments, the lyophilized composition comprises at least about 50% by weight of poloxamer. In some embodiments, the lyophilized composition comprises at least about 60% by weight of poloxamer. In some embodiments, the lyophilized composition comprises about 50% by weight to about 70% by weight of poloxamer. In some embodiments, the lyophilized composition comprises about 1.5% to about 2% by weight of CHIR99021, about 42.5% by weight to about 47.5% by weight of sodium valproate, and the remaining percentage is Poloxamer 407.

In some embodiments, the level of an impurity present in the lyophilized pharmaceutical composition is less than about 10000 parts per million (ppm), less than about 1000 ppm, less than about 100 ppm, less than about 10 ppm, less than about 1 ppm, or less than about 0.1 ppm.

In some embodiments, the total level of all the impurities present in the lyophilized pharmaceutical composition is less than about 10000 parts per million (ppm), less than about 1000 ppm, less than about 100 ppm, less than about 10 ppm, less than about 1 ppm, or less than about 0.1 ppm.

In some embodiments, the impurity is a residual solvent. In some embodiments, the impurity is selected from the group consisting of 1-acetate-2-formate-1,2-propanediol, acetic acid, formic acid, formaldehyde, acetaldehyde, and propionaldehyde.

In some embodiments, the level of polyethylene oxide presented in the lyophilized pharmaceutical composition is below about 3%, below about 2%, below about 1%, below about 0.5%, or below about 0.1%, as measured by high-performance liquid chromatography (HPLC).

In some embodiments, the total level of one or more impurities with c Log P of about 1 or less presented in the lyophilized pharmaceutical composition is from about 30% to about 35%, from about 25% to about 29%, from about 20% to about 25%, from about 15% to about 19%, from about 10% to about 14%, from about 5% to about 9%, or from about 0% to about 4%, as measured by high-performance liquid chromatography (HPLC).

In some embodiments, the total level of one or more impurities having a boiling point of about 220° C. or less presented in the lyophilized pharmaceutical composition is from about 35% to about 40%, from about 30% to about 34%, from about 25% to about 29%, from about 20% to about 25%, from about 15% to about 19%, from about 10% to about 14%, from about 5% to about 9%, or from about 0% to about 4%, as measured by high-performance liquid chromatography (HPLC).

In some embodiments, the lyophilized pharmaceutical composition comprises purified poloxamer (e.g., purified Poloxamer 407), and wherein the level of the one or more otic therapeutic agents (e.g., hearing loss treatment agents) presented in the lyophilized pharmaceutical composition is about 1.5 fold or higher, about 1.8 fold or higher, about 2 fold or higher, about 2.5 fold or higher, about 3 fold or higher, about 5 fold or higher, or about 10 fold or higher as compared to a comparable lyophilized pharmaceutical composition without purified poloxamer (e.g., purified Poloxamer 407). In some embodiments, the comparable lyophilized pharmaceutical composition comprises unpurified poloxamer (e.g., unpurified Poloxamer 407).

In some embodiments, the lyophilized pharmaceutical composition comprises purified poloxamer (e.g., purified Poloxamer 407), and wherein the dissolved concentration of the one or more otic therapeutic agents (e.g., hearing loss treatment agents) presented in the lyophilized pharmaceutical composition is about 1.5 fold or higher, about 1.8 fold or higher, about 2 fold or higher, about 2.5 fold or higher, about 3 fold or higher, about 5 fold or higher, or about 10 fold or higher as compared to an otherwise identical lyophilized pharmaceutical composition without purified poloxamer (e.g., purified Poloxamer 407). In some embodiments, the otherwise identical lyophilized pharmaceutical composition comprises unpurified poloxamer (e.g., unpurified Poloxamer 407).

In some embodiments, the lyophilized pharmaceutical composition comprises purified poloxamer (e.g., purified Poloxamer 407), and wherein the lyophilized pharmaceutical composition has lower batch-to-batch variability of one or more gelation properties (e.g., gelation temperature, viscosity, and/or stability) as compared to a comparable lyophilized pharmaceutical composition without purified poloxamer (e.g., purified Poloxamer 407). In some embodiments, the comparable lyophilized pharmaceutical composition comprises unpurified poloxamer (e.g., unpurified Poloxamer 407).

In some embodiments, the lyophilized pharmaceutical composition comprises purified poloxamer (e.g., purified Poloxamer 407), and wherein the lyophilized pharmaceutical composition has a lower gelation temperature, a narrower temperature range for gelation, and/or a higher viscosity as compared to a comparable lyophilized pharmaceutical composition without purified poloxamer (e.g., purified Poloxamer 407). In some embodiments, the comparable lyophilized pharmaceutical composition comprises unpurified poloxamer (e.g., unpurified Poloxamer 407).

In some embodiments, the lyophilized pharmaceutical composition comprises purified poloxamer (e.g., purified Poloxamer 407), and wherein the lyophilized pharmaceutical composition has a reduced degradation rate as compared to a comparable lyophilized pharmaceutical composition without purified poloxamer (e.g., purified Poloxamer 407). In some embodiments, the comparable lyophilized pharmaceutical composition comprises unpurified poloxamer (e.g., unpurified Poloxamer 407).

In some embodiments, the lyophilized pharmaceutical composition comprises one or more of a bulking agent (e.g., purified Poloxamer 407); a stabilizing agent; a tonicity-adjusting agent; and a soothing agent

In some embodiments, the lyophilized pharmaceutical composition is prepared by lyophilizing the pharmaceutical composition of the present disclosure.

In some embodiments, the lyophilized pharmaceutical composition is prepared by the method of the present disclosure.

In some embodiments, the lyophilized pharmaceutical composition is suitable for preparing a reconstituted solution by a reconstitution process.

In some embodiments, the reconstitution process is less than about 1 hour. In some embodiments, the reconstitution process is less than about 30 minutes.

In some embodiments, the reconstituted solution is suitable for injection (e.g., intratympanic injection).

In some embodiments, the reconstituted solution maintains one or more rheometric properties of a pre-lyophilized solution which is used for preparing the lyophilized pharmaceutical composition.

In some embodiments, the reconstituted solution has a reduced degradation rate as compared to a reconstituted solution prepared from a comparable lyophilized pharmaceutical composition without purified poloxamer (e.g., purified Poloxamer 407). In some embodiments, the comparable lyophilized pharmaceutical composition comprises unpurified poloxamer (e.g., unpurified Poloxamer 407). In some embodiments, the reconstituted solution maintains one or more rheometric properties of a pre-lyophilized solution which is used for preparing the lyophilized pharmaceutical composition, when the reconstituted solution is prepared at the same solids content as the pre-lyophilized solution.

### Other Aspects of the Pharmaceutical Compositions

In some embodiments, the pharmaceutical composition is a pre-lyophilized pharmaceutical composition.

In some embodiments, the pharmaceutical composition may be formed by reconstituting the lyophilized compositions disclosed herein, for example to form an aqueous composition, for example a thermoreversible gel. It will be appreciated that components of the composition will have a certain concentration when the composition is aqueous (e.g. prior to lyophilization) which will change when the composition is lyophilized since, for example, water is removed. However, for ease, it may be convenient to refer to the components of the lyophilized form by reference to their concentration when aqueous since this may be how the composition is initially produced. Reconstitution of the lyophilized composition may substantially restore a component's concentration to that in the composition prior to lyophilization.

In some embodiments, the composition comprises a gelling agent and a compound of formula (I) (as described above and in the numbered embodiments).

In some embodiments, the pharmaceutical composition comprises a gelling agent, valproic acid or a pharmaceutically acceptable salt thereof at a concentration of greater than about 70 mg/ml, and one or more otic therapeutic agents.

In some embodiments, the pharmaceutical composition comprising a poloxamer, wherein at least 85% by weight of the poloxamer has an average molecular weight of greater than about 7250 Da, and valproic acid or a pharmaceutically acceptable salt thereof at greater than 70 mg/mL.

In some embodiments, the pharmaceutical composition comprises a poloxamer, wherein less than 20% by wt. % of the poloxamer has an average molecular weight less about 7250 Da, and valproic acid or a pharmaceutically acceptable salt thereof at greater than 70 mg/mL.

In some embodiments, the composition is suitable for intratympanic injection.

In some embodiments, the gelling agent is a poloxamer (as described above and in the numbered embodiments). In some embodiments, the poloxamer comprises purified poloxamer. In some embodiments, the poloxamer comprises purified poloxamer the poloxamer is purified poloxamer. In some embodiments, the poloxamer is defined as above (as defined above and in the numbered embodiments). In some embodiments, the compositions comprises one or more otic therapeutic agents (as defined above and in the numbered embodiments). In other embodiments, the composition gelling agent comprises a hyaluronic acid. In other embodiments, the composition gelling agent comprises a cellulosic derivative.

In some embodiments, the one or more otic therapeutic agents include a GSK3 inhibitor.

In some embodiments, the one or more otic therapeutic agents include an HDAC inhibitor.

In some embodiments, the one or more otic therapeutic agents are selected from the tables above

In some embodiments, the one or more otic therapeutic agents include CHIR99021 or a pharmaceutically acceptable salt thereof. In some embodiments, the concentration of CHIR99021 or a pharmaceutically acceptable salt thereof is less than about 10 mg/mL. In some embodiments, the concentration of CHIR99021 or a pharmaceutically acceptable salt thereof is less than about 7.5 mg/mL. In some embodiments, the concentration of CHIR99021 or a pharmaceutically acceptable salt thereof is about 3 to about 7 mg/mL. In some embodiments, the concentration of CHIR99021 or a pharmaceutically acceptable salt thereof is about 4 to about 6 mg/mL. In some embodiments, the concentration of CHIR99021 or a pharmaceutically acceptable salt thereof is about 1 to about 5 mg/mL. In some embodiments, the concentration of CHIR99021 or a pharmaceutically acceptable salt thereof is about 2 to about 4 mg/mL. In some embodiments the one or more otic therapeutic agents are one or more hearing loss treatment agents.

In some embodiments, the one or more otic therapeutic agents include valproic acid or a pharmaceutically acceptable salt thereof. In some embodiments, the one or more otic therapeutic agents include valproic acid or a pharmaceutically acceptable salt thereof and CHIR99021 or a pharmaceutically acceptable salt thereof.

In some embodiments, the composition comprises a compound of formula (I) (as described above and in the numbered embodiments). In some embodiments, the compound of formula (I) and/or the one or more otic therapeutic agents are valproic acid or a pharmaceutically acceptable salt thereof.

In some embodiments, the pharmaceutically acceptable salt of valproic acid is sodium valproate. In some embodiments, the concentration of valproic acid or a pharmaceutically acceptable salt thereof is greater than about 100 mg/ml. In some embodiments, the concentration of valproic acid or a pharmaceutically acceptable salt thereof is about 100 to about 500 mg/mL. In some embodiments, the concentration of valproic acid or a pharmaceutically acceptable salt thereof is about 100 to about 350 mg/mL. In some embodiments, the concentration of valproic acid or a pharmaceutically acceptable salt thereof is about 110 to about 160 mg/ml. In some embodiments, the concentration of valproic acid or a pharmaceutically acceptable salt thereof is about 130 to about 140 mg/ml. In some embodiments, the concentration of valproic acid or a pharmaceutically acceptable salt thereof is about 125 to about 145 mg/ml. In some embodiments, the concentration of valproic acid or a pharmaceutically acceptable salt thereof is about 128 to about 138 mg/ml. In some embodiments, the concentration of valproic acid or a pharmaceutically acceptable salt thereof is about 133 mg/ml.

However, in other embodiments, the compound of formula (I) and/or the one or more otic therapeutic agents is not valproic acid or a pharmaceutically acceptable salt thereof. In some embodiments, the compound of formula (I) and/or the one or more otic therapeutic agents includes 2-(prop-2-yn-1-yl)-octanoic acid or a pharmaceutically acceptable salt thereof. In some embodiments, the compound of formula (I) and/or the one or more otic therapeutic agents includes phenylbutyric acid or a pharmaceutically acceptable salt thereof. In some embodiments, the compound of formula (I) and/or the one or more otic therapeutic agents includes linoleic acid or a pharmaceutically acceptable salt thereof.

In other embodiments, the one or more otic therapeutic agents can be different. In other embodiments, the one or more otic therapeutic agents do not include CHIR99021 or a pharmaceutically acceptable salt thereof. In some embodiments the one or more otic therapeutic agents includes LY2090314 or a pharmaceutically acceptable salt thereof. In some embodiments the one or more otic therapeutic agents includes AZD1080 or a pharmaceutically acceptable salt thereof. In some embodiments the one or more otic therapeutic agents includes GSK3 XXII or a pharmaceutically acceptable salt thereof. In some embodiments the one or more otic therapeutic agents includes Compound I-7 or a pharmaceutically acceptable salt thereof. In some embodiments the one or more otic therapeutic agents includes Compound I-1 or a pharmaceutically acceptable salt thereof.

As described above, the composition may comprise a poloxamer. While the poloxamer may vary (PEO content, purity, molecular weight range), the poloxamer may comprise the following weight percentage of the composition. In some embodiments, the concentration of poloxamer is about 2% to about 50% w/v. In some embodiments, the concentration of poloxamer is about 2% to about 40% w/v. In some embodiments, the concentration of poloxamer is about 2% to about 30% w/v. In some embodiments, the concentration of poloxamer is about 2% to about 20% w/v. In some embodiments, the concentration of poloxamer is about 10% to about 20% w/v. In some embodiments, the concentration of poloxamer is about 12.5% to about 17.5% w/v. In some embodiments, the concentration of poloxamer is about 13% to about 17.5% w/v. In some embodiments, the concentration of poloxamer is about 13% to about 17% w/v. In some embodiments, the concentration of poloxamer is about 13.5% to about 17% w/v. In some embodiments, the concentration of poloxamer is about 13.5% to about 16.5% w/v. In some embodiments, the concentration of poloxamer is about 14% to about 16.5% w/v. In some embodiments, the concentration of poloxamer is about 14% to about 16% w/v. In some embodiments, the concentration of poloxamer is about 15% to about 17.5% w/v.

In some embodiments, the disclosure relates to a method for preparing a pharmaceutical composition (for example the compositions described above or by the numbered embodiments) comprising the steps of: (a) having an aqueous solution comprising a gelling agent; and (b) adding a solution of one or more otic therapeutic agents or a pharmaceutically acceptable salt thereof.

In some embodiments, the aqueous solution further comprises valproic acid or a pharmaceutically acceptable salt thereof to the first solution. In some embodiments, the one or more otic therapeutic agents is CHIR99021 or a pharmaceutically acceptable salt thereof. In some embodiments, the one or more otic therapeutic agents is LY2090314 or a pharmaceutically acceptable salt thereof. In some embodiments, in step (b), the solution comprises a polar aprotic solvent. In some embodiments, in step (b), the polar aprotic solvent comprises DMSO. In some embodiments, in step (b), the polar aprotic solvent is DMSO. In some embodiments, in step (b), the polar aprotic solvent comprises dimethylformamide. In some embodiments, in step (b), the polar aprotic solvent comprises dimethylacetamide. In some embodiments, in step (b), the polar aprotic solvent comprises N-methyl-2-pyrrolidone. The method of any preceding embodiment, wherein the gelling agent comprises a poloxamer.

In some embodiments, the pharmaceutical composition is suitable for preparing the lyophilized pharmaceutical composition of the present disclosure (e.g., by a lyophilization process disclosed herein).

In some embodiments, the pre-lyophilized pharmaceutical composition comprises:

i) CHIR99021 or a pharmaceutically acceptable salt thereof being present at a concentration ranging from 0.025 mg/ml to about 25 mg/ml;

ii) valproic acid or a pharmaceutically acceptable salt thereof being present at a concentration ranging from 0.5 mg/ml to about 500 mg/ml;

iii) poloxamer 407 being present at a concentration ranging from 1 wt % to about 25 wt %; and

iv) dimethyl sulfoxide (DMSO) being present at a concentration below 7.5 wt %.

In some embodiments, the pre-lyophilized pharmaceutical composition comprises:

i) CHIR99021 or a pharmaceutically acceptable salt thereof being present at a concentration ranging from 0.025 mg/ml to about 25 mg/ml;

ii) valproic acid or a pharmaceutically acceptable salt thereof being present at a concentration ranging from 0.5 mg/ml to about 500 mg/ml;

iii) poloxamer 407 being present at a concentration ranging from 1 wt % to about 25 wt %; and

iv) dimethyl sulfoxide (DMSO) being present at a concentration below 25 wt %.

In some embodiments, the pharmaceutically acceptable salt of valproic acid is a sodium salt (e.g., sodium valproate).

In some embodiments, the pre-lyophilized pharmaceutical composition described in “Other Aspects of the Pharmaceutical Compositions” (or any embodiments described above), the pharmaceutically acceptable salt of valproic acid is a sodium salt (e.g., sodium valproate).

Any individual component of a composition may be present at a given concentration. Concentration can have the units of percent weight per volume (w/v) which can also be expressed as g/mL.

In some embodiments, the concentration of CHIR99021 or the pharmaceutically acceptable salt thereof in the pre-lyophilized pharmaceutical composition ranges from about 0.05 mg/ml to about 5 mg/ml, from about 0.25 mg/ml to about 2.5 mg/ml, from about 0.5 mg/ml to about 1.75 mg/ml, or from about 1.45 mg/ml to about 1.65 mg/ml. In some embodiments, the concentration of CHIR99021 or the pharmaceutically acceptable salt thereof is about 1.55 mg/ml.

In some embodiments, the concentration of valproic acid or the pharmaceutically acceptable salt thereof in the pre-lyophilized pharmaceutical composition ranges from about 2.5 mg/ml to about 200 mg/ml, from about 5 mg/ml to about 100 mg/ml, from about 15 mg/ml to about 50 mg/ml, or from about 43 mg/ml to about 46 mg/ml. In some embodiments, the concentration of valproic acid or the pharmaceutically acceptable salt thereof is about 44.5 mg/ml.

In some embodiments, the concentration of poloxamer 407 in the pre-lyophilized pharmaceutical composition ranges from about 2.5 wt % to about 12.5 wt %, from about 5 wt % to about 11 wt %, from about 6 wt % to about 10 wt %, or from about 7 wt % to about 8.5 wt %. In some embodiments, the concentration of poloxamer 407 is about 8 wt %.

In some embodiments, the concentration of DMSO in the pre-lyophilized pharmaceutical composition ranges from about 0.5 wt % to about 5 wt %, from about 1 wt % to about 4 wt %, from about 1.5 wt % to about 3.5 wt %, or from about 2 wt % to about 3 wt %. In some embodiments, the concentration of DMSO is about 2.5 wt %.

In some embodiments, the concentration of DMSO in the composition is about less than 5 wt %, as described above. However, in other embodiments, it will be appreciated that the concentration of DMSO may be less than about 25 wt %. In some embodiments, the concentration of DMSO is about less than 25 wt %. In some embodiments, the concentration of DMSO is about less than 20 wt %. In some embodiments, the concentration of DMSO is about less than 15 wt %. In some embodiments, the concentration of DMSO is about less than 10 wt %. In some embodiments, the concentration of DMSO is about less than 5 wt %. In some embodiments, wherein the concentration of DMSO is about 25 to about 15 wt %. In some embodiments, wherein the concentration of DMSO is about 20 to about 10 wt %. In some embodiments, wherein the concentration of DMSO is about 15 to about 5 wt %. In some embodiments, wherein the concentration of DMSO is about 10 to about 5 wt %.

In some embodiments, the weight ratio between CHIR99021 or the pharmaceutically acceptable salt thereof and valproic acid or the pharmaceutically acceptable salt thereof in the pre-lyophilized pharmaceutical composition ranges from about 1:5 to about 1:10, from about 1:10 to about 1:50, from about 1:20 to about 1:35, from about 1:25 to about 1:31, or from about 1:27 to about 1:29. One skilled in the art will understand that the weight ratio of CHIR99012 and valproic acid (or pharmaceutically acceptable salts thereof) will be substantially unchanged in the lyophilized and reconstituted pharmaceutical composition.

In some embodiments, the weight ratio between poloxamer 407 and the DMSO in the pre-lyophilized pharmaceutical composition ranges from about 1:5 to about 40:1, from about 1:2 to about 15:1, from about 1:1 to about 8:1, from about 2:1 to about 4:1, or from about 2.5:1 to about 3.5:1. In some embodiments, the weight ratio between poloxamer 407 and the DMSO is about 3:1.

In some embodiments, the weight ratio between CHIR99021 and poloxamer 407 in the pre-lyophilized pharmaceutical composition is about 0.02:1; the weight ratio between CHIR99021 and the DMSO is about 0.06:1; the weight ratio between valproic acid sodium salt and poloxamer 407 is about 0.54:1; and/or the weight ratio between valproic acid sodium salt and the DMSO is about 3.2:1.

In some embodiments, the concentration of CHIR99021 or the pharmaceutically acceptable salt thereof in the pre-lyophilized pharmaceutical composition ranges from about 1.45 mg/ml to about 1.65 mg/ml; the concentration of valproic acid or the pharmaceutically acceptable salt thereof ranges from about 43 mg/ml to about 46 mg/ml; the concentration of poloxamer 407 ranges from about 7 wt % to about 8.5 wt %; and the concentration of DMSO ranges from about 2 wt % to about 3 wt %.

In some embodiments, the concentration of CHIR99021 or the pharmaceutically acceptable salt thereof in the pre-lyophilized pharmaceutical composition is about 1.55 mg/ml; the concentration of valproic acid or the pharmaceutically acceptable salt thereof is about 44.5 mg/ml; the concentration of poloxamer 407 is about 8 wt %; and the concentration of DMSO is about 2.5 wt %.

In some embodiments, the pre-lyophilized pharmaceutical composition comprises:

i) CHIR99021 or a pharmaceutically acceptable salt thereof being present at a concentration ranging from 0.025 mg/ml to about 25 mg/ml;

ii) valproic acid or a pharmaceutically acceptable salt thereof being present at a concentration ranging from 1 mg/ml to about 500 mg/ml;

iii) poloxamer 407 being present at a concentration ranging from 1 wt % to about 25 wt %; and

iv) dimethyl sulfoxide (DMSO) being present at a concentration below 7.5 wt %.

In some embodiments, the pharmaceutically acceptable salt of valproic acid is a sodium salt (e.g., sodium valproate).

In some embodiments, the concentration of CHIR99021 or the pharmaceutically acceptable salt in the pre-lyophilized pharmaceutical composition thereof ranges from about 0.05 mg/ml to about 10 mg/ml, from about 0.25 mg/ml to about 2.5 mg/ml, from about 0.5 mg/ml to about 1.75 mg/ml, from about 0.85 mg/ml to about 1.15 mg/ml. In some embodiments, the concentration of CHIR99021 or the pharmaceutically acceptable salt thereof is about 1.05 mg/ml.

In some embodiments, the concentration of valproic acid or the pharmaceutically acceptable salt thereof in the pre-lyophilized pharmaceutical composition ranges from about 2.5 mg/ml to about 200 mg/ml, from about 5 mg/ml to about 100 mg/ml, from about 15 mg/ml to about 50 mg/ml, from about 28 mg/ml to about 31 mg/ml. In some embodiments, the concentration of valproic acid or the pharmaceutically acceptable salt thereof is about 29.5 mg/ml.

In some embodiments, the concentration of poloxamer 407 in the pre-lyophilized pharmaceutical composition ranges from about 2.5 wt % to about 12.5 wt %, from about 5 wt % to about 11 wt %, from about 11 wt % to about 10 wt %, from about 7 wt % to about 8.5 wt %. In some embodiments, the concentration of poloxamer 407 is about 7.5 wt %.

In some embodiments, the concentration of DMSO in the pre-lyophilized pharmaceutical composition ranges from about 0.5 wt % to about 5 wt %, from about 1 wt % to about 4 wt %, from about 1.5 wt % to about 3.5 wt %, from about 2 wt % to about 3 wt %. In some embodiments, the concentration of DMSO is about 2.5 wt %.

In some embodiments, the weight ratio between CHIR99021 or the pharmaceutically acceptable salt thereof and valproic acid or the pharmaceutically acceptable salt thereof in the pre-lyophilized pharmaceutical composition ranges from about 1:5 to about 1:10, from about 1:10 to about 1:50, from about 1:20 to about 1:35, from about 1:25 to about 1:31, or from about 1:27 to about 1:29. One skilled in the art will understand that the weight ratio of CHIR99012 and valproic acid (or pharmaceutically acceptable salts thereof) will be substantially unchanged in the lyophilized and reconstituted pharmaceutical composition.

In some embodiments, the weight ratio between poloxamer 407 and the DMSO in the pre-lyophilized pharmaceutical composition ranges from about 1:5 to about 40:1, from about 1:2 to about 15:1, from about 1:1 to about 8:1, from about 2:1 to about 4:1, from about 2.5:1 to about 3.5:1. In some embodiments, the weight ratio between poloxamer 407 and the DMSO is about 3:1.

In some embodiments, the weight ratio between CHIR99021 and poloxamer 407 in the pre-lyophilized pharmaceutical composition is about 0.016:1; the weight ratio between the CHIR99021 and the DMSO is about 0.06:1; the weight ratio between valproic acid sodium salt and poloxamer 407 is about 0.42:1; and/or the weight ratio between valproic acid sodium salt and the DMSO is about 1.5:1.

In some embodiments, the concentration of CHIR99021 or the pharmaceutically acceptable salt thereof ranges from about 0.95 mg/ml to about 1.15 mg/ml in the pre-lyophilized pharmaceutical composition; the concentration of valproic acid or the pharmaceutically acceptable salt thereof ranges from about 28 mg/ml to about 31 mg/ml; the concentration of poloxamer 407 ranges from about 7 wt % to about 8.5 wt %; and the concentration of DMSO ranges from about 2 wt % to about 3 wt %.

In some embodiments, the concentration of CHIR99021 or the pharmaceutically acceptable salt thereof in the pre-lyophilized pharmaceutical composition is about 1.05 mg/ml; the concentration of valproic acid or the pharmaceutically acceptable salt thereof is about 29.5 mg/ml; the concentration of poloxamer 407 is about 7.5 wt %; and the concentration of DMSO is about 2.5 wt %.

In some embodiments, the pre-lyophilized pharmaceutical composition comprises:

i) CHIR99021 or a pharmaceutically acceptable salt thereof being present at a concentration ranging from 0.025 mg/ml to about 25 mg/ml;

ii) valproic acid or a pharmaceutically acceptable salt thereof being present at a concentration ranging from 0.5 mg/ml to about 500 mg/ml;

iii) poloxamer 407 being present at a concentration ranging from 1 wt % to about 25 wt %; and

iv) dimethyl sulfoxide (DMSO) being present at a concentration below 7.5 wt %.

In some embodiments, the pharmaceutically acceptable salt of valproic acid is a sodium salt (e.g., sodium valproate).

In some embodiments, the concentration of CHIR99021 or the pharmaceutically acceptable salt thereof in the pre-lyophilized pharmaceutical composition ranges from about 0.05 mg/ml to about 5 mg/ml, from about 0.25 mg/ml to about 2.5 mg/ml, from about 0.5 mg/ml to about 1.75 mg/ml, or from about 0.6 mg/ml to about 0.75 mg/ml. In some embodiments, the concentration of CHIR99021 or the pharmaceutically acceptable salt thereof ranges is about 0.7 mg/ml.

In some embodiments, the concentration of valproic acid or the pharmaceutically acceptable salt thereof in the pre-lyophilized pharmaceutical composition ranges from about 2.5 mg/ml to about 200 mg/ml, from about 5 mg/ml to about 100 mg/ml, from about 15 mg/ml to about 50 mg/ml, or from about 18 mg/ml to about 21 mg/ml. In some embodiments, the concentration of valproic acid or the pharmaceutically acceptable salt thereof is about 19.5 mg/ml.

In some embodiments, the concentration of poloxamer 407 in the pre-lyophilized pharmaceutical composition ranges from about 2.5 wt % to about 12.5 wt %, from about 5 wt % to about 11 wt %, from about 6 wt % to about 10 wt %, or from about 7 wt % to about 8.5 wt %. In some embodiments, the concentration of poloxamer 407 is about 7.5 wt %.

In some embodiments, the concentration of DMSO in the pre-lyophilized pharmaceutical composition ranges from about 0.5 wt % to about 5 wt %, from about 1 wt % to about 4 wt %, from about 1.5 wt % to about 3.5 wt %, or from about 2 wt % to about 3 wt %. In some embodiments, the concentration of DMSO is about 5 wt %.

In some embodiments, the weight ratio between CHIR99021 or the pharmaceutically acceptable salt thereof and valproic acid or the pharmaceutically acceptable salt thereof in the pre-lyophilized pharmaceutical composition ranges from about 1:5 to about 1:10, from about 1:10 to about 1:50, from about 1:20 to about 1:35, from about 1:25 to about 1:31, or from about 1:27 to about 1:29. One skilled in the art will understand that the weight ratio of CHIR99012 and valproic acid (or pharmaceutically acceptable salts thereof) will be substantially unchanged in the lyophilized and reconstituted pharmaceutical composition.

In some embodiments, the weight ratio between poloxamer 407 and the DMSO in the pre-lyophilized pharmaceutical composition ranges from about 1:5 to about 40:1, from about 1:2 to about 15:1, from about 1:1 to about 8:1, from about 2:1 to about 4:1, from about 2.5:1 to about 3.5:1.

In some embodiments, the weight ratio between poloxamer 407 and the DMSO in the pre-lyophilized pharmaceutical composition is about 3:1; the weight ratio between the CHIR99021 and poloxamer 407 is about 0.013:1; the weight ratio between CHIR99021 and the DMSO is about 0.06:1; the weight ratio between valproic acid sodium salt and poloxamer 407 is about 0.23:1; and/or the weight ratio between valproic acid sodium salt and the DMSO is about 1.8:1.

In some embodiments, the concentration of CHIR99021 or the pharmaceutically acceptable salt thereof in the pre-lyophilized pharmaceutical composition ranges from about 0.6 mg/ml to about 0.75 mg/ml; the concentration of valproic acid or the pharmaceutically acceptable salt thereof ranges from about 18 mg/ml to about 21 mg/ml; the concentration of poloxamer 407 ranges from about 7 wt % to about 8.5 wt %; and the concentration of DMSO ranges from about 2 wt % to about 3 wt %.

In some embodiments, the concentration of CHIR99021 or the pharmaceutically acceptable salt thereof in the pre-lyophilized pharmaceutical composition is about 0.7 mg/ml; the concentration of valproic acid or the pharmaceutically acceptable salt thereof is about 19.5 mg/ml; the concentration of poloxamer 407 is about 7.5 wt %; and the concentration of DMSO is about 2.5 wt %.

In some embodiments, the pre-lyophilized pharmaceutical composition comprises one or more of water or a buffering agent; a bulking agent; a stabilizing agent (e.g., purified Poloxamer 407); a tonicity-adjusting agent; and a soothing agent

### Methods of Preparing Lyophilized Pharmaceutical Compositions

In some aspects, the present disclosure provides a method of preparing a lyophilized pharmaceutical composition of the present disclosure.

In some aspects, the present disclosure provides a method of processing the pharmaceutical composition of the present disclosure to form a lyophilized pharmaceutical composition (e.g., the pharmaceutical composition of the present disclosure).

In embodiments, the method involves a lyophilization process.

In embodiments, the disclosure relates to a method of lyophilizing a pharmaceutical composition as described by the pharmaceutical composition above and the numbered embodiments, wherein the method comprises: (a) providing a pharmaceutical composition; (b) lyophilizing the composition by: (i) reducing the temperature in the lyophilizer to −45° C. at a rate of 0.5° C. per minute, and then holding it at −45° C. for 3 hours; (ii) applying a vacuum of 80 mTorr; (iii) increasing the temperature to −30° C. (at a rate of 0.5° C. per minute) and holding it at −30° C. for 15 hours under a vacuum of 80 mTorr; (iv) increasing the temperature to 15° C. (at a rate of 0.5° C. per minute); and/or (v) holding the temperature at 15° C. for 20 hours under a vacuum of 80 mTorr; and (d) obtaining a lyophilized pharmaceutical composition. In some embodiments, the composition is subjected to a temperature of at least −50° C. prior to lyophilization. In some embodiments, the method can be varied by any one or more of the numbered embodiments below.

In embodiments, the disclosure relates to a method of lyophilizing a pharmaceutical composition as described by the pharmaceutical composition above and the numbered embodiments, wherein the method comprises: (a) providing a pharmaceutical composition; (b) lyophilizing the composition by: (i) reducing the temperature in the lyophilizer to about −45° C. at a rate of about 0.5° C. per minute, and then holding it at about −45° C. for about 3 hours; (ii) applying a vacuum of about 80 mTorr; (iii) increasing the temperature to about −30° C. (at a rate of about 0.5° C. per minute) and holding it at about −30° C. for about 15 hours under a vacuum of about 80 mTorr; (iv) increasing the temperature to about 15° C. (at a rate of about 0.5° C. per minute); and/or (v) holding the temperature at about 15° C. for about 20 hours under a vacuum of about 80 mTorr; and (d) obtaining a lyophilized pharmaceutical composition. In some embodiments, the composition is subjected to a temperature of at least about −50° C. prior to lyophilization. In some embodiments, the method can be varied by any one or more of the numbered embodiments below.

In some embodiments, the pharmaceutical composition is sterilized prior to the lyophilization process. In some embodiments, the pharmaceutical composition is sterilized through filtration (e.g., a sterile filtration) using a filter, for example a microporous membrane.

In some embodiments, the filter comprises a nylon, polycarbonate, cellulose acetate, polyvinylidene fluoride (PVDF), polytetrafluoroethylene (PTFE), polyethersulfone (PES), or any combination thereof.

In some embodiments, the filter is a polyethersulfone (PES) membrane filter or a polytetrafluoroethylene (PTFE) membrane filter. In some embodiments, the filter has a pore size of about 0.01 μm, about 0.02 μm, about 0.05 μm, about 0.08 μm, about 0.1 μm, about 0.2 μm, about 0.3 μm, about 0.4 μm, about 0.5 μm, or about 1 μm.

In some embodiments, one or more of microorganisms (e.g., bacteria, mold, or yeast) and particles are substantially removed from the pharmaceutical composition by the filtration.

In some embodiments, the method comprises the steps of:

i) cooling the pharmaceutical composition at a first temperature below 0° C. for a first period of time;

ii) removing one or more solvents from the resulting mixture of step (i) at a second temperature below 0° C., and at a reduced pressure below 760 Torr, for a second period of time.

In some embodiments, the method comprises one or more steps selected from:

0a) dispensing the pharmaceutical composition in a sterile vial;

ia) cooling the pharmaceutical composition at a rate ranging from about 0.1° C. per minute to about 5° C. per minute to the first temperature ranging from about −20° C. to about −80° C.;

ib) holding the pharmaceutical composition at the first temperature for the first period of time ranging from about 1 hour to about 6 hours;

iia) subjecting the pharmaceutical composition to the reduced pressure ranging from about 1 mTorr to 1000 mTorr and warming the pharmaceutical composition at a rate ranging from about 0.1° C. per minute to about 5° C. per minute to the second temperature ranging from about −10° C. to −50° C.;

iib) holding the pharmaceutical composition at the second temperature and under the reduced pressure or the second period of time ranging from about 10 hours to about 30 hours;

iiia) filling the sterile vial with nitrogen; and

iiib) capping and crimping the sterile vial.

In some embodiments, the pharmaceutical composition comprises the one or more otic therapeutic agents (e.g., hearing loss treatment agents) and the poloxamer. In some embodiments, the pharmaceutical composition comprises the one or more otic therapeutic agents (e.g., hearing loss treatment agents) and poloxamer 407. In some embodiments, the pharmaceutical composition comprises the one or more otic therapeutic agents (e.g., hearing loss treatment agents) and purified poloxamer 407.

In some embodiments, the pharmaceutical composition comprises CHIR99021, valproic acid sodium salt, the poloxamer, DMSO, and water. In some embodiments, the pharmaceutical composition comprises CHIR99021, valproic acid sodium salt, poloxamer 407, DMSO, and water. In some embodiments, the pharmaceutical composition comprises CHIR99021, valproic acid sodium salt, purified poloxamer 407, DMSO, and water.

In some embodiments, the method comprises one or more steps selected from:

0a) dispensing the pharmaceutical composition in a sterile vial;

ia) cooling the pharmaceutical composition at a rate of about 0.5° C. per minute to the first temperature of about −45° C.;

ib) holding the pharmaceutical composition at the first temperature for the first period of time of about 3 hours;

iia) subjecting the pharmaceutical composition to the reduced pressure of about 80 mTorr to 1000 mTorr and warming the pharmaceutical composition at a rate of about 0.5° C. per minute to the second temperature of about −30° C.;

iib) holding the pharmaceutical composition at the second temperature and under the reduced pressure for the second period of time ranging from about 10 hours to about 15 hours;

iic) warming the pharmaceutical composition at a rate of about 0.5° C. per minute to 20° C.;

iid) holding the pharmaceutical composition at 20° C. and under the reduced pressure for 20 hours,

iiia) filling the sterile vial with nitrogen; and

iiib) capping and crimping the sterile vial.

### Other Aspects of the Reconstituted Solutions

In some embodiments, the reconstituted solution is prepared by adding a diluent to the lyophilized pharmaceutical composition of the present disclosure.

In some embodiments, the disclosure relates to a method for reconstituting a lyophilized pharmaceutical composition (as described above or in the numbered embodiments), the method comprising: (a) providing the lyophilized pharmaceutical composition of any preceding embodiment; (b) reconstituting the lyophilized pharmaceutical composition with a pharmaceutically acceptable diluent; and (c) obtaining a reconstituted pharmaceutical composition.

In some embodiments, reconstituting the lyophilized pharmaceutical composition comprises dissolving the lyophilized pharmaceutical composition in the pharmaceutically acceptable diluent. In some embodiments, dissolving the lyophilized pharmaceutical composition in the pharmaceutically acceptable diluent takes less than about 1 hour. In some embodiments, dissolving the lyophilized pharmaceutical composition in the pharmaceutically acceptable diluent takes less than about 45 minutes. In some embodiments, dissolving the lyophilized pharmaceutical composition in the pharmaceutically acceptable diluent takes less than about 30 minutes. In some embodiments, dissolving the lyophilized pharmaceutical composition in the pharmaceutically acceptable diluent takes less than about 15 minutes. In some embodiments, dissolving the lyophilized pharmaceutical composition in the pharmaceutically acceptable diluent takes less than about 10 minutes.

In some embodiments, a reconstituted pharmaceutical composition can be obtained by the method for reconstituting a lyophilized pharmaceutical composition.

In some embodiments, a reconstituted pharmaceutical composition comprises the lyophilized composition of the present disclosure and a diluent.

In some embodiments, the composition reconstitutes in less about 1 hour. In some embodiments, the composition reconstitutes in less than about 45 minutes. In some embodiments, the composition reconstitutes in less than about 30 minutes. In some embodiments, the composition reconstitutes in less than about 15 minutes. In some embodiments, the composition reconstitutes in less than about 10 minutes.

In some embodiments, the lyophilized pharmaceutical composition is prepared by lyophilizing the pharmaceutical composition of the present disclosure.

In some embodiments, the lyophilized pharmaceutical composition is prepared by the method of the present disclosure.

In some embodiments, the lyophilized pharmaceutical composition comprises one or more otic therapeutic agents (e.g., hearing loss treatment agents) and a gelling agent.

In some embodiments, the diluent comprises water and dimethyl sulfoxide (DMSO).

In some embodiments, the concentration of DMSO in the diluent ranges from about 1% w/w to about 15% w/w, from about 2% w/w to about 12% w/w, from about 3% w/w to about 10% w/w, from about 4% w/w to about 9% w/w, from about 5% w/w to about 8% w/w, from about 5.5% w/w to about 7.5% w/w, from about 5.8% w/w to about 7% w/w, from about 6% w/w to about 6.8% w/w, or from about 6.2% w/w to about 6.6% w/w. In some embodiments, the concentration of DMSO in the diluent is about 6.4% w/w. In some embodiments, the diluent is 6.4 w/w % DMSO in water.

In some embodiments, the amount of the diluent added during the reconstitution ranges from about 1 μL to about 6 μL, from about 2 μL to about 5 μL, from about 2.5 μL to about 4.5 μL, from about 2.8 μL to about 4 μL, from about 3 μL to about 3.8 μL, or from about 3.2 μL to about 3.6 μL per mg of the lyophilized pharmaceutical composition. In some embodiments, the amount of the diluent added during the reconstitution is about 3.4 μL per mg of the lyophilized pharmaceutical composition.

In some embodiments, the amount of the diluent added during the reconstitution is about 20 grams, about 30 grams, about 40 grams, about 50 grams, about 60 grams, about 70 grams, about 80 grams, about 90 grams, about 100 grams, about 120 grams, about 150 grams, about 200 grams, about 300 grams, about 500 grams, about 800 grams, or about 1000 grams.

In some embodiments, the amount of the diluent added during the reconstitution is about 0.1 mL-about 1.5 mL, about 0.3 mL-about 1.3 mL, about 0.5 mL-about 1.1 mL or about 0.7 mL-about 0.9 mL. In some embodiments, the amount of the diluent added during the reconstitution is about 0.85 mL.

In some embodiments, the diluent is sparged with nitrogen for about 10 seconds to about 30 minutes, from about 20 seconds to about 20 minutes, from about 30 seconds, to about 10 minutes, from about 40 seconds to about 5 minutes, from about 50 seconds to about 3 minutes, or from about 1 minute to about 2 minutes prior to being added to the lyophilized pharmaceutical composition.

In some embodiments, the diluent is sterile filtered (e.g., using a PES 0.2 μm filter and/or a 10 mL syringe) prior to being added to the lyophilized pharmaceutical composition

In some embodiments, upon addition, the mixture of the lyophilized pharmaceutical composition and the diluent is held at temperature lower than ambient temperature for a period time, thereby forming the reconstituted solution. In various embodiments, the reconstitution process is conducted without any agitation of the mixture of the lyophilized pharmaceutical composition and the diluent (e.g., shaking, sonication, or vortexing). In some embodiments, the reconstitution process comprises gently rotating the container (e.g., the vial) to mix the lyophilized pharmaceutical composition and the diluent, and/or gently tapping the container (e.g., the vial) until the lyophilized pharmaceutical composition and the diluent form a homogeneous solution.

In some embodiments, the mixture of the lyophilized pharmaceutical composition and the diluent is held at a temperature ranging from about −10° C. to about 20° C., from about −5° C. to about 15° C., from about 0° C. to about 10° C., from about 1° C. to about 9° C., or from about 2° C. to about 8° C. In some embodiments, the mixture of the lyophilized pharmaceutical composition and the diluent is held at a temperature ranging from about 5-8° C.

In some embodiments, the mixture of the lyophilized pharmaceutical composition and the diluent is held for a period of time (e.g., reconstitution time) being about 6 hours or less, about 3 hours or less, about 2 hours or less, about 1 hours or less, about 50 minutes or less, about 40 minutes or less, about 30 minutes or less, about 20 minutes or less, or about 10 minutes or less. In certain embodiments, the mixture of the lyophilized pharmaceutical composition and the diluent is held for 20 minutes.

In some embodiments, the reconstitution process comprises addition of the diluent to the lyophilized pharmaceutical composition and storing the vial at 2-8° C. In some embodiments, the reconstitution process comprises addition of the diluent to the lyophilized pharmaceutical composition and storing the vial at 2-8° C. and gently tapping the container (e.g., the vial) until the lyophilized pharmaceutical composition and the diluent form a homogeneous solution. In some embodiments, the reconstitution process comprises addition of the diluent to the lyophilized pharmaceutical composition and storing the vial at 2-8° C. and gently tapping the container (e.g., the vial) until the lyophilized pharmaceutical composition and the diluent form a homogeneous solution without sonication or vortexing (for example in order to avoid poloxamer degradation or drug precipitation). In some embodiments, the reconstitution process comprises addition of about 0.85 mL of diluent to the lyophilized pharmaceutical composition and storing the vial at 2-8° C. and gently tapping the container (e.g., the vial) until the lyophilized pharmaceutical composition and the diluent form a homogeneous solution without sonication or vortexing. In some embodiments, the reconstitution process comprises addition of about 0.85 mL of diluent to the lyophilized pharmaceutical composition and storing the vial at 2-8° C. and gently tapping the container (e.g., the vial) until the lyophilized pharmaceutical composition and the diluent form a homogeneous solution without sonication or vortexing where the diluent is 6.4 w/w % DMSO in water. In some embodiments any of the reconstitution processes can be used to measure improved reconstitution time, for example the improvements discussed herein e.g. relative to non-lyophilized solid forms. In certain embodiments, the improvement in reconstitution time disclosed herein is specifically measured using a reconstitution process in which about 0.85 mL of diluent is added to the lyophilized pharmaceutical composition, the vial is stored at 2-8° C. and gently tapped until the lyophilized pharmaceutical composition and the diluent form a homogeneous solution without sonication or vortexing, where the diluent is 6.4 w/w % DMSO in water. Improvements could be observed after a fixed reconstitution time, e.g. 20 minutes.

In some embodiments, the reconstituted solution is a clear solution at ambient temperature (e.g., between 20° C. and 26° C.).

In some embodiments, the reconstituted solution is suitable for injection at ambient temperature (e.g., between 20° C. and 26° C.).

In some embodiments, the reconstituted solution has a gelation temperature being higher than ambient temperature (e.g., between 20° C. and 26° C., preferably 25° C.) and being lower than the temperature of human body (e.g., between 36° C. and 39° C., preferably 37° C.).

In some embodiments, the reconstituted solution has a gelation temperature range of about 2° C. or about 3° C.

In some embodiment, the reconstituted solution is stable upon storage of at a temperature ranging from about −10° C. to about 20° C., from about −5° C. to about 15° C., from about 0° C. to about 10° C., from about 1° C. to about 9° C., or from about 2° C. to about 8° C.

In some embodiment, the reconstituted solution is stored for about 10 minutes or longer, about 20 minutes or longer, about 30 minutes or longer, about 40 minutes or longer, about 50 minutes or longer, about 1 hour or longer, about 2 hours or longer, about 3 hours or longer, about 4 hours or longer, about 5 hours or longer, or about 6 hours or longer prior to use.

In some embodiment, about 0.1% or less, about 0.09% or less, about 0.08% or less, about 0.07% or less, about 0.06% or less, about 0.05% or less, about 0.04% or less, about 0.03% or less, about 0.02% or less, or about 0.01% or less of one or more otic therapeutic agents (e.g., CHIR99021 and/or sodium valproate) degrades during the storage.

In some embodiments, the reconstituted solution has a pH value ranging from about 4 to about 13, from about 5 to about 12, from about 6 to about 11, from about 6.5 to about 10.5, or from about 7 to about 10.

In some embodiments, the reconstituted solution is suitable for injection at ambient temperature (e.g., between 20° C. and 26° C.) through a needle (e.g., a needle having an inner diameter of about 3.81 mm or less, about 3.43 mm or less, about 3.00 mm or less, about 2.69 mm or less, about 2.39 mm or less, about 2.16 mm or less, about 1.80 mm or less, about 160 mm or less, about 1.37 mm or less, about 1.19 mm or less, about 1.07 mm or less, about 0.84 mm or less, about 0.69 mm or less, about 0.60 mm or less, about 0.51 mm or less, about 0.41 mm or less, about 0.34 mm or less, about 0.31 mm or less, or about 0.26 mm or less).

In some embodiments, the reconstituted solution is formulated for injection in a volume of about 1 ml or less, about 900 μl or less, about 800 μl or less, about 700 μl or less, about 600 μl or less, about 500 μl or less, about 400 μl or less, about 300 μl or less, about 200 μl or less, or about 100 or less.

In some embodiments, the reconstituted solution comprises:

i) CHIR99021 or a pharmaceutically acceptable salt thereof being present at a concentration ranging from 0.05 mg/ml to about 50 mg/ml;

ii) valproic acid or a pharmaceutically acceptable salt thereof being present at a concentration ranging from 1 mg/ml to about 1000 mg/ml;

iii) poloxamer 407 being present at a concentration ranging from 2 wt % to about 50 wt %; and

iv) dimethyl sulfoxide (DMSO) being present at a concentration below 15 wt %.

In some embodiments, the pharmaceutically acceptable salt of valproic acid is a sodium salt. In some embodiments, the pharmaceutically acceptable salt of valproic acid is sodium valproate.

In some embodiments, the concentration of CHIR99021 or the pharmaceutically acceptable salt thereof in the reconstituted solution ranges from about 0.1 mg/ml to about 10 mg/ml, from about 0.5 mg/ml to about 5 mg/ml, from about 1 mg/ml to about 3.5 mg/ml, or from about 2.9 mg/ml to about 3.3 mg/ml. In some embodiments, the concentration of CHIR99021 or the pharmaceutically acceptable salt thereof is about 3.1 mg/ml.

In some embodiments, the concentration of valproic acid or the pharmaceutically acceptable salt thereof in the reconstituted solution ranges from about 5 mg/ml to about 400 mg/ml, from about 10 mg/ml to about 200 mg/ml, from about 30 mg/ml to about 100 mg/ml, or from about 86 mg/ml to about 92 mg/ml. In some embodiments, the concentration of valproic acid or the pharmaceutically acceptable salt thereof is about 89 mg/ml.

In some embodiments, the concentration of poloxamer 407 in the reconstituted solution ranges from about 5 wt % to about 25 wt %, from about 10 wt % to about 22 wt %, from about 12 wt % to about 20 wt %, or from about 14 wt % to about 17 wt %. In some embodiments, the concentration of poloxamer 407 is about 16 wt %.

In some embodiments, the concentration of DMSO in the reconstituted solution ranges from about 1 wt % to about 10 wt %, from about 2 wt % to about 8 wt %, from about 3 wt % to about 7 wt %, or from about 4 wt % to about 6 wt %. In some embodiments, the concentration of DMSO is about 5 wt %.

In some embodiments, the weight ratio between CHIR99021 or the pharmaceutically acceptable salt thereof and valproic acid or the pharmaceutically acceptable salt thereof in the reconstituted solution ranges from about 1:5 to about 1:10, from about 1:10 to about 1:50, from about 1:20 to about 1:35, from about 1:25 to about 1:31, or from about 1:27 to about 1:29.

In some embodiments, the weight ratio between poloxamer 407 and the DMSO ranges in the reconstituted solution from about 1:5 to about 40:1, from about 1:2 to about 15:1, from about 1:1 to about 8:1, from about 2:1 to about 4:1, or from about 2.5:1 to about 3.5:1. In some embodiments, the weight ratio between poloxamer 407 and the DMSO is about 3:1.

In some embodiments, the weight ratio between CHIR99021 and poloxamer 407 in the reconstituted solution is about 0.02:1; the weight ratio between CHIR99021 and the DMSO is about 0.06:1; the weight ratio between valproic acid sodium salt and poloxamer 407 is about 0.54:1; and/or the weight ratio between valproic acid sodium salt and the DMSO is about 3.2:1.

In some embodiments, the concentration of CHIR99021 or the pharmaceutically acceptable salt thereof in the reconstituted solution ranges from about 2.9 mg/ml to about 3.3 mg/ml; the concentration of valproic acid or the pharmaceutically acceptable salt thereof ranges from about 86 mg/ml to about 92 mg/ml; the concentration of poloxamer 407 ranges from about 14 wt % to about 17 wt %; and the concentration of DMSO ranges from about 4 wt % to about 6 wt %.

In some embodiments, the concentration of CHIR99021 or the pharmaceutically acceptable salt thereof in the reconstituted solution ranges from about 3.2 mg/ml to about 3.3 mg/ml; the concentration of valproic acid or the pharmaceutically acceptable salt thereof ranges from about 87 mg/ml to about 90 mg/ml; the concentration of poloxamer 407 ranges from about 14 wt % to about 16 wt %; and the concentration of DMSO ranges from about 4 wt % to about 5 wt %.

In some embodiments, the concentration of CHIR99021 or the pharmaceutically acceptable salt thereof in the reconstituted solution is about 3.1 mg/ml; the concentration of valproic acid or the pharmaceutically acceptable salt thereof is about 89 mg/ml; the concentration of poloxamer 407 is about 16 wt %; and the concentration of DMSO is about 5 wt %.

In some embodiments, the reconstituted solution comprises:

i) CHIR99021 or a pharmaceutically acceptable salt thereof being present at a concentration ranging from 0.05 mg/ml to about 50 mg/ml;

ii) valproic acid or a pharmaceutically acceptable salt thereof being present at a concentration ranging from 1 mg/ml to about 1000 mg/ml;

iii) poloxamer 407 being present at a concentration ranging from 2 wt % to about 50 wt %; and

iv) dimethyl sulfoxide (DMSO) being present at a concentration below 15 wt %.

In some embodiments, the pharmaceutically acceptable salt of valproic acid is a sodium salt. In some embodiments, the pharmaceutically acceptable salt of valproic acid is sodium valproate.

In some embodiments, the concentration of CHIR99021 or the pharmaceutically acceptable salt thereof in the reconstituted solution ranges from about 0.1 mg/ml to about 10 mg/ml, from about 0.5 mg/ml to about 5 mg/ml, from about 1 mg/ml to about 3.5 mg/ml, from about 1.9 mg/ml to about 2.3 mg/ml. In some embodiments, the concentration of CHIR99021 or the pharmaceutically acceptable salt thereof is about 2.1 mg/ml.

In some embodiments, the concentration of valproic acid or the pharmaceutically acceptable salt thereof in the reconstituted solution ranges from about 5 mg/ml to about 400 mg/ml, from about 10 mg/ml to about 200 mg/ml, from about 30 mg/ml to about 100 mg/ml, from about 56 mg/ml to about 62 mg/ml. In some embodiments, the concentration of valproic acid or the pharmaceutically acceptable salt thereof is about 59 mg/ml.

In some embodiments, the concentration of poloxamer 407 in the reconstituted solution ranges from about 5 wt % to about 25 wt %, from about 10 wt % to about 22 wt %, from about 12 wt % to about 20 wt %, from about 14 wt % to about 17 wt %. In some embodiments, the concentration of poloxamer 407 is about 15 wt %.

In some embodiments, the concentration of DMSO in the reconstituted solution ranges from about 1 wt % to about 10 wt %, from about 2 wt % to about 8 wt %, from about 3 wt % to about 7 wt %, from about 4 wt % to about 6 wt %. In some embodiments, the concentration of DMSO is about 5 wt %.

In some embodiments, the weight ratio between CHIR99021 or the pharmaceutically acceptable salt thereof and valproic acid or the pharmaceutically acceptable salt thereof in the reconstituted solution ranges from about 1:5 to about 1:10, from about 1:10 to about 1:50, from about 1:20 to about 1:35, from about 1:25 to about 1:31, or from about 1:27 to about 1:29.

In some embodiments, the weight ratio between poloxamer 407 and the DMSO in the reconstituted solution ranges from about 1:5 to about 40:1, from about 1:2 to about 15:1, from about 1:1 to about 8:1, from about 2:1 to about 4:1, from about 2.5:1 to about 3.5:1. In some embodiments, the weight ratio between poloxamer 407 and the DMSO is about 3:1.

In some embodiments, the weight ratio between CHIR99021 and poloxamer 407 in the reconstituted solution is about 0.016:1; the weight ratio between the CHIR99021 and the DMSO is about 0.06:1; the weight ratio between valproic acid sodium salt and poloxamer 407 is about 0.42:1; and/or the weight ratio between valproic acid sodium salt and the DMSO is about 1.5:1.

In some embodiments, the concentration of CHIR99021 or the pharmaceutically acceptable salt thereof in the reconstituted solution ranges from about 1.9 mg/ml to about 2.3 mg/ml; the concentration of valproic acid or the pharmaceutically acceptable salt thereof ranges from about 56 mg/ml to about 62 mg/ml; the concentration of poloxamer 407 ranges from about 14 wt % to about 17 wt %; and the concentration of DMSO ranges from about 4 wt % to about 6 wt %.

In some embodiments, the concentration of CHIR99021 or the pharmaceutically acceptable salt thereof in the reconstituted solution is about 2.1 mg/ml; the concentration of valproic acid or the pharmaceutically acceptable salt thereof is about 59 mg/ml; the concentration of poloxamer 407 is about 15 wt %; and the concentration of DMSO is about 5 wt %.

In some embodiments, the reconstituted solution comprises:

i) CHIR99021 or a pharmaceutically acceptable salt thereof being present at a concentration ranging from 0.05 mg/ml to about 50 mg/ml;

ii) valproic acid or a pharmaceutically acceptable salt thereof being present at a concentration ranging from 1 mg/ml to about 1000 mg/ml;

iii) poloxamer 407 being present at a concentration ranging from 2 wt % to about 50 wt %; and

iv) dimethyl sulfoxide (DMSO) being present at a concentration below 15 wt %.

In some embodiments, the pharmaceutically acceptable salt of valproic acid is a sodium salt (e.g., sodium valproate).

In some embodiments, the concentration of CHIR99021 or the pharmaceutically acceptable salt thereof in the reconstituted solution ranges from about 0.1 mg/ml to about 10 mg/ml, from about 0.5 mg/ml to about 5 mg/ml, from about 1 mg/ml to about 3.5 mg/ml, or from about 1.2 mg/ml to about 1.5 mg/ml. In some embodiments, the concentration of CHIR99021 or the pharmaceutically acceptable salt thereof ranges is about 1.4 mg/ml.

In some embodiments, the concentration of valproic acid or the pharmaceutically acceptable salt thereof in the reconstituted solution ranges from about 5 mg/ml to about 400 mg/ml, from about 10 mg/ml to about 200 mg/ml, from about 30 mg/ml to about 100 mg/ml, or from about 36 mg/ml to about 42 mg/ml. In some embodiments, the concentration of valproic acid or the pharmaceutically acceptable salt thereof is about 39 mg/ml.

In some embodiments, the concentration of poloxamer 407 in the reconstituted solution ranges from about 5 wt % to about 25 wt %, from about 10 wt % to about 22 wt %, from about 12 wt % to about 20 wt %, or from about 14 wt % to about 17 wt %. In some embodiments, the concentration of poloxamer 407 is about 15 wt %.

In some embodiments, the concentration of DMSO in the reconstituted solution ranges from about 1 wt % to about 10 wt %, from about 2 wt % to about 8 wt %, from about 3 wt % to about 7 wt %, or from about 4 wt % to about 6 wt %. In some embodiments, the concentration of DMSO is about 5 wt %.

In some embodiments, the weight ratio between CHIR99021 or the pharmaceutically acceptable salt thereof and valproic acid or the pharmaceutically acceptable salt thereof in the reconstituted solution ranges from about 1:5 to about 1:10, from about 1:10 to about 1:50, from about 1:20 to about 1:35, from about 1:25 to about 1:31, or from about 1:27 to about 1:29.

In some embodiments, the weight ratio between poloxamer 407 and the DMSO in the reconstituted solution ranges from about 1:5 to about 40:1, from about 1:2 to about 15:1, from about 1:1 to about 8:1, from about 2:1 to about 4:1, from about 2.5:1 to about 3.5:1.

In some embodiments, the weight ratio between poloxamer 407 and the DMSO in the reconstituted solution is about 3:1; the weight ratio between the CHIR99021 and poloxamer 407 is about 0.013:1; the weight ratio between CHIR99021 and the DMSO is about 0.06:1; the weight ratio between valproic acid sodium salt and poloxamer 407 is about 0.23:1; and/or the weight ratio between valproic acid sodium salt and the DMSO is about 1.8:1.

In some embodiments, the concentration of CHIR99021 or the pharmaceutically acceptable salt thereof in the reconstituted solution ranges from about 1.2 mg/ml to about 1.5 mg/ml; the concentration of valproic acid or the pharmaceutically acceptable salt thereof ranges from about 36 mg/ml to about 42 mg/ml; the concentration of poloxamer 407 ranges from about 14 wt % to about 17 wt %; and the concentration of DMSO ranges from about 4 wt % to about 6 wt %.

In some embodiments, the concentration of CHIR99021 or the pharmaceutically acceptable salt thereof in the reconstituted solution is about 1.4 mg/ml; the concentration of valproic acid or the pharmaceutically acceptable salt thereof is about 39 mg/ml; the concentration of poloxamer 407 is about 15 wt %; and the concentration of DMSO is about 5 wt %.

In some embodiments, the reconstituted solution comprises, in addition to the active agents, one or more of water or a buffering agent; a bulking agent (e.g., purified Poloxamer 407); a stabilizing agent; a tonicity-adjusting agent; and a soothing agent.

In some embodiments, the reconstituted solution comprises, in addition to the active agents, purified poloxamer (e.g., purified Poloxamer 407), and wherein the reconstituted solution has a higher stability to oxygen and/or light as compared to a comparable reconstituted solution without (e.g., purified Poloxamer 407). In some embodiments, the comparable reconstituted solution comprises unpurified Poloxamer (e.g., unpurified Poloxamer 407).

In some embodiments, the level of an impurity present in the reconstituted solution is less than about 10000 parts per million (ppm), less than about 1000 ppm, less than about 100 ppm, less than about 10 ppm, less than about 1 ppm, or less than about 0.1 ppm.

In some embodiments, the impurity is selected from the group consisting of 1-acetate-2-formate-1,2-propanediol, acetic acid, formic acid, formaldehyde, acetaldehyde, and propionaldehyde.

In some embodiments, the level of polyethylene oxide present in the reconstituted solution is below about 3%, below about 2%, below about 1%, below about 0.5%, or below about 0.1%, as measured by high-performance liquid chromatography (HPLC).

In some embodiments, the total level of one or more impurities with c Log P of about 1 or less present in the reconstituted solution is from about 30% to about 35%, from about 25% to about 29%, from about 20% to about 25%, from about 15% to about 19%, from about 10% to about 14%, from about 5% to about 9%, or from about 0% to about 4%, as measured by high-performance liquid chromatography (HPLC).

In some embodiments, the total level of one or more impurities having a boiling point of about 220° C. or less present in the reconstituted solution is from about 35% to about 40%, from about 30% to about 34%, from about 25% to about 29%, from about 20% to about 25%, from about 15% to about 19%, from about 10% to about 14%, from about 5% to about 9%, or from about 0% to about 4%, as measured by high-performance liquid chromatography (HPLC).

In some embodiments, the reconstituted solution comprises purified poloxamer (e.g., purified Poloxamer 407), and wherein the level of the one or more otic therapeutic agents (e.g., hearing loss treatment agents) present in the reconstituted solution is about 1.5 fold or higher, about 1.8 fold or higher, about 2 fold or higher, about 2.5 fold or higher, about 3 fold or higher, about 5 fold or higher, or about 10 fold or higher as compared to a comparable reconstituted solution without purified poloxamer (e.g., purified Poloxamer 407). In some embodiments, the comparable reconstituted solution comprises unpurified poloxamer (e.g., unpurified Poloxamer 407).

In some embodiments, the reconstituted solution comprises purified poloxamer (e.g., purified Poloxamer 407), and wherein the reconstituted solution has lower batch-to-batch variability of one or more gelation properties (e.g., gelation temperature, viscosity, and/or stability) as compared to a comparable reconstituted solution without purified poloxamer (e.g., purified Poloxamer 407). In some embodiments, the comparable reconstituted solution comprises unpurified poloxamer (e.g., unpurified Poloxamer 407).

In some embodiments, the reconstituted solution comprises purified poloxamer (e.g., purified Poloxamer 407), and wherein the reconstituted solution has a lower gelation temperature, a narrower gelation temperature range, a more sustained release of the hearing loss treatment agent, and/or a higher viscosity as compared to a reconstituted solution without purified poloxamer (e.g., purified Poloxamer 407). In some embodiments, the comparable reconstituted solution comprises unpurified poloxamer (e.g., unpurified Poloxamer 407).

In some embodiments, the reconstituted solution comprises purified poloxamer (e.g., purified Poloxamer 407), and wherein the reconstituted solution has a lower gelation temperature than the gelation temperature of an otherwise identical composition with unpurified poloxamer rather than purified poloxamer, wherein the temperature is about 1° C. lower, about 2° C. lower, about 3° C. lower, about 4° C. lower, about 5° C. lower, about 6° C. lower, about 7° C. lower, about 8° C. lower, about 9° C. lower, about 10° C. lower, about 11° C. lower, about 12° C. lower, or about 13° C. lower than the gelation temperature of an otherwise identical reconstituted solution with unpurified poloxamer (e.g., unpurified Poloxamer 407) rather than purified poloxamer as described herein. In other embodiments, the reconstituted solution comprises purified poloxamer (e.g., purified Poloxamer 407), and wherein the reconstituted solution has a narrower gelation temperature range compared to the gelation temperature range of an otherwise identical composition with unpurified poloxamer rather than purified poloxamer. The gelation temperature range is the range of temperatures over which the formulation transitions from being a fluid to being a gel. Composition with unpurified poloxamer generally transition from a fluid to a gel over a range of about 10° C., whereas otherwise identical compositions with purified poloxamer (e.g., purified Poloxamer 407) transition from a fluid to a gel over a range of about 2° C. to about 3° C.

In some embodiments, the reconstituted solution comprises purified poloxamer (e.g., purified Poloxamer 407), and wherein the reconstituted solution has a reduced degradation rate as compared to a comparable reconstituted solution without purified poloxamer (e.g., purified Poloxamer 407). In some embodiments, the comparable reconstituted solution comprises unpurified poloxamer (e.g., unpurified Poloxamer 407).

In some embodiments, the reconstituted solution is suitable for injection (e.g., intratympanic injection).

In some embodiments, the reconstituted solution maintains one or more rheometric properties of a pharmaceutical composition which is used for preparing the lyophilized pharmaceutical composition.

In some embodiments, the reconstituted solution has a reduced degradation rate as compared to a reconstituted solution prepared from a comparable lyophilized pharmaceutical composition without purified poloxamer (e.g., purified Poloxamer 407). In some embodiments, the comparable lyophilized pharmaceutical composition comprises unpurified poloxamer (e.g., unpurified Poloxamer 407).

In some embodiments, the reconstituted solution comprises one or more of water or a buffering agent; a bulking agent (e.g., purified Poloxamer 407); a stabilizing agent; a tonicity-adjusting agent; and a soothing agent

### Other Components

In some embodiments, the pharmaceutical composition or reconstituted solution of the present disclosure comprises water.

In some embodiments, the pharmaceutical composition or reconstituted solution of the present disclosure comprises a buffering agent. The buffer controls the pH of the reconstituted solution to a range of from about 4 to about 13, from about 5 to about 12, from about 6 to about 11, from about 6.5 to about 10.5, or from about 7 to about 10.

Examples of the buffering agent include, but are not limited to, citrate buffering agents, acetate buffering agents, phosphate buffering agents, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may be selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

In some embodiments, the buffering agent comprises phosphate buffered saline, TRIS, tris acetate, tris HCl-65, sodium citrate, histidine, arginine, sodium phosphate, tris base-65, hydroxyethyl starch, or any combination thereof.

As discussed above, a poloxamer can be used in certain embodiments as a gelling agent. An aldehyde is a compound containing a functional group with the structure —CHO, consisting of a carbon double-bonded to oxygen with the carbon atom also bonded to a hydrogen atom. Aldehydes, including formaldehyde, acetaldehyde, and propionaldehyde, are potential impurities and degradation products of poloxamers and may be formed e.g. when the poloxamer is present in a gel. Lyophilization beneficially removes aldehydes present in the test composition. Lyophilized compositions disclosed herein can also be more stable than the gel form, for example in relation to the levels of aldehyde present over time.

In some embodiments, lyophilization removes aldehydes from the compositions of the present disclosure.

In some embodiments, preservatives such as antioxidants are not required in the lyophilized compositions of the present disclosure, for example because of the low levels of aldehydes present.

In some embodiments of the lyophilized pharmaceutical composition, the concentration of aldehydes is less than about 1, about 2, about 3, about 4, about 5 or about 10 ppm (μg/g). In some embodiments of the lyophilized pharmaceutical composition, the concentration of aldehydes is less than about 10 ppm (μg/g). In some embodiments of the lyophilized pharmaceutical composition, the concentration of aldehydes is less than about 5 ppm (μg/g). In some embodiments of the lyophilized pharmaceutical composition, the concentration of aldehydes is less than about 4 ppm (μg/g). In some embodiments of the lyophilized pharmaceutical composition, the concentration of aldehydes is less than about 3 ppm (μg/g). In some embodiments of the lyophilized pharmaceutical composition, the concentration of aldehydes is less than about 2 ppm (μg/g). In some embodiments of the lyophilized pharmaceutical composition, the concentration of aldehydes is less than about 1 ppm (μg/g).

In some embodiments, the aldehydes are volatile aldehydes.

In some embodiments, the aldehydes comprise molecules where each individual molecule has a molecular weight of less than 300 Da. In some embodiments, the aldehydes comprise molecules where each individual molecule has a molecular weight of less than 200 Da. In some embodiments, the aldehydes comprise molecules where each individual molecule has a molecular weight of less than 100 Da.

In some embodiments, the aldehydes comprise formaldehyde, acetaldehyde, and/or propionaldehyde.

Some examples of antioxidants include, but are not limited to, RRR-Alpha-Tocopherol, d-Alpha tocopherol; d-alpha tocopheryl acetate; dl-alpha tocopheryl acetate; d-alpha tocopheryl acid succinate; dl-alpha tocopheryl acid succinate; beta tocopherol; delta tocopherol; gamma tocopherol; tocopherols excipient, Ascorbic Acid; Ascorbyl palmitate; erythorbic acid; sodium ascorbate; sodium erythorbate; butylated hydroxytoluene; Butylated Hydroxyanisole; Anhydrous citric acid; fumaric acid; malic acid; sodium citrate; dihydrate; tartaric acid; Citric Acid Monohydrate; Edetic Acid; Dipotassium edetate; disodium edetate; edetate calcium disodium; sodium edetate; trisodium edetate; propyl gallate; Methionine; Monothioglycerol; Pentetic Acid; Potassium Metabisulphite; Potassium bisulfite; sodium metabisulfite; Propionic Acid; Calcium propionate; sodium propionate; Dodecyl gallate; ethyl gallate; octyl gallate; Sodium Formaldehyde Sulfoxylate; Anhydrous Sodium Sulphite; Sodium Thiosulphate; Sulfur Dioxide; Vitamin E Polyethylene Glycol Succinate.

In some embodiments, the pharmaceutical composition of the present disclosure does not comprise an antioxidant.

In some embodiments, the lyophilized pharmaceutical composition of the present disclosure does not comprise an antioxidant.

In some embodiments, the reconstituted lyophilized pharmaceutical composition of the present disclosure does not comprise an antioxidant.

In some embodiments, the pharmaceutical composition of the present disclosure does not comprise an antioxidant and has a concentration of aldehydes which is less than about 1, about 2, about 3, about 4, about 5 or about 10 ppm (μg/g).

In some embodiments, the lyophilized pharmaceutical composition of the present disclosure does not comprise an antioxidant and has a concentration of aldehydes which is less than about 1, about 2, about 3, about 4, about 5 or about 10 ppm (μg/g).

In some embodiments, the reconstituted pharmaceutical composition of the present disclosure does not comprise an antioxidant and has a concentration of aldehydes which is less than about 1, about 2, about 3, about 4, about 5 or about 10 ppm (μg/g).

In some embodiments, the the pharmaceutical composition or reconstituted solution of the present disclosure comprises a bulking agent.

In some embodiments, the bulking agent comprises poloxamer (e.g., poloxamer 407), mannitol, sucrose, maltose, trehalose, dextrose, sorbitol, glucose, raffinose, glycine, histidine, polyvinylpyrrolidone (e.g., polyvinylpyrrolidone K12 or polyvinylpyrrolidone K17), lactose, or any combination thereof.

In some embodiments, the poloxamer (e.g., poloxamer 407) is the gelling agent and/or the bulking agent. In some embodiments, the poloxamer (e.g., poloxamer 407) is the gelling agent and the bulking agent.

In some embodiments, where the composition comprises a gelling agent (such as poloxamer, e.g. Poloxamer 407), the composition does not comprise an additional bulking agent (such as mannitol, sucrose, maltose, trehalose, dextrose, sorbitol, glucose, raffinose, glycine, histidine, polyvinylpyrrolidone (e.g., polyvinylpyrrolidone K12 or polyvinylpyrrolidone K17), lactose, or any combination thereof).

A bulking agent can positively enhance the lyophilization process, leading to an improved dried/lyophilized product in terms of appearance and characteristics.

However, a solution of poloxamer 407 can be lyophilized in the absence of a bulking agent to form a porous cake of substantial volume (e.g. see FIG. 9) and not a flat sheet of dried mass (e.g. see FIG. 10). The same effect was noted when a molecule such as sodium valproate (NaVPA) was added to poloxamer 407 solution. A polymeric lyophilized cake mass produced in this way (e.g. see Step 7 of Example 10) reconstituted well and retained the rheological properties similar to the pre-lyophilized solution.

In some embodiments, the pharmaceutical composition of the present disclosure does not comprise bulking agent in addition to the gelling agent.

In some embodiments, the lyophilized pharmaceutical composition of the present disclosure does not comprise a bulking agent in addition to the gelling agent.

In some embodiments, the reconstituted lyophilized pharmaceutical composition of the present disclosure does not comprise a bulking agent in addition to the gelling agent.

In some embodiments, the pharmaceutical composition or reconstituted solution of the present disclosure comprises a stabilizing agent. In some embodiments, the stabilizing agent comprises Polyethylene Glycol, saccharides, ascorbic acid, acetylcysteine, bisulfite, metabisulfite, monothioglyercol, inositol, oleic acid, or any combination thereof.

In some embodiments, the stabilizing agent comprises a cryoprotectant. In some embodiments, the cryoprotectant is a polyol (e.g., a diol or a triol such as propylene glycol (i.e., 1,2-propanediol), 1,3-propanediol, glycerol, (+/−)-2-methyl-2,4-pentanediol, 1,6-hexanediol, 1,2-butanediol, 2,3-butanediol, ethylene glycol, or diethylene glycol), a nondetergent sulfobetaine (e.g., NDSB-201 (3-(1-pyridino)-1-propane sulfonate), an osmolyte (e.g., L-proline or trimethylamine N-oxide dihydrate), a polymer (e.g., polyethylene glycol 200 (PEG 200), PEG 400, PEG 600, PEG 1000, PEG 3350, PEG 4000, PEG 8000, PEG 10000, PEG 20000, polyethylene glycol monomethyl ether 550 (mPEG 550), mPEG 600, mPEG 2000, mPEG 3350, mPEG 4000, mPEG 5000, polyvinylpyrrolidone (e.g., polyvinylpyrrolidone K 15), pentaerythritol propoxylate, or polypropylene glycol P 400), an organic solvent (e.g., dimethyl sulfoxide (DMSO) or ethanol), a sugar (e.g., D-(+)-sucrose, D-sorbitol, trehalose, D-(+)-maltose monohydrate, meso-erythritol, xylitol, myo-inositol, D-(+)-raffinose pentahydrate, D-(+)-trehalose dihydrate, or D-(+)-glucose monohydrate), or a salt (e.g., lithium acetate, lithium chloride, lithium formate, lithium nitrate, lithium sulfate, magnesium acetate, sodium chloride, sodium formate, sodium malonate, sodium nitrate, sodium sulfate, or any hydrate thereof) or any combination thereof.

In some embodiments, the stabilizing agent comprises a salt. In some embodiment, the salt is selected from the group consisting of lithium salts (e.g., lithium acetate, lithium chloride, lithium formate, lithium nitrate, lithium sulfate, or any hydrate thereof), magnesium salts (e.g., magnesium acetate or a hydrate thereof), and sodium salts (e.g., sodium chloride, sodium formate, sodium malonate, sodium nitrate, sodium sulfate, or any hydrate thereof). For another example, the formulation comprises one or more sodium salts. For yet another example, the formulation comprises sodium chloride.

In some embodiment, the stabilizing agent comprises a surfactant. In some embodiments, the surfactant comprises one or more anionic surfactants (e.g., 2-acrylamido-2-methylpropane sulfonic acid, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium dodecylbenzenesulfonate, sodium laurate, sodium laureth sulfate, sodium lauroyl sarcosinate, sodium myreth sulfate, sodium nonanoyloxybenzenesulfonate, sodium pareth sulfate, sodium stearate, or sulfolipid), one or more cationic surfactants (e.g., behentrimonium chloride, benzalkonium chloride, benzethonium chloride, benzododecinium bromide, bronidox, carbethopendecinium bromide, cetalkonium chloride, cetrimonium bromide, cetrimonium chloride, cetylpyridinium chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, octenidine dihydrochloride, olaflur, n-oleyl-1,3-propanediamine, pahutoxin, stearalkonium chloride, tetramethylammonium hydroxide, or thonzonium bromide), one or more zwitterionic surfactants (e.g., cocamidopropyl betaine, cocamidopropyl hydroxysultaine, dipalmitoylphosphatidylcholine, egg lecithin, hydroxysultaine, lecithin, myristamine oxide, peptitergents, or sodium lauroamphoacetate), and/or one or more non-ionic surfactants (e.g., alkyl polyglycoside, cetomacrogol 1000, cetostearyl alcohol, cetyl alcohol, cocamide dea, cocamide mea, decyl glucoside, decyl polyglucose, glycerol monostearate, igepal ca-630, isoceteth-20, lauryl glucoside, maltosides, monolaurin, mycosubtilin, narrow-range ethoxylate, nonidet p-40, nonoxynol-9, nonoxynols, np-40, octaethylene glycol monododecyl ether, n-octyl beta-d-thioglucopyranoside, octyl glucoside, oleyl alcohol, peg-10 sunflower glycerides, pentaethylene glycol monododecyl ether, polidocanol, α-tocopheryl polyethylene glycol succinate (TPGS), poloxamer (e.g., poloxamer 407), polyethoxylated tallow amine, polyglycerol polyricinoleate, polysorbate (e.g., polysorbate 20, polysorbate 40, polysorbate 60, or polysorbate 80), sorbitan, sorbitan monolaurate, sorbitan monostearate, sorbitan tristearate, stearyl alcohol, surfactin, triton x-100).

In some embodiments, the the pharmaceutical composition or reconstituted solution of the present disclosure comprises a tonicity-adjusting agent.

In some embodiments, the tonicity-adjusting agent comprises NaCl, dextrose, dextran, ficoll, gelatin, mannitol, sucrose, glycine, glycerol, or any combination thereof.

In some embodiments, the the pharmaceutical composition or reconstituted solution of the present disclosure comprises a soothing agent. In some embodiments, the soothing agent comprises lidocaine

In addition to these components, the pharmaceutical composition or reconstituted solution of the present disclosure may include any substance useful in pharmaceutical compositions. In some embodiments, the pharmaceutical composition or reconstituted solution of the present disclosure may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21a Edition, A. R Gennaro; Lippincott, Williams & Wilkins, Baltimore, Md., 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (ST FS), sodium bisulfite, sodium metabisulfite, potassium sulfite, potassium metabisulfite, GLYDANT PLUS®, PHENONIP®, methylparaben, GERMALL® 115, GERMABEN®II, NEOLONE™ KATHON™, and/or EUXYL®.

Examples of buffering agents include, but are not limited to, citrate buffering agents, acetate buffering agents, phosphate buffering agents, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may be selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or

As used herein, the term “pharmaceutically acceptable salt” takes its normal meaning in the alt. In certain embodiments it refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal and alkaline earth metal. Representative alkali or alkaline earth metal salts include sodium, lithium, potassium, calcium, magnesium, iron, zinc, copper, manganese, aluminum, and the like. Further pharmaceutically acceptable salts include, when appropriate, potassium, sodium, calcium, and magnesium salts.

“Alkyl” refers to a straight or branched hydrocarbon chain radical consisting solely of carbon and hydrogen atoms, containing no unsaturation, having from one to ten carbon atoms (e.g., C₁₋₁₀ alkyl). Whenever it appears herein, a numerical range such as “1 to 10” refers to each integer in the given range; e.g., “1 to 10 carbon atoms” means that the alkyl group can consist of 1 carbon atom, 2 carbon atoms, 3 carbon atoms, etc., up to and including 10 carbon atoms, although the present definition also covers the occurrence of the term “alkyl” where no numerical range is designated. In some embodiments, “alkyl” can be a C₁₋₆ alkyl group. In some embodiments, alkyl groups have 1 to 10, 1 to 8, 1 to 6, or 1 to 3 carbon atoms. Representative saturated straight chain alkyls include, but are not limited to, -methyl, -ethyl, -n-propyl, -n-butyl, -n-pentyl, and -n-hexyl; while saturated branched alkyls include, but are not limited to, -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, and the like. The alkyl is attached to the parent molecule by a single bond. Unless stated otherwise in the specification, an alkyl group is optionally substituted by one or more of substituents which independently include: alkyl, alkenyl, alkynyl, alkoxy, cycloalkyl, aryl, or halo. In a non-limiting embodiment, a substituted alkyl can be selected from fluoromethyl, difluoromethyl, trifluoromethyl, 2-fluoroethyl, 3-fluoropropyl, hydroxymethyl, 2-hydroxyethyl, 3-hydroxypropyl, benzyl, and phenethyl.

“Alkenyl” refers to a straight or branched hydrocarbon chain radical group consisting solely of carbon and hydrogen atoms, containing at least one double bond, and having from two to ten carbon atoms (i.e., C₂₋₁₀ alkenyl). Whenever it appears herein, a numerical range such as “2 to 10” refers to each integer in the given range; e.g., “2 to 10 carbon atoms” means that the alkenyl group can consist of 2 carbon atoms, 3 carbon atoms, etc., up to and including 10 carbon atoms. In certain embodiments, an alkenyl comprises two to eight carbon atoms. In other embodiments, an alkenyl comprises two to six carbon atoms (e.g., C₂₋₆ alkenyl). The alkenyl is attached to the parent molecular structure by a single bond, for example, ethenyl (i.e., vinyl), prop-1-enyl (i.e., allyl), but— 1-enyl, pent-1-enyl, penta-1,4-dienyl, and the like. The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), 2-methylprop-2-enyl (C₄), butadienyl (C₄) and the like. Examples of C₂₋₆ alkenyl groups include the aforementioned C₂₋₄ alkenyl groups as well as pentenyl (C₅), pentadienyl (C₅), hexenyl (C₆), 2,3-dimethyl-2-butenyl (C₆) and the like. Additional examples of alkenyl include heptenyl (C₇), octenyl (C₈), octatrienyl (C₈) and the like. Unless stated otherwise in the specification, an alkenyl group can be optionally substituted by one or more substituents which independently include: alkyl, alkenyl, alkynyl, alkoxy, cycloalkyl, awl, or halo.

“Alkynyl” refers to a straight or branched hydrocarbon chain radical group consisting solely of carbon and hydrogen atoms, containing at least one triple bond, having from two to ten carbon atoms (i.e., C₂₋₁₀ alkynyl). Whenever it appears herein, a numerical range such as “2 to 10” refers to each integer in the given range; e.g., “2 to 10 carbon atoms” means that the alkynyl group can consist of 2 carbon atoms, 3 carbon atoms, etc., up to and including 10 carbon atoms. In certain embodiments, an alkynyl comprises two to eight carbon atoms. In other embodiments, an alkynyl has two to six carbon atoms (e.g., C₂-6 alkynyl). The alkynyl is attached to the parent molecular structure by a single bond, for example, ethynyl, propynyl, butynyl, pentynyl, 3-methyl-4-pentenyl, hexynyl, and the like. Unless stated otherwise in the specification, an alkynyl group can be optionally substituted by one or more substituents which independently include: alkyl, alkenyl, alkynyl, alkoxy, cycloalkyl, aryl, and halo.

“Alkoxy” refers to the group —O-alkyl, including from 1 to 10 carbon atoms of a straight, branched, saturated cyclic configuration and combinations thereof, attached to the parent molecular structure through an oxygen. Examples include methoxy, ethoxy, propoxy, isopropoxy, butoxy, t-butoxy, pentoxy, cyclopropyloxy, cyclohexyloxy and the like. “Lower alkoxy” refers to alkoxy groups containing one to six carbons. In some embodiments, C₁₋₄ alkoxy is an alkoxy group which encompasses both straight and branched chain alkyls of from 1 to 4 carbon atoms. Unless stated otherwise in the specification, an alkoxy group can be optionally substituted by one or more substituents which independently include: alkyl, alkenyl, alkynyl, alkoxy, cycloalkyl, awl, and halo.

“Aryl” refers to a radical with 6 to 14 ring atoms (e.g., C₆₋₁₄ aromatic or C₆₋₁₄ aryl) which has at least one ring having a conjugated pi electron system which is carbocyclic (e.g., phenyl, fluorenyl, and naphthyl). In some embodiments, the aryl is a C₆₋₁₀ aryl group. For example, bivalent radicals formed from substituted benzene derivatives and having the free valences at ring atoms are named as substituted phenylene radicals. In other embodiments, bivalent radicals derived from univalent polycyclic hydrocarbon radicals whose names end in“-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 14 aryl” refers to each integer in the given range; e.g., “6 to 14 ring atoms” means that the aryl group can consist of 6 ring atoms, 7 ring atoms, etc., up to and including 14 ring atoms. The term includes monocyclic or fused-ring polycyclic (i.e., rings which share adjacent pairs of ring atoms) groups. Polycyclic awl groups include bicycles, tricycles, tetracycles, and the like. In a multi-ring group, only one ring is required to be aromatic, so groups such as indanyl are encompassed by the aryl definition. Non-limiting examples of awl groups include phenyl, phenalenyl, naphthalenyl, tetrahydronaphthyl, phenanthrenyl, anthracenyl, fluorenyl, indolyl, indanyl, and the like. Unless stated otherwise in the specification, an awl moiety can be optionally substituted by one or more substituents which independently include: alkyl, alkenyl, alkynyl, alkoxy, cycloalkyl, awl, and halo. When “aryl” is “tolyl” this term includes any of o-tolyl, m-tolyl, and p-tolyl groups. In other words, “tolyl” includes any of the three isomeric univalent aromatic radicals derived from toluene. When “awl” is “xylyl” this term includes the univalent radicals, of formula (CH₃)₂C₆H₃— derived from the three isomers of xylene: ortho-, meta- and para- (di-methyl benzene).

“Cycloalkyl” and “carbocyclyl” each refer to a monocyclic or polycyclic radical that contains only carbon and hydrogen, and can be saturated or partially unsaturated. Partially unsaturated cycloalkyl groups can be termed “cycloalkenyl” if the carbocycle contains at least one double bond, or “cycloalkynyl” if the carbocycle contains at least one triple bond. Cycloalkyl groups include groups having from 3 to 13 ring atoms (i.e., C₃-13 cycloalkyl). Whenever it appears herein, a numerical range such as “3 to 10” refers to each integer in the given range; e.g., “3 to 13 carbon atoms” means that the cycloalkyl group can consist of 3 carbon atoms, 4 carbon atoms, 5 carbon atoms, etc., up to and including 13 carbon atoms. The term “cycloalkyl” also includes bridged and spiro-fused cyclic structures containing no heteroatoms. The term also includes monocyclic or fused-ring polycyclic (i.e., rings which share adjacent pairs of ring atoms) groups. Polycyclic awl groups include bicycles, tricycles, tetracycles, and the like. In some embodiments, “cycloalkyl” can be a C₃₋₈ cycloalkyl radical. In some embodiments, “cycloalkyl” can be a C₃₋₅ cycloalkyl radical. Illustrative examples of cycloalkyl groups include, but are not limited to the following moieties: C₃₋₆ carbocyclyl groups include, without limitation, cyclopropyl (C₃), cyclobutyl (CO, cyclopentyl (C₅), cyclopentenyl (C₅), cyclohexyl (C₆), cyclohexenyl (C₆), cyclohexadienyl (C₆) and the like. Examples of C₃₋₇ carbocyclyl groups include norbornyl (C₇). Examples of C₃₋₈ carbocyclyl groups include the aforementioned C₃₋₇ carbocyclyl groups as well as cycloheptyl(C₇), cycloheptadienyl (C₇), cycloheptatrienyl (C₇), cyclooctyl (C₈), bicyclo[2.2.1]heptanyl, bicyclo[2.2.2]octanyl, and the like. Examples of C₃₋₁₃ carbocyclyl groups include the aforementioned C₃₋₈ carbocyclyl groups as well as octahydro-1H indenyl, decahydronaphthalenyl, spiro[4.5]decanyl and the like. Unless stated otherwise in the specification, a cycloalkyl group can be optionally substituted by one or more substituents which independently include: alkyl, alkenyl, alkynyl, alkoxy, cycloalkyl, awl, and halo. The terms “cycloalkenyl” and “cycloalkynyl” mirror the above description of “cycloalkyl” wherein the prefix “alk” is replaced with “alken” or “alkyn” respectively, and the parent “alkenyl” or “alkynyl” terms are as described herein. For example, a cycloalkenyl group can have 3 to 13 ring atoms, such as 5 to 8 ring atoms. In some embodiments, a cycloalkynyl group can have 5 to 13 ring atoms.

As used herein, a “covalent bond” or “direct bond” refers to a single bond joining two groups.

“Halo”, “halide”, or, alternatively, “halogen” means fluoro, chloro, bromo or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine, such as, but not limited to, trifluoromethyl, difluoromethyl, 2,2,2-trifluoroethyl, 1-fluoromethyl-2-fluoroethyl, and the like. Each of the alkyl, alkenyl, alkynyl and alkoxy groups are as defined herein and can be optionally further substituted as defined herein.

### Methods of Use

In certain embodiments, the present disclosure relates to inducing, promoting, or enhancing the growth, proliferation or regeneration of inner ear tissue, particularly inner ear supporting cells and hair cells by using the composition disclosed herein. Some embodiments relate to methods for controlled proliferation of stem cells comprising an initial phase of inducing sternness while inhibiting differentiation and a subsequent phase of differentiation of the stem cells into tissue cells.

When cochlear supporting cell or vestibular supporting cell populations are treated with a hair cell regeneration agent in accordance to the methods of the disclosure, whether the population is in vivo or in vitro, the treated supporting cells exhibit stem-like behavior in that the treated supporting cells have the capacity to proliferate and differentiate and, more specifically, differentiate into cochlear hair cells or vestibular hair cells. In some instances, an agent induces and maintains the supporting cells to produce daughter stem cells that can divide for many generations and maintain the ability to have a high proportion of the resulting cells differentiate into hair cells. In certain embodiments, the proliferating stem cells express stem cell marker(s) selected from one or more of Lgr5, Sox2, Opem1, Phex, lin28, Lgr6, cyclin D1, Msx1, Myb, Kit, Gdnf3, Zic3, Dppa3, Dppa4, Dppa5, Nanog, Esrrb, Rex1, Dnmt3a, Dnmt3b, Dnmt31, Utf1, Tcl1, Oct4, Klf4, Pax6, Six2, Zic1, Zic2, Otx2, Bmi1, CDX2, STAT3, Smadl, Smad2, smad2/3, smad4, smad5, and smad7. Preferably, the proliferating stem cells express stem cell marker(s) selected from one or more of Lgr5, the

In some embodiments, the methods may be used to maintain, or even transiently increase stemness (i.e., self-renewal) of a pre-existing supporting cell population prior to significant hair cell formation. In some embodiments, the pre-existing supporting cell population comprises inner pillar cells, outer pillar cells, inner phalangeal cells, Deiter cells, Hensen cells, Boettcher cells, and/or Claudius cells. Morphological analyses with immunostaining (including cell counts) and lineage tracing across a Representative Microscopy Samples may be used to confirm expansion of one or more of these cell-types. In some embodiments, the pre-existing supporting cells comprise Lgr5+ cells. Morphological analyses with immunostaining (including cell counts) and qPCR and RNA hybridization may be used to confirm Lgr5 upregulation amongst the cell population.

Advantageously, methods described herein can achieve these goals without the use of genetic manipulation. Germ-line manipulation used in many academic studies is not a therapeutically desirable approach to treating hearing loss. In general, the therapy preferably involves the administration of a small molecule, peptide, antibody, or other non-nucleic acid molecule or nucleic acid delivery vector unaccompanied by gene therapy. In certain embodiments, the therapy involves the administration of a small organic molecule. In some instances, hearing protection or restoration is achieved through the use of a (non-genetic) therapeutic that is injected in the middle ear and diffuses into the cochlea.

The cochlea relies heavily on all present cell types, and the organization of these cells is important to their function. Supporting cells play an important role in neurotransmitter cycling and cochlear mechanics. Thus, maintaining a rosette patterning within the organ of Corti may be important for function. Cochlear mechanics of the basilar membrane activate hair cell transduction. Due to the high sensitivity of cochlear mechanics, it is also desirable to avoid masses of cells. In all, maintaining proper distribution and relation of hair cells and supporting cells along the basilar membrane, even after proliferation, is likely a desired feature for hearing as supporting cell function and proper mechanics is necessary for normal hearing.

In some embodiments the hearing loss treated by using a composition as disclosed herein is senorineural hearing loss or hidden hearing loss.

Sensorineural hearing loss accounts for approximately 90% of hearing loss and it often arises from damage or loss of hair cells in the cochlea. There are numerous causes of hair cell damage and loss, and the agents and treatments described herein may be used in the context of sensorineural hearing loss arising from any cause of hair cell damage or loss. For example, hair cells may be damage and loss may be induced by noise exposure, leading to noise-induced sensorineural hearing loss. Thus, in some embodiments sensorineural hearing loss is noise-induced sensorineural hearing loss. Noise-induced sensorineural hearing loss can be a result of chronic noise exposure or acute noise exposure. Ototoxic drugs, for example cisplatin and its analogs, aminoglycoside antibiotics, salicylate and its analogs, or loop diuretics, can also cause sensorineural hearing loss. In some embodiments sensorineural hearing loss is drug-induced sensorineural hearing loss. Infection may damage cochlear hair cells, and may be a cause of sudden sensorineural hearing loss. In some embodiments sensorineural hearing loss is sudden sensorineural hearing loss (SSNHL). Sudden sensorineural hearing can also be idiopathic. Hair cells can also be lost or damaged over time as part of the ageing process in humans. In some embodiments, sensorineural hearing loss is age-related sensorineural hearing loss (also known as presbycusis).

In some aspects, the present disclosure provides a method of facilitating the regeneration of a tissue and/or a cell, comprising delivering a pharmaceutically effective amount of the pharmaceutical composition or the reconstituted solution of the present disclosure to the tissue and/or the cell.

In some aspects, the present disclosure provides a method of treating a subject who has, or is at risk of developing, a disease associated with absence or a lack of a tissue and/or a cell, comprising administering to the subject a pharmaceutically effective amount of the pharmaceutical composition or the reconstituted solution of the present disclosure.

In some aspects, the present disclosure provides a method of increasing a population of vestibular cells in a vestibular tissue, comprising delivering a pharmaceutically effective amount of the pharmaceutical composition or the reconstituted solution of the present disclosure to the population.

In some aspects, the present disclosure provides a method of treating a subject who has, or is at risk of developing a vestibular condition, comprising administering to the subject a pharmaceutically effective amount of the pharmaceutical composition or the reconstituted solution of the present disclosure.

In some aspects, the present disclosure provides a method of increasing a population of cochlear cells in a cochlear tissue, comprising delivering a pharmaceutically effective amount of the pharmaceutical composition or the reconstituted solution of the present disclosure to the population.

In some aspects, the present disclosure provides a method of treating a subject who has, or is at risk of developing a cochlear condition, comprising administering to the subject a pharmaceutically effective amount of the pharmaceutical composition or the reconstituted solution of the present disclosure.

In some aspects, the present disclosure provides a method of increasing a population of cells found in the Organ of Corti, comprising delivering a pharmaceutically effective amount of the pharmaceutical composition or the reconstituted solution of the present disclosure to the population.

In some aspects, the present disclosure provides a method of increasing a population of hair cells found in the Organ of Corti, comprising delivering a pharmaceutically effective amount of the pharmaceutical composition or the reconstituted solution of the present disclosure to the population.

In some aspects, the present disclosure provides a method of increasing a population of inner hair cells found in the Organ of Corti, comprising delivering a pharmaceutically effective amount of the pharmaceutical composition or the reconstituted solution of the present disclosure to the population.

In some aspects, the present disclosure provides a method of increasing a population of outer hair cells found in the Organ of Corti, comprising delivering a pharmaceutically effective amount of the pharmaceutical composition or the reconstituted solution of the present disclosure to the population.

In some aspects, the present disclosure provides a method of increasing a population of neuronal cells found in the Organ of Corti, comprising delivering a pharmaceutically effective amount of the pharmaceutical composition or the reconstituted solution of the present disclosure to the population.

In some aspects, the present disclosure provides a method of treating a subject who has, or is at risk of developing a hearing condition, comprising administering to the subject a pharmaceutically effective amount of the pharmaceutical composition or the reconstituted solution of the present disclosure.

In some aspects, the present disclosure provides the pharmaceutical composition or the reconstituted solution of the present disclosure for use in facilitating the generation of a tissue and/or a cell.

In some aspects, the present disclosure provides the pharmaceutical composition or the reconstituted solution of the present disclosure for use in treating a subject who has, or is at risk of developing, a disease associated with absence or a lack of a tissue and/or a cell.

In some aspects, the present disclosure provides the pharmaceutical composition or the reconstituted solution of the present disclosure for use in increasing a population of vestibular cells in a vestibular tissue.

In some aspects, the present disclosure provides the pharmaceutical composition or the reconstituted solution of the present disclosure for use in treating a subject who has, or is at risk of developing a vestibular condition.

In some aspects, the present disclosure provides the pharmaceutical composition or the reconstituted solution of the present disclosure for use in increasing a population of cochlear cells in a cochlear tissue.

In some aspects, the present disclosure provides the pharmaceutical composition or the reconstituted solution of the present disclosure for use in treating a subject who has, or is at risk of developing a cochlear condition.

In some aspects, the present disclosure provides the pharmaceutical composition or the reconstituted solution of the present disclosure for use in increasing a population of cells found in the Organ of Corti.

In some aspects, the present disclosure provides the pharmaceutical composition or the reconstituted solution of the present disclosure for use in increasing a population of hair cells found in the Organ of Corti.

In some aspects, the present disclosure provides the pharmaceutical composition or the reconstituted solution of the present disclosure for use in increasing a population of inner hair cells found in the Organ of Corti.

In some aspects, the present disclosure provides the pharmaceutical composition or the reconstituted solution of the present disclosure for use in increasing a population of outer hair cells found in the Organ of Corti.

In some aspects, the present disclosure provides the pharmaceutical composition or the reconstituted solution of the present disclosure for use in increasing a population of neuronal cells found in the Organ of Corti.

In some aspects, the present disclosure provides the pharmaceutical composition or the reconstituted solution of the present disclosure for use in treating a subject who has, or is at risk of developing a hearing condition.

In some aspects, the present disclosure provides for use of the pharmaceutical composition or the reconstituted solution of the present disclosure in the manufacture of a medicament for facilitating the generation of a tissue and/or a cell.

In some aspects, the present disclosure provides for use of the pharmaceutical composition or the reconstituted solution of the present disclosure in the manufacture of a medicament for in treating a subject who has, or is at risk of developing, a disease associated with absence or a lack of a tissue and/or a cell.

In some aspects, the present disclosure provides for use of the pharmaceutical composition or the reconstituted solution of the present disclosure in the manufacture of a medicament for increasing a population of vestibular cells in a vestibular tissue.

In some aspects, the present disclosure provides for use of the pharmaceutical composition or the reconstituted solution of the present disclosure in the manufacture of a medicament for treating a subject who has, or is at risk of developing a vestibular condition.

In some aspects, the present disclosure provides for use of the pharmaceutical composition or the reconstituted solution of the present disclosure in the manufacture of a medicament for increasing a population of cochlear cells in a cochlear tissue.

In some aspects, the present disclosure provides for use of the pharmaceutical composition or the reconstituted solution of the present disclosure in the manufacture of a medicament for treating a subject who has, or is at risk of developing a cochlear condition.

In some aspects, the present disclosure provides for use of the pharmaceutical composition or the reconstituted solution of the present disclosure in the manufacture of a medicament for increasing a population of cells found in the Organ of Corti.

In some aspects, the present disclosure provides for use of the pharmaceutical composition or the reconstituted solution of the present disclosure in the manufacture of a medicament for increasing a population of hair cells found in the Organ of Corti.

In some aspects, the present disclosure provides for use of the pharmaceutical composition or the reconstituted solution of the present disclosure in the manufacture of a medicament for increasing a population of inner hair cells found in the Organ of Corti.

In some aspects, the present disclosure provides for use of the pharmaceutical composition or the reconstituted solution of the present disclosure in the manufacture of a medicament for increasing a population of outer hair cells found in the Organ of Corti.

In some aspects, the present disclosure provides for use of the pharmaceutical composition or the reconstituted solution of the present disclosure in the manufacture of a medicament for increasing a population of neuronal cells found in the Organ of Corti.

Use of the lyophilized pharmaceutical composition or the reconstituted solution of the present disclosure in the manufacture of a medicament for treating a subject who has, or is at risk of developing a hearing condition.

In some embodiments, the pharmaceutical composition or reconstituted solution of the present disclosure is delivered extratympanically (i.e., onto the eardrum).

In some embodiments, the pharmaceutical composition or reconstituted solution of the present disclosure is delivered intratympanically (i.e., into the middle ear).

In some embodiments, the pharmaceutical composition or reconstituted solution of the present disclosure is delivered continuously.

In some embodiments, the pharmaceutical composition or reconstituted solution of the present disclosure is delivered as a bolus injection.

In some embodiments, about about 1 ml or less, about 900 μl or less, about 800 μl or less, about 700 μl or less, about 600 μl or less, about 500 μl or less, about 400 μl or less, about 300 μl or less, about 200 μl or less, or about 100 or less of the pharmaceutical composition or reconstituted solution is injected.

In some embodiments, the pharmaceutical composition or reconstituted solution of the present disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.05 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, from about 1 mg/kg to about 5 mg/kg, from about 2 mg/kg to about 5 mg/kg, from about 0.0001 mg/kg to about 2.5 mg/kg, from about 0.001 mg/kg to about 2.5 mg/kg, from about 0.005 mg/kg to about 2.5 mg/kg, from about 0.01 mg/kg to about 2.5 mg/kg, from about 0.05 mg/kg to about 2.5 mg/kg, from about 0.1 mg/kg to about 2.5 mg/kg, from about 1 mg/kg to about 2.5 mg/kg, from about 2 mg/kg to about 2.5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, from about 0.05 mg/kg to about 1 mg/kg, from about 0.1 mg/kg to about 1 mg/kg, from about 0.0001 mg/kg to about 0.25 mg/kg, from about 0.001 mg/kg to about 0.25 mg/kg, from about 0.005 mg/kg to about 0.25 mg/kg, from about 0.01 mg/kg to about 0.25 mg/kg, from about 0.05 mg/kg to about 0.25 mg/kg, or from about 0.1 mg/kg to about 0.25 mg/kg of a therapeutic and/or prophylactic (e.g., an mRNA) in a given dose, where a dose of 1 mg/kg (mpk) provides 1 mg of a therapeutic and/or prophylactic per 1 kg of subject body weight. In some embodiments, a dose of about 0.001 mg/kg to about 10 mg/kg of a therapeutic and/or prophylactic (e.g., mRNA) of a LNP may be administered. In some embodiments, a dose of about 0.005 mg/kg to about 2.5 mg/kg of a therapeutic and/or prophylactic may be administered. In some embodiments, a dose of about 0.1 mg/kg to about 1 mg/kg may be administered. In some embodiments, a dose of about 0.05 mg/kg to about 0.25 mg/kg may be administered. A dose may be administered one or more times per day, in the same or a different amount, to obtain a desired level of mRNA expression and/or therapeutic, diagnostic, prophylactic, or imaging effect. The desired dosage may be delivered, for example, three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). In some embodiments, a single dose may be administered, for example, prior to or after a surgical procedure or in the instance of an acute disease, disorder, or condition.

In some embodiments, the administration of the pharmaceutical composition or reconstituted solution results in a plasma concentration for the one or more otic therapeutic agents (e.g., CHIR99021 and sodium valproate) having a maximum plasma concentration at at time ranging from 10 minutes to about 3 hours, from about 20 minutes to about 2 hours, or form about 30 minutes to about 1 hour.

### Definitions

Articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all, of the group members are present in, employed in, or otherwise relevant to a given product or process.

As used herein, the terms “approximately” and “about,” as applied to one or more values of interest, refer to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). In some embodiments, the term “approximately” or “about” refers to +/−10% of the recited value. In some embodiments, when used in the context of an amount of a given compound in a lipid component of a LNP, “about” may mean+/−10% of the recited value.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

As used herein, the term “bulking agent” refers to an agent that adds bulk to a pharmaceutical composition and/or modifies one or more the properties of the pharmaceutical composition (e.g., the appearance of the cake, the porosity, drug stability, and/or the reconstitution time).

As used herein, the term “comprising” is intended to be open and permits but does not require the inclusion of additional elements or steps. When the term “comprising” is used herein, the terms “consisting essentially of” and “consisting of” are thus also encompassed and disclosed. Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

As used herein, the term “comparable pharmaceutical composition” refers to a pharmaceutical composition with comparable parameters, as of the pharmaceutical composition being compared (e.g., the one or more otic therapeutic agents (e.g., hearing loss treatment agents) and gelling agents therein, and/or the concentration of the one or more otic therapeutic agents (e.g., hearing loss treatment agents) and gelling agents). In some embodiments, the “comparable pharmaceutical composition” comprises a poloxamer (e.g., Poloxamer 407) with lower purity as compared to pharmaceutical composition being compared. In some embodiments, the “comparable pharmaceutical composition” does not comprise a purified poloxamer (e.g., purified Poloxamer 407). In some embodiments, the “comparable pharmaceutical composition” comprise a unpurified poloxamer (e.g., unpurified Poloxamer 407).

As used herein, the term “comparable reconstituted solution” refers to a reconstituted solution with comparable parameters as of the reconstituted solution being compared (e.g., the one or more otic therapeutic agents (e.g., hearing loss treatment agents) and gelling agents therein, and/or the concentration of the one or more otic therapeutic agents (e.g., hearing loss treatment agents) and gelling agents). In some embodiments, the “comparable reconstituted solution” comprises a poloxamer (e.g., Poloxamer 407) with lower purity as compared to reconstituted solution being compared. In some embodiments, the “comparable reconstituted solution” does not comprise a purified poloxamer (e.g., purified Poloxamer 407). In some embodiments, the “comparable reconstituted solution” comprise a unpurified poloxamer (e.g., unpurified Poloxamer 407).

In some embodiments, the “comparable reconstituted solution” is prepared from a pharmaceutical composition comprising a poloxamer (e.g., Poloxamer 407) with lower purity as compared to pharmaceutical composition used for preparing the reconstituted solution being compared. In some embodiments, the “comparable reconstituted solution” is prepared from a pharmaceutical composition not comprising a purified poloxamer (e.g., purified Poloxamer 407). In some embodiments, the “comparable reconstituted solution” is prepared from a pharmaceutical composition comprising a unpurified poloxamer (e.g., unpurified Poloxamer 407).

As used herein, the term “impurity” refers to a compound that is undesirable for the pharmaceutical composition. In some embodiments, the impurity is selected from solvents, 1-acetate-2-formate-1,2-propanediol, acetic acid, formic acid, formaldehyde, acetaldehyde, propionaldehyde, low MW poloxamers, and degradants from CHIR99021 and valproic acid.

As used herein, the term “soothing agent” refers to an agent capable of mitigating the discomfort from administration of the formulation to patients.

As used herein, the term “stabilizing agent” refers to an agent capable of maintaining the one or more desirable properties of the pharmaceutical composition (e.g., reduced suspetability to degradation by heat, light, or air).

As used herein, the term “unpurified poloxamer” refers to a poloxamer not being purified (e.g., by the process disclosed herein). In some embodiments, the unpurified poloxamer (e.g., unpurified Poloxamer 407) has an average molecular weight of about 12 kDa or lower, about 11 kDa or lower, about 10 kDa or lower, about 9 kDa or lower, about 8 kDa or lower, or about 7 kDa or lower. In some embodiments, the unpurified poloxamer (e.g., unpurified Poloxamer 407) is not purified by any liquid-liquid extraction or size exclusion chromatography.

As used herein, the term “purified poloxamer” may in some embodiments refer to poloxamer that is at least 85% by weight poloxamer that has a molecular weight of at least 7250 Da. Purified poloxamer can in some embodiments be prepared by following the method of: A. Fakhari, M Corcoran, A Schwarz, Thermogelling Properties of Purified Poloxamer 407, Heliyon (2017), 3(8), e00390. Many further options for how the purified poloxamer can be defined are set out herein, including in the numbered clauses and embodiments.

It is to be understood that the present disclosure provides methods for preparing any of the pharmaceutical compositions and reconstituted solutions described herein. The present disclosure also provides detailed methods for preparing various pharmaceutical compositions and reconstituted solutions following the procedures described in the Examples.

It is to be understood that, throughout the description, where compositions are described as having, including, or comprising specific components, it is contemplated that compositions also consist essentially of, or consist of, the recited components. Similarly, where methods or processes are described as having including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that, unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

As used herein, the term “sterile” refers to solutions, products, equipment, or glass ware that are treated and/or handled to be free from bacteria or other living microorganisms.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human. A subject in need thereof can be one who has been previously diagnosed or identified as having an imprinting disorder. A subject in need thereof can also be one who has (e.g., is suffering from) an imprinting disorder. Alternatively, a subject in need thereof can be one who has an increased risk of developing such disorder relative to the population at large (i.e., a subject who is predisposed to developing such disorder relative to the population at large). A subject in need thereof can have a refractory or resistant imprinting disorder (i.e., an imprinting disorder that doesn't respond or hasn't yet responded to treatment). The subject may be resistant at start of treatment or may become resistant during treatment. In some embodiments, the subject in need thereof received and failed all known effective therapies for an imprinting disorder. In some embodiments, the subject in need thereof received at least one prior therapy. In a preferred embodiment, the subject has an imprinting disorder.

As used herein, the term “sterilization” refers to process for ensuring the removal of undesired contamination including bacteria, mold and yeast and particles using e.g., a 0.2-micron filter. Filter materials used in the sterilization of liquids include, but are not limited to, nylon, polycarbonate, cellulose acetate, polyvinylidene fluoride (PVDF), and polyethersulfone (PES).

As used herein, the term “tonicity” refers to a measured level of effective osmolarity. In some embodiments, the tonicity refers to a measured level of the effective osmotic pressure gradient, as defined by the water potential of two solutions separated by a semipermeable membrane.

As used herein, the term “tonicity-adjusting agent” refers to an agent capable of changing the tonicity of the pharmaceutical composition or solution to a desired level.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, N.Y. (2000); Coligan et al, Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18^{th} edition (1990). These texts can, of course, also be referred to in making or using an aspect of the disclosure.

As used herein, the term “pharmaceutical composition” is a formulation containing one or more otic therapeutic agents (e.g., hearing loss treatment agents) of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., imprinting disorders, and the like) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is an imprinting disorder.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

General guidelines for the formulation and manufacture of pharmaceutical compositions and agents are available, for example, in Remington's The Science and Practice of Pharmacy, 21^{st} Edition, A. R Gennaro; Lippincott, Williams & Wilkins, Baltimore, Md., 2006. Conventional excipients and accessory ingredients may be used in any pharmaceutical composition.

In some embodiments, the pharmaceutical composition or reconstituted solution of the present disclosure is refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). In some embodiments, the present disclosure also relates to a method of increasing stability of the pharmaceutical composition or reconstituted solution and by storing the pharmaceutical composition or reconstituted solution at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ET™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-king that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the bather to be permeated are used in the formulation. Such penetrants are generally known in the art, and include, for example, for transmucosal administration, detergents, bile salts, and fusidic acid derivatives. Transmucosal administration can be accomplished through the use of nasal sprays or patches, thin films, tablets to be used for buccal or sublingual application or suppositories. For transdermal administration, the active compounds are formulated into ointments, salves, creams, gels, patches or microneedle delivery systems as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylacticglycolic acid and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the imprinting disorder and also preferably causing complete regression of the imprinting disorder. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ration other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

It is to be understood that the compounds of the present disclosure can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

In certain embodiments, it is to be understood that the compounds of the present disclosure can be a prodrug (that may include an ester) of any compound disclosed herein.

In certain embodiments, it is to be understood that the compounds of the present disclosure can also be prepared as co-crystals with other compounds.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

Where ranges are given, endpoints are included. Furthermore, it is to be understood that unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same.

The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1: Preparation of Composition of CHIR99021, Valproic Acid Poloxamer 407, and DMSO

Preparation of poloxamer 407 solution: 17 g of poloxamer 407 was slowly added to 70 ml of ice cold phosphate buffered saline that is constantly stirred. The resulting mixture was stirred overnight over ice (or in a cold room) to dissolve the poloxamer. Additional phosphate buffered saline was added until a total volume of 100 ml is reached. The resulting solution of poloxamer was filtered using a 0.2 um filter prior to test article formulation. This solution can be then stored at 4° C.

Preparation of CHIR99021 solution: 55.6 mg of CHIR99021 was dissolved in DMSO to a final volume of 1 mL. The resulting mixture may be gently heated to about 37° C. and vortexed to ensure dissolution of CHIR99021.

Preparation of composition of CHIR99021, valproic acid, poloxamer 407, and DMSO: 87.6 mg of valproic acid was added to 0.95 ml of the prepared poloxamer 407 solution at about 4° C., and the resulting mixture was at about 350 rpm for 15 minutes to dissolve the valproic acid.

To prepare 1 ml of gel, 25 μl of CHIR99021 solution and 25 μl of DMSO were added to the poloxamer 407 solution that contains valproic acid. CHIR99021 may come out of solution and the mixture may be incubated at 37° C. to re-dissolve CHIR99021, and then cooled to about 4° C. to form a flowable mixture. Final concentrations: CHIR99021 at 1.39 mg/mL, WA at 87.6 mg/mL, DMSO at 5 wt %, and Poloxamer at 407 16 wt %. The composition forms a gel at about 37° C. Valproic acid means sodium valproate in this example.

### Example 2: Preparation and Stability Analysis of Composition of CHIR99021, Sodium Valproate, Poloxamer 407, and DMSO for Lyophilization

Preparation of poloxamer 407 water solution: To an Erlenmeyer flask was added 249.0 g of cold purified water. The water was stirred with an overhead stirrer while small increments of a total of 51.0 g of poloxamer 407 powder was added over 1 hour. The flask containing the poloxamer 407-Water mixture was then sealed and cooled at 0-8° C. overnight to allow complete dissolution of the poloxamer 407 affording a clear homogenous solution.

Preparation of CHIR99021 DMSO solution: To a 20 mL volumetric flask was added 1.32 g of CHIR99021 followed by 18 mL of DMSO. The mixture was stirred or vortexed gently. The mixture was then warmed to about 37° C. (but not above 37° C.) for 10 minutes or until a clear solution was obtained. Gentle vortexing and warming were repeated until a clear yellow solution was formed. Additional DMSO was added to the solution to reach a final volume of 20 ml, and the solution was gently mixed in the flask to obtain a clear yellow solution. The solution may be stored in the refrigerator at 0-8° C. until needed. The solution must be thawed before use by heating at 37° C. until no turbidity or precipitate is observed, and intermittent vortexing may be needed.

Preparation of poloxamer 407, sodium valproate, CHIR99021, and DMSO solution: To a 1-liter glass beaker with a magnetic stir bar was add 291.6 g of cold poloxamer 407 water solution. The beaker containing the poloxamer solution was then placed into an ice bath, and the solution was gently mixed. 26.6 g of sodium valproate powder was added in small portions to the poloxamer water solution while stirring continuously. The resulting mixture was stirred until complete dissolution of sodium valproate. 15 mL (16.5 grams) of CHIR99021 DMSO solution was added dropwise to the poloxamer/sodium valproate solution while stirring. The resulting mixture was stirred until a clear, yellow solution was formed. The solution was then diluted by adding an equal weight of water (334.7 grams). The solution was sparged with nitrogen for 1 minute and was then sterile filtered using a 0.2 μM PES membrane filter and PTFE syringe.

Stability Analysis: Prior to lyophilization, the stability of the filtered solution was analyzed by HPLC. The solution was held at room temperature for 4 hours, and samples of the solution was analyzed by HPLC during the period of time. For CHIR99021, a total of about 4.6% impurities was formed over 4 hours, indicating that the processing time between preparation and lyophilization of the solution may affect the purity of the lyophilized composition. Further, about 100% of sodium valproate remained in the solution.

### Example 3: Lyophilization of Composition of CHIR99021, Sodium Valproate, Poloxamer 407, and DMSO, and Stability Analysis of the Lyophilized Composition

Filling glass vials with individual doses: A tray of sterile glass vials and sterile stoppers were transferred in a sterile environment. For each 5 mL glass vial, 2.2 grams of sterile poloxamer 407 sodium valproate, and CHIR99021 solution was dispensed as an individual dose. The dispense was performed using a micropipette or suitable dispenser. The stoppers were then partially inserted into the necks of each vial aseptically.

Lyophilization of poloxamer 407, sodium valproate, CHIR99021, and DMSO solution: The tray of filled glass vials was placed into a lyophilizer in a sterile environment. The temperature in the lyophilizer was slowly reduced to −45° C. (at a rate of 0.5° C. per minute) and then held at −45° C. for 3 hours. A vacuum of 80 mTorr was applied to the lyophilizer. The temperature was then slowly increased to −30° C. (at a rate of 0.5° C. per minute) and then held at −30° C. for 15 hours under a vacuum of 80 mTorr. The temperature was then slowly increased to 15° C. (at a rate of 0.5° C. per minute). The temperature was held at 15° C. for 20 hours under a vacuum of 80 mTorr. At the end of the cycle, the glass vials were stoppered under nitrogen and vacuum, and then the vacuum was then released completely while backfilling the lyophilizer with nitrogen. The glass vials were removed from the lyophilizer, capped, and crimped in a sterile environment. The 5 ml glass vials containing individual doses of formulated cake may be stored at −20° C. until use.

Stability Analysis: The stability of the lyophilized composition formulation (Dosage 1) was analyzed upon storage at 5° C., room temperature, or 40° C. under 75% relative humidity in the dark Samples were taken at various time points, reconstituted as described in Example 4 and samples analyzed by HPLC. As shown in Tables A and B below, CHIR99021 degrades at a reduced rate at 5° C. or room temperature (Ambient) compared with 40° C. under 75% relative humidity. About 100% of sodium valproate remained.

TABLE A
Percentage of Remaining CHIR99021 at Testing Conditions.

| Time (hours) | 40° C./75% Relative Humidity | Ambient | 5° C. |
|---|---|---|---|
| 0 | 96.97% | 96.97% | 96.97% |
| 19.5 | | 97.05% | |
| 22 | 96.36% | | |
| 47 | 95.28% | | |
| 115.5 | | 96.82% | |
| 118 | 93.25% | | |
| 168 | 92.27% | | 96.63% |
| 2 months | | 95.18% | |

TABLE B
Percentage of CHIR99021 Impurity at Relative Retention Times.

| Time (hours) | 40° C./75% Relative Humidity | Ambient | 5° C. |
|---|---|---|---|
| 0 | 0.04% | 0.04% | 0.04% |
| 19.5 | | 0.07% | |
| 22 | 0.58% | | |
| 47 | 1.10% | | |
| 115.5 | | 0.13% | |
| 118 | 2.40% | | |
| 168 | 2.68% | | 0.06% |
| 2 months | | 1.26% | |

Long-term stability of the lyophilized composition formulation (Dosage 1) was tested. The composition was held at room temperature (ambient) in the dark for two months and reconstituted as described in Example 4 below. The analysis showed degradants of CHIR99021 at various retention times as compared to 0.04% at Tom. CHIR99021 had an increased rate of impurity formation in solution formulations compared to lyophilized formulations.

### Example 4. Formulation and Stability Analysis of Injection Dosages from Lyophilized Composition of CHIR99021, Sodium Valproate, Poloxamer 407, and DMSO

Formulation of Dosage 1: 6.4 grams of DMSO was added to a beaker containing 93.6 grams of purified water. The mixture was stirred for 3-5 minutes until homogeneous. The solution was sparged with nitrogen for 1-2 minutes, and was then sterile filtered into a clean container using PES 0.2 um filter and 10-ml syringe. 0.85 ml of the filtered solution was added to the lyophilized composition (Example 3) in the 5 ml vial, and the mixture was held at 2-8° C. for 20 minutes or until a clear solution was formed.

Formulation of Dosage 2: 4.6 grams of poloxamer 407 was added to a beaker containing 89.5 grams of purified water. The mixture was stirred and then held at 2-8° C. overnight until complete dissolution of poloxamer 407 to form a clear solution 5.9 grams of DMSO was then added to the beaker, and the solution was stirred until homogeneous. The solution was sparged with nitrogen for 1-2 minutes, and was then sterile filtered into a clean container using PES 0.2 um filter and 10-ml syringe. 1.4 ml of the filtered solution was added to the lyophilized composition (Example 3) in the 5 ml vial, and the mixture was held at 2-8° C. for 20 minutes or until a clear solution was formed.

Formulation of Dosage 3: 8.4 grams of poloxamer 407 was added to a beaker containing 85.7 grams of purified water. The mixture was stirred and then held at 2-8° C. overnight until complete dissolution of poloxamer 407 to form a clear solution 5.9 grams of DMSO was then added to the beaker, and the solution was stirred until homogeneous. The solution was sparged with nitrogen for 1-2 minutes, and was then sterile filtered into a clean container using PES 0.2 um filter and 10-ml syringe. 2.1 ml of the filtered solution was added to the lyophilized composition (Example 3) in the 5 ml vial, and the mixture was held at 2-8° C. for 20 minutes or until a clear solution was formed.

Formulation of Dosage 4: 6.47 grams of DMSO was added to a beaker containing 92.35 grams of purified water and 1.18 grams of benzyl alcohol. The mixture was stirred for 3-5 minutes until homogeneous. The solution was sparged with nitrogen for 1-2 minutes, and was then sterile filtered into a clean container using polyethersulfone (PES) 0.2 um filter and 10-ml syringe. 0.85 ml of the filtered solution was added to the lyophilized composition (Example 3) in the 5 ml vial, and the mixture was held at 2-8° C. for 20 minutes or until a clear solution was formed.

The representative composition of Dosages 1-3 are presented in Table C below. The ranges for Dosage 1 are based on observed amounts from three batches of lyophilized product and reconstituted product ready for dosing. The ranges for Dosages 2 and 3 are based on observed amounts from two batches of lyophilized product and reconstituted product ready for dosing.

TABLE C

| | Dosage 1 | Dosage 2 | Dosage 3 |
|---|---|---|---|
| CHIR99021 (mg/mL) | 3.24-3.27 | 2.09-2.10 | 1.44-1.45 |
| Valproic acid (mg/mL) | 87.16-89.33 | 56.23-56.51 | 48.73-38.93 |
| Poloxamer 407 (% by weight) | 14.87-15.2 | 13.44-13.48 | 14.19-14.22 |
| DMSO (% by weight) | 4.91-4.95 | 5.02-5.03 | 5.29 |

Stability Analysis: The stability of a higher concentration of CHIR99021 and VPA similar to formulated Dosage 1 above was analyzed by visual inspection and HPLC upon storage in a refrigerator at 2-8° C. for 8 hours. The higher concentration lyophilized formulation showed effective reconstitution of both agents in solution for 8 hours. Results of the analysis are shown in the Table D below.

TABLE D

| | CHIR99021 | % Rel | Sodium Valproate | % Rel |
|---|---|---|---|---|
| Testing Condition | Conc. (mg/mL) | Error | Conc. (mg/mL) | Error |
| Post Lyo | 3.47 | 2.2% | 102.9 | 7.3% |
| Post Lyo, 24 Hrs | 3.36 | −1.3% | 101.0 | 5.3% |
| Post Lyo | 3.48 | 2.4% | 101.1 | 5.5% |
| Post Lyo, 24 Hrs | 3.42 | 0.6% | 103.1 | 7.5% |

### Example 5: Preparation of Composition of CHIR99021, Sodium Valproate, Poloxamer 407, and Benzyl Alcohol

Preparation of poloxamer 407 water solution: To an Erlenmeyer flask was added 81.0 g of cold purified water. The water was stirred with an overhead stirrer while small increments of a total of 19.0 g of poloxamer 407 powder was added over 1 hour. The flask containing the poloxamer 407-Water mixture was then sealed and cooled at 0-8° C. overnight to allow complete dissolution of the poloxamer 407 affording a clear homogenous solution.

Preparation of CHIR99021 DMSO solution: To a 20 mL volumetric flask was added 330 mg of CHIR99021 followed by 4 mL of DMSO. The mixture was stirred or vortexed gently. The mixture was then warmed to 37° C. (but not above 37° C.) for 10 minutes or until a clear solution was obtained. Gentle vortexing and warming were repeated until a clear yellow solution was formed. Additional DMSO was added to the solution to reach a final volume of 5 ml, and the solution was gently mixed in the flask to obtain a clear yellow solution. The solution may be stored in the refrigerator at 0-8° C. until needed. If the solution was frozen upon storage, it must be thawed before use (by heating at 37° C. until no turbidity or precipitate is observed, and intermittent vortexing may be needed).

Preparation of poloxamer 407, sodium valproate, CHIR99021, and benzyl alcohol solution: To a beaker with a magnetic stir bar was add 2.91 g of the cold poloxamer 407 water solution. The beaker containing the poloxamer solution was then placed into an ice bath, and the solution was gently mixed. 280 mg of sodium valproate powder was added in small portions to the poloxamer water solution while stirring continuously. The resulting mixture was stirred until complete dissolution of sodium valproate. 150 μl of CHIR99021 DMSO solution was added dropwise (to prevent large precipitate aggregates from forming) to the poloxamer/sodium valproate solution while stirring. The resulting mixture was stirred until a clear, yellow solution was formed. 150 μl of benzyl alcohol was added, and the mixture was stirred until a homogeneous solution was formed. The solution was sparged with nitrogen for 1 minute and was then sterile filtered using a 0.22 μM polyethersulfone (PES) membrane filter and polytetrafluoroethylene (P IFE) syringe.

### Example 6: Preparation of a Composition of CHIR99021, Sodium Valproate, Poloxamer 407, and DMSO for Injection (Low and Isotonic Compositions)

Preparation of poloxamer 407 solution: 17 g of poloxamer 407 was slowly added to 83 g of ice-cold phosphate buffered saline that is constantly stirred. The resulting mixture was stirred overnight over ice (or in a cold room) to dissolve the poloxamer. The resulting solution of poloxamer was filtered using a 0.2 um filter prior to test article formulation. This solution can be then stored at 4° C.

Preparation of CHIR99021 DMSO solution: To a 2 mL volumetric flask was added 0.112 g of CHIR99021 followed by 1.5 mL of DMSO. The mixture was stirred or vortexed gently. The mixture was then warmed to about 37° C. for 10 minutes or until a clear solution was obtained Gentle vortexing and warming were repeated until a clear yellow solution was formed. Additional DMSO was added to the solution to reach a final volume of 2 ml, and the solution was gently mixed in the flask to obtain a clear yellow solution. The solution may be stored in the refrigerator at 0-8° C. until needed. The solution must be thawed before use by heating at 37° C. until no turbidity or precipitate is observed, and intermittent vortexing may be needed.

Preparation of poloxamer 407, sodium valproate, CHIR99021, and DMSO solution (Low): To a 100-mL glass beaker with a magnetic stir bar was add 14.685 g of cold poloxamer 407 water solution. The beaker containing the poloxamer solution was then placed into an ice bath, and the solution was gently mixed. To it, 0.6 mL of phosphate buffered saline was added and resulting mixture was stirred. 0.1575 g of sodium valproate powder was added in small portions to the poloxamer water solution while stirring continuously. The resulting mixture was stirred until complete dissolution of sodium valproate. 50.2 uL of DMSO was added dropwise to the poloxamer/sodium valproate solution while stirring. Then, 99.8 uL of CHIR99021 DMSO solution was added dropwise to the poloxamer/sodium valproate/DMSO solution while stirring. The resulting mixture was stirred until a clear, yellow solution was formed. The solution was sparged with nitrogen for 1 minute and was then sterile filtered using a 0.2 μM PES membrane filter and PTFE syringe. The composition forms a gel at about 37° C.

Preparation of poloxamer 407, sodium valproate, CHIR99021, and DMSO solution (Isotonic): To a 100-mL glass beaker with a magnetic stir bar was add 14.685 g of cold poloxamer 407 water solution. The beaker containing the poloxamer solution was then placed into an ice bath, and the solution was gently mixed. To it, 0.713 mL of phosphate buffered saline was added and resulting mixture was stirred. 0.0395 g of sodium valproate powder was added in small portions to the poloxamer water solution while stirring continuously. The resulting mixture was stirred until complete dissolution of sodium valproate. 13.2 uL of DMSO was added dropwise to the poloxamer/sodium valproate solution while stirring. Then, 24.3 uL of CHIR99021 DMSO solution was added dropwise to the poloxamer/sodium valproate/DMSO solution while stirring. The resulting mixture was stirred until a clear, yellow solution was formed. The solution was sparged with nitrogen for 1 minute and was then sterile filtered using a 0.2 μM PES membrane filter and PTFE syringe. The composition forms a gel at about 37° C.

### Example 7: In Vivo Mouse Hearing Loss Model

The effects of CHIR99021 and valproic acid (VPA) on hearing in mice with SNHL were examined. Ten-week-old CBA/CaJ mice were deafened using an established method where mice were exposed to 8-16 kHz octave band noise for 2 hours at >116 dB (Wang et al, 2002). This model was shown to cause immediate and extensive hair cell loss, but also to cause damage to other structures, such as the lateral wall, supporting cells, and spiral ganglion, all of which could limit the extent of possible hearing recovery (Wang et al, 2002). Auditory brainstem responses (ABRs) were obtained using tone-burst stimuli for frequencies spanning ˜80% of the cochlea 24 hours after noise administration to establish a baseline for recovery. Animals were dosed once following the 24-hour ABR Distortion product octoacoustic emissions (DPOAEs) were not routinely analyzed since thresholds after treatment were above DPOAE detection levels. CHIR99021 and WA were delivered locally by intra-tympanic injection into the middle ear using a pulled glass pipette that mimics the standard clinical middle ear injection technique. The delivery vehicle was adapted from previous work using thermo-reversible poloxamer gels to deliver drugs into the middle ear for diffusion into the cochlea (Salt et al, 2011; Wang et al, 2009). Doses of CHIR99021 and WA were scaled several hundred-fold above the active in vitro concentration to account for the gradient of drug entry through the round window membrane described in previous studies (Plontke et al, 2008). Specifically, mice were administered 10 μL of a composition containing 87.6 mg/ml NaVPA (527 mM) and 1.39 mg/ml CHIR99021 (approximately 3 mM). Using established techniques (Hirose et al, 2014), perilymph was sampled from 7 animals and analyzed using mass spectrometry to determine entry of CHIR99021 and WA into the cochlea. Within 0.5 hours, CHIR99021 was detected at 3.5 04±1.5 μM and VPA was detected at 310.3 04±51.8 μM. Thus, concentrations that were active in the Lgr5+ cell assay in vitro were achieved within the cochlea using an intra-tympanic injection.

Consistent with previous reports of rapid HC death using this noise-damage model (Wang et al., 2002), total HC numbers observed prior to injection (24 hours after damage) did not significantly differ from those observed in vehicle-injected animals at 5 weeks (data not shown, n=6, p=0.11). This supports prior work demonstrating a lack of spontaneous hair cell regeneration in post-natal mammals (Cox et al., 2014; Bramhall et al., 2014). Five weeks after injection, animals that received CHIR99021/VPA showed significantly lower absolute ABR thresholds relative to vehicle-injected animals at 5, 10, 20 (p<0.0001), and 28.3 (p<0.05) kHz (FIG. 1). Average threshold changes from post-damage to 5 weeks were significantly greater across all frequencies tested in treated animals, with some demonstrating threshold recoveries up to 35 dB (FIG. 1).

### Studies of Low or Isotonic Test Compositions in Guinea Pigs

The studies used pigmented, NIH-strain guinea pigs weighing 400-600 g. Experiments were performed under protocol 20180054, approved by the Institutional Animal Care and Use Committee of Washington University. Animal use followed policies in accordance with the United States Department of Agriculture and National Institutes of Health guidelines for the handling and use of laboratory animals.

Animal were administered either 20 ml or 50 ml of the test composition by intra-tympanic injection into the middle ear using a pulled glass pipette that mimics the standard clinical middle ear injection technique. All experiments were performed as non-recovery procedures. Animals were anesthetized with 100 mg/kg sodium thiobutabarbital (Inactin, Sigma, St Louis, Mo.). A polyethylene tracheal cannula was inserted, and the animal was maintained on 0.8 to 1.2% isofluorane in oxygen using a mechanical ventilator. End-tidal CO2 level was maintained close to 5% by adjustment of the ventilator tidal volume. Heart rate and oxygen saturation were monitored, and core body temperature was maintained at 38° C. with a thermistor-controlled heating blanket.

When histology was analyzed after final physiological testing, total hair cell (total HC), inner hair cell (IHC), and outer hair cell (OHC) numbers increased in CHIR99021+VPA-treated animals relative to vehicle-treated animals (Total HCs=376.0±18.5, IHCs=245.9±7.9, OHCs=130.1±18.8; mean±SE) vs (Total HCs=259.3±29.0 [p<0.01], IHCs=188.6±16.5 [p<0.01], OHCs=75.3±12.4 [p<0.05]) (FIG. 2).

This in vivo study showed that the components of the composition, CHIR99021 and WA, improved auditory thresholds and restored hair cells in a mouse model of noise-induced hearing loss.

### Example 8: Treatment with Reconstituted Lyophilized Test Compositions Containing CHIR99021, NaVPA and Poloxamer Leads to Improvement in Hearing in an Animal Model

To confirm that a reconstituted lyophilized composition containing NaVPA and CHIR99021 demonstrated treatment of hearing loss, the effects of a test composition (Example 9) and NaVPA+CHIR99021 in an animal efficacy model were evaluated.

### Method:

### Noise Exposure

10-week-old CBA/CaJ mice were exposed fully awake to 8-16 kHz octave band noise for two hours at 120 dB SPL. Noise exposure was performed in an Industrial Acoustics double walled sound booth. Two 30×19×13 cm reverberant plastic enclosures open at the top, each containing two mice isolated in separate compartments were suspended with bottoms 28 cm below an exponential horn (Selenium Cometa HM4750-SLF). Noise was generated digitally using custom Labview routines, then presented using a TDT RZ6 in combination with a Crown power amplifier. Sound level was monitored in real time by Brüel & Kjaer Type 2203 sound level meter and tracked by custom software.

### ABR Recordings

Animals were anesthetized with a mixture of ketamine and xylazine (80/15 mg/kg, IP) and placed dorsally in a custom head-holder with an ES-1 free-field speaker (Tucker-Davis Technologies) 7 cm directly lateral from the right ear. Subdermal platinum electrodes (Grass) were placed behind the right pinna (reference), on the vertex (active), and under the skin of the back (ground). A rectal probe was used to monitor temperature, which was maintained near 38° C. using a DC current-based isothermal pad (FHC). Tone bursts 5 ms in duration (0.5 ms cos² R/F) were presented 500-1000 times at 20/s in descending intensity using a 5 dB minimum step size until wave I of the ABR could no longer be visually discerned. The stimulus level was then increased until the response re-appeared. Recording utilized Biosig32 and TDT hardware. ABR thresholds were obtained at 5, 10, 20, 28.3, and 40 kHz, 24 h and 5 weeks after post-exposure by an operator blinded to experimental treatment ABRs were plotted and reported using standard error. Comparisons from 24 h to 5 wks, both within and across groups, were compared using a two-tailed t-test

### Intratympanic Injection

Intratympanic injections were performed after ABR testing, 24 hrs after noise exposure. The therapeutic composition was maintained at a cold temperature in order reduce viscosity and allow loading of the syringe and injection through the pipette. 1 ml allotments of the composition 1 ml allotments of therapeutic agents dissolved in cold Poloxamer 407 plus Evans blue were drawn into a 1 ml disposable syringe to which 34 cm of polyethylene plastic tubing was attached. The tubing was sized to fit snuggly to a glass 1.5 mm OD microcapillary tube (WPI), pulled to a fine point in a custom pipette puller and broken with forceps to a tip width of 40-60 μm. The assembly was kept in the dark at 4° C. until use. Immediately before use, the pulled pipette was attached to a micro-positioner. Mice were injected with ketamine/xylazine (80/15 mg/kg) and positioned ventrally in a custom head-holder. Under an operating scope, the cartilage ring at the base of the right pinna was slightly expanded by a bloodless cut on the ventral side and a retractor and forceps were used to reveal a full view of the pinna. The positioner-tubing assembly was then used to make two holes in the tympanic membrane just large enough to admit the pipette. The first hole served as a vent hole and was made just anterior to the umbo. For the entry hole, the pipette was aimed at the posterior margin of the tympanic membrane, just ventral to the incus (pars flacida). Leaving the pipette in place, the middle ear was filled slowly by depressing the syringe, from postero-dorsal to antero-ventral until excess Poloxamer began to emerge from the vent hole. Topical Lidocaine was then applied, and mice were allowed to recover under a warming lamp. Mice show normal activity levels within 24 hrs and no middle ear infections or surgical complications.

### Tissue Processing

At the end of all procedures, mice were deeply anesthetized using pentobarbital and perfused transcardially with 4% paraformaldehyde in 0.1 M phosphate buffer. Cochleae were then removed and placed in the same fixative for 24 hrs, after which they were transferred to 0.12 M sodium EDTA and stored at 4° C. for later processing.

### Histology

After decalcification in sodium EDTA for 72 hrs, the organ of Corti is then removed for histological analyses. Cochleae were permeabilized and blocked for 1 h in blocking solution (0.3% Triton X-100 and 15% heat-inactivated donkey serum in PBS), and exposed to diluted primary antibody in blocking solution overnight at 4° C. The organ of Corti was subsequently immunoassayed for Myosin Vila to detect hair cells. The primary antibody for Myosin Vila (Proteus Biosciences, anti-rabbit, #25-6790) was used at a 1:500 dilution, and the AlexFlour 568 secondary antibody was used at 1:500 dilution. Invitrogen) were diluted 1:500 for detection of primary antibodies. Nuclei were visualized with 4,6-diamidino-2-phenylindole (DAPI; Vector Laboratories).

FIG. 5 shows the results of a NaVPA+CHIR99021 solution with a low concentration of NaVPA (e.g. see Example 6, however the poloxamer is omitted). As seen from FIG. 5, a significant improvement in thresholds is seen at all frequencies.

FIG. 4 shows the results of a NaVPA+CHIR99021 solution with an isotonic concentration of NaVPA (e.g. see Example 6 however the poloxamer is omitted). As seen from FIG. 4, a significant improvement in thresholds is seen at all frequencies.

FIG. 3 shows the results of a reconstituted test composition (e.g. see Example 10). As seen from FIG. 3, a significant improvement in thresholds is seen at 20 kHz and 28.3 kHz. This validates that reconstituting lyophilized compositions comprising NaVPA, CHIR99021 and Poloxamer 407 is a viable strategy for administering NaVPA and CHIR99021 since the effects on hearing improvement are similar to administering NaVPA and CHIR99021 as a solution.

### Example 9: Increased Levels of CHIR99021 and NaVPA Observed in the Cochlear Perilymph

NaVPA pharmacokinetics (FIG. 6).

Different concentrations of CHIR99021 and NaVPA were tested with and without NaCl to adjust osmolality to account for NaVPA's contribution to osmolality within the composition. Three hours after application of the composition to the round window, the cochlear perilymph was sequentially sampled from the apex to the base to test the overall concentration of NaVPA along the cochlear length, where sample 1 is the extreme apex, each subsequent sample is closer to the base, and the 5th sample is considered to be the extreme base. All samples after the 5th are cerebral spinal fluid. Represented by line (4) on FIG. 6, a high osmolality composition (by NaCl addition) with low NaVPA (14.4 mg/ml) shows that little NaVPA entered the cochlea, and that NaVPA was only present near the drug application site (base). Represented by line (3) on FIG. 6, a high osmolality formulation with higher levels of NaVPA (88.6 mg/ml) shows increased drug entry of NaVPA that traveled further up the cochlea. Increasing the concentration NaVPA approximately 1.5× higher (˜130 mg/ml) surprisingly shows an approximately 10-fold increase in NaVPA concentration within the cochlea, with increased distribution along the cochlear length (lines (2) and (1) on FIG. 6). The test compositions in this example contain P407 at −15.5% w/v % (e.g. see Table 34) and can be made following the method of Example 10.

CHIR99021 (CHIR) Pharmacokinetics (FIG. 7).

Different concentrations of CHIR99021 and NaVPA were tested with and without NaCl to adjust osmolality to account for NaVPA's contribution to osmolality within the composition. Three hours after application of the composition to the round window, the cochlear perilymph was sequentially sampled from the apex to test the overall concentration along the cochlear length, where sample 1 is the extreme apex, each subsequent sample is closer to the base, and the 5th sample is considered to be the extreme base. All samples after the 5th are cerebral spinal fluid. Represented by line (5) on FIG. 7, removing NaVPA entirely from the formulation shows low CHIR99021 entry into the cochlea. Represented by line (6) on FIG. 7, a high osmolality formulation (by NaCl addition) with low NaVPA (14.4 mg/ml) similarly shows low concentrations of CHIR99021 entering the cochlea, which did not meet the apex. Represented by line (4) on FIG. 7, removing NaVPA entirely and adjusting osmolality with NaCl (24 mg/ml) shows low CHIR99021 entry.

Represented by line (3) on FIG. 7, a high osmolality formulation with higher levels of NaVPA (88.6 mg/ml) shows increased drug entry of CHIR99021, which also traveled further up the cochlea. Increasing the concentration of NaVPA in the composition by approximately 1.5× (˜130 mg/ml) (lines (1) and (2) on FIG. 7) shows similar but slightly better entry of CHIR99021 in comparison to the composition with 88.6 mg/ml NaVPA (line (3)). These data suggest that at a certain threshold (optimal at −130 mg/mL NaVPA), NaVPA surprisingly facilitates CHIR99021 entry into the cochlear in the range of 4-14 fold. The test compositions in this example contain P407 at −15.5% w/v % (e.g. see Table 34) and can be made following the method of Example 10.

### Example 10: Preparation and Lyophilization of a Composition of CHIR99021, Sodium Valproate, Poloxamer 407, and DMSO for Injection (Test Composition) (GMP)

Step 1: 6453 g of water for injection (WFI) was added to a 20 L jacketed formulation vessel. The temperature of the water was controlled between 2-4° C. using the jacketed vessel assembly. 1322 g of P407 was added to the chilled water in portions while stirring the solution at 300 rpm using an overhead stirrer for initial mixing (speed was adjusted to ensure no frothing while mixing). The temperature of P407 in water was maintained cold to ensure that the solution was free flowing during the actual compounding step although at this step, temperature control was not critical. The stock solution of P407 was then used in the next steps of compounding the test composition.

Step 2: 709 g of solid sodium valproate was weighed and added in small portions, to the P407 aqueous solution from step 1 above, while maintaining temperature of the solution to 2-8° C. and continuously stirring the solution using an overhead stirrer. The mixing speed was adjusted to ensure no frothing while mixing. The required solution temperature was achieved by setting the temperature of the jacketed vessel to 15° C. Mixing was completed in 60 minutes.

Step 3: 26.3 g of CHIR99021 was weighed and added to pre-warmed (at 32-35° C.) 407 g of DMSO in a labeled clean glass container. The solution was stirred using magnetic stir plate and stir bar and mixed for a maximum of 60 minutes, until clear solution was obtained. While maintaining the temperature of the NaVPA-P407-water solution at ˜15-20° C., CHIR99021-DMSO solution was slowly added to it using a peristaltic pump at ˜10 g/min.

Step 4: The clear solution obtained in Step 4 was then diluted by addition of 8917.4 g of WFI at 2-8° C. while maintaining temperature of the solution in vessel at 20° C. The diluted solution was sparged with nitrogen gas for 1-2 minutes.

Step 5: The diluted solution was filtered using a polyethersulfone (PES) membrane based standard Sartopore® 2, 0.2 μm pore size, 1000 cm² capsule filter under aseptic conditions.

Step 6: Filling glass vials with individual doses: A tray of sterile glass vials and sterile stoppers were transferred in a sterile environment. For each 5 mL glass vial, 2.2 grams of sterile poloxamer 407, sodium valproate, and CHIR99021 solution was dispensed as an individual dose. The dispense was performed using filling machine. The stoppers were then partially inserted into the necks of each vial aseptically. The composition forms a gel at about 37° C.

Steps 1-6 take about 12 hours to complete.

Step 7: Lyophilization of poloxamer 407, sodium valproate, CHIR99021, and DMSO solution: The tray of filled glass vials was placed into a lyophilizer in a sterile environment. The temperature in the lyophilizer was slowly reduced to −45° C. (at a rate of 0.5° C. per minute) and then held at −45° C. for 3 hours. A vacuum of 80 mTorr was applied to the lyophilizer. The temperature was then slowly increased to −30° C. (at a rate of 0.5° C. per minute) and then held at −30° C. for 15 hours under a vacuum of 80 mTorr. The temperature was then slowly increased to 15° C. (at a rate of 0.5° C. per minute). The temperature was held at 15° C. for 20 hours under a vacuum of 80 mTorr. At the end of the cycle, the glass vials were stoppered under nitrogen and vacuum, and then the vacuum was then released completely while backfilling the lyophilizer with nitrogen. The glass vials were removed from the lyophilizer, capped, and crimped in a sterile environment. The 5 ml glass vials containing individual doses of the cake test composition may be stored at −20° C. until use.

Notes on the lyophilization cycle: for the formation of a useful lyophilized product after step 6, it is important to use an appropriate lyophilization cycle that consists of suitable temperature and suitable vacuum pressure used during the drying process (e.g. see step 7). In absence of a proper lyophilization cycle, the dried mass can result in a flat sheet, e.g. see FIG. 8. The flat sheet visible in FIG. 9 was generated by freezing the “wet” test composition (i.e. the product from step 6) in a sample vial with liquid nitrogen. The sample was then lyophilized by king at room temperature under a vacuum pressure at 400 mTorr. The test composition produced using a suitable lyophilization cycle, such as outlined in Step 7 above, produces a lyophilized cake as shown in FIG. 9.

### Example 11: Order of Compounding

The order in which the components of the test composition are added (i.e. compounded) in the example was studied (see Table 26).

Table 26 shows the results when CHIR99021 is added at different stages in the manufacture of the test composition

TABLE 26
order of addition of components in the test composition

| | Preferred approach, | | | |
|---|---|---|---|---|
| Order of | as described in the | | | |
| addition | Examples above | Prophetic example 1 | Prophetic example 2 | Prophetic example 3 |
| 1 | Poloxamer | Water | Poloxamer | Poloxamer |
| 2 | Water | NaVPA | NaVPA | Water |
| 3 | NaVPA | Poloxamer | Water | CHIR99021 in DMSO |
| | | | | (Predissolved) |
| 4 | CHIR99021 in DMSO | CHIR99021 in DMSO | CHIR99021 in DMSO | NaVPA |
| | (Predissolved) | (Predissolved) | (Predissolved) | |
| 5 | | | | |
| Result | Clear composition | Prophetic result: most likely | Prophetic result most | Prophetic result: most likely a |
| | | a clear composition (no | likely a clear | clear composition. However, |
| | | additional perceived benefit | composition (no | less ideal since the time that |
| | | over the preferred approach). | additional perceived | CHIR99021 is in solution is |
| | | Deemed that the poloxamer | benefit over the | increased, causing possible |
| | | would take more time to | preferred approach). | degradation. |
| | | dissolve than the current | | Deemed that the poloxamer |
| | | approach. | | would take more time to |
| | | | | dissolve than the current |
| | | | | approach. |

| Order of | | | |
|---|---|---|---|
| addition | Prophetic example 4 | Prophetic example 5 | Alternate approach |
| 1 | Poloxamer | Poloxamer | Poloxamer |
| 2 | CHIR99021 in DMSO | NaVPA | Water |
| | (Predissolved) | | |
| 3 | Water | CHIR99021 | NaVPA |
| 4 | NaVPA | DMSO | DMSO |
| 5 | | Water | CHIR99021 |
| Result | Insoluble mixture | Insoluble mixture | Insoluble mixture |
| | based on the | based on the | |
| | solubility of each | solubility of each | |
| | component. | component. | |

For acceptable compositions, a CHIR99021 stock solution was prepared at the given concentration in DMSO because the solubility of CHIR99021 is very limited in almost all organic solvents except polar solvents, such as DMSO. The first step in the sequence of compounding was to add pre-weighed solid sodium valproate to the stock P407 aqueous solution over ice. The temperature of P407 solution is important to keep the viscosity of this thermoreversible polymer in a liquid free-flowing state. After a clear solution was obtained, the CHIR99021-DMSO stock solution was added slowly to the NaVPA-P407-water solution. The slow addition of CHIR99021-DMSO was necessary to avoid precipitation of CHIR99021 from the solution. The CHIR99021-DMSO stock solution is added after the NaVPA has dissolved to minimize the time that CHIR99021 is in solution. It has been established that for the test composition, compounding must be completed in under 12 hours otherwise CHIR99021 can begin to precipitate out of solution. CHIR99021 also begins to degrade after extended periods of time in solution.

### Example 12: Preparation of Reconstitution Fluid/Diluent

Preparation of a water and DMSO Diluent: Step 1: To a 15 L jacketed vessel, water for injection (WFI) (9547 g) was added. DMSO (653 g) was weighed in a separate 1 L container. DMSO was added slowly over 4-6 minutes to WFI in the vessel at ambient temperature (˜20° C.). The solution was mixed continuously for 15-20 minutes using a magnetic stir plate and stir bar while avoiding frothing or splashing of solution. The clear solution was sparged with nitrogen gas for 8-10 minutes.

Step 2: The sparged solution was then sterile filtered using PES membrane based standard Sartopore® 2, 0.2 μm pore size, 1000 cm² capsule filter under aseptic conditions.

Step 3: The filtered solution was filled aseptically into 3-mL sterile glass vials, stoppered using sterile Teflon faced rubber stoppers and crimp sealed with aluminum seals to obtain sterile Diluent.

The Diluent can be used in the following reconstitution procedures.

The lyophilized test composition (CHIR99021, NaVPA and poloxamer):

Diluent 1, 0.85 mL+lyophilized cake. Using a syringe, add 0.85 mL to the lyophilized cake of the test composition, and rest in a fridge or ice bath (2-8° C.) for 20 minutes or until clear solution is formed. Gentle tapping on the exterior of the vessel to help the cake dissolve in the Diluent may be required while keeping it in fridge (or ice bath). To avoid degradation of the composition, minimize stirring and/or vortexing.

Placebo composition:

Diluent 1, 0.95 mL+Placebo cake: Using a syringe, add the Diluent to the Placebo cake, and rest in a fridge or ice bath (2-8° C.) for 60 minutes or until clear solution is formed. Gentle tapping on the exterior of the vessel to help the cake dissolve in the Diluent may be required while keeping it in fridge (or ice bath). To avoid degradation of the composition, minimize stirring and/or vortexing.

### Example 13: Stability of Non-Lyophilized Compositions Versus Stability of Lyophilized Compositions

The “wet” test composition (i.e. non-lyophilized—see Example 10, steps 1-6) has poor stability when stored under refrigerated conditions (2-8° C.). In contrast, the lyophilized composition (see Example 10, steps 1-7) has been tested for stability for 6 months at refrigerated conditions and for 2 years at −20° C. storage, and remains stable. (Note: The “wet” test composition will freeze at −20° C. which negatively impacts the test composition and so the stability of the frozen composition was not studied).

Stability of the non-lyophilized test composition (using a freshly compounded solution, lot NBK29-75)

The test composition was compounded fresh (see steps 1-6 of Example 10), stored in the refrigerator and tested at time intervals of 0, 5, 24, 48, 54, 120 hours for NaVPA and CHIR99021 content and appearance. See FIGS. 10 and 11 for the results.

As seen from FIGS. 10 and 11, the assay level of CHIR99021 decreased from its initial level over time, while the assay level of NaVPA remained constant over this time. The solution of the test composition developed precipitate and solution turned hazy somewhere between 24 and 48 hours and then developed precipitate after 120 hours of solution storage under refrigerated conditions. Thus, the “wet” test composition CHIR99021 and NaVPA is not stable during storage.

Stability Data for the Reconstituted Composition

The lyophilized test composition was reconstituted with 0.85 mL of Diluent under refrigeration for approximately 30 minutes. The reconstituted solution was stored in the refrigerator and tested at time intervals of 0, 1, 2, 6, 8, and 24 hours for NaVPA and CHIR99021 drug content and appearance.

NaVPA and CHIR99021 assay levels within the reconstituted test composition were stored inside polypropylene syringes and kept refrigerated remained stable with initial levels for 6 hours as shown in FIG. 12. Sometime between 8 and 24 hours, the assay level of CHIR99021 decreased 37% from its initial level, while the assay level of NaVPA remained constant over this time.

Stability Data for the Lyophilized Test Composition (See Example 10, Steps 1-7).

The lyophilized test composition was studied for storage stability. The lyophilized composition was stored in a glass container with rubber closure and crimp seal under refrigeration for 6 months and at −20° C. for 24 months. At each time interval as noted in Table 27 below, the lyophilized test composition was tested for drug content. It is seen that the lyophilized test composition remains stable (without signs of decomposition) for at least 6 months under refrigeration and 24 months at −20° C. as shown below.

TABLE 27
the lyophilized composition stability data for GMP lot B17030018.

| Time point | | | |
|---|---|---|---|
| (Month) | Storage Condition | Assay NaVPA | Assay CHIR99021 |
| 0 | Release | 100% | 103% |
| 1 | Refrigerated | 100% | 105% |
| 2 | Refrigerated | 100% | 105% |
| 3 | Refrigerated | 100% | 104% |
| 6 | Refrigerated | 99% | 102% |
| 1 | −20 | 101% | 107% |
| 2 | −20 | 100% | 107% |
| 3 | −20 | 100% | 104% |
| 6 | −20 | 100% | 103% |
| 9 | −20 | 99% | 102% |
| 12 | −20 | 99% | 99% |
| 18 | −20 | 101% | 100% |
| 24 | −20 | 98% | 100% |

### Example 14: Reduced Reconstitution Time of Lyophilized Polaxamer

A Lyophilized test composition was prepared according to Examples 2 and 3. Lyophilized P407 was prepared according to Examples 2 and 3; however, the steps of adding NaVPA and CHIR99021 were omitted. Powder P407 (i.e. non-lyophilized) can be obtained from BASF.

A): Comparison of Reconstitution Time of the Lyophilized Test Composition Vs. Reconstitution Time of Lyophilized Poloxamer Vs. Dissolution Time of Non-Lyophilized Poloxamer Powder.

Reconstitution of lyophilized P407 (Placebo) and powder P407 was done using 850 μL Diluent (see above for example preparations of a Diluent) in a vial. The samples had an effective concentration of 16.1% P407 and 5.4% DMSO.

Table E shows the reconstitution time (or dissolution time in the case of powdered P407) using 850 μL of Diluent

TABLE E

| P407 added as | Reconstitution time using 850 μL of Diluent | |
|---|---|---|
| Lyophilized test | 20 | minutes |
| composition (Ex 2 and 3) | | |
| Lyophilized P407 | 1 | hour |
| Powder P407 | 1 | hour |

The lyophilized test composition reconstitutes within 20 minutes of the addition of Diluent. Reconstitution time of Lyophilized P407 or dissolution time of P407 Powder in equivalent amount of diluent was found to be 1 hour. Without wishing to be bound by any particular theory, it is believed that salts of organic acids reduce the reconstitution time of lyophilized poloxamer.

### Details B): The Effect of Organic Acid Salts (Such as Sodium Valproate) and Other Salts on the Reconstitution Time of Lyophilized Poloxamer.

The test composition has a poloxamer (P407) concentration of 16.15% w/v and a sodium valproate concentration of 533.25 mM. Therefore, to probe the effect of salts other than sodium valproate on reconstitution time, a poloxamer solution at 16.15% concentration either alone or including a salt (e.g. an inorganic salt or organic acid salt) at a concentration of 533.25 mM, was lyophilized. The lyophilized material was then reconstituted using Diluent for reconstitution (6.4% DMSO by wt. % in water) or with water. The results are displayed in Table 28.

TABLE 28

| | Salt added (g) for 15 | | Reconstitution time (minutes) | |
|---|---|---|---|---|
| Salt added | ml of solution | mM Concentration | with water | water + DMSO |
| 16.15% w/v P407 (no salt) | Control | | 102 | 90 |
| The test composition (i.e. NaVPA) | Control | 533.25 | 20 | 20 |
| Sodium Valproate | 1.329 g | 533.25 | 20 | 25 |
| Sodium 2-(prop-2-yn-l-yl) Octanoate | 1.634 g | 533.25 | 4 | 4 |
| Magnesium Valproate | 2.485 g | 533.25 | 14 | 14 |
| Sodium Chloride | 0.468 g | 533.25 | 150 | 150 |
| Potassium Phosphate | 1.392 g | 533.25 | undissolved at end of 180 | |
| Calcium Chloride | 0.888 g | 533.25 | plugs remain at end of 180 | |
| Magnesium Chloride | 0.762 g | 533.25 | 180 | 180 |
| Potassium Chloride | 0.597 g | 533.25 | 180 | 168 |

| | | | |
|---|---|---|---|
| Sodium Bicarbonate | 0.670 g | 533.25 | undissolved at end of 180 |

The reconstitution time of lyophilized poloxamer is largely improved due to the addition of organic acid salts such as Sodium valproate, Magnesium divalproate (magnesium valproate), Sodium 2-(prop-2-yn-1-yl) octanoate (also referred to as the sodium salt of 2-hexyl-4-pentynoic acid) and is found to be 420 min. It can be seen that the organic acid salts such as sodium valproate (NaVPA), Magnesium divalproate, and Sodium 2-(prop-2-yn-1-yl) octanoate had the greatest effect on improving the reconstitution time of lyophilized poloxamer. The order of rate of reconstitution of lyophilized poloxamer is approximately in the order of (Poloxamer+Organic acid salts) >Poloxamer alone>(Poloxamer+Inorganic acid salts).

### Example 15A: Purification of Poloxamer 407 (P407)

Purification of Poloxamer 407 refers to the removal of residual smaller chains of polymer, such as monomers and dimers, therefore providing more consistent gelation properties. P407 was purified accordance with a published procedure: A. Fakhari, M Corcoran, A Schwarz, Thermogelling Properties of Purified Poloxamer 407, Heliyon (2017), 3(8), e00390. Unless otherwise stated, unpurified P407 means that no purification methods have been performed. In contrast, purified means that some form of purification method has been performed. In most cases, unpurified P407 is material obtained directly from the supplier and used without any further manipulation.

Poloxamer 407 was characterized using two HPLC methods: RPLC-CAD (reverse phase liquid chromatography with a corona charged aerosol detector) and SEC-CAD (size exclusion chromatography with the same corona CAD detector). Poloxamer 407 does not have a chromophore and so it is undetectable by traditional ultraviolet (UV) detectors. The CAD detector is a mass sensitive detector that works in a manner similar to a mass spectrometer detector.

Size Exclusion Chromatography (SEC)

Size exclusion chromatography (SEC) was used to characterize Poloxamer 407 by separating components based on molecular size (hydrodynamic radius) followed by detection with a CAD. As such, larger molecules elute before smaller molecules. P407 may contain residual block and diblock polymer impurities which are separated and detected by this method. The SEC method for Poloxamer 407 characterization is detailed in Table 29. The peak area percent determined with this method is representative of mass percent. All Poloxamer 407 lots tested show a bimodal distribution with a desired MW peak (triblock copolymer) and a low MW peak (thought to be PEO block and PEO-PPO diblock impurities) as shown in FIGS. 13-15. Some batches of P407 also show a HMW shoulder eluting before the main peak.

The analytical conditions for both HPLC methods are below.

TABLE 29
SEC-CAD Method for P407 characterization

| Parameter | Value |
|---|---|
| Column | Yarra SEC-2000, 3 mm, 145A pore size, |
| | 4.6 × 300 mm with guard column |
| Column Temperature | 30° C. |
| Detection with Corona CAD | 35° C., Power function = 1.70, |
| | Data = 10/sec |
| Flow Rate | 0.4 mL/min |
| Mobile Phase A | 90:10:0.05 ACN:H2O:Formic Acid |
| Isocratic | 100% A for 15 minutes |
| Total Run Time | 15.00 minutes |
| Injection Volume | 5 mL |

Within a batch of Poloxamer 407 there are both higher (HMW) and lower molecular weight (LMW) impurities (see FIGS. 13-15). The analyses have shown that within a batch of P407 that there are more LMW impurities than HMW impurities (see FIGS. 13-15). A six point molecular weight calibration curve was generated using polyethylene glycol standards ranging from 1,450 Da to 35,000 Da—see FIG. 16. The PEG standards are Polyethylene glycol EasiVials (2 mL), Agilent, part number PL 2070-0201. HMW fraction corresponds to molecular weights >17,350 Da relative to PEG standards. Desired MW fraction corresponds to components eluting between 7,250 and 17,350 Da relative to PEG standards. LMW fraction corresponds to molecular weights <7,250 Da relative to PEG standards.

SEC-CAD analysis for Poloxamer 407 lots are summarized in the following Table (peak area % is representative of mass percent). The purification process employed shows removal of HMW impurities and reduction in the amount of LMW impurities, which results in a higher percentage of the desired MW species in the purified lot

TABLE 30
Comparison of impurities in purified poloxamer
and commercially available product.

| | HMW | Desired | LMW |
|---|---|---|---|
| Description | Impurities | MW | Impurities |
| Purified Poloxamer 013-180 | 0.0% | 91.8% | 8.2% |
| (Starting lot GNA17521C) | | | |
| BASF 50424592, lot GNA17521C | 0.0% | 79.6% | 20.4% |
| BASF 50424592, lot GNA17822C | 0.2% | 81.3% | 18.5% |
| Spectrum P1166, lot 2EJ0206 | 0.3% | 76.9% | 22.8% |
| BASF 50259531, lot WPYJ595B | 0.1% | 76.1% | 23.8% |

Mp (Peak molecular weight), Mn (Number-average molecular weight), Mw (Weight-average molecular weight), and PDI (Polydispersity Index, Pd=Mw/Mn) were calculated relative to the PEG calibration standards for the purified and unpurified P407 lots. Lot 013-180 is poloxamer purified by liquid-liquid extraction technique.

TABLE XBa
Mn, Mw and PDI for unpurified and purified P407

| | Mn (Number | Mw (Weight | PDI |
|---|---|---|---|
| | average molecular | average molecular | (polydispersity |
| Sample | weight, Da)* | weight, Da)* | index) |
| BASF 50424592, | 10638 | 11561 | 1.09 |
| lot GNA17822C | | | |
| In house Purified | 11277 | 11787 | 1.05 |
| lot 013-180 | | | |
| In house Purified | 11170 | 11801 | 1.06 |
| lot NBK29-81 | | | |

*Molecular weight relative to PEG Standards

TABLE XBb
Mp for unpurified and purified P407

| | Mp peak 1 (Molecular | Mp peak 2 (Molecular |
|---|---|---|
| | weight, peak maxima for | weight, peak maxima for |
| Sample | desired MW peak, Da)* | LMW peak, Da)* |
| BASF 50424592, | 12299 | 5010 |
| lot GNA17822C | | |
| In house Purified | 12292 | 5508 |
| lot 013-180 | | |
| In house Purified | 12290 | 5299 |
| lot NBK29-81 | | |

*Molecular weight relative to PEG Standards

Cumulative molecular weight distributions for unpurified and purified Poloxamer 407 is shown in FIG. 17. Purification results in decreased LMW impurities. It can be seen that the purified Poloxamer 407 has about 10% less LMW impurities by mass.

Reverse-Phase HPLC (RP-HPLC)

Reverse phase HPLC (RP-HPLC) is used to characterize Poloxamer 407 (P407) purity by separating components based on hydrophobicity followed by detection with a Corona Charged Aerosol Detector (CAD). The RP-HPLC method detailed in the Table below was employed to characterize the hydrophobic character of P407, including low molecular weight (LMW) impurities.

TABLE 31
RP-HPLC-CAD Method for P407 characterization

| Parameter | Value | | | |
|---|---|---|---|---|
| Column | Phenomenex Hydro-RP C18, 3.5 mm, | | | |
| | 4.6 × 50 mm, with guard column | | | |
| Column Temperature | 40° C. | | | |
| Detection with Corona CAD | 35° C., Power function = 1.50, | | | |
| | Data = 10/sec | | | |
| Flow Rate | 1.0 mL/min | | | |
| Mobile Phase A | Deionized H2O | | | |
| Mobile Phase B | Acetonitrile (LC-MS grade) | | | |
| Mobile Phase C | 2-propanol (LC-MS grade) | | | |
| Gradient Condition | Time | %A | %B | %C |
| | 0.00 | 90 | 10 | 0 |
| | 3.00 | 75 | 25 | 0 |
| | 25.00 | 37.5 | 12.5 | 50 |
| | 33.00 | 7.5 | 2.5 | 90 |
| | 34.00 | 90 | 10 | 0 |
| | 37.00 | 90 | 10 | 0 |

Inverse gradient used to make the solvent composition applied
to the detector consistent throughout the gradient run

| | |
|---|---|
| Total Run Time | 37.00 minutes |
| Injection Volume | 10 mL |

See FIG. 18 for a chromatogram produced by RP-HPLC. Impurities are divided into “zones” in the chromatogram (FIG. 19). Based on experimental results obtained during method development, it is hypothesized that Zones 1 and 2 correspond to LMW block PEO, Zones 3 and 4 correspond to diblock PEO-PPO, and Zone 5 corresponds to the desired triblock PEO-PPO-PEO. The peak area percent is used as a relative benchmark for lot-to-lot comparison or for monitoring purification.

Earlier eluting zones are expected to be more hydrophilic, while later eluting zones are expected to be more hydrophobic. Additionally, lower molecular weight polymers will have shorter retention times than higher molecular weight polymers of the same composition. The peak area percent for several lots of Poloxamer 407 is shown in the Table below. Lot 013-180 is poloxamer purified by liquid-liquid extraction technique, while all other lots listed in the table were unmodified and analyzed as received from the supplier.

TABLE 32
RP-HPLC Peak Area Percent by Zone
for Different Lots of Poloxamer 407

| | Unpurified P407 | Purified P407 (013-180) |
|---|---|---|
| Zone | (Lot GNA17521C) | (Starting lot GNA17521C) |
| 1 | 0.0% | 0.0% |
| 2 | 2.1% | 0.0% |
| 3 | 4.8% | 0.3% |
| 4 | 6.7% | 3.4% |
| 5 (Desired P407) | 86.4% | 96.2% |
| Total | 100.0 | 99.9 |

It can be seen that purification of P407 is effective in removing the LMW impurities.

### Example 15B: Poloxamer 407 (P407) Purification Analysis

Three lots of purified Poloxamer 407 were produced and analyzed. The lots were purified following the same liquid-liquid extraction method (A. Fakhari, M Corcoran, A Schwarz, Thermogelling Properties of Purified Poloxamer 407, Heliyon (2017), 3(8), e00390). The lots were analyzed by SEC (as described above). The three lots are: Lot 013-180 (from BASF lot GNA17521C); Lot NBK29-81 (from BASF lot GNA17822C); and Lot NBK29-81A (from BASF lot GNA17822C).

TABLE
SEC Characterization of Poloxamer 407 lots

| | HMW | | LMW |
|---|---|---|---|
| Description | Impurities | Desired MW | Impurities |
| BASF 50424592, lot GNA17822C | 0.6% | 79.4% | 19.9% |
| BASF 50424592, lot GNA17521C | 0.1% | 77.6% | 22.3% |
| BASF 50424592, lot GNC22321B | 0.0% | 80.4% | 19.6% |
| BASF 50259531, lot WPYJ595B | 0.0% | 74.9% | 25.1% |
| Spectrum P1166, lot 2EJ0206 | 0.2% | 74.0% | 25.8% |
| Spectrum P1166, lot 2GD0159 | 0.3% | 76.0% | 23.7% |
| Purified P407 lot 013-180 | 0.1% | 90.2% | 9.7% |
| Purified P407 lot NBK29-81 | 0.7% | 87.2% | 12.1% |
| Purified P407 lot NBK29-81A | 0.6% | 87.4% | 12.0% |

Peak area percent (expected to be approximately equivalent to mass percent). Molecular weight relative to PEO standards. Purification results in increased Mn and Mw for the entire polymer distribution and overall decrease in PDI. The desired MW fraction changes by about 10% by weight with purification. The LMW fraction shows slight increase in Mp, Mn, Mw and a decrease in PDI indicating that within the lower molecular weight fraction the smaller molecules are being preferentially removed.

Table G (below) shows Mn, Mw, Mp and PDI for the entire P407 polymer, desired MW fraction, and the LMWLWM fraction.

TABLE G
Comparison of Poloxamer Polymer Distributions

| | Entire Polymer Distribution | | | Desired MW Fraction | | | | LMW Fraction | | | |
|---|---|---|---|---|---|---|---|---|---|---|---|
| Sample | Mn (Da) | Mw (Da) | PDI | Mp (Da) | Mn (Da) | Mw (Da) | PDI | Mp (Da) | Mn(Da) | Mw (Da) | PDI |
| BASF 50424592, lot GNA17822C | 10441 | 11395 | 1.09 | 12156 | 11755 | 11976 | 1.02 | 4992 | 4916 | 5067 | 1.03 |
| BASF 50424592, lot GNA17521C | 10095 | 11110 | 1.10 | 11939 | 11606 | 11818 | 1.02 | 4902 | 4755 | 4956 | 1.04 |
| BASF 50424592, lot GNC22321B | 10164 | 11041 | 1.09 | 11867 | 11467 | 11664 | 1.02 | 4788 | 4829 | 4981 | 1.03 |
| BASF 50259531, lot WPYJ595B | 9786 | 10837 | 1.11 | 11997 | 11431 | 11657 | 1.02 | 5053 | 4834 | 5006 | 1.04 |
| Spectrum P1166, lot 2EJ0206 | 9539 | 10625 | 1.11 | 11719 | 11179 | 11395 | 1.02 | 4863 | 4675 | 4897 | 1.05 |
| Spectrum P1166, lot 2GD0159 | 9753 | 10765 | 1.10 | 11516 | 11207 | 11415 | 1.02 | 4863 | 4730 | 4928 | 1.04 |
| Purified P407 lot 013-180 | 11063 | 11571 | 1.05 | 12081 | 11652 | 11855 | 1.02 | 5383 | 5342 | 5440 | 1.02 |
| Purified P407 lot NBK29-81 | 10985 | 11629 | 1.06 | 12106 | 11727 | 11937 | 1.02 | 5265 | 5217 | 5322 | 1.02 |
| Purified P407 lot NBK29-81A | 10967 | 11600 | 1.06 | 12130 | 11711 | 11925 | 1.02 | 5261 | 5241 | 5340 | 1.02 |
| P188 WPYJ540B | 6723 | 6965 | 1.04 | 6884 | N/A | N/A | N/A | 3467 | N/A | N/A | N/A |

Desired MW Fraction corresponds to fraction eluting between 6.7 and 7.8 minutes. LMW Fraction refers to portion eluting ≥7.8 minutes, HMW Fraction refers to portion eluting ≤6.7 minutes For this analysis, 6.7 minutes corresponds to 16590 Da and 7.8 minutes corresponds to 6840 Da
Mp (molecular weight at peak, Da)
Mn (Number average molecular weight Da)
Mw (Weight average molecular weight Da)
PDI (polydispersity index)

It will be appreciated that experimental data may inherently vary between different experimental runs because of experimental error. For example, it may be necessary to consider that two data values that are within about +1-3% are the same because they are within an acceptable tolerance. However, this may depend on the nature of the experiment and the instrument used.

### Example 16A: Rheology of Purified and Unpurified P407

The rheology of a Poloxamer 407 solution was analyzed. Table 33: Rheology of 17% (w/w) aqueous stock made with purified poloxamer 407 and unpurified poloxamer 407.

| | Gelation | Viscosity at |
|---|---|---|
| Poloxamer 407 | Temperature (° C.) | 37° C. (mPa-s) |
| Unpurified (i.e. commercially | 22.7 | 3.55 × 10⁶ |
| available P407) | | |
| Purified | 21.4 | 4.77 × 10⁶ |

Rheology was performed using Ki nexus lab+rheometer by Malvern, Model KNX2110. SN #MAL1147460. Kinexus is a rotational rheometer system that applies controlled shear deformation to a sample under test, to enable measurement of flow properties (such as shear viscosity from flow tests), and dynamic material properties (such as viscoelastic modulus and phase angle from oscillation tests). For the determination of the sol/gel-transition temperature, the instrument was run in oscillatory mode and a temperature sweep performed in the range from 5-45° C. at a frequency of 0.75 Hz and a heating rate of 3° C./min. The parameters used to characterize the sol/gel transition are the elastic modulus G′ and the viscous modulus G″. The sol/gel transition (or gelation temperature) is regarded as the point of intersection of the G′ and G″ curves. Sol-to-gel transition temperature and complex viscosity measurements were conducted using a roughened spindle-plate geometry (radius 40 mm), a 640 ul sample volume, and a 20 s−1 shear rate. Viscosity was measured by a roughened spindle-plate geometry (radius 40 mm), a 640 ul sample volume, and a 20 s−1 shear rate using a Ki nexus lab+rheometer by Malvern, Model KNX2110. SN #MAL1147460. Viscosity was measured using a Ki nexus lab+rheometer by Malvern, Model KNX2110. SN #MAL1147460.

While it can be seen that a solution of purified poloxamer 407 has a similar gelation temperature in comparison to a solution of unpurified poloxamer, the purified poloxamer has a higher viscosity in comparison to a solution of unpurified poloxamer.

Effects of Using Purified Poloxamer 407 in the Composition

TABLE 34
Rheology of compositions with purified and unpurified P407

| | | | | Composition | |
|---|---|---|---|---|---|
| Poloxamer 407 | | | | Gelation | Composition |
| (P407) | NaVPA | CHIR99021 | DMSO | temperature | Viscosity at 37° C. |
| 15.5% w/v | 83 mg/ml | 3.1 mg/mL | 5% w/v | 24.55° C. | 1.536 × 10⁶ mPa · s |
| Unpurified P407 | | | | | |
| 15.5% w/v | 133 mg/mL | 5.1 mg/mL | 5% w/v | 28.95° C. | 3.17 × 10⁶ mPa · s |
| Purified P407 | | | | | |

Rheology was performed using Kinexus lab+rheometer by Malverm, Model KNX2110. SN #MAL1147460, as described above.

The test composition made with unpurified P407 can accommodate ˜80 mg/mL of NaVPA while maintaining characteristics suitable for use. In this sense, the composition must be able to form a viscous gel composition, where viscous means formation of immobile gel when heated to temperature of 37° C. (body temperature) so it can be administered via injection in the ear. The addition of components to a solution of P407, such as salts and/or actives, will affect the gelation temperature and the viscosity, thus affecting whether a composition remains suitable for use.

Surprisingly, it can be seen that a test composition made with purified P407 can accommodate a large increase in the amount of NaVPA (83 vs. 133 mg/ml) while still being suitable for use. The amount of CHIR99021 can also be adjusted, for example, 3.1 vs. 5.1 mg/mL. However, the amount of NaVPA, or an equivalent species, can be varied independently of the amount of any other active(s), e.g., in this case CHIR99021.

Without wishing to be bound by any particular theory, a composition containing purified poloxamer increases the overall viscosity and decreases the gelation temperature because unfavorable interactions with LMW impurities have been reduced. Indeed, in this example, it can be seen that a composition made with purified poloxamer 407 imparts improved gel viscosity. This allows for higher drug pay loads compared to an otherwise identical composition containing unpurified poloxamer. This is especially true for a drug component, such as sodium valproate, which is ionic in nature and limits the ability of poloxamer to form a gel with enough viscosity at higher amounts (enough viscosity refers to formation of immobile gel when heated to body temperature of 37° C.).

Lyophilized compositions made with purified poloxamer reconstitute in a similar time compared to the otherwise identical test compositions made with unpurified poloxamer.

### Example 16B: NaVPA Concentration in Compositions with Unpurified P407 vs. Purified P407

As discussed in Example 16A, a test composition made from unpurified P407 cannot accommodate high concentrations of NaVPA (e.g. great than 90 mg/mL) and maintain a suitable gelation temperature (e.g. 37° C.). The following data exemplifies this observation.

When using poloxamer as the gelling agent in the compositions of the present disclosure, the maximum concentration of NaVPA that can be accommodated is limited to about 90 mg/mL while a suitable gelation temperature is maintained. At a concentration of NaVPA greater than −90 mg/mL, the resulting composition is compromised for its gelation property. As such the composition does not gel at the desired temperature. For example, a composition containing 130 mg/mL NaVPA and 2.2 mg/mL CHIR99021 in 16.15% unpurified poloxamer 407 and 5% DMSO (sample 008-39A) did not form a gel at 37° C. It will also be appreciated that gelation temperature is also linked to viscosity. In contrast, a composition containing 133.7 mg/mL NaVPA and 5.39 mg/mL CHIR99021 in 15.6% w/v purified poloxamer 407 and 4.9% w/v DMSO (sample NBK29-80-2A) formed a gel at 37° C. (body temperature). Therefore, a composition comprising purified Poloxamer 407 allows for an increased concentration of NaVPA up to about 138 mg/mL while still forming a gel at an acceptable temperature (e.g. body temperature). It can therefore be seen that changing the poloxamer from unpurified poloxamer to purified poloxamer results in the effect of being able to accommodate an increased amount of NaVPA in the composition.

### Example 17: Further Lyophilized Compositions and Reconstitution Thereof

Building on the results of Examples 14 to 16, further lyophilized compositions were manufactured and had their reconstitution times evaluated. The test composition from Table 28 is included in the table below for comparison (entry 1). The further lyophilized compositions were made in accordance with Example 10, and reconstituted in accordance with Example 12.

TABLE 35
Further test compositions and reconstitution times. The compositions in Table 35 took comparable
times (~12 hours) to reach the pre-lyophilization stage (c.f steps 1-6, example 10).

| Entry (Test | | Second otic therapeutic | | |
|---|---|---|---|---|
| composition | First otic therapeutic | agent or other | | Reconstitution |
| reference) | agent (concentration mg/mL) | agent (concentration mg/mL) | Gelling agent (w/v %) | time (minutes) |
| 1 | CHIR99021 (3.1) | NaVPA (83) | Poloxamer 407 (16.5) | 20 |
| 2 (A) | CHIR99021 (5.1) | NaVPA (130) | Purified poloxamer 407 (15.5) | 15 |
| 3 (B) | LY2090314 (0.77) | NaVPA (130) | Purified poloxamer 407 (15.5) | 13 |
| 4 (C) | Compound I-7 (0.8) | NaVPA (130) | Purified poloxamer 407 (15.5) | 17 |
| 5 (D) | GSK3 XXII (0.26) | NaVPA (130) | Purified poloxamer 407 (15.5) | 17 |
| 6 (E) | CHIR99021 (3.14) | Sodium salt of linoleic add | Purified poloxamer 407 (15.5) | 22 |
| | | (112.18) | | |

The compositions exemplified above generally comprise purified poloxamer 407, which allows for an increased concentration of NaVPA of −130 mg/mL. A composition with 130 mg/mL concentration of NaVPA cannot be made when using unpurified poloxamer 407 because the desirable gelling characteristics are not maintained. Hence the test composition (e.g. Example 10) has a concentration of NaVPA of 83 mg/mL. Furthermore, and independently to the purified poloxamer/NaVPA effect, the compositions can feature two different concentrations of an otic therapeutic agent, e.g. when the composition includes CHIR99021, the concentration can be 3.1 mg/mL or about a dose and a half greater, 5.1 mg/mL. Entries 2-5 in Table 35 exemplify the 1.5× dose. Entry 6 in Table 35 exemplifies the 1× dose. Entries 2-6 (test compositions A-E), lyophilized using the lyophilization method of the present disclosure, lyophilized well to give porous and fluffy product cakes. FIG. 20 displays lyophilized test composition A (entry 2, Table 35). FIG. 21 displays lyophilized test composition B (entry 3, Table 35). FIG. 22 displays lyophilized test composition C (entry 4, Table 35). FIG. 23 displays lyophilized test composition D (entry 5, Table 35). FIG. 24 displays lyophilized test composition E (entry 6, Table 35). FIG. 25 shows reconstituted compositions A (A1), B (B-1), C (C-1), D (F-1), E (G-1) from Table 35.

As can be seen from Table 35, entries 2-5 reconstituted in around 20 minutes or less, thereby demonstrating the general methodology that lyophilized compositions comprising poloxamer and valproic acid or a pharmaceutically acceptable salt thereof display improved reconstitution times. Furthermore, it can be seen from Table 28 and 35, that the valproic acid component can be substituted (e.g. sodium 2-(prop-2-yn-1-yl) octanoate), and that the effect of improved reconstitution time is still observed. Indeed, entry 6 of Table 35, shows that a lyophilized composition comprising linoleic acid or a pharmaceutically acceptable salt thereof displays improved reconstitution time.

Based on the data from this Example and Examples 14-16, the following hypothetical compositions fall within the present disclosure. (Some of these companions have been exemplified above.)

| Entry (Test | | Second otic therapeutic | |
|---|---|---|---|
| composition | First otic therapeutic | agent or other | |
| number) | agent (concentration mg/mL) | agent (concentration mg/mL) | Gelling agent (15-17 w/v %) |
| 1 | CHIR99021 (3.1) | NaVPA (83) | Unpurified poloxamer 407 |
| 2 | CHIR99021 (3.1) | NaVPA (83) | Purified poloxamer 407 |
| 3 | CHIR99021 (5.1) | NaVPA (83) | Purified poloxamer 407 |
| 4 | CHIR99021 (5.1) | NaVPA (130) | Purified poloxamer 407 |
| 5 | LY2090314 (0.51) | NaVPA (83) | Unpurified poloxamer 407 |
| 6 | LY2090314 (0.51) | NaVPA (83) | Purified poloxamer 407 |
| 7 | LY2090314 (0.77) | NaVPA (83) | Purified poloxamer 407 |
| 8 | LY2090314 (0.77) | NaVPA (130) | Purified poloxamer 407 |
| 9 | Compound I-7 (0.55) | NaVPA (83) | Unpurified poloxamer 407 |
| 10 | Compound I-7 (0.55) | NaVPA (83) | Purified poloxamer 407 |
| 11 | Compound I-7 (0.77) | NaVPA (83) | Purified poloxamer 407 |
| 12 | Compound I-7 (0.77) | NaVPA (130) | Purified poloxamer 407 |
| 13 | GSK3 XXII (0.17) | NaVPA (83) | Unpurified poloxamer 407 |
| 14 | GSK3 XXII (0.17) | NaVPA (83) | Purified poloxamer 407 |
| 15 | GSK3 XXII (0.26) | NaVPA (83) | Purified poloxamer 407 |
| 16 | GSK3 XXII (0.26) | NaVPA (130) | Purified poloxamer 407 |
| 17 | CHIR99021 (3.14) | Sodium salt of linoleic acid (112.18) | Unpurified poloxamer 407 |
| 18 | CHIR99021 (3.14) | Sodium salt of linoleic acid (112.18) | Purified poloxamer 407 |
| 19 | CHIR99021 (5.1) | Sodium salt of linoleic acid (112.18) | Purified poloxamer 407 |
| 20 | CHIR99021 (5.1) | Sodium salt of linoleic acid (168.3) | Purified poloxamer 407 |
| 21 | CHIR99021 (3.14) | Sodium 2-(prop-2-yn-1-yl) octanoate (102) | Unpurified poloxamer 407 |
| 22 | CHIR99021 (3.14) | Sodium 2-(prop-2-yn-1-yl) octanoate (102) | Purified poloxamer 407 |
| 23 | CHIR99021 (5.1) | Sodium 2-(prop-2-yn-1-yl) octanoate (102) | Purified poloxamer 407 |
| 24 | CHIR99021 (5.1) | Sodium 2-(prop-2-yn-1-yl) octanoate (163) | Purified poloxamer 407 |

It will further be appreciated that a composition can be made with any one of CHIR99021, LY2090314, GSK3 XXII, or Compound I-7; and with any of one NaVPA, Sodium 2-(prop-2-yn-1-yl) octanoate or Sodium salt of linoleic acid; with purified or unpurified Poloxamer 407.

### Example 18: Lyophilized Composition Percentages

Some lyophilized compositions of the present disclosure may have approximately the following percentages of components. For example, the test composition referred to in entry 1 of Table 35 may have about 165.24 mg Poloxamer 407 (about 64.25%), about 88.63 mg NaVPA (about 34.5%) and about 3.3 mg CHIR99021 (about 1.25%). For example, the composition referred to in entry 2 of Table 35 may have about 174.96 mg purified Poloxamer 407 (about 53%), about 150 mg NaVPA (about 45.25%) and about 6.05 mg CHIR99021 (about 1.75%) It will be appreciated that these values may vary by about 10%, and the lyophilized compositions are substantially free from water and/or DMSO.

### Example 19: Determination of Lan Molecular Weight Aldehydes in the Test Composition and Placebo

Sample Preparation

Samples were diluted, processed with SPE (solid phase extraction) to isolate the aldehydes, derivatized with DNPH (2,4-dinitrophenylhydrazine), and quantified using HPLC-UV.

Sample Reconstitution and Dilution

The lyophilized composition samples were reconstituted with 0.85 mL Diluent 1 (6.4% w/w DMSO in H₂O) for at least 30 minutes at 4° C. Placebo lyophilized samples were reconstituted with 0.95 mL Diluent 1 (6.4% w/w DMSO in H₂O) for at least 1 hour at 4° C. After reconstitution was complete, 0.75 g of sample was weighed into a 7 mL glass scintillation vial. 1.5 mL of the dilution buffer was added to the sample and the resulting solution was vortexed until fully dissolved. The total volume of this solution is approximately 2.25 mL. Diluent 1 (6.4% w/w DMSO in H₂O) was tested for aldehyde content without further dilution

Preparation of Poloxamer 407 Samples

Poloxamer 407 was prepared as a 60 mg/mL solution by dissolving 300 mg of Poloxamer 407 into 4.7 mL DI H₂O to create a total solution volume of approximately 5.0 mL. Solutions were stirred until completely dissolved and analysed within 8 hours of preparation.

Solid Phase Extraction

Solid Phase Extraction (SPE) was used to separate aldehydes from sample matrix prior to derivatization. C18 SPE cartridges (Agilent Bond Elut 12102028, 500 mg bed, 3 mL) were used to retain Poloxamer 407 matrix components while eluting aldehydes. SPE cartridges were attached to a 12 position vacuum manifold (Agilent, 5982-9110) and flow was modulated to be approximately 1 to 2 mL/min. SPE cartridges were conditioned with MeOH (Fisher, HPLC grade), washed with ACN (Honeywell, carbonyl-free), and equilibrated with the dilution buffer (90% 10 mM acetate buffer, 10% ACN) as described in Table 36. Excess solvent was removed from the cartridge and a 4 mL amber vial was placed under each cartridge. 0.5 mL of the diluted sample prepared in Section 6.4.1 was added to cartridge. Aldehydes were eluted using two rounds of 1.25 mL of the dilution buffer. All liquid was removed from the cartridge and the total collected volume was 3.0 mL. The SPE procedure is detailed in Table 36.

TABLE 36
Solid Phase Extraction Procedure

| SPE Elution Step | Solvent | Collection |
|---|---|---|
| 1-Condition | 2.4 mL MeOH, repeat 2 times | Discard |
| 2-Wash | 2.4 mL ACN, repeat 2 times | Discard |
| 3-Equilibration | 2 mL 90:10 (acetate buffer:ACN), repeat 2 times | Discard |
| 4-Sample Loading | 0.5 mL sample | Collect in 4 mL |
| and Elution | 1.25 mL 90:10 (acetate buffer:ACN) | vial |
| | 1.25 mL 90:10 (acetate buffer:ACN) | |

For select experiments, elution was performed with an 85:15 elution buffer (85% 10 mM acetate buffer, 15% ACN) as detailed in Table 37.

TABLE 37
Solid Phase Extraction Procedure with 85:15 Elution Buffer

| SPE Elution Step | Solvent | Collection |
|---|---|---|
| 1-Condition | 2.4 mL MeOH, repeat 2 times | Discard |
| 2-Wash | 2.4 mL ACN, repeat 2 times | Discard |
| 3-Equilibration | 2 mL 85:15 (acetate buffer:ACN), repeat 2 times | Discard |
| 4-Sample Loading | 0.5 mL sample | Collect in 4 mL |
| and Elution | 1.25 mL 85:15 (acetate buffer:ACN) | vial |
| | 1.25 mL 85:15 (acetate buffer:ACN) | |

Derivtization with DNPH

The DNPH derivatization solution was created by combining 98.5 mL carbonyl-free ACN (acetonitrile), 1 mL phosphoric acid (85% w/w, EMD Millipore), and 0.5 mL DNPH solution (70% DNPH in 30% H2O, Spectrum D1149). To the 3.0 mL of collected eluate from the “Solid Phase Extraction” Section above, 1.5 mL of the DNPH derivatization solution was added. Solutions were vortexed for 5 seconds and derivatization reaction proceeded at room temperature for at least 30 minutes. Derivatized aldehydes can be quantified by HPLC-UV.

Calculations Used in Determining Aldehyde Content

Aldehyde content in μg/g, i.e. weight of aldehydes per weight of reconstituted composition, Test composition or Placebo, was calculated using the equation below.

$$Aldehyde Content ( \frac{µg}{g} ) = Conc ( \frac{µg}{mL} ) \times \frac{Dilution Volume}{Sample Weight} \times \frac{0.5 mL}{3.0 mL}$$

Where “Conc (μg/mL)” is the solution concentration for a given aldehyde determined by HPLC-UV. Sample weight refers to the weight recorded in the “Sample Reconstitution and Dilution” Section above, which is approximately 0.75 g for the composition. Dilution volume refers to the total volume into which the sample is diluted prior to SPE as described in the “Sample Reconstitution and Dilution” Section above; for the composition samples, the total volume is 2.25 mL. 0.5 mL/3.0 mL represents the dilution factor from SPE, where 0.5 mL of sample is loaded onto the cartridge and eluted with 2.5 mL resulting in a total collected volume of 3.0 mL. Aldehyde content in μM was calculated using the equation below.

$$Aldehyde Content ( µM ) = \frac{Aldehyde Content ( \frac{µg}{g} ) \times Density ( \frac{g}{mL} ) \times \frac{1000 mL}{1 L}}{Molecular Weight ( \frac{µg}{µmol} )}$$

Where density refers to the density of the original sample solution. Density of the composition, Placebo, and Diluent 1 were determined to be respectively 1.04, 1.03, and 1.01 g/mL. Molecular weight refers to the molecular weight of each aldehyde with formaldehyde, acetaldehyde, and propionaldehyde having molecular weights of 30.03, 44.05, and 58.08 μg/μmol respectively.

Aldehydes present in a clinical dose of 200 μL of reconstituted composition were calculated using the equations below.

$$Aldehyde in clinical dose ( µmol ) = Aldehyde Content ( µM ) \times 0.2 mL \times \frac{1 L}{1000 mL}$$

$$Aldehyde in clinical dose ( µg ) = Aldehyde in clinical dose ( µmol ) \times Molecular Weight ( \frac{µg}{µmol} )$$

Determination of Low Molecular Weight Aldehydes in the Test Composition and Placebo

In one aspect, the composition (comprising NaVPA, CHIR99021 and Poloxamer 407) is pharmaceutical composition for use in a method of treating chronic noise-induced hearing loss (CNIHL). Poloxamer 407 is used as an excipient in the test composition for its ability to form a thermo-reversible gel. Aldehydes including formaldehyde, acetaldehyde, and propionaldehyde are potential impurities and degradation products of Poloxamer 407.

Formaldehyde (FA), acetaldehyde (AA), and propionaldehyde (PA) levels were determined for GMP and GLP Test compositions, Placebo, and Diluent retains using the optimized conditions for solid phase extraction sample preparation and DNPH derivatization. Aldehyde content was quantified in four lots of commercially available Poloxamer 407. Aldehyde levels in accelerated and nonaccelerated liquid placebo were compared before and after lyophilization

Aldehyde Determination in Test Composition and Placebo

FA, AA, and PA content was determined for the test composition and Placebo. 1 vial of each lot of the Test composition and Placebo was reconstituted (i.e. the test composition/placebo is stored in the lyophilized form) and tested for aldehydes following the protocol in the “Sample Preparation” Section above. For Test composition Lot 300006, 3 vials were reconstituted and tested for aldehydes. Diluent 1 was directly tested without further dilution as described in the “Sample Reconstitution and Dilution” Section above. Two replicates were analyzed for each vial prepared and results are shown in Table 38, Table 39, and Table 40.

TABLE 38
Aldehyde Content in the Test composition

| | # of | | FA Content | AA Content | PA Content |
|---|---|---|---|---|---|
| Lot # | Samples | Storage Condition | (ppm = μg/g) | (ppm = μg/g) | (ppm = μg/g) |
| Lot B1730018 | 2 | 6 months at 5° C. 60\hat{ } RH, | <18* | 0.39^{□} ± 0.02 | <0.18* |
| | | 7 months at −20° C. | | | |
| Lot B17010006 | 2 | 15 months at 5° C. | <18* | 0.33^{□} ± 0.01 | <0.18* |
| FT-021-086 | 2 | 5 months at −20° C. | <18* | <18* | 0.18^{□} ± 0.00 |
| Lot 300006 | 6 | 1 year at 5° C., | <18* | 0.23^{□} ± 0.01 | <18* |
| | | 1 month at −20° C. | | | |
| | 6 | 1 year at 5° C., followed by | <18* | 0.26^{□} ± 0.01 | <18* |
| | | 1 month at 40° C. 75% RH | | | |

*Indicates sample was below the LOD.
^{□}Indicates sample was below LLOQ.

LOD=Limit of detection. LLOQ=lower limit of quantification.

TABLE 39
Aldehyde Content in Placebo

| | # of | | FA Content | AA Content | PA Content |
|---|---|---|---|---|---|
| Lot # | Samples | Storage Condition | (ppm = μg/g) | (ppm = μg/g) | (ppm = μg/g) |
| Lot B17020015 | 2 | 14 months at 5° C. | <18* | 0.37^{□} ± 0.00 | <18* |
| | 2 | 6 months at 25° C. 60% | <18* | 0.62^{□} ± 0.01 | <18* |
| | | RH, 8 months at 5° C. | | | |

*Indicates sample was below the LOD.
^{□}Indicates sample was below LLOQ.

TABLE 40
Aldehyde Content in Diluent 1

| | # of | | FA Content | AA Content | PA Content |
|---|---|---|---|---|---|
| Lot # | Samples | Storage Condition | (ppm = μg/g) | (ppm = μg/g) | (ppm = μg/g) |
| Lot B17020013 | 2 | 14 months at 5° C. | <0.06* | <0.06* | <0.06* |
| | 2 | 6 months at 25° C. 60% | <0.06* | <0.06* | <0.06* |
| | | RH, 8 months at 5° C. | | | |

*Indicates sample was below the LOD.
^{□} Indicates sample was below LLOQ.

Aldehyde content in a 200 μL, clinical dose was calculated following the equation specified in the “Calculations Used in Determining Aldehyde Content” Section above and is detailed for Test composition in Table 9.1-4 and for Placebo in Table 42.

TABLE 41
Aldehyde Content in a Clinical Dose of Test composition

| | # of | | FA in Clinical | AA in Clinical | PA in Clinical |
|---|---|---|---|---|---|
| Lot # | Samples | Storage Condition | Dose (μg) | Dose (μg) | Dose (μg) |
| Lot B17030018 | 2 | 6 months at 5° C. 60% RH, | <0.04* | 0.08^{□} ± 0.00 | <0.04* |
| | | 7 months at −20° C. | | | |
| Lot B17030006 | 2 | 15 months at 5° C. | <0.04* | 0.07^{□} ± 0.00 | <0.04* |
| FT-021-086 | 2 | 5 months at −20° C. | <0.04* | <0.04* | 0.04^{□} ± 0.00 |
| Lot 300006 | 6 | 1 year at 5° C. | <0.04* | 0.05^{□} ± 0.00 | <0.04* |
| | | 1 month at −20° C. | | | |
| | 6 | 1 year at 5° C., followed by | <0.04* | 0.06^{□} ± 0.01 | <0.04* |
| | | 1 month at 40° C. 75% RH | | | |

*Indicates sample was below the LOD.
^{□}Indicates sample was below LLOQ.

TABLE 42
Aldehyde Content in a Clinical Dose of Placebo

| | # of | | FA in Clinical | AA in Clinical | PA in Clinical |
|---|---|---|---|---|---|
| Lot # | Samples | Storage Condition | Dose (μg) | Dose (μg) | Dose (μg) |
| Lot B17020015 | 2 | 15 months at 5° C. | <0.04* | 0.08^{□} ± 0.00 | <0.04* |
| | 2 | 6 months at 25° C. 60%, | <0.04* | 0.13^{□} ± 0.00 | <0.04* |
| | | RH, 5° C. thereafter | | | |

*Indicates sample was below the LOD.
^{□}Indicates sample was below LLOQ.

No formaldehyde was detected at levels above the LOD in the Test composition, Placebo, or Diluent exposed to primary storage conditions for 1 year or accelerated storage conditions for 6 months. Test composition and Placebo stored as lyophilized cakes demonstrate PA levels below the LOD and AA levels below the LLOQ of 0.7 μg/g sample. Diluent 1 contains 6.4% w/w DMSO in H₂O and no Poloxamer 407; as expected no aldehydes were detected in Diluent 1 samples.

Poloxamer 407 when stored as a lyophilized cake shows minimal levels of aldehydes and no significant increase in aldehyde content under accelerated storage conditions. No evidence of higher aldehyde homologs were observed in the RP-HPLC chromatograms.

Aldehyde Determination in Poloxamer 407

Aldehyde content was determined in commercially available Poloxamer 407 following the protocol specified in the “Preparation of Poloxamer 407 Samples” Section above. Formaldehyde, acetaldehyde, and propionaldehyde were quantified by HPLC-UV and the results are tabulated in Table 43.

TABLE 43
Aldehyde Content in Poloxamer 407 (N = 2)

| | Formaldehyde | Acetaldehyde | Propionaldehyde |
|---|---|---|---|
| | Concentration | Concentration | Concentration |
| Poloxamer 407 PN, Lot # | (ppm = μg/g) | (ppm = μg/g) | (ppm = μg/g) |
| BASF 50424592, lot GNA17822C | <1.0* | 18.9 ± 1.7 | 18.2 ± 0.4 |
| BASF 50424592, lot GNA17521C | <1.0* | 25.2 ± 3.5 | 19.0 ± 1.7 |
| BASF 50259531, lot WPYJ595B | <1.0* | 32.8 ± 0.6 | 22.8 ± 0.2 |
| Spectrum P1166, lot 2EJ0206 | <1.0* | 31.5 ± 0.1 | 22.0 ± 0.1 |

*Indicates sample was below the LOD.

Commercially available Poloxamer 407 does not contain any detectable formaldehyde and contains detectable levels of acetaldehyde and propionaldehyde. It is hypothesized that lyophilization of the test composition removes aldehyde impurities present in Poloxamer 407 at the time of manufacture.

Effect of Lyophilization on Aldehyde Content

The effect of lyophilization on aldehyde content was studied for two lots of liquid placebo. The lyophilization process was developed to remove H₂O and DMSO from the Test composition and Placebo formulations. While formaldehyde, acetaldehyde, and propionaldehyde are volatile compounds, it was unclear whether the concentration of these would be reduced during the lyophilization process. The composition and storage condition of the liquid placebos are detailed in Table 44.

TABLE 44
Composition and Storage Conditions for Liquid Placebo

| Sample ID | Composition | Storage Condition |
|---|---|---|
| FT-021-094 | 15.5% w/w P407 lot GNA17822C, | 6 months at 40° C. |
| | 5.0% w/w DMSO in H₂O | 75% RH |
| FT-032-081 | 14.75% w/w P407 lot GNA17822C, | 1 week at 5° C. |
| | 5.0% w/w DMSO in H₂O | |

For FT-021-094, three vials containing 1 mL each were stored under accelerated conditions for 6 months. After 6 months storage, a pre-lyophilization sample was taken by weighing 400 mg from each vial into a 2 mL HPLC vial and was diluted with 800 μl of the 90:10 dilution buffer. The remaining solutions each with approximately 600 μl were lyophilized (e.g. see above).

For FT-032-081, a 3 L batch of liquid placebo was created and stored at 4° C. for 1 week. Three samples were taken pre-lyophilization by weighing 400 mg into a 2 mL HPLC vial and was diluted with 800 μl of the 90:10 dilution buffer. Three vials were prepared with 1 mL of FT-032-081 and lyophilized (e.g. see above).

After lyophilization, Diluent 1 (4.6% w/w DMSO in MO) was added to replace the weight lost during lyophilization and samples were allowed to reconstitute for at least 1 hr at 4° C. 500 mg from each reconstituted vial was weighed into a 2 mL HPLC vial and 1 mL of the 90:10 dilution buffer was added. The pre-lyophilization and post-lyophilization samples were vortexed until fully dissolved and analysed in duplicate for aldehyde content following the method detailed in the “Sample preparation” Section above. Aldehyde content before and after lyophilization is shown in Table 45 and FIG. 26.

TABLE 45
Aldehyde Content for Liquid Placebo Before and After Lyophilization

| | Vial | Formaldehyde Concentration | | Acetaldehyde Concentration | | Propionaldehyde Concentration | |
|---|---|---|---|---|---|---|---|
| Sample | # | (ppm = μg/g) | | (ppm = μg/g) | | (ppm = μg/g) | |
| FT-021-094 | 1 | 2.17 ± 0.04 | <0.18* | 41.20 ± 0.64 | 0.19^{□} ± 0.01 | 76.94 ± 0.24 | 0.24^{□} ± 0.02 |
| | 2 | 5.57 ± 0.07 | <0.18* | 49.55 ± 1.00 | 0.21^{□} ± 0.01 | 85.57 ± 1.94 | <0.18* |
| | 3 | 11.81 ± 0.08 | <0.18* | 65.89 ± 0.12 | 0.99 ± 0.01 | 89.80 ± 0.21 | 0.23^{□} ± 0.01 |
| FT-021-094 | | 6.52 ± 4.89 | <0.18* | 52.21 ± 12.56 | 0.46 ± 0.46 | 84.77 ± 6.87 | 0.21^{□} ± 0.05 |
| Average | | | | | | | |
| FT-032-081 | 1 | <0.18* | <0.18* | 1.71 ± 0.04 | 0.27^{□} ± 0.00 | 1.88 ± 0.03 | 0.25^{□} ± 0.01 |
| | 2 | <0.18* | <0.18* | 1.61 ± 0.05 | 0.25^{□} ± 0.00 | 1.75 ± 0.02 | 0.23^{□} ± 0.02 |
| | 3 | <0.18* | <0.18* | 1.70 ± 0.01 | 0.26^{□} ± 0.01 | 1.86 ± 0.03 | 0.24^{□} ± 0.01 |

| | | | | | | |
|---|---|---|---|---|---|---|
| FT-032-081 | <0.18* | <0.18* | 1.67 ± 0.05 | 0.26^{□} ± 0.01 | 1.83 ± 0.07 | 0.24^{□} ± 0.01 |
| Average | | | | | | |

*Indicates sample was below the LOD.
^{□}Indicates sample was below LLOQ.

Storage of aqueous Poloxamer 407 solutions at 40° C. 75% RH for 6 months results in an increase in FA, AA, and PA impurities (see FIG. 26). Lyophilization reduces the aldehyde impurities present in FT-021-094 by 98.8%, 99.1%, and 99.7% for FA, AA, and PA respectively. Lyophilization also reduced AA and PA present in FT-032-081 to levels below 0.3 μg/g (see FIG. 26). Despite having different aldehyde profiles pre-lyophilization, the post-lyophilization samples have similar aldehyde content below 1.0 μg/g total aldehydes.

In summary, aldehyde content was determined for the Test composition, Placebo formulations, and Poloxamer 407 solutions. The Test composition and Placebo stored as a lyophilized cake demonstrated minimal aldehyde levels with total aldehyde content being below 1 μg/g for all storage conditions tested. Aqueous Poloxamer 407 solutions, including non-lyophilized placebo formulations, demonstrated higher aldehyde levels which increased when samples were exposed to accelerated stability conditions. Lyophilization of Poloxamer 407 solutions containing >100 μg/g aldehydes results in removal of 99% of formaldehyde, acetaldehyde, and propionaldehyde. Storage of Poloxamer 407 as a lyophilized cake rather than an aqueous solution reduces the formation of aldehydes; moreover, the lyophilization process actively reduces aldehyde content.

It can be seen that total aldehyde levels in lyophilized Placebo and lyophilized test composition samples were determined to be less than 1 μg aldehyde per gram of sample, i.e. 1 ppm, or less than 0.2 μg total aldehydes in one clinical dose. Poloxamer 407 when stored as an aqueous solution is prone to degradation and formation of aldehyde impurities. Lyophilization efficiently removes aldehyde impurities from Poloxamer 407 solutions and the resulting lyophilized cake when stored at a primal)/storage condition for up to 12 months shows no evidence for aldehyde formation.

### Example 20: Compositions with Other Gelling Agents

The following lyophilized composition was produced using sodium hyaluronate as the gelling agent. The weights and measurements relate to the lyophilized hyaluronic acid composition

| Component | Amount (mg/gram) |
|---|---|
| CHIR99021 | 5.96 |
| NaVPA | 168.0 |
| Sodium hyaluronate | 14.4 |
| Polyethylene glycol (15)-hydroxystearate (Solutol) | 50.6 |
| DMSO | 104.2 |
| Dilute hydrochloric acid | 94.8 |
| Water | (enough to 1 gram) |

The hyaluronic acid used to make this composition had a MW Average of 8.23×10\hat{ }5 Daltons as per its certificate of analysis provided by the vendor. It is sold as ‘HA1M’ by Lifecore Bio, where 1M stands for 1 million Dalton MW. 1-3% aq. solutions were prepared and used to make this composition.

Therefore, further compositions based on hyaluronic acid are feasible and envisaged by the present disclosure

### EQUIVALENTS

It is to be understood that the invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

The disclosure also includes the following numbered clauses.

1. A lyophilized pharmaceutical composition comprising one or more otic therapeutic agents and a gelling agent 2. The lyophilized pharmaceutical composition of clause 1, wherein the one or more otic therapeutic agents are one or more hearing loss treatment agents.
3. The lyophilized pharmaceutical composition of clause 1 of clause 2, wherein the one or more otic therapeutic agents are modulators of one or more biological pathways and biological targets associated with hearing loss.
4. The lyophilized pharmaceutical composition of any one of the preceding clauses, wherein the one or more otic therapeutic agents are selected from the group consisting of Wnt pathway agonists, histone deacetylase (HDAC) inhibitors, Dkk1 inhibitors, Axin inhibitors, SFRP1 inhibitors, bone morphogenetic protein (BMP) inhibitors, beta-catenin agonists, CyclinD1 activators, REST corepressor 1 (CoREST) inhibitors, NOTCH agonists, TGF-beta inhibitors, cAMP response element binding protein (CREB) activators, cyclin-dependent kinase (CDK) activators, CDK inhibitors, PI3K-AKT activators, PI3K-AKT inhibitors, PTEN inhibitors, ATOH1 agonists, ATOH1 antagonists, POU4F3 agonists, POU4F3 antagonists, GFI1 agonists, GFI1 antagonists, ERK/MAPK agonists, ERK/MAPK antagonists, FGF agonists, FGF antagonists, γ-aminobutyric acids (GABAs), voltage-gated Na+ channel antagonists, inositol, PKC agonists, PKC antagonists, FOXO inhibitors, FOXO agonists, Kv3 channel antagonists, p27Kip1 inhibitors, IL-1β, N-Methyl-D-aspartate (NMDA) receptor antagonists, NADPH quinone oxidoreductase 1, gamma secretase inhibitors, gamma secretase activators, NK1 receptor antagonist, NK1 receptor agonist, AMPA receptor agonist, AMPA receptor antagonist, Toll-Like Receptor (TLR) agonist, Receptor (TLR) antagonist, histamine H4 receptor agonist, H4 receptor antagonist, 5-HT3 receptor agonist, 5-HT3 receptor antagonist, Oct4 activators, Sox2 activators, Sox17 inducers, Klf4 inducers, cMyc activators, Sonic Hedgehog agonists, Sonic Hedgehog antagonists, Epidermal Growth Factor (EGF), Insulin Like Growth Factor (IGF), vascular endothelial growth factor (VEGF), endothelial nitric oxide synthase (eNOS), prostaglandin E (PGE), Brain-derived neurotrophic factor (BDNF), SMAD inhibitors, Sall4 inducers, Gata4 inducers, Gata6 inducers, proteasome inhibitors, retinoic acid receptor agonists, mTOR inhibitors, mTOR activators, Ascorbic acid, 2-phospho-1-ascorbic acid, KDM inhibitors, TTNPB, neurotrophin 3, DNA-modifying enzymes, LSD-1 inhibitors, Nicotinomide, Sirtuin, Histone methyl transferase inhibitors, Histone demethylase inhibitors, Histone Lysine Methyltransferase inhibitors, DNMT inhibitors, p53 inhibitors, p21 inhibitors, AMPK activators, Hippo activators, Hippo inhibitors, YAP/TAZ inhibitors, Mst1/2 inhibitors, CK1 activators, CK1 inhibitors, Noggin, R-spondin 1, BET activators, Sirt1 activators, Sirt1 inhibitors, Sirt2 activators, Sirt2 inhibitors, Sirt3 activators, Sirt3 inhibitors, JMJD3 inhibitors, DMNT inhibitors, Stat3 inhibitors, LSD1 inhibitors, active prostaglandins, cAMP activators, Oxidative phosphorylation uncouplers, arginine methyltransferase inhibitors, ALK4 inhibitors, Peroxisome proliferator-activated receptor gamma activators, EGFR inhibitors, SHH inhibitors, VitD activators, DOT1L inhibitors, Thyroid hormones, E box-dependent transcriptional activators, and protein degradation inhibitors.
5. The lyophilized pharmaceutical composition of any one of the preceding clauses, wherein the one or more otic therapeutic agents are hair cell regeneration agents and/or octoprotective agents.
6. The lyophilized pharmaceutical composition of any one of the preceding clauses, wherein the one or more otic therapeutic agents are selected from the group consisting of the agents described in Tables 1-13, and pharmaceutical salts thereof.
7. The lyophilized pharmaceutical composition of any one of the preceding clauses, wherein the one or more otic therapeutic agents are CHIR99021 or a pharmaceutical acceptable salt thereof, and valproic acid or a pharmaceutical acceptable salt thereof.
8. The lyophilized pharmaceutical composition of clause 7, wherein the pharmaceutically acceptable salt of valproic acid is a sodium salt;

optionally, the pharmaceutically acceptable salt of valproic acid is sodium valproate.

9. The lyophilized pharmaceutical composition of any one of the preceding clauses, wherein the gelling agent is a thermoreversible gelling agent.
10. The lyophilized pharmaceutical composition of clause 9, wherein the thermoreversible gelling agent comprises a poloxamer.
11. The lyophilized pharmaceutical composition of clause 10, wherein the poloxamer is selected from the group consisting of Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, and Poloxamer 407;

optionally, the poloxamer is Poloxamer 188 or Poloxamer 407; and

optionally, the poloxamer is Poloxamer 407.

12. The lyophilized pharmaceutical composition of any one of the preceding clauses, wherein the poloxamer is a purified poloxamer;

optionally, the poloxamer is purified Poloxamer 407.

13. The lyophilized pharmaceutical composition of any one of the preceding clauses, wherein the purified Poloxamer 407 has an average molecular weight of about 9 kDa or greater, about 9.2 kDa or greater, about 9.4 kDa or greater, about 9.6 kDa or greater, about 9.8 kDa or greater, about 10 kDa or greater, about 10.2 kDa or greater, about 10.4 kDa or greater, about 10.6 kDa or greater, about 10.8 kDa or greater, about 11 kDa or greater, about 11.2 kDa or greater, about 11.4 kDa or greater, about 11.6 kDa or greater, about 11.8 kDa or greater, about 12 kDa or greater, or about 12.1 kDa or greater.
14. The lyophilized pharmaceutical composition of any one of the preceding clauses, wherein the purified Poloxamer 407 is prepared by liquid-liquid extraction or size exclusion chromatography.
15. The lyophilized pharmaceutical composition of any one of the preceding clauses, wherein about 10% or more, about 20% or more, about 30% or more, about 40% or more, about 50% or more, about 60% or more, about 70% or more, about 80% or more, about 90% or more, about 95% or more, about 98% or more, or about 99% or more of the one or more impurities having molecular weights below 9 kDa are removed from the Poloxamer 407 during the purification.
16. The lyophilized pharmaceutical composition of any one of the preceding clauses, being in the form of a lyophilized cake.
17. The lyophilized pharmaceutical composition of any one of the preceding clauses, having a higher stability to oxygen and/or light as compared to a comparable pharmaceutical composition comprising one or more solvents.
18. The lyophilized pharmaceutical composition of any one of the preceding clauses, wherein the level of an impurity presented in the lyophilized pharmaceutical composition is less than about 10000 parts per million (ppm), less than about 1000 ppm, less than about 100 ppm, less than about 10 ppm, less than about 1 ppm, or less than about 0.1 ppm.
19. The lyophilized pharmaceutical composition of any one of the preceding clauses, wherein impurity is a residual solvent.
20. The lyophilized pharmaceutical composition of any one of the preceding clauses, wherein impurity is selected from the group consisting of 1-acetate-2-formate-1,2-propanediol, acetic acid, formic acid, formaldehyde, acetaldehyde, and propionaldehyde.
21. The lyophilized pharmaceutical composition of any one of the preceding clauses, wherein the level of polyethylene oxide presented in the lyophilized pharmaceutical composition is below about 3%, below about 2%, below about 1%, below about 0.5%, or below about 0.1%, as measured by high-performance liquid chromatography (HPLC).
22. The lyophilized pharmaceutical composition of any one of the preceding clauses, wherein the total level of one or more impurities with c Log P of about 1 or less presented in the lyophilized pharmaceutical composition is from about 30% to about 35%, from about 25% to about 29%, from about 20% to about 25%, from about 15% to about 19%, from about 10% to about 14%, from about 5% to about 9%, or from about 0% to about 4%, as measured by high-performance liquid chromatography (HPLC).
23. The lyophilized pharmaceutical composition of any one of the preceding clauses, wherein the total level of one or more impurities having a boiling of about 220° C. or less presented in the lyophilized pharmaceutical composition is from about 35% to about 40%, from about 30% to about 34%, from about 25% to about 29%, from about 20% to about 25%, from about 15% to about 19%, from about 10% to about 14%, from about 5% to about 9%, or from about 0% to about 4%, as measured by high-performance liquid chromatography (HPLC).
24. The lyophilized pharmaceutical composition of any one of the preceding clauses, wherein the lyophilized pharmaceutical composition comprises purified Poloxamer 407, and wherein the level of the one or more otic therapeutic agents presented in the lyophilized pharmaceutical composition is about 1.5 fold or higher, about 1.8 fold or higher, about 2 fold or higher, about 2.5 fold or higher, about 3 fold or higher, about 5 fold or higher, or about 10 fold or higher as compared to a comparable lyophilized pharmaceutical composition without purified Poloxamer 407;

optionally, the comparable lyophilized pharmaceutical composition comprises unpurified Poloxamer 407.

25. The lyophilized pharmaceutical composition of any one of the preceding clauses, wherein the lyophilized pharmaceutical composition comprises purified Poloxamer 407, and wherein the lyophilized pharmaceutical composition has lower batch-to-batch variability of one or more gelation properties (e.g., gelation temperature, viscosity, and/or stability) as compared to a comparable lyophilized pharmaceutical composition without purified Poloxamer 407;

optionally, the comparable lyophilized pharmaceutical composition comprises unpurified Poloxamer 407.

26. The lyophilized pharmaceutical composition of any one of the preceding clauses, wherein the lyophilized pharmaceutical composition comprises purified Poloxamer 407, and wherein the lyophilized pharmaceutical composition has a lower gelation temperature, a narrower gelation temperature range, and/or a higher viscosity as compared to a comparable lyophilized pharmaceutical composition without purified Poloxamer 407;

optionally, the comparable lyophilized pharmaceutical composition comprises unpurified Poloxamer 407.

27. The lyophilized pharmaceutical composition of any one of the preceding clauses, wherein the lyophilized pharmaceutical composition comprises purified Poloxamer 407, and wherein the lyophilized pharmaceutical composition has a reduced degradation rate as compared to a comparable lyophilized pharmaceutical composition without purified Poloxamer 407;

optionally, the comparable lyophilized pharmaceutical composition comprises unpurified Poloxamer 407.

28. The lyophilized pharmaceutical composition of any one of the preceding clauses, being suitable for preparing a reconstituted solution by a reconstitution process.
29. The lyophilized pharmaceutical composition of any one of the preceding clauses, wherein the reconstitution process is of less than about 30 minutes.
30. The lyophilized pharmaceutical composition of any one of the preceding clauses, wherein the reconstituted solution is suitable for injection;

optionally, the reconstituted solution is suitable for intratympanic injection.

31. The lyophilized pharmaceutical composition of any one of the preceding clauses, wherein the reconstituted solution maintains one or more rheometric properties of a pre-lyophilized solution which is used for preparing the lyophilized pharmaceutical composition.
32. The lyophilized pharmaceutical composition of any one of the preceding clauses, wherein the reconstituted solution has a reduced degradation rate as compared to a reconstituted solution prepared from a comparable lyophilized pharmaceutical composition without purified Poloxamer 407;

optionally, the comparable lyophilized pharmaceutical composition comprises unpurified Poloxamer 407.

33. The lyophilized pharmaceutical composition of any one of the preceding clauses for treating hearing loss in a subject in need thereof.
34. Use of the lyophilized pharmaceutical composition of any one of the preceding clauses in the preparation of a reconstituted solution for treating hearing loss in a subject in need thereof.
35. A method of treating hearing loss, comprising administering to a subject in need thereof a pharmaceutically acceptable amount of a reconstituted solution, wherein the reconstituted solution is prepared by a reconstitution process using the lyophilized pharmaceutical composition of any one of the preceding clauses.
36. A pharmaceutical composition, comprising:

i) CHIR99021 or a pharmaceutically acceptable salt thereof being present at a concentration ranging from 0.025 mg/ml to about 25 mg/ml;

ii) valproic acid or a pharmaceutically acceptable salt thereof being present at a concentration ranging from 0.5 mg/ml to about 500 mg/ml;

iii) poloxamer 407 being present at a concentration ranging from 1 wt % to about 25 wt %; and

iv) dimethyl sulfoxide (DMSO) being present at a concentration below 7.5 wt %.

37. The pharmaceutical composition of clause 36, wherein the pharmaceutically acceptable salt of valproic acid is a sodium salt;

optionally, the pharmaceutically acceptable salt of valproic acid is sodium valproate.

38. The pharmaceutical composition of any one of the preceding clauses, wherein the concentration of CHIR99021 or the pharmaceutically acceptable salt thereof ranges from about 0.05 mg/ml to about 5 mg/ml, from about 0.25 mg/ml to about 2.5 mg/ml, from about 0.5 mg/ml to about 1.75 mg/ml, or from about 1.45 mg/ml to about 1.65 mg/ml;

optionally, the concentration of CHIR99021 or the pharmaceutically acceptable salt thereof is about 1.55 mg/ml.

39. The pharmaceutical composition of any one of the preceding clauses, wherein the concentration of valproic acid or the pharmaceutically acceptable salt thereof ranges from about 2.5 mg/ml to about 200 mg/ml, from about 5 mg/ml to about 100 mg/ml, from about 15 mg/ml to about 50 mg/ml, or from about 43 mg/ml to about 46 mg/ml;

optionally, the concentration of valproic acid or the pharmaceutically acceptable salt thereof is about 44.5 mg/ml.

40. The pharmaceutical composition of any one of the preceding clauses, wherein the concentration of poloxamer 407 ranges from about 2.5 wt % to about 12.5 wt %, from about 5 wt % to about 11 wt %, from about 6 wt % to about 10 wt %, or from about 7 wt % to about 8.5 wt %;

optionally, the concentration of poloxamer 407 is about 8 wt %.

41. The pharmaceutical composition of any one of the preceding clauses, wherein the concentration of DMSO ranges from about 0.5 wt % to about 5 wt %, from about 1 wt % to about 4 wt %, from about 1.5 wt % to about 3.5 wt %, or from about 2 wt % to about 3 wt %;

optionally, the concentration of DMSO is about 2.5 wt %.

42. The pharmaceutical composition of any one of the preceding clauses, wherein the weight ratio between CHIR99021 or the pharmaceutically acceptable salt thereof and valproic acid or the pharmaceutically acceptable salt thereof ranges from about 1:5 to about 1:10, from about 1:10 to about 1:50, from about 1:20 to about 1:35, from about 1:25 to about 1:31, or from about 1:27 to about 1:29.
43. The pharmaceutical composition of any one of the preceding clauses, wherein the weight ratio between poloxamer 407 and the DMSO ranges from about 1:5 to about 40:1, from about 1:2 to about 15:1, from about 1:1 to about 8:1, from about 2:1 to about 4:1, or from about 2.5:1 to about 3.5:1;

optionally, the weight ratio between poloxamer 407 and the DMSO is about 3:1.

44. The pharmaceutical composition of any one of the preceding clauses, wherein:

the weight ratio between CHIR99021 and poloxamer 407 is about 0.02:1;

the weight ratio between CHIR99021 and the DMSO is about 0.06:1;

the weight ratio between valproic acid sodium salt and poloxamer 407 is about 0.54:1; and/or

the weight ratio between valproic acid sodium salt and the DMSO is about 3.2:1.

45. The pharmaceutical composition of any one of the preceding clauses, wherein:

the concentration of CHIR99021 or the pharmaceutically acceptable salt thereof ranges from about 1.45 mg/ml to about 1.65 mg/ml;

the concentration of valproic acid or the pharmaceutically acceptable salt thereof ranges from about 43 mg/ml to about 46 mg/ml;

the concentration of poloxamer 407 ranges from about 7 wt % to about 8.5 wt %; and

the concentration of DMSO ranges from about 2 wt % to about 3 wt %.

46. The pharmaceutical composition of any one of the preceding clauses, wherein:

the concentration of CHIR99021 or the pharmaceutically acceptable salt thereof is about 1.55 mg/ml;

the concentration of valproic acid or the pharmaceutically acceptable salt thereof is about 44.5 mg/ml;

the concentration of poloxamer 407 is about 8 wt %; and

the concentration of DMSO is about 2.5 wt %.

47. A pharmaceutical composition, comprising:

i) CHIR99021 or a pharmaceutically acceptable salt thereof being present at a concentration ranging from 0.025 mg/ml to about 25 mg/ml;

ii) valproic acid or a pharmaceutically acceptable salt thereof being present at a concentration ranging from 1 mg/ml to about 500 mg/ml;

iii) poloxamer 407 being present at a concentration ranging from 1 wt % to about 25 wt %; and

iv) dimethyl sulfoxide (DMSO) being present at a concentration below 7.5 wt %.

48. The pharmaceutical composition of clause 47, wherein the pharmaceutically acceptable salt of valproic acid is a sodium salt;

optionally, the pharmaceutically acceptable salt of valproic acid is sodium valproate.

49. The pharmaceutical composition of any one of the preceding clauses, wherein the concentration of CHIR99021 or the pharmaceutically acceptable salt thereof ranges from about 0.05 mg/ml to about 10 mg/ml, from about 0.25 mg/ml to about 2.5 mg/ml, from about 0.5 mg/ml to about 1.75 mg/ml, from about 0.85 mg/ml to about 1.15 mg/ml;

optionally, the concentration of CHIR99021 or the pharmaceutically acceptable salt thereof is about 1.05 mg/ml.

50. The pharmaceutical composition of any one of the preceding clauses, wherein the concentration of valproic acid or the pharmaceutically acceptable salt thereof ranges from about 2.5 mg/ml to about 200 mg/ml, from about 5 mg/ml to about 100 mg/ml, from about 15 mg/ml to about 50 mg/ml, from about 28 mg/ml to about 31 mg/ml;

optionally, the concentration of valproic acid or the pharmaceutically acceptable salt thereof is about 29.5 mg/ml.

51. The pharmaceutical composition of any one of the preceding clauses, wherein the concentration of poloxamer 407 ranges from about 2.5 wt % to about 12.5 wt %, from about 5 wt % to about 11 wt %, from about 11 wt % to about 10 wt %, from about 7 wt % to about 8.5 wt %;

optionally, the concentration of poloxamer 407 is about 7.5 wt %.

52. The pharmaceutical composition of any one of the preceding clauses, wherein the concentration of DMSO ranges from about 0.5 wt % to about 5 wt %, from about 1 wt % to about 4 wt %, from about 1.5 wt % to about 3.5 wt %, from about 2 wt % to about 3 wt %;

optionally, the concentration of DMSO is about 2.5 wt %.

53. The pharmaceutical composition of any one of the preceding clauses, wherein the weight ratio between CHIR99021 or the pharmaceutically acceptable salt thereof and valproic acid or the pharmaceutically acceptable salt thereof ranges from about 1:5 to about 1:10, from about 1:10 to about 1:50, from about 1:20 to about 1:35, from about 1:25 to about 1:31, or from about 1:27 to about 1:29.
54. The pharmaceutical composition of any one of the preceding clauses, wherein the weight ratio between poloxamer 407 and the DMSO ranges from about 1:5 to about 40:1, from about 1:2 to about 15:1, from about 1:1 to about 8:1, from about 2:1 to about 4:1, from about 2.5:1 to about 3.5:1;

optionally, the weight ratio between poloxamer 407 and the DMSO is about 3:1.

55. The pharmaceutical composition of any one of the preceding clauses, wherein:

the weight ratio between CHIR99021 and poloxamer 407 is about 0.016:1;

the weight ratio between the CHIR99021 and the DMSO is about 0.06:1;

the weight ratio between valproic acid sodium salt and poloxamer 407 is about 0.42:1; and/or the weight ratio between valproic acid sodium salt and the DMSO is about 1.5:1.

56. The pharmaceutical composition of any one of the preceding clauses, wherein:

the concentration of CHIR99021 or the pharmaceutically acceptable salt thereof ranges from about 0.95 mg/ml to about 1.15 mg/ml;

the concentration of valproic acid or the pharmaceutically acceptable salt thereof ranges from about 28 mg/ml to about 31 mg/ml;

the concentration of poloxamer 407 ranges from about 7 wt % to about 8.5 wt %; and

the concentration of DMSO ranges from about 2 wt % to about 3 wt %.

57. The pharmaceutical composition of any one of the preceding clauses, wherein:

the concentration of CHIR99021 or the pharmaceutically acceptable salt thereof is about 1.05 mg/ml;

the concentration of valproic acid or the pharmaceutically acceptable salt thereof is about 29.5 mg/ml;

the concentration of poloxamer 407 is about 7.5 wt %; and

the concentration of DMSO is about 2.5 wt %.

58. A pharmaceutical composition, comprising:

i) CHIR99021 or a pharmaceutically acceptable salt thereof being present at a concentration ranging from 0.025 mg/ml to about 25 mg/ml;

ii) valproic acid or a pharmaceutically acceptable salt thereof being present at a concentration ranging from 0.5 mg/ml to about 500 mg/ml;

iii) poloxamer 407 being present at a concentration ranging from 1 wt % to about 25 wt %; and

iv) dimethyl sulfoxide (DMSO) being present at a concentration below 7.5 wt %.

59. The pharmaceutical composition of clause 58, wherein the pharmaceutically acceptable salt of valproic acid is a sodium salt;

optionally, the pharmaceutically acceptable salt of valproic acid is sodium valproate.

60. The pharmaceutical composition of any one of the preceding clauses, wherein the concentration of CHIR99021 or the pharmaceutically acceptable salt thereof ranges from about 0.05 mg/ml to about 5 mg/ml, from about 0.25 mg/ml to about 2.5 mg/ml, from about 0.5 mg/ml to about 1.75 mg/ml, or from about 0.6 mg/ml to about 0.75 mg/ml;

optionally, the concentration of CHIR99021 or the pharmaceutically acceptable salt thereof ranges is about 0.7 mg/ml.

61. The pharmaceutical composition of any one of the preceding clauses, wherein the concentration of valproic acid or the pharmaceutically acceptable salt thereof ranges from about 2.5 mg/ml to about 200 mg/ml, from about 5 mg/ml to about 100 mg/ml, from about 15 mg/ml to about 50 mg/ml, or from about 18 mg/ml to about 21 mg/ml;

optionally, the concentration of valproic acid or the pharmaceutically acceptable salt thereof is about 19.5 mg/ml.

62. The pharmaceutical composition of any one of the preceding clauses, wherein the concentration of poloxamer 407 ranges from about 2.5 wt % to about 12.5 wt %, from about 5 wt % to about 11 wt %, from about 6 wt % to about 10 wt %, or from about 7 wt % to about 8.5 wt %;

optionally, the concentration of poloxamer 407 is about 7.5 wt %.

63. The pharmaceutical composition of any one of the preceding clauses, wherein the concentration of DMSO ranges from about 0.5 wt % to about 5 wt %, from about 1 wt % to about 4 wt %, from about 1.5 wt % to about 3.5 wt %, or from about 2 wt % to about 3 wt %;

optionally, the concentration of DMSO is about 5 wt %.

64. The pharmaceutical composition of any one of the preceding clauses, wherein the weight ratio between CHIR99021 or the pharmaceutically acceptable salt thereof and valproic acid or the pharmaceutically acceptable salt thereof ranges from about 1:5 to about 1:10, from about 1:10 to about 1:50, from about 1:20 to about 1:35, from about 1:25 to about 1:31, or from about 1:27 to about 1:29.
65. The pharmaceutical composition of any one of the preceding clauses, wherein the weight ratio between poloxamer 407 and the DMSO ranges from about 1:5 to about 40:1, from about 1:2 to about 15:1, from about 1:1 to about 8:1, from about 2:1 to about 4:1, from about 2.5:1 to about 3.5:1.
66. The pharmaceutical composition of any one of the preceding clauses, wherein:

the weight ratio between poloxamer 407 and the DMSO is about 3:1;

the weight ratio between the CHIR99021 and poloxamer 407 is about 0.013:1;

the weight ratio between CHIR99021 and the DMSO is about 0.06:1;

the weight ratio between valproic acid sodium salt and poloxamer 407 is about 0.23:1; and/or

the weight ratio between valproic acid sodium salt and the DMSO is about 1.8:1.

67. The pharmaceutical composition of any one of the preceding clauses, wherein:

the concentration of CHIR99021 or the pharmaceutically acceptable salt thereof ranges from about 0.6 mg/ml to about 0.75 mg/ml;

the concentration of valproic acid or the pharmaceutically acceptable salt thereof ranges from about 18 mg/ml to about 21 mg/ml;

the concentration of poloxamer 407 ranges from about 7 wt % to about 8.5 wt %; and

the concentration of DMSO ranges from about 2 wt % to about 3 wt %.

68. The pharmaceutical composition of any one of the preceding clauses, wherein:

the concentration of CHIR99021 or the pharmaceutically acceptable salt thereof is about 0.7 mg/ml;

the concentration of valproic acid or the pharmaceutically acceptable salt thereof is about 19.5 mg/ml;

the concentration of poloxamer 407 is about 7.5 wt %; and

the concentration of DMSO is about 2.5 wt %.

69. The pharmaceutical composition of any one of the preceding clauses, comprising one or more of:

water or a buffering agent;

a bulking agent;

a stabilizing agent;

a tonicity-adjusting agent; and

a soothing agent.

70. A method of processing the pharmaceutical composition of any one of clauses 36-69 to form a lyophilized pharmaceutical composition.
71. The method of clause 70, comprising the steps of:

i) cooling the pharmaceutical composition at a first temperature below 0° C. for a first period of time;

ii) removing one or more solvents from the resulting mixture of step (i) at a second temperature below 0° C., and at a reduced pressure below 760 Torr, for a second period of time.

72. The method of clause 70 or clause 71, comprising one or more steps selected from:

0a) dispensing the pharmaceutical composition in a sterile vial;

ia) cooling the pharmaceutical composition at a rate ranging from about 0.1° C. per minute to about 5° C. per minute to the first temperature ranging from about −20° C. to about −80° C.;

ib) holding the pharmaceutical composition at the first temperature for the first period of time ranging from about 1 hour to about 6 hours;

iia) subjecting the pharmaceutical composition to the reduced pressure ranging from about 1 mTorr to 1000 mTorr and warming the pharmaceutical composition at a rate ranging from about 0.1° C. per minute to about 5° C. per minute to the second temperature ranging from about −10° C. to −50° C.;

iib) holding the pharmaceutical composition at the second temperature and under the reduced pressure or the second period of time ranging from about 10 hours to about 30 hours;

iiia) filling the sterile vial with nitrogen; and

iiib) capping and crimping the sterile vial.

73. The method of any one of clauses 70-72, wherein the pharmaceutical composition comprises the one or more otic therapeutic agents and the poloxamer;

optionally, the pharmaceutical composition comprises the one or more otic therapeutic agents and poloxamer 407; and

optionally, the pharmaceutical composition comprises the one or more otic therapeutic agents and purified poloxamer 407.

74. The method of any one of clauses 70-73, wherein the pharmaceutical composition comprises CHIR99021, valproic acid sodium salt, the poloxamer, DMSO, and water;

optionally, the pharmaceutical composition comprises CHIR99021, valproic acid sodium salt, poloxamer 407, DMSO, and water;

optionally, the pharmaceutical composition comprises CHIR99021, valproic acid sodium salt, purified poloxamer 407, DMSO, and water.

75. The method of any one of clauses 70-74, comprising one or more steps selected from:

0a) dispensing the pharmaceutical composition in a sterile vial;

ia) cooling the pharmaceutical composition at a rate of about 0.5° C. per minute to the first temperature of about −45° C.;

ib) holding the pharmaceutical composition at the first temperature for the first period of time of about 3 hours;

iia) subjecting the pharmaceutical composition to the reduced pressure of about 80 mTorr to 1000 mTorr and warming the pharmaceutical composition at a rate of about 0.5° C. per minute to the second temperature of about −30° C.;

iib) holding the pharmaceutical composition at the second temperature and under the reduced pressure for the second period of time ranging from about 10 hours to about 15 hours;

iic) warming the pharmaceutical composition at a rate of about 0.5° C. per minute to 20° C.;

iid) holding the pharmaceutical composition at 20° C. and under the reduced pressure for 20 hours,

iiia) filling the sterile vial with nitrogen; and

iiib) capping and crimping the sterile vial.

76. A lyophilized pharmaceutical composition being prepared by lyophilizing the pharmaceutical composition of any one of clauses 36-69.
77. A lyophilized pharmaceutical composition being prepared by the method of any one of clauses 70-75.
78. A reconstituted solution being prepared by adding a diluent to the lyophilized pharmaceutical composition of any one of clauses 1-24 and 76-77.
79. A reconstituted solution being prepared by adding a diluent to a lyophilized pharmaceutical composition which is prepared by lyophilizing the pharmaceutical composition of any one of clauses 36-69.
80. A reconstituted solution being prepared by adding a diluent to a lyophilized pharmaceutical composition which is prepared by the method of any one of clauses 70-75.
81. A reconstituted solution being prepared by adding a diluent to a lyophilized pharmaceutical composition, comprising one or more otic therapeutic agents and a gelling agent.
82. The reconstituted solution of any one of the preceding clauses, wherein the one or more otic therapeutic agents are one or more hearing loss treatment agents.
83. The reconstituted solution of any one of the preceding clauses, wherein the one or more otic therapeutic agents are CHIR99021 or a pharmaceutical acceptable salt thereof, and valproic acid or a pharmaceutical acceptable salt thereof.
84. The reconstituted solution of clause 83, wherein the pharmaceutical acceptable salt of valproic acid is a sodium salt;

optionally, the pharmaceutically acceptable salt of valproic acid is sodium valproate.

85. The reconstituted solution of any one of the preceding clauses, wherein the gelling agent is a thermoreversible gelling agent.
86. The reconstituted solution of clause 85, wherein the thermoreversible gelling agent comprises a poloxamer.
87. The reconstituted solution of clause 86, wherein the poloxamer is selected from the group consisting of Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, and Poloxamer 407;

optionally, the poloxamer is Poloxamer 188 or Poloxamer 407; and

optionally, the poloxamer is Poloxamer 407.

88. The reconstituted solution of any one of the preceding clauses, wherein the poloxamer is a purified poloxamer;

optionally, the poloxamer is purified Poloxamer 407.

89. The reconstituted solution of any one of the preceding clauses, wherein the purified Poloxamer 407 has an average molecular weight of about 9 kDa or greater, about 9.2 kDa or greater, about 9.4 kDa or greater, about 9.6 kDa or greater, about 9.8 kDa or greater, about 10 kDa or greater, about 10.2 kDa or greater, about 10.4 kDa or greater, about 10.6 kDa or greater, about 10.8 kDa or greater, about 11 kDa or greater, about 11.2 kDa or greater, about 11.4 kDa or greater, about 11.6 kDa or greater, about 11.8 kDa or greater, about 12 kDa or greater, or about 12.1 kDa or greater.
90. The reconstituted solution of any one of the preceding clauses, wherein the purified Poloxamer 407 is prepared by liquid-liquid extraction or size exclusion chromatography.
91. The reconstituted solution of any one of the preceding clauses, wherein about 10% or more, about 20% or more, about 30% or more, about 40% or more, about 50% or more, about 60% or more, about 70% or more, about 80% or more, about 90% or more, about 95% or more, about 98% or more, or about 99% or more of the one or more impurities having molecular weights below 9 kDa are removed from the Poloxamer 407 during the purification.
92. The reconstituted solution of any one of the preceding clauses, wherein the diluent comprises water and dimethyl sulfoxide (DMSO).
93. The reconstituted solution of any one of the preceding clauses, wherein the concentration of DMSO in the diluent ranges from about 1% w/w to about 15% w/w, from about 2% w/w to about 12% w/w, from about 3% w/w to about 10% w/w, from about 4% w/w to about 9% w/w, from about 5% w/w to about 8% w/w, from about 5.5% w/w to about 7.5% w/w, from about 5.8% w/w to about 7% w/w, from about 6% w/w to about 6.8% w/w, or from about 6.2% w/w to about 6.6% w/w;

optionally, the concentration of DMSO in the diluent is about 6.4% w/w.

94. The reconstituted solution of any one of the preceding clauses, wherein the amount of the diluent added during the constitution ranges from about 1 μL to about 6 μL, from about 2 μL to about 5 μL, from about 2.5 μL to about 4.5 μL, from about 2.8 μL to about 4 μL, from about 3 μL to about 3.8 μL, or from about 3.2 μL to about 3.6 per mg of the lyophilized pharmaceutical composition;

optionally, the amount of the diluent added during the constitution is about 3.4 μL per mg of the lyophilized pharmaceutical composition.

95. The reconstituted solution of any one of the preceding clauses, comprising:

i) CHIR99021 or a pharmaceutically acceptable salt thereof being present at a concentration ranging from 0.05 mg/ml to about 50 mg/ml;

ii) valproic acid or a pharmaceutically acceptable salt thereof being present at a concentration ranging from 1 mg/ml to about 1000 mg/ml;

iii) poloxamer 407 being present at a concentration ranging from 2 wt % to about 50 wt %; and

iv) dimethyl sulfoxide (DMSO) being present at a concentration below 15 wt %.

96. The reconstituted solution of clause 95, wherein the pharmaceutically acceptable salt of valproic acid is a sodium salt;

optionally, the pharmaceutically acceptable salt of valproic acid is sodium valproate.

97. The reconstituted solution of any one of the preceding clauses, wherein the concentration of CHIR99021 or the pharmaceutically acceptable salt thereof ranges from about 0.1 mg/ml to about 10 mg/ml, from about 0.5 mg/ml to about 5 mg/ml, from about 1 mg/ml to about 3.5 mg/ml, or from about 2.9 mg/ml to about 3.3 mg/ml;

optionally, the concentration of CHIR99021 or the pharmaceutically acceptable salt thereof is about 3.1 mg/ml.

98. The reconstituted solution of any one of the preceding clauses, wherein the concentration of valproic acid or the pharmaceutically acceptable salt thereof ranges from about 5 mg/ml to about 400 mg/ml, from about 10 mg/ml to about 200 mg/ml, from about 30 mg/ml to about 100 mg/ml, or from about 86 mg/ml to about 92 mg/ml;

optionally, the concentration of valproic acid or the pharmaceutically acceptable salt thereof is about 89 mg/ml.

99. The reconstituted solution of any one of the preceding clauses, wherein the concentration of poloxamer 407 ranges from about 5 wt % to about 25 wt %, from about 10 wt % to about 22 wt %, from about 12 wt % to about 20 wt %, or from about 14 wt % to about 17 wt %;

optionally, the concentration of poloxamer 407 is about 16 wt %.

100. The reconstituted solution of any one of the preceding clauses, wherein the concentration of DMSO ranges from about 1 wt % to about 10 wt %, from about 2 wt % to about 8 wt %, from about 3 wt % to about 7 wt %, or from about 4 wt % to about 6 wt %;

optionally, the concentration of DMSO is about 5 wt %.

101. The reconstituted solution of any one of the preceding clauses, wherein the weight ratio between CHIR99021 or the pharmaceutically acceptable salt thereof and valproic acid or the pharmaceutically acceptable salt thereof ranges from about 1:5 to about 1:10, from about 1:10 to about 1:50, from about 1:20 to about 1:35, from about 1:25 to about 1:31, or from about 1:27 to about 1:29.
102. The reconstituted solution of any one of the preceding clauses, wherein the weight ratio between poloxamer 407 and the DMSO ranges from about 1:5 to about 40:1, from about 1:2 to about 15:1, from about 1:1 to about 8:1, from about 2:1 to about 4:1, or from about 2.5:1 to about 3.5:1;

optionally, the weight ratio between poloxamer 407 and the DMSO is about 3:1.

103. The reconstituted solution of any one of the preceding clauses, wherein:

the weight ratio between CHIR99021 and poloxamer 407 is about 0.02:1;

the weight ratio between CHIR99021 and the DMSO is about 0.06:1;

the weight ratio between valproic acid sodium salt and poloxamer 407 is about 0.54:1; and/or the weight ratio between valproic acid sodium salt and the DMSO is about 3.2:1.

104. The reconstituted solution of any one of the preceding clauses, wherein:

the concentration of CHIR99021 or the pharmaceutically acceptable salt thereof ranges from about 2.9 mg/ml to about 3.3 mg/ml;

the concentration of valproic acid or the pharmaceutically acceptable salt thereof ranges from about 86 mg/ml to about 92 mg/ml;

the concentration of poloxamer 407 ranges from about 14 wt % to about 17 wt %; and

the concentration of DMSO ranges from about 4 wt % to about 6 wt %.

105. The reconstituted solution of any one of the preceding clauses, wherein:

the concentration of CHIR99021 or the pharmaceutically acceptable salt thereof is about 3.1 mg/ml;

the concentration of valproic acid or the pharmaceutically acceptable salt thereof is about 89 mg/ml;

the concentration of poloxamer 407 is about 16 wt %; and

the concentration of DMSO is about 5 wt %.

106. The reconstituted solution of any one of the preceding clauses, comprising:

i) CHIR99021 or a pharmaceutically acceptable salt thereof being present at a concentration ranging from 0.05 mg/ml to about 50 mg/ml;

ii) valproic acid or a pharmaceutically acceptable salt thereof being present at a concentration ranging from 1 mg/ml to about 1000 mg/ml;

iii) poloxamer 407 being present at a concentration ranging from 2 wt % to about 50 wt %; and

iv) dimethyl sulfoxide (DMSO) being present at a concentration below 15 wt %.

107. The reconstituted solution of clause 106, wherein the pharmaceutically acceptable salt of valproic acid is a sodium salt;

optionally, the pharmaceutically acceptable salt of valproic acid is sodium valproate.

108. The reconstituted solution of any one of the preceding clauses, wherein the concentration of CHIR99021 or the pharmaceutically acceptable salt thereof ranges from about 0.1 mg/ml to about 10 mg/ml, from about 0.5 mg/ml to about 5 mg/ml, from about 1 mg/ml to about 3.5 mg/ml, from about 1.9 mg/ml to about 2.3 mg/ml;

optionally, the concentration of CHIR99021 or the pharmaceutically acceptable salt thereof is about 2.1 mg/ml.

109. The reconstituted solution of any one of the preceding clauses, wherein the concentration of valproic acid or the pharmaceutically acceptable salt thereof ranges from about 5 mg/ml to about 400 mg/ml, from about 10 mg/ml to about 200 mg/ml, from about 30 mg/ml to about 100 mg/ml, from about 56 mg/ml to about 62 mg/ml;

optionally, the concentration of valproic acid or the pharmaceutically acceptable salt thereof is about 59 mg/ml.

110. The reconstituted solution of any one of the preceding clauses, wherein the concentration of poloxamer 407 ranges from about 5 wt % to about 25 wt %, from about 10 wt % to about 22 wt %, from about 12 wt % to about 20 wt %, from about 14 wt % to about 17 wt %;

optionally, the concentration of poloxamer 407 is about 15 wt %.

111. The reconstituted solution of any one of the preceding clauses, wherein the concentration of DMSO ranges from about 1 wt % to about 10 wt %, from about 2 wt % to about 8 wt %, from about 3 wt % to about 7 wt %, from about 4 wt % to about 6 wt %;

optionally, the concentration of DMSO is about 5 wt %.

112. The reconstituted solution of any one of the preceding clauses, wherein the weight ratio between CHIR99021 or the pharmaceutically acceptable salt thereof and valproic acid or the pharmaceutically acceptable salt thereof ranges from about 1:5 to about 1:10, from about 1:10 to about 1:50, from about 1:20 to about 1:35, from about 1:25 to about 1:31, or from about 1:27 to about 1:29.
113. The reconstituted solution of any one of the preceding clauses, wherein the weight ratio between poloxamer 407 and the DMSO ranges from about 1:5 to about 40:1, from about 1:2 to about 15:1, from about 1:1 to about 8:1, from about 2:1 to about 4:1, from about 2.5:1 to about 3.5:1;

optionally, the weight ratio between poloxamer 407 and the DMSO is about 3:1.

114. The reconstituted solution of any one of the preceding clauses, wherein:

the weight ratio between CHIR99021 and poloxamer 407 is about 0.016:1;

the weight ratio between the CHIR99021 and the DMSO is about 0.06:1;

the weight ratio between valproic acid sodium salt and poloxamer 407 is about 0.42:1; and/or

the weight ratio between valproic acid sodium salt and the DMSO is about 1.5:1.

115. The reconstituted solution of any one of the preceding clauses, wherein:

the concentration of CHIR99021 or the pharmaceutically acceptable salt thereof ranges from about 1.9 mg/ml to about 2.3 mg/ml;

the concentration of valproic acid or the pharmaceutically acceptable salt thereof ranges from about 56 mg/ml to about 62 mg/ml;

the concentration of poloxamer 407 ranges from about 14 wt % to about 17 wt %; and

the concentration of DMSO ranges from about 4 wt % to about 6 wt %.

116. The reconstituted solution of any one of the preceding clauses, wherein:

the concentration of CHIR99021 or the pharmaceutically acceptable salt thereof is about 2.1 mg/ml;

the concentration of valproic acid or the pharmaceutically acceptable salt thereof is about 59 mg/ml;

the concentration of poloxamer 407 is about 15 wt %; and

the concentration of DMSO is about 5 wt %.

117. The reconstituted solution of any one of the preceding clauses, comprising:

i) CHIR99021 or a pharmaceutically acceptable salt thereof being present at a concentration ranging from 0.05 mg/ml to about 50 mg/ml;

ii) valproic acid or a pharmaceutically acceptable salt thereof being present at a concentration ranging from 1 mg/ml to about 1000 mg/ml;

iii) poloxamer 407 being present at a concentration ranging from 2 wt % to about 50 wt %; and

iv) dimethyl sulfoxide (DMSO) being present at a concentration below 15 wt %.

118. The reconstituted solution of clause 117, wherein the pharmaceutically acceptable salt of valproic acid is a sodium salt;

optionally, the pharmaceutically acceptable salt of valproic acid is sodium valproate.

119. The reconstituted solution of any one of the preceding clauses, wherein the concentration of CHIR99021 or the pharmaceutically acceptable salt thereof ranges from about 0.1 mg/ml to about 10 mg/ml, from about 0.5 mg/ml to about 5 mg/ml, from about 1 mg/ml to about 3.5 mg/ml, or from about 1.2 mg/ml to about 1.5 mg/ml;

optionally, the concentration of CHIR99021 or the pharmaceutically acceptable salt thereof ranges is about 1.4 mg/ml.

120. The reconstituted solution of any one of the preceding clauses, wherein the concentration of valproic acid or the pharmaceutically acceptable salt thereof ranges from about 5 mg/ml to about 400 mg/ml, from about 10 mg/ml to about 200 mg/ml, from about 30 mg/ml to about 100 mg/ml, or from about 36 mg/ml to about 42 mg/ml;

optionally, the concentration of valproic acid or the pharmaceutically acceptable salt thereof is about 39 mg/ml.

121. The reconstituted solution of any one of the preceding clauses, wherein the concentration of poloxamer 407 ranges from about 5 wt % to about 25 wt %, from about 10 wt % to about 22 wt %, from about 12 wt % to about 20 wt %, or from about 14 wt % to about 17 wt %;

optionally, the concentration of poloxamer 407 is about 15 wt %.

122. The reconstituted solution of any one of the preceding clauses, wherein the concentration of DMSO ranges from about 1 wt % to about 10 wt %, from about 2 wt % to about 8 wt %, from about 3 wt % to about 7 wt %, or from about 4 wt % to about 6 wt %;

optionally, the concentration of DMSO is about 5 wt %.

123. The reconstituted solution of any one of the preceding clauses, wherein the weight ratio between CHIR99021 or the pharmaceutically acceptable salt thereof and valproic acid or the pharmaceutically acceptable salt thereof ranges from about 1:5 to about 1:10, from about 1:10 to about 1:50, from about 1:20 to about 1:35, from about 1:25 to about 1:31, or from about 1:27 to about 1:29.
124. The reconstituted solution of any one of the preceding clauses, wherein the weight ratio between poloxamer 407 and the DMSO ranges from about 1:5 to about 40:1, from about 1:2 to about 15:1, from about 1:1 to about 8:1, from about 2:1 to about 4:1, from about 2.5:1 to about 3.5:1.
125. The reconstituted solution of any one of the preceding clauses, wherein:

the weight ratio between poloxamer 407 and the DMSO is about 3:1;

the weight ratio between the CHIR99021 and poloxamer 407 is about 0.013:1;

the weight ratio between CHIR99021 and the DMSO is about 0.06:1;

the weight ratio between valproic acid sodium salt and poloxamer 407 is about 0.23:1; and/or the weight ratio between valproic acid sodium salt and the DMSO is about 1.8:1.

126. The reconstituted solution of any one of the preceding clauses, wherein:

the concentration of CHIR99021 or the pharmaceutically acceptable salt thereof ranges from about 1.2 mg/ml to about 1.5 mg/ml;

the concentration of valproic acid or the pharmaceutically acceptable salt thereof ranges from about 36 mg/ml to about 42 mg/ml;

the concentration of poloxamer 407 ranges from about 14 wt % to about 17 wt %; and

the concentration of DMSO ranges from about 4 wt % to about 6 wt %.

127. The reconstituted solution of any one of the preceding clauses, wherein:

the concentration of CHIR99021 or the pharmaceutically acceptable salt thereof is about 1.4 mg/ml;

the concentration of valproic acid or the pharmaceutically acceptable salt thereof is about 39 mg/ml;

the concentration of poloxamer 407 is about 15 wt %; and

the concentration of DMSO is about 5 wt %.

128. The reconstituted solution of any one of the preceding clauses, comprising one or more of:

water or a buffering agent;

a bulking agent;

a stabilizing agent;

a tonicity-adjusting agent; and

a soothing agent.

129. The reconstituted solution of any one of the preceding clauses, wherein the reconstituted solution comprises purified Poloxamer 407, and wherein the reconstituted solution has a higher stability to oxygen and/or light as compared to a comparable reconstituted solution without purified Poloxamer 407;

optionally, the comparable reconstituted solution comprises unpurified Poloxamer 407.

130. The reconstituted solution of any one of the preceding clauses, wherein the level of an impurity presented in the reconstituted solution is less than about 10000 parts per million (ppm), less than about 1000 ppm, less than about 100 ppm, less than about 10 ppm, less than about 1 ppm, or less than about 0.1 ppm.
131. The reconstituted solution of any one of the preceding clauses, wherein impurity is selected from the group consisting of 1-acetate-2-formate-1,2-propanediol, acetic acid, formic acid, formaldehyde, acetaldehyde, and propionaldehyde.
132. The reconstituted solution of any one of the preceding clauses, wherein the level of polyethylene oxide presented in the reconstituted solution is below about 3%, below about 2%, below about 1%, below about 0.5%, or below about 0.1%, as measured by high-performance liquid chromatography (HPLC).
133. The reconstituted solution of any one of the preceding clauses, wherein the total level of one or more impurities with c Log P of about 1 or less presented in the reconstituted solution is from about 30% to about 35%, from about 25% to about 29%, from about 20% to about 25%, from about 15% to about 19%, from about 10% to about 14%, from about 5% to about 9%, or from about 0% to about 4%, as measured by high-performance liquid chromatography (HPLC).
134. The reconstituted solution of any one of the preceding clauses, wherein the total level of one or more impurities having a boiling of about 220° C. or less presented in the reconstituted solution is from about 35% to about 40%, from about 30% to about 34%, from about 25% to about 29%, from about 20% to about 25%, from about 15% to about 19%, from about 10% to about 14%, from about 5% to about 9%, or from about 0% to about 4%, as measured by high-performance liquid chromatography (HPLC).
135. The reconstituted solution of any one of the preceding clauses, wherein the reconstituted solution comprises purified Poloxamer 407, and wherein the level of the one or more otic therapeutic agents presented in the reconstituted solution is about 1.5 fold or higher, about 1.8 fold or higher, about 2 fold or higher, about 2.5 fold or higher, about 3 fold or higher, about 5 fold or higher, or about 10 fold or higher as compared to a comparable reconstituted solution without purified Poloxamer 407;

optionally, the comparable reconstituted solution comprises unpurified Poloxamer 407.

136. The reconstituted solution of any one of the preceding clauses, wherein the reconstituted solution comprises purified Poloxamer 407, and wherein the reconstituted solution has lower batch-to-batch variability of one or more gelation properties (e.g., gelation temperature, viscosity, and/or stability) as compared to a comparable reconstituted solution without purified Poloxamer 407;

optionally, the comparable reconstituted solution comprises unpurified Poloxamer 407.

137. The reconstituted solution of any one of the preceding clauses, wherein the reconstituted solution comprises purified Poloxamer 407, and wherein the reconstituted solution has a lower gelation temperature, a narrower gelation temperature range, a more sustained release of the hearing loss treatment agent, and/or a higher viscosity as compared to a reconstituted solution without purified Poloxamer 407;

optionally, the comparable reconstituted solution comprises unpurified Poloxamer 407.

138. The reconstituted solution of any one of the preceding clauses, wherein the reconstituted solution comprises purified Poloxamer 407, and wherein the reconstituted solution has a reduced degradation rate as compared to a comparable reconstituted solution without purified Poloxamer 407;

optionally, the comparable reconstituted solution comprises unpurified Poloxamer 407.

139. The reconstituted solution of any one of the preceding clauses, being suitable for injection;

optionally, the reconstituted solution is suitable for intratympanic injection.

140. The reconstituted solution of any one of the preceding clauses, wherein the reconstituted solution maintains one or more rheometric properties of a pharmaceutical composition which is used for preparing the lyophilized pharmaceutical composition.
141. The reconstituted solution of any one of the preceding clauses, wherein the reconstituted solution has a reduced degradation rate as compared to a reconstituted solution prepared from a comparable lyophilized pharmaceutical composition without purified Poloxamer 407;

optionally, the comparable lyophilized pharmaceutical composition comprises unpurified Poloxamer 407.

142. The reconstituted solution of any one of the preceding clauses, comprising one or more of water or a buffering agent;

a bulking agent;

a stabilizing agent;

a tonicity-adjusting agent; and

a soothing agent.

143. A method of facilitating the generation of a tissue and/or a cell, comprising delivering a pharmaceutically effective amount of the lyophilized pharmaceutical composition of any one of clauses 1-35 and 76-77, the pharmaceutical composition of any one of 36-69, or the reconstituted solution of any one of clauses 78-142 to the tissue and/or the cell.
144. A method of treating a subject who has, or is at risk of developing, a disease associated with absence or a lack of a tissue and/or a cell, comprising administering to the subject a pharmaceutically effective amount of the lyophilized pharmaceutical composition of any one of clauses 1-35 and 76-77, the pharmaceutical composition of any one of 36-69, or the reconstituted solution of any one of clauses 78-142.
145. A method of increasing a population of vestibular cells in a vestibular tissue, comprising delivering a pharmaceutically effective amount of the lyophilized pharmaceutical composition of any one of clauses 1-35 and 76-77, the pharmaceutical composition of any one of 36-69, or the reconstituted solution of any one of clauses 78-142.
146. A method of treating a subject who has, or is at risk of developing a vestibular condition, comprising administering to the subject a pharmaceutically effective amount of the lyophilized pharmaceutical composition of any one of clauses 1-35 and 76-77, the pharmaceutical composition of any one of 36-69, or the reconstituted solution of any one of clauses 78-142.
147. A method of increasing a population of cochlear cells in a cochlear tissue, comprising delivering a pharmaceutically effective amount of the lyophilized pharmaceutical composition of any one of clauses 1-35 and 76-77, the pharmaceutical composition of any one of 36-69, or the reconstituted solution of any one of clauses 78-142.
148. A method of treating a subject who has, or is at risk of developing a cochlear condition, comprising administering to the subject a pharmaceutically effective amount of the lyophilized pharmaceutical composition of any one of clauses 1-35 and 76-77, the pharmaceutical composition of any one of 36-69, or the reconstituted solution of any one of clauses 78-142.
149. A method of increasing a population of cells found in the Organ of Corti, comprising delivering a pharmaceutically effective amount of the lyophilized pharmaceutical composition of any one of clauses 1-35 and 76-77, the pharmaceutical composition of any one of 36-69, or the reconstituted solution of any one of clauses 78-142 to the population.
150. A method of increasing a population of hair cells found in the Organ of Corti, comprising delivering a pharmaceutically effective amount of the lyophilized pharmaceutical composition of any one of clauses 1-35 and 76-77, the pharmaceutical composition of any one of 36-69, or the reconstituted solution of any one of clauses 78-142 to the population.
151. A method of increasing a population of inner hair cells found in the Organ of Corti, comprising delivering a pharmaceutically effective amount of the lyophilized pharmaceutical composition of any one of clauses 1-35 and 76-77, the pharmaceutical composition of any one of 36-69, or the reconstituted solution of any one of clauses 78-142 to the population.
152. A method of increasing a population of outer hair cells found in the Organ of Corti, comprising delivering a pharmaceutically effective amount of the lyophilized pharmaceutical composition of any one of clauses 1-35 and 76-77, the pharmaceutical composition of any one of 36-69, or the reconstituted solution of any one of clauses 78-142 to the population.
153. A method of increasing a population of neuronal cells found in the Organ of Corti, comprising delivering a pharmaceutically effective amount of the lyophilized pharmaceutical composition of any one of clauses 1-35 and 76-77, the pharmaceutical composition of any one of 36-69, or the reconstituted solution of any one of clauses 78-142 to the population.
154. A method of treating a subject who has, or is at risk of developing a hearing condition, comprising administering to the subject a pharmaceutically effective amount of the lyophilized pharmaceutical composition of any one of clauses 1-35 and 76-77, the pharmaceutical composition of any one of 36-69, or the reconstituted solution of any one of clauses 78-142.
155. The lyophilized pharmaceutical composition of any one of clauses 1-35 and 76-77, the pharmaceutical composition of any one of 36-69, or the reconstituted solution of any one of clauses 78-142, for use in facilitating the generation of a tissue and/or a cell.
156. The lyophilized pharmaceutical composition of any one of clauses 1-35 and 76-77, the pharmaceutical composition of any one of 36-69, or the reconstituted solution of any one of clauses 78-142, for use in in treating a subject who has, or is at risk of developing, a disease associated with absence or a lack of a tissue and/or a cell.
157. The lyophilized pharmaceutical composition of any one of clauses 1-35 and 76-77, the pharmaceutical composition of any one of 36-69, or the reconstituted solution of any one of clauses 78-142, for use in increasing a population of vestibular cells in a vestibular tissue.
158. The lyophilized pharmaceutical composition of any one of clauses 1-35 and 76-77, the pharmaceutical composition of any one of 36-69, or the reconstituted solution of any one of clauses 78-142, for use in treating a subject who has, or is at risk of developing a vestibular condition.
159. The lyophilized pharmaceutical composition of any one of clauses 1-35 and 76-77, the pharmaceutical composition of any one of 36-69, or the reconstituted solution of any one of clauses 78-142, for use in increasing a population of cochlear cells in a cochlear tissue.
160. The lyophilized pharmaceutical composition of any one of clauses 1-35 and 76-77, the pharmaceutical composition of any one of 36-69, or the reconstituted solution of any one of clauses 78-142, for use in treating a subject who has, or is at risk of developing a cochlear condition.
161. The lyophilized pharmaceutical composition of any one of clauses 1-35 and 76-77, the pharmaceutical composition of any one of 36-69, or the reconstituted solution of any one of clauses 78-142, for use in increasing a population of cells found in the Organ of Corti.
162. The lyophilized pharmaceutical composition of any one of clauses 1-35 and 76-77, the pharmaceutical composition of any one of 36-69, or the reconstituted solution of any one of clauses 78-142, for use in increasing a population of hair cells found in the Organ of Corti.
163. The lyophilized pharmaceutical composition of any one of clauses 1-35 and 76-77, the pharmaceutical composition of any one of 36-69, or the reconstituted solution of any one of clauses 78-142, for use in increasing a population of inner hair cells found in the Organ of Corti.
164. The lyophilized pharmaceutical composition of any one of clauses 1-35 and 76-77, the pharmaceutical composition of any one of 36-69, or the reconstituted solution of any one of clauses 78-142, for use in increasing a population of outer hair cells found in the Organ of Corti.
165. The lyophilized pharmaceutical composition of any one of clauses 1-35 and 76-77, the pharmaceutical composition of any one of 36-69, or the reconstituted solution of any one of clauses 78-142, for use in increasing a population of neuronal cells found in the Organ of Corti.
166. The lyophilized pharmaceutical composition of any one of clauses 1-35 and 76-77, the pharmaceutical composition of any one of 36-69, or the reconstituted solution of any one of clauses 78-142, for use in treating a subject who has, or is at risk of developing a hearing condition 167. Use of the lyophilized pharmaceutical composition of any one of clauses 1-35 and 76-77, the pharmaceutical composition of any one of 36-69, or the reconstituted solution of any one of clauses 78-142, in the manufacture of a medicament for facilitating the generation of a tissue and/or a cell.
168. Use of the lyophilized pharmaceutical composition of any one of clauses 1-35 and 76-77, the pharmaceutical composition of any one of 36-69, or the reconstituted solution of any one of clauses 78-142, in the manufacture of a medicament for in treating a subject who has, or is at risk of developing, a disease associated with absence or a lack of a tissue and/or a cell.
169. Use of the lyophilized pharmaceutical composition of any one of clauses 1-35 and 76-77, the pharmaceutical composition of any one of 36-69, or the reconstituted solution of any one of clauses 78-142, in the manufacture of a medicament for increasing a population of vestibular cells in a vestibular tissue.
170. Use of the lyophilized pharmaceutical composition of any one of clauses 1-35 and 76-77, the pharmaceutical composition of any one of 36-69, or the reconstituted solution of any one of clauses 78-142, in the manufacture of a medicament for treating a subject who has, or is at risk of developing a vestibular condition 171. Use of the lyophilized pharmaceutical composition of any one of clauses 1-35 and 76-77, the pharmaceutical composition of any one of 36-69, or the reconstituted solution of any one of clauses 78-142, in the manufacture of a medicament for increasing a population of cochlear cells in a cochlear tissue.
172. Use of the lyophilized pharmaceutical composition of any one of clauses 1-35 and 76-77, the pharmaceutical composition of any one of 36-69, or the reconstituted solution of any one of clauses 78-142, in the manufacture of a medicament for treating a subject who has, or is at risk of developing a cochlear condition.
173. Use of the lyophilized pharmaceutical composition of any one of clauses 1-35 and 76-77, the pharmaceutical composition of any one of 36-69, or the reconstituted solution of any one of clauses 78-142, in the manufacture of a medicament for increasing a population of cells found in the Organ of Corti.
174. Use of the lyophilized pharmaceutical composition of any one of clauses 1-35 and 76-77, the pharmaceutical composition of any one of 36-69, or the reconstituted solution of any one of clauses 78-142, in the manufacture of a medicament for increasing a population of hair cells found in the Organ of Corti.
175. Use of the lyophilized pharmaceutical composition of any one of clauses 1-35 and 76-77, the pharmaceutical composition of any one of 36-69, or the reconstituted solution of any one of clauses 78-142, in the manufacture of a medicament for increasing a population of inner hair cells found in the Organ of Corti.
176. Use of the lyophilized pharmaceutical composition of any one of clauses 1-35 and 76-77, the pharmaceutical composition of any one of 36-69, or the reconstituted solution of any one of clauses 78-142, in the manufacture of a medicament for increasing a population of outer hair cells found in the Organ of Corti.
177. Use of the lyophilized pharmaceutical composition of any one of clauses 1-35 and 76-77, the pharmaceutical composition of any one of 36-69, or the reconstituted solution of any one of clauses 78-142, in the manufacture of a medicament for increasing a population of neuronal cells found in the Organ of Corti.
178. Use of the lyophilized pharmaceutical composition of any one of clauses 1-35 and 76-77, the pharmaceutical composition of any one of 36-69, or the reconstituted solution of any one of clauses 78-142, in the manufacture of a medicament for treating a subject who has, or is at risk of developing a hearing condition.

The disclosure also includes the following numbered embodiments.
1. A pharmaceutical composition comprising a gelling agent and a compound of formula (I):

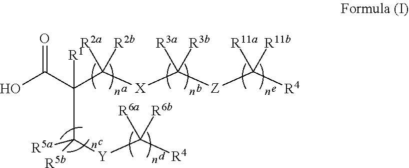

or a pharmaceutically acceptable salt thereof,
wherein:
R¹ is selected from H, alkyl, alkoxy, halo, cycloalkyl, alkenyl, alkynyl, carbocyclyl, and aryl;
R^{2a} is independently selected from H, alkyl, alkoxy, halo, cycloalkyl, alkenyl, alkynyl, carbocyclyl, and aryl;
stop R^{2b} is independently selected from H, alkyl, alkoxy, halo, cycloalkyl, alkenyl, alkynyl, carbocyclyl, and aryl;
X is selected from

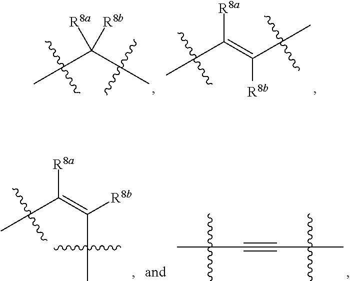

or is not present;
R^{3a} is independently selected from H, alkyl, alkoxy, halo, cycloalkyl, alkenyl, alkynyl, carbocyclyl, and aryl;
R^{3b} is independently selected from H, alkyl, alkoxy, halo, cycloalkyl, alkenyl, alkynyl, carbocyclyl, and aryl;
R⁴ is selected from H, alkyl, alkoxy, halo, cycloalkyl, alkenyl, alkynyl, carbocyclyl, and aryl;
R^{5a} is independently selected from H, alkyl, alkoxy, halo, cycloalkyl, alkenyl, alkynyl, carbocyclyl, and aryl;
R^{5b} is independently selected from H, alkyl, alkoxy, halo, cycloalkyl, alkenyl, alkynyl, carbocyclyl, and aryl;
Y is selected from

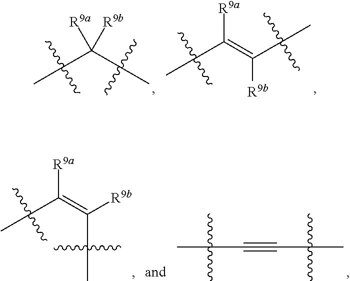

or is not present;
R^{6a} is selected from H, alkyl, alkoxy, halo, cycloalkyl, alkenyl, alkynyl, carbocyclyl, and aryl;
R^{6b} is selected from H, alkyl, alkoxy, halo, cycloalkyl, alkenyl, alkynyl, carbocyclyl, and aryl;
each R⁷ is independently selected from H, alkyl, alkoxy, halo, cycloalkyl, alkenyl, alkynyl, carbocyclyl, and aryl;
R^{8a} is independently selected from H, alkyl, alkoxy, halo, cycloalkyl, alkenyl, alkynyl, carbocyclyl, and aryl;
R^{8b} is independently selected from H, alkyl, alkoxy, halo, cycloalkyl, alkenyl, alkynyl, carbocyclyl, and aryl;
Z is selected from

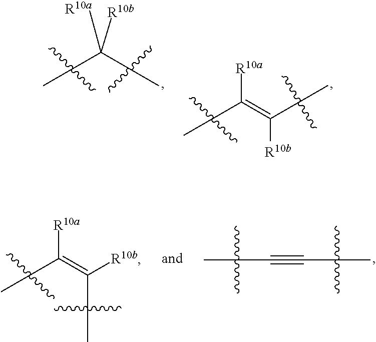

or is not present;
R^{10a} is independently selected from H, alkyl, alkoxy, halo, cycloalkyl, alkenyl, alkynyl, carbocyclyl, and aryl;
R^{10b} is independently selected from H, alkyl, alkoxy, halo, cycloalkyl, alkenyl, alkynyl, carbocyclyl, and aryl;
R^{11a} is selected from H, alkyl, alkoxy, halo, cycloalkyl, alkenyl, alkynyl, carbocyclyl, and aryl;
R^{1lb} is selected from H, alkyl, alkoxy, halo, cycloalkyl, alkenyl, alkynyl, carbocyclyl, and aryl;
n^{a} is selected from 0, 1, 2, 3, 4, 5, 6, 7, and 8;
n^{b} is selected from 0, 1, 2, 3, and 4;
n^{c} is selected from 0, 1, and 2;
n^{d} is selected from 0, 1, and 2; and;
if is selected from 0, 1, 2, 3, 4, 5, and 6.
2. The pharmaceutical composition of embodiment 1, wherein the pharmaceutical composition is a lyophilized composition
3. A lyophilized pharmaceutical composition comprising one or more otic therapeutic agents and a gelling agent
4. The composition of embodiment 3, wherein the one or more otic therapeutic agents includes a compound of formula (I).
5. The composition of embodiments 1 or 2, wherein the compound of formula (I) is an otic therapeutic agent
6. The composition of any preceding embodiment, wherein R¹ is H.
7. The composition of any preceding embodiment, wherein R¹ is alkyl.
8. The composition of any preceding embodiment, wherein R¹ is alkoxy.
9. The composition of any preceding embodiment, wherein R¹ is halo.
10. The composition of any preceding embodiment, wherein R¹ is cycloalkyl.
11. The composition of any preceding embodiment, wherein R¹ is alkenyl.
12. The composition of any preceding embodiment, wherein R¹ is alkynyl.
13. The composition of any preceding embodiment, wherein R¹ is carbocyclyl.
14. The composition of any preceding embodiment, wherein R¹ is awl.
15. The composition of any preceding embodiment, wherein R^{2a} is H.
16. The composition of any preceding embodiment, wherein R^{2a} is alkyl.
17. The composition of any preceding embodiment, wherein R^{2a} is alkoxy.
18. The composition of any preceding embodiment, wherein R^{2a} is halo.
19. The composition of any preceding embodiment, wherein R^{2a} is cycloalkyl.
20. The composition of any preceding embodiment, wherein R^{2a} is alkenyl.
21. The composition of any preceding embodiment, wherein R^{2a} is alkynyl.
22. The composition of any preceding embodiment, wherein R^{2a} is carbocyclyl.
23. The composition of any preceding embodiment, wherein R^{2a} is aryl.
24. The composition of any preceding embodiment, wherein R^{2b} is H.
25. The composition of any preceding embodiment, wherein R^{2b} is alkyl.
26. The composition of any preceding embodiment, wherein R^{2b} is alkoxy.
27. The composition of any preceding embodiment, wherein R^{2b} is halo.
28. The composition of any preceding embodiment, wherein R^{2b} is cycloalkyl.
29. The composition of any preceding embodiment, wherein R^{2b} is alkenyl.
30. The composition of any preceding embodiment, wherein R^{2b} is alkynyl.
31. The composition of any preceding embodiment, wherein R^{2b} is carbocyclyl.
32. The composition of any preceding embodiment, wherein R^{2b} is aryl.
33. The composition of any preceding embodiment, wherein R^{3a} is H.
34. The composition of any preceding embodiment, wherein R^{3a} is alkyl.
35. The composition of any preceding embodiment, wherein R^{3a} is alkoxy.
36. The composition of any preceding embodiment, wherein R^{3a} is halo.
37. The composition of any preceding embodiment, wherein R^{3a} is cycloalkyl.
38. The composition of any preceding embodiment, wherein R^{3a} is alkenyl.
39. The composition of any preceding embodiment, wherein R^{3a} is alkynyl.
40. The composition of any preceding embodiment, wherein R^{3a} is carbocyclyl.
41. The composition of any preceding embodiment, wherein R^{3a} is aryl.
42. The composition of any preceding embodiment, wherein R^{3b} is H.
43. The composition of any preceding embodiment, wherein R^{3b} is alkyl.
44. The composition of any preceding embodiment, wherein R^{3b} is alkoxy.
45. The composition of any preceding embodiment, wherein R^{3b} is halo.
46. The composition of any preceding embodiment, wherein R^{3b} is cycloalkyl.
47. The composition of any preceding embodiment, wherein R^{3b} is alkenyl.
48. The composition of any preceding embodiment, wherein R^{3b} is alkynyl.
49. The composition of any preceding embodiment, wherein R^{3b} is carbocyclyl.
50. The composition of any preceding embodiment, wherein R^{3b} is aryl.
51. The composition of any preceding embodiment, wherein R⁴ is H.
52. The composition of any preceding embodiment, wherein R⁴ is alkyl.
53. The composition of any preceding embodiment, wherein R⁴ is alkoxy.
54. The composition of any preceding embodiment, wherein R⁴ is halo.
55. The composition of any preceding embodiment, wherein R⁴ is cycloalkyl.
56. The composition of any preceding embodiment, wherein R⁴ is alkenyl.
57. The composition of any preceding embodiment, wherein R⁴ is alkynyl.
58. The composition of any preceding embodiment, wherein R⁴ is carbocyclyl.
59. The composition of any preceding embodiment, wherein R⁴ is awl.
60. The composition of any preceding embodiment, wherein R^{5a} is H.
61. The composition of any preceding embodiment, wherein R^{5a} is alkyl.
62. The composition of any preceding embodiment, wherein R^{5a} is alkoxy.
63. The composition of any preceding embodiment, wherein R^{5a} is halo.
64. The composition of any preceding embodiment, wherein R^{5a} is cycloalkyl.
65. The composition of any preceding embodiment, wherein R^{5a} is alkenyl.
66. The composition of any preceding embodiment, wherein R^{5a} is alkynyl.
67. The composition of any preceding embodiment, wherein R^{5a} is carbocyclyl.
68. The composition of any preceding embodiment, wherein R^{5a} is aryl.
69. The composition of any preceding embodiment, wherein R^{5b} is H.
70. The composition of any preceding embodiment, wherein R^{5b} is alkyl.
71. The composition of any preceding embodiment, wherein R^{5b} is alkoxy.
72. The composition of any preceding embodiment, wherein R^{5b} is halo.
73. The composition of any preceding embodiment, wherein R^{5b} is cycloalkyl.
74. The composition of any preceding embodiment, wherein R^{5b} is alkenyl.
75. The composition of any preceding embodiment, wherein R^{5b} is alkynyl.
76. The composition of any preceding embodiment, wherein R^{5b} is carbocyclyl.
77. The composition of any preceding embodiment, wherein R^{5b} is aryl.
78. The composition of any preceding embodiment, wherein R^{6a} is H.
79. The composition of any preceding embodiment, wherein R^{6a} is alkyl.
80. The composition of any preceding embodiment, wherein R^{6a} is alkoxy.
81. The composition of any preceding embodiment, wherein R^{6a} is halo.
82. The composition of any preceding embodiment, wherein R^{6a} is cycloalkyl.
83. The composition of any preceding embodiment, wherein R^{6a} is alkenyl.
84. The composition of any preceding embodiment, wherein R^{6a} is alkynyl.
85. The composition of any preceding embodiment, wherein R^{6a} is carbocyclyl.
86. The composition of any preceding embodiment, wherein R^{6a} is aryl.
87. The composition of any preceding embodiment, wherein R^{6b} is H. 88. The composition of any preceding embodiment, wherein R^{6b} is alkyl.
89. The composition of any preceding embodiment, wherein R^{6b} is alkoxy.
90. The composition of any preceding embodiment, wherein R^{6b} is halo.
91. The composition of any preceding embodiment, wherein R^{6b} is cycloalkyl.
92. The composition of any preceding embodiment, wherein R^{6b} is alkenyl.
93. The composition of any preceding embodiment, wherein R^{6b} is alkynyl.
94. The composition of any preceding embodiment, wherein R^{6b} is carbocyclyl.
95. The composition of any preceding embodiment, wherein R^{6b} is aryl.
96. The composition of any preceding embodiment, wherein R⁷ is H.
97. The composition of any preceding embodiment, wherein R⁷ is alkyl.
98. The composition of any preceding embodiment, wherein R⁷ is alkoxy.
99. The composition of any preceding embodiment, wherein R⁷ is halo.
100. The composition of any preceding embodiment, wherein R⁷ is cycloalkyl.
101. The composition of any preceding embodiment, wherein R⁷ is alkenyl.
102. The composition of any preceding embodiment, wherein R⁷ is alkynyl.
103. The composition of any preceding embodiment, wherein R⁷ is carbocyclyl.
104. The composition of any preceding embodiment, wherein R⁷ is awl.
105. The composition of any preceding embodiment, wherein X is

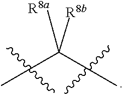

106. The composition of any preceding embodiment, wherein X is

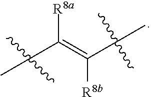

107. The composition of any preceding embodiment, wherein X is

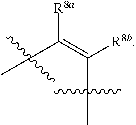

108. The composition of any preceding embodiment, wherein X is

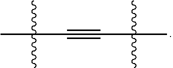 [SMILES: *C#C*]

109. The composition of any preceding embodiment, wherein Xis not present
110. The composition of any preceding embodiment, wherein Y is

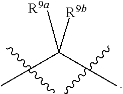

111. The composition of any preceding embodiment, wherein Y is

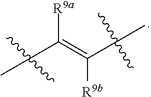

112. The composition of any preceding embodiment, wherein Y is

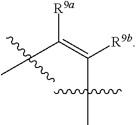

113. The composition of any preceding embodiment, wherein Y is

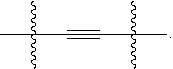 [SMILES: *C#C*]

114. The composition of any preceding embodiment, wherein Y is not present
115. The composition of any preceding embodiment, wherein R^{8a} is H.
116. The composition of any preceding embodiment, wherein R^{8a} is alkyl.
117. The composition of any preceding embodiment, wherein R^{8a} is alkoxy.
118. The composition of any preceding embodiment, wherein R₈ₐ is halo.
119. The composition of any preceding embodiment, wherein R^{8a} is cycloalkyl.
120. The composition of any preceding embodiment, wherein R^{8a} is alkenyl.
121. The composition of any preceding embodiment, wherein R^{8a} is alkynyl.
122. The composition of any preceding embodiment, wherein R^{8a} is carbocyclyl.
123. The composition of any preceding embodiment, wherein R^{8a} is aryl.
124. The composition of any preceding embodiment, wherein R^{8b} is H.
125. The composition of any preceding embodiment, wherein R^{8b} is alkyl.
126. The composition of any preceding embodiment, wherein R^{8b} is alkoxy.
127. The composition of any preceding embodiment, wherein R^{8b} is halo.
128. The composition of any preceding embodiment, wherein R^{8b} is cycloalkyl.
129. The composition of any preceding embodiment, wherein R^{8b} is alkenyl.
130. The composition of any preceding embodiment, wherein R^{8b} is alkynyl.
131. The composition of any preceding embodiment, wherein R^{8b} is carbocyclyl.
132. The composition of any preceding embodiment, wherein R^{8b} is aryl.
133. The composition of any preceding embodiment, wherein R^{9a} is H.
134. The composition of any preceding embodiment, wherein R^{9a} is alkyl.
135. The composition of any preceding embodiment, wherein R^{9a} is alkoxy.
136. The composition of any preceding embodiment, wherein R^{9a} is halo.
137. The composition of any preceding embodiment, wherein R^{9a} is cycloalkyl.
138. The composition of any preceding embodiment, wherein R^{9a} is alkenyl.
139. The composition of any preceding embodiment, wherein R^{9a} is alkynyl.
140. The composition of any preceding embodiment, wherein R^{9a} is carbocyclyl.
141. The composition of any preceding embodiment, wherein R^{9a} is aryl.
142. The composition of any preceding embodiment, wherein R^{9b} is H.
143. The composition of any preceding embodiment, wherein R^{9b} is alkyl.
144. The composition of any preceding embodiment, wherein R^{9b} is alkoxy.
145. The composition of any preceding embodiment, wherein R^{9b} is halo.
146. The composition of any preceding embodiment, wherein R^{9b} is cycloalkyl.
147. The composition of any preceding embodiment, wherein R^{9b} is alkenyl.
148. The composition of any preceding embodiment, wherein R^{9b} is alkynyl.
149. The composition of any preceding embodiment, wherein R^{9b} is carbocyclyl.
150. The composition of any preceding embodiment, wherein R^{9b} is aryl.
151. The composition of any preceding embodiment, wherein Z is

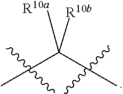

152. The composition of any preceding embodiment, wherein Z

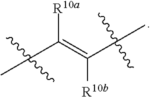

153. The composition of any preceding embodiment, wherein Z is

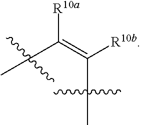

154. The composition of any preceding embodiment, wherein Z is

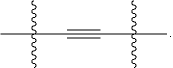 [SMILES: *C#C*]

155. The composition of any preceding embodiment, wherein Z is not present
156. The composition of any preceding embodiment, wherein R^{10a} is H.
157. The composition of any preceding embodiment, wherein R^{10a} is alkyl.
158. The composition of any preceding embodiment, wherein R^{10a} is alkoxy.
159. The composition of any preceding embodiment, wherein R^{10a} is halo.
160. The composition of any preceding embodiment, wherein R^{10a} is cycloalkyl.
161. The composition of any preceding embodiment, wherein R^{10a} is alkenyl.
162. The composition of any preceding embodiment, wherein R^{10a} is alkynyl.
163. The composition of any preceding embodiment, wherein R^{10a} is carbocyclyl.
164. The composition of any preceding embodiment, wherein R^{10a} is aryl.
165. The composition of any preceding embodiment, wherein R^{10b} is H.
166. The composition of any preceding embodiment, wherein R^{10b} is alkyl.
167. The composition of any preceding embodiment, wherein R^{10b} is alkoxy.
168. The composition of any preceding embodiment, wherein R^{10b} is halo.
169. The composition of any preceding embodiment, wherein R^{10b} is cycloalkyl.
170. The composition of any preceding embodiment, wherein R^{10b} is alkenyl.
171. The composition of any preceding embodiment, wherein R^{10b} is alkynyl.
172. The composition of any preceding embodiment, wherein R^{10b} is carbocyclyl.
173. The composition of any preceding embodiment, wherein R^{10b} is aryl.
174. The composition of any preceding embodiment, wherein R^{11a} is H.
175. The composition of any preceding embodiment, wherein R^{11b} is alkyl.
176. The composition of any preceding embodiment, wherein R^{11a} is alkoxy.
177. The composition of any preceding embodiment, wherein R^{11a} is halo.
178. The composition of any preceding embodiment, wherein R^{11a} is cycloalkyl.
179. The composition of any preceding embodiment, wherein R^{11a} is alkenyl.
180. The composition of any preceding embodiment, wherein R^{11a} is alkynyl.
181. The composition of any preceding embodiment, wherein R^{11a} is carbocyclyl.
182. The composition of any preceding embodiment, wherein R^{11a} is aryl.
183. The composition of any preceding embodiment, wherein R^{11b} is H.
184. The composition of any preceding embodiment, wherein R^{11b} is alkyl.
185. The composition of any preceding embodiment, wherein R^{11b} is alkoxy.
186. The composition of any preceding embodiment, wherein R^{11b} is halo.
187. The composition of any preceding embodiment, wherein R^{11b} is cycloalkyl.
188. The composition of any preceding embodiment, wherein R^{11b} is alkenyl.
189. The composition of any preceding embodiment, wherein R^{11b} is alkynyl.
190. The composition of any preceding embodiment, wherein R^{11b} is carbocyclyl.
191. The composition of any preceding embodiment, wherein R^{11b} is aryl.
192. The composition of any preceding embodiment, wherein n^{a} is O.
193. The composition of any preceding embodiment, wherein n^{a} is 1.
194. The composition of any preceding embodiment, wherein n^{a} is 2.
195. The composition of any preceding embodiment, wherein n^{a} is 3.
196. The composition of any preceding embodiment, wherein n^{a} is 4.
197. The composition of any preceding embodiment, wherein n^{a} is 5.
198. The composition of any preceding embodiment, wherein n^{a} is 6.
199. The composition of any preceding embodiment, wherein n^{a} is 7.
200. The composition of any preceding embodiment, wherein n^{a} is 8.
201. The composition of any preceding embodiment, wherein n^{b} is 0.
202. The composition of any preceding embodiment, wherein n^{b} is 1.
203. The composition of any preceding embodiment, wherein n^{b} is 2.
204. The composition of any preceding embodiment, wherein n^{b} is 3.
205. The composition of any preceding embodiment, wherein n^{b} is 4.
206. The composition of any preceding embodiment, wherein n^{c} is 0.
207. The composition of any preceding embodiment, wherein n^{c} is 1.
208. The composition of any preceding embodiment, wherein n^{c} is 2.
209. The composition of any preceding embodiment, wherein n^{d} is 0.
210. The composition of any preceding embodiment, wherein n^{d} is 1.
211. The composition of any preceding embodiment, wherein n^{d} is 2.
212. The composition of any preceding embodiment, wherein n^{e} is 0.
213. The composition of any preceding embodiment, wherein n^{e} is 1.
214. The composition of any preceding embodiment, wherein n^{e} is 2.
215. The composition of any preceding embodiment, wherein n^{e} is 3.
216. The composition of any preceding embodiment, wherein n^{e} is 4.
217. The composition of any preceding embodiment, wherein n^{e} is 5.
218. The composition of any preceding embodiment, wherein n^{e} is 6.
219. The composition of any preceding embodiment, wherein R¹ is Me.
220. The composition of any preceding embodiment, wherein R^{2a} is Me.
221. The composition of any preceding embodiment, wherein R^{2b} is Me.
222. The composition of any preceding embodiment, wherein R^{3a} is Me.
223. The composition of any preceding embodiment, wherein R^{3b} is Me.
224. The composition of any preceding embodiment, wherein R⁴ is Me.
225. The composition of any preceding embodiment, wherein R^{5a} is Me.
226. The composition of any preceding embodiment, wherein R^{5b} is Me.
227. The composition of any preceding embodiment, wherein R^{6a} is Me.
228. The composition of any preceding embodiment, wherein R^{6b} is Me.
229. The composition of any preceding embodiment, wherein R⁷ is Me.
230. The composition of any preceding embodiment, wherein R^{8a} is Me.
231. The composition of any preceding embodiment, wherein R^{8b} is Me.
232. The composition of any preceding embodiment, wherein R^{9a} is Me.
233. The composition of any preceding embodiment, wherein R^{9b} is Me.
234. The composition of any preceding embodiment, wherein R^{10a} is Me.
235. The composition of any preceding embodiment, wherein R^{10b} is Me.
236. The composition of any preceding embodiment, wherein R^{11a} is Me.
237. The composition of any preceding embodiment, wherein R^{11b} is Me.
238. The composition of any preceding embodiment, wherein R¹ is F.
239. The composition of any preceding embodiment, wherein R^{2a} is F.
240. The composition of any preceding embodiment, wherein R^{2b} is F.
241. The composition of any preceding embodiment, wherein R^{3a} is F.
242. The composition of any preceding embodiment, wherein R^{3b} is F.
243. The composition of any preceding embodiment, wherein R⁴ is F.
244. The composition of any preceding embodiment, wherein R^{5a} is F.
245. The composition of any preceding embodiment, wherein R^{5b} is F.
246. The composition of any preceding embodiment, wherein R^{6a} is F.
247. The composition of any preceding embodiment, wherein R^{8b} is F.
248. The composition of any preceding embodiment, wherein R⁷ is F.
249. The composition of any preceding embodiment, wherein R^{8a} is F.
250. The composition of any preceding embodiment, wherein R^{8b} is F.
251. The composition of any preceding embodiment, wherein R^{9a} is F.
252. The composition of any preceding embodiment, wherein R^{9b} is F.
253. The composition of any preceding embodiment, wherein R^{10a} is F.
254. The composition of any preceding embodiment, wherein R^{10b} is F.
255. The composition of any preceding embodiment, wherein R^{11a} is F.
256. The composition of any preceding embodiment, wherein R^{11b} is F.
257. The composition of any preceding embodiment, wherein R¹ is alkyl.
258. The composition of any preceding embodiment, wherein R^{2a} is alkyl.
259. The composition of any preceding embodiment, wherein R^{2b} is alkyl.
260. The composition of any preceding embodiment, wherein R^{3a} is alkyl.
261. The composition of any preceding embodiment, wherein R^{ab} is alkyl.
262. The composition of any preceding embodiment, wherein R⁴ is alkyl.
263. The composition of any preceding embodiment, wherein R^{5a} is alkyl.
264. The composition of any preceding embodiment, wherein R^{5b} is alkyl.
265. The composition of any preceding embodiment, wherein R^{6a} is alkyl.
266. The composition of any preceding embodiment, wherein R^{6b} is alkyl.
267. The composition of any preceding embodiment, wherein R⁷ is alkyl.
268. The composition of any preceding embodiment, wherein R^{8a} is alkyl.
269. The composition of any preceding embodiment, wherein R^{8b} is alkyl.
270. The composition of any preceding embodiment, wherein R^{9a} is alkyl.
271. The composition of any preceding embodiment, wherein R^{9b} is alkyl.
272. The composition of any preceding embodiment, wherein R^{10a} is alkyl.
273. The composition of any preceding embodiment, wherein R^{10b} is alkyl.
274. The composition of any preceding embodiment, wherein R^{11a} is alkyl.
275. The composition of any preceding embodiment, wherein R^{11b} is alkyl.
276. The composition of any preceding embodiment, wherein alkyl is methyl.
277. The composition of any preceding embodiment, wherein alkyl is ethyl.
278. The composition of any preceding embodiment, wherein alkyl is n-propyl.
279. The composition of any preceding embodiment, wherein alkyl is iso-propyl.
280. The composition of any preceding embodiment, wherein alkyl is n-butyl.
281. The composition of any preceding embodiment, wherein alkyl is sec-butyl.
282. The composition of any preceding embodiment, wherein alkyl is iso-butyl.
283. The composition of any preceding embodiment, wherein alkyl is tert-butyl.
284. The composition of any preceding embodiment, wherein alkoxy is methoxy.
285. The composition of any preceding embodiment, wherein alkoxy is ethoxy.
286. The composition of any preceding embodiment, wherein alkoxy is n-propoxy.
287. The composition of any preceding embodiment, wherein alkoxy is iso-propoxy.
288. The composition of any preceding embodiment, wherein alkoxy is n-butoxy.
289. The composition of any preceding embodiment, wherein alkoxy is sec-butoxy.
290. The composition of any preceding embodiment, wherein alkoxy is iso-butoxy.
291. The composition of any preceding embodiment, wherein alkoxy is tert-butoxy.
292. The composition of any preceding embodiment, wherein halo is F.
293. The composition of any preceding embodiment, wherein halo is Cl.
294. The composition of any preceding embodiment, wherein halo is Br.
295. The composition of any preceding embodiment, wherein halo is I.
296. The composition of any preceding embodiment, wherein cycloalkyl is cyclopropyl.
297. The composition of any preceding embodiment, wherein cycloalkyl is cyclobutyl.
298. The composition of any preceding embodiment, wherein cycloalkyl is cyclopentyl.
299. The composition of any preceding embodiment, wherein cycloalkyl is cyclohexyl.
300. The composition of any preceding embodiment, wherein aryl is phenyl.
301. The composition of any preceding embodiment, wherein aryl is tolyl.
302. The composition of any preceding embodiment, wherein aryl is xylyl.
303. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with methyl.
304. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with ethyl.
305. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with n-propyl.
306. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with iso-propyl.
307. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with n-butyl.
308. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with sec-butyl.
309. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with iso-butyl.
310. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with tert-butyl.
311. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with methoxy.
312. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with ethoxy.
313. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with n-propoxy.
314. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b}, is further substituted with iso-propoxy.
315. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with n-butoxy.
316. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with sec-butoxy.
317. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with iso-butoxy.
318. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with tert-butoxy.
319. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with F.
320. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with Cl.
321. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with Br.
322. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with I.
323. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with cycloalkyl.
324. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with alkenyl.
325. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with alkynyl.
326. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with carbocyclyl.
327. The composition of any preceding embodiment, wherein one of R¹, R^{2a}, R^{2b}, R^{3a}, R^{3b}, R⁴, R^{5a}, R^{5b}, R^{6a}, R^{6b}, R⁷, R^{8a}, R^{8b}, R^{9a}, R^{9b}, R^{10a}, R^{10b}, R^{11a}, and R^{11b} is further substituted with aryl.
328. The composition of embodiment 1, wherein the compound of formula (I) is valproic acid or a pharmaceutically acceptable salt thereof 329. The composition of embodiment 1, wherein the compound of formula (I) is 2-(prop-2-yn-1-yl)-octanoic acid or a pharmaceutically acceptable salt thereof 330. The composition of embodiment 1, wherein the compound of formula (I) is phenylbutyric acid or a pharmaceutically acceptable salt thereof 331. The composition of embodiment 1, wherein the compound of formula (I) is linoleic acid or a pharmaceutically acceptable salt thereof 332. The composition of any preceding embodiment, wherein the compound of formula (I) is an HDAC inhibitor.
333. A pharmaceutical composition comprising a gelling agent, valproic acid or a pharmaceutically acceptable salt thereof at a concentration of greater than about 70 mg/ml, and one or more otic therapeutic agents.
334. A composition of any preceding embodiment, wherein the composition is suitable for intratympanic injection 335. The composition of any preceding embodiment, wherein the gelling agent is a poloxamer.
336. A pharmaceutical composition comprising a poloxamer, wherein at least 85% by wt % of the poloxamer has an average molecular weight of greater than about 7250 Da, and valproic acid or a pharmaceutically acceptable salt thereof at greater than 70 mg/ml.
337. The composition of any preceding embodiment, wherein at least 85% by weight of the poloxamer has an average molecular weight of about 7250 to about 17350 Da.
338. The composition of any preceding embodiment, wherein at least 86% by weight of the poloxamer has an average molecular weight of about 7250 to about 17350 Da.
339. The composition of any preceding embodiment, wherein at least 87% by weight of the poloxamer has an average molecular weight of about 7250 to about 17350 Da.
340. The composition of any preceding embodiment, wherein at least 88% by weight of the poloxamer has an average molecular weight of about 7250 to about 17350 Da.
341. The composition of any preceding embodiment, wherein at least 89% by weight of the poloxamer has an average molecular weight of about 7250 to about 17350 Da.
342. The composition of any preceding embodiment, wherein at least 90% by weight of the poloxamer has an average molecular weight of about 7250 to about 17350 Da.
343. The composition of any preceding embodiment, wherein at least 86% by weight of the poloxamer has an average molecular weight of greater than about 7250 Da.
344. The composition of any preceding embodiment, wherein at least 87% by weight of the poloxamer has an average molecular weight of greater than about 7250 Da.
345. The composition of any preceding embodiment, wherein at least 88% by weight of the poloxamer has an average molecular weight of greater than about 7250 Da.
346. The composition of any preceding embodiment, wherein at least 89% by weight of the poloxamer has an average molecular weight of greater than about 7250 Da.
347. The composition of any preceding embodiment, wherein at least 90% by weight of the poloxamer has an average molecular weight of greater than about 7250 Da.
348. The composition of any preceding embodiment, wherein at least 91% by weight of the poloxamer has an average molecular weight of greater than about 7250 Da.
349. The composition of any preceding embodiment, wherein at least 92% by weight of the poloxamer has an average molecular weight of greater than about 7250 Da.
350. The composition of any of embodiments 337-349, wherein the poloxamer has a peak molecular weight of about 12,000 to about 12,500 Da.
351. The composition of any of embodiments 337-350, wherein the poloxamer has a peak molecular weight of about 11,500 to about 13,000 Da.
352. The composition of any of embodiments 337-351, wherein the poloxamer has a number average molecular weight of about 11,500 to about 12,000 Da.
353. The composition of any of embodiments 337-352, wherein the poloxamer has a number average molecular weight of about 11,000 to about 12,500 Da.
354. The composition of any of embodiments 337-353, wherein the poloxamer has a weight average molecular weight of about 11,750 to about 12,250 Da.
355. The composition of any of embodiments 337-354, wherein the poloxamer has a weight average molecular weight of about 11,250 to about 12,750 Da.
356. The composition of any of embodiments 337-355, wherein the poloxamer has a polydispersity index of about 1.02.
357. The composition of any preceding embodiment, wherein the poloxamer comprises purified poloxamer.
358. The composition of any preceding embodiment, wherein the poloxamer is purified poloxamer.
359. The composition of any preceding embodiment, further comprising one or more otic therapeutic agents.
360. A pharmaceutical composition comprising a poloxamer, wherein less than 20% by weight of the poloxamer has an average molecular weight less about 7250 Da, and valproic acid or a pharmaceutically acceptable salt thereof at greater than 70 mg/ml.
361. The composition of any preceding embodiment, wherein less than 19% by weight of the poloxamer has an average molecular weight less about 7250 Da.
362. The composition of any preceding embodiment, wherein less than 18% by weight of the poloxamer has an average molecular weight less about 7250 Da.
363. The composition of any preceding embodiment, wherein less than 17% by weight of the poloxamer has an average molecular weight less about 7250 Da.
364. The composition of any preceding embodiment, wherein less than 16% by weight of the poloxamer has an average molecular weight less about 7250 Da.
365. The composition of any preceding embodiment, wherein less than 15% by weight of the poloxamer has an average molecular weight less about 7250 Da.
366. The composition of any preceding embodiment, wherein less than 14% by weight of the poloxamer has an average molecular weight less about 7250 Da.
367. The composition of any preceding embodiment, wherein less than 13% by weight of the poloxamer has an average molecular weight less about 7250 Da.
368. The composition of any preceding embodiment, wherein less than 12% by weight of the poloxamer has an average molecular weight less about 7250 Da.
369. The composition of any preceding embodiment, wherein less than 11% by weight of the poloxamer has an average molecular weight less about 7250 Da.
370. The composition of any preceding embodiment, wherein less than 1810% by weight of the poloxamer has an average molecular weight less about 7250 Da.
371. The composition of any preceding embodiment, wherein less than 179% by weight of the poloxamer has an average molecular weight less about 7250 Da.
372. The composition of any preceding embodiment, wherein less than 16% by weight of the poloxamer has an average molecular weight less about 7250 Da.
373. The composition of any preceding embodiment, wherein less than 15% by weight of the poloxamer has an average molecular weight less about 7250 Da.
374. The composition of any preceding embodiment, wherein less than 14% by weight of the poloxamer has an average molecular weight less about 7250 Da.
375. The composition of any preceding embodiment, wherein less than 13% by weight of the poloxamer has an average molecular weight less about 7250 Da.
376. The composition of any preceding embodiment, wherein less than 12% by weight of the poloxamer has an average molecular weight less about 7250 Da.
377. The composition of any preceding embodiment, wherein less than 11% by weight of the poloxamer has an average molecular weight less about 7250 Da.
378. The composition of any preceding embodiment, wherein less than 10% by weight of the poloxamer has an average molecular weight less about 7250 Da.
379. The composition of any preceding embodiment, wherein less than 9% by weight of the poloxamer has an average molecular weight less about 7250 Da.
380. The composition of any of embodiments 361-379, wherein the poloxamer has a peak molecular weight of about 5,000 to about 5,500 Da.
381. The composition of any of embodiments 361-380, wherein the poloxamer has a peak molecular weight of about 4,500 to about 6,000 Da.
382. The composition of any of embodiments 361-381, wherein the poloxamer has a number average molecular weight of about 5,000 to about 5,500 Da.
383. The composition of any of embodiments 361-382, wherein the poloxamer has a number average molecular weight of about 4,500 to about 6,000 Da.
384. The composition of any of embodiments 361-383, wherein the poloxamer has a weight average molecular weight of about 5,000 to about 5,500 Da.
385. The composition of any of embodiments 361-384, wherein the poloxamer has a weight average molecular weight of about 4,500 to about 6,000 Da.
386. The composition of any preceding embodiment, wherein the poloxamer comprises purified poloxamer.
387. The composition of any preceding embodiment, wherein the poloxamer is purified poloxamer.
388. The composition of any preceding embodiment, wherein the concentration of valproic acid or a pharmaceutically acceptable salt thereof is greater than about 100 mg/ml.
389. The composition of any preceding embodiment, wherein the concentration of valproic acid or a pharmaceutically acceptable salt thereof is about 100 to about 500 mg/mL.
390. The composition of any preceding embodiment, wherein the concentration of valproic acid or a pharmaceutically acceptable salt thereof is about 100 to about 350 mg/mL.
391. The composition of any preceding embodiment, wherein the concentration of valproic acid or a pharmaceutically acceptable salt thereof is about 110 to about 160 mg/ml.
392. The composition of any preceding embodiment, wherein the concentration of valproic acid or a pharmaceutically acceptable salt thereof is about 130 to about 140 mg/ml.
393. The composition of any preceding embodiment, wherein the concentration of valproic acid or a pharmaceutically acceptable salt thereof is about 125 to about 145 mg/ml.
394. The composition of any preceding embodiment, wherein the concentration of valproic acid or a pharmaceutically acceptable salt thereof is about 128 to about 138 mg/ml.
395. The composition of any preceding embodiment, wherein the concentration of valproic acid or a pharmaceutically acceptable salt thereof is about 133 mg/ml.
396. The composition of any preceding embodiment, further comprising more than one otic therapeutic agents.
397. A method for preparing a pharmaceutical composition comprising the steps of:
   (a) having an aqueous solution comprising a gelling agent; and
   (b) adding a solution of one or more otic therapeutic agents or a pharmaceutically acceptable salt thereof
398. The method of embodiment 397, wherein the aqueous solution further comprises valproic acid or a pharmaceutically acceptable salt thereof to the first solution
399. The method of embodiment 397 or 398, wherein the one or more otic therapeutic agents is CHIR99021 or a pharmaceutically acceptable salt thereof
400. The method of any preceding embodiment, wherein the one or more otic therapeutic agents is LY2090314 or a pharmaceutically acceptable salt thereof
401. The method of any preceding embodiment, wherein in step (b), the solution comprises a polar aprotic solvent 402. The method of embodiment 401, wherein in step (b), the polar aprotic solvent comprises DMSO.
403. The method of embodiment 401 or 402, wherein in step (b), the polar aprotic solvent is DMSO.
404. The method of embodiment 401, wherein in step (b), the polar aprotic solvent comprises dimethylformamide.
405. The method of embodiment 401, wherein in step (b), the polar aprotic solvent comprises dimethylacetamide.
406. The method of embodiment 401, wherein in step (b), the polar aprotic solvent comprises N-methyl-2-pyrrolidone.
407. The method of any preceding embodiment, wherein the gelling agent comprises a poloxamer.
408. A lyophilized pharmaceutical composition comprising a gelling agent and one or more otic therapeutic agents, wherein in the composition does not contain an additional bulking agent
409. A lyophilized pharmaceutical composition comprising a poloxamer and one or more otic agents, wherein in the composition does not contain an antioxidant
410. The composition of any preceding embodiment, wherein the compound of formula (I) is valproic acid or a pharmaceutically acceptable salt thereof
411. The composition of any preceding embodiment, further comprising a compound of formula (I) or a pharmaceutically acceptable salt thereof
412. The composition of any preceding embodiment, wherein the one or more otic therapeutic agents includes valproic acid or a pharmaceutically acceptable salt thereof
413. The composition of any preceding embodiment, wherein the pharmaceutically acceptable salt of valproic acid is sodium valproate.
414. The composition of any preceding embodiment, further comprising one or more otic therapeutic agents.
415. The composition of any preceding embodiment, wherein the one or more otic therapeutic agents comprise a GSK3 inhibitor.
416. The composition of any preceding embodiment, wherein the one or more otic therapeutic agents comprise an HDAC inhibitor.
417. The composition of any preceding embodiment, wherein the one or more otic therapeutic agents includes CHIR99021.
418. The composition of any preceding embodiment claim embodiment, wherein the concentration of CHIR99021 or a pharmaceutically acceptable salt thereof is less than about 10 mg/mL.
419. The composition of any preceding embodiment claim embodiment, wherein the concentration of CHIR99021 or a pharmaceutically acceptable salt thereof is less than about 7.5 mg/mL.
420. The composition of any preceding embodiment claim embodiment, wherein the concentration of CHIR99021 or a pharmaceutically acceptable salt thereof is about 3 to about 7 mg/mL.
421. The composition of any preceding embodiment claim embodiment, wherein the concentration of CHIR99021 or a pharmaceutically acceptable salt thereof is about 4 to about 6 mg/mL.
422. The composition of any preceding embodiment claim embodiment, wherein the concentration of CHIR99021 or a pharmaceutically acceptable salt thereof is about 1 to about 5 mg/mL.
423. The composition of any preceding embodiment claim embodiment, wherein the concentration of CHIR99021 or a pharmaceutically acceptable salt thereof is about 2 to about 4 mg/mL.
424. The composition of any preceding embodiment, wherein the one or more otic therapeutic agents are one or more hearing loss treatment agents.
425. The composition of any preceding embodiment, further comprising an additional otic therapeutic agent
426. The composition of any preceding embodiment, wherein the one or more otic therapeutic agents includes LY2090314 or a pharmaceutically acceptable salt thereof
427. The composition of any preceding embodiment, wherein the one or more otic therapeutic agents includes AZD1080 or a pharmaceutically acceptable salt thereof
428. The composition of any preceding embodiment, wherein the one or more otic therapeutic agents includes GSK3 XXII or a pharmaceutically acceptable salt thereof
429. The composition of any preceding embodiment, wherein the one or more otic therapeutic agents includes Compound I-7 or a pharmaceutically acceptable salt thereof
430. The composition of any preceding embodiment, wherein the one or more otic therapeutic agents includes Compound I-1 or a pharmaceutically acceptable salt thereof
431. The composition of any preceding embodiment, wherein the gelling agent comprises a thermoreversible gelling agent
432. The composition of any preceding embodiment, wherein the thermoreversible gelling agent comprises sodium hyaluronate.
433. The composition of any preceding embodiment, wherein the thermoreversible gelling agent comprises a cellulosic derivative.
434. The composition of any preceding embodiment, wherein the thermoreversible gelling agent comprises a poloxamer.
435. The composition of any preceding embodiment, wherein the poloxamer comprises a polyethylene oxide-polypropylene oxide-polyethylene oxide triblock copolymer.
436. The composition of any preceding embodiment, wherein the poloxamer comprises at least 50% polyethylene oxide by molecular mass.
437. The composition of any preceding embodiment, wherein the poloxamer comprises at least 55% polyethylene oxide by molecular mass.
438. The composition of any preceding embodiment, wherein the poloxamer comprises at least 60% polyethylene oxide by molecular mass.
439. The composition of any preceding embodiment, wherein the poloxamer comprises at least 65% polyethylene oxide by molecular mass.
440. The composition of any preceding embodiment, wherein the poloxamer comprises at least 66% polyethylene oxide by molecular mass.
441. The composition of any preceding embodiment, wherein the poloxamer comprises at least 67% polyethylene oxide by molecular mass.
442. The composition of any preceding embodiment, wherein the poloxamer comprises at least 68% polyethylene oxide by molecular mass.
443. The composition of any preceding embodiment, wherein the poloxamer comprises at least 69% polyethylene oxide by molecular mass.
444. The composition of any preceding embodiment, wherein the poloxamer comprises at least 70% polyethylene oxide by molecular mass.
445. The composition of any preceding embodiment, wherein the poloxamer comprises 60-80% polyethylene oxide by molecular mass.
446. The composition of any preceding embodiment, wherein the poloxamer comprises 65-75% polyethylene oxide by molecular mass.
447. The composition of any preceding embodiment, wherein the poloxamer comprises:

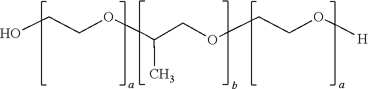

where a is 2-130 and b is 15-70.
448. The composition of any preceding embodiment, wherein a is 10-120.
449. The composition of any preceding embodiment, wherein a is 20-120.
450. The composition of any preceding embodiment, wherein a is 30-120.
451. The composition of any preceding embodiment, wherein a is 40-120.
452. The composition of any preceding embodiment, wherein a is 50-120.
453. The composition of any preceding embodiment, wherein a is 60-120.
454. The composition of any preceding embodiment, wherein a is 70-120.
455. The composition of any preceding embodiment, wherein a is 80-120.
456. The composition of any preceding embodiment, wherein a is 90-120.
457. The composition of any preceding embodiment, wherein a is 100-120.
458. The composition of any preceding embodiment, wherein a is 110-120.
459. The composition of any preceding embodiment, wherein a is 10-110.
460. The composition of any preceding embodiment, wherein a is 20-110.
461. The composition of any preceding embodiment, wherein a is 30-110.
462. The composition of any preceding embodiment, wherein a is 40-110.
463. The composition of any preceding embodiment, wherein a is 50-110.
464. The composition of any preceding embodiment, wherein a is 60-110.
465. The composition of any preceding embodiment, wherein a is 70-110.
466. The composition of any preceding embodiment, wherein a is 80-110.
467. The composition of any preceding embodiment, wherein a is 90-110.
468. The composition of any preceding embodiment, wherein a is 100-110.
469. The composition of any preceding embodiment, wherein a is 10-100.
470. The composition of any preceding embodiment, wherein a is 20-100.
471. The composition of any preceding embodiment, wherein a is 30-100.
472. The composition of any preceding embodiment, wherein a is 40-100.
473. The composition of any preceding embodiment, wherein a is 50-100.
474. The composition of any preceding embodiment, wherein a is 60-100.
475. The composition of any preceding embodiment, wherein a is 70-100.
476. The composition of any preceding embodiment, wherein a is 80-100.
477. The composition of any preceding embodiment, wherein a is 90-100.
478. The composition of any preceding embodiment, wherein a is 95-105.
479. The composition of any preceding embodiment, wherein a is 95-115.
480. The composition of any preceding embodiment, wherein a is 85-105.
481. The composition of any preceding embodiment, wherein a is 85-115.
482. The composition of any preceding embodiment, wherein b is 25-70.
483. The composition of any preceding embodiment, wherein b is 35-70.
484. The composition of any preceding embodiment, wherein b is 45-70.
485. The composition of any preceding embodiment, wherein b is 55-70.
486. The composition of any preceding embodiment, wherein b is 60-70.
487. The composition of any preceding embodiment, wherein b is 65-70.
488. The composition of any preceding embodiment, wherein b is 56+/−10%, and each a is 101+/−10%.
489. The composition of any preceding embodiment, wherein b is 61+/−15%, and each a is 101+/−10%.
490. The composition of any preceding embodiment, wherein b is 70+/−20%, and each a is 101+/−20%.
491. The composition of any preceding embodiment, wherein b is 56+/−10%, and each a is 100+/−10%.
492. The composition of any preceding embodiment, wherein b is 61+/−15%, and each a is 100+/−10%.
493. The composition of any preceding embodiment, wherein b is 70+/−20%, and each a is 100+1-10%.
494. The composition of any preceding embodiment, wherein the poloxamer has an average molecular weight of about 7250 to about 17350 Daltons.
495. The composition of any preceding embodiment, wherein the poloxamer has an average molecular weight of about 8000 to about 17000 Daltons.
496. The composition of any preceding embodiment, wherein the poloxamer has an average molecular weight of about 8000 to about 16000 Daltons.
497. The composition of any preceding embodiment, wherein the poloxamer has an average molecular weight of about 9000 to about 16000 Daltons.
498. The composition of any preceding embodiment, wherein the poloxamer has an average molecular weight of about 9000 to about 15000 Daltons.
499. The composition of any preceding embodiment, wherein the poloxamer has an average molecular weight of about 9800 to about 14600 Daltons.
500. The composition of any preceding embodiment, wherein the poloxamer has an average molecular weight of about 10000 to about 14000 Daltons.
501. The composition of any preceding embodiment, wherein the poloxamer has an average molecular weight of about 10500 to about 14000 Daltons.
502. The composition of any preceding embodiment, wherein the poloxamer has an average molecular weight of about 10500 to about 13500 Daltons.
503. The composition of any preceding embodiment, wherein the poloxamer has an average molecular weight of about 11000 to about 14000 Daltons.
504. The composition of any preceding embodiment, wherein the poloxamer has an average molecular weight of about 11000 to about 13500 Daltons.
505. The composition of any preceding embodiment, wherein the poloxamer has an average molecular weight of about 11500 to about 14000 Daltons.
506. The composition of any preceding embodiment, wherein the poloxamer has an average molecular weight of about 11500 to about 13000 Daltons.
507. The composition of any preceding embodiment, wherein the poloxamer has an average molecular weight of about 12000 to about 14000 Daltons.
508. The composition of any preceding embodiment, wherein the poloxamer has an average molecular weight of about 12000 to about 13000 Daltons.
509. The composition of any preceding embodiment, wherein the average molecular weight is the number-average molecular weight
510. The composition of any preceding embodiment, wherein the average molecular weight is the weight-average molecular weight
511. The composition of any preceding embodiment, wherein the poloxamer comprises Poloxamer 407.
512. The composition of any preceding embodiment, wherein Poloxamer 407 is at least 10% by weight of the poloxamer.
513. The composition of any preceding embodiment, wherein Poloxamer 407 is at least 20% by weight of the poloxamer.
514. The composition of any preceding embodiment, wherein Poloxamer 407 is at least 30% by weight of the poloxamer.
515. The composition of any preceding embodiment, wherein Poloxamer 407 is at least 40% by weight of the poloxamer.
516. The composition of any preceding embodiment, wherein Poloxamer 407 is at least 50% by weight of the poloxamer.
517. The composition of any preceding embodiment, wherein Poloxamer 407 is at least 60% by weight of the poloxamer.
518. The composition of any preceding embodiment, wherein Poloxamer 407 is at least 70% by weight of the poloxamer.
519. The composition of any preceding embodiment, wherein Poloxamer 407 is at least 75% by weight of the poloxamer.
520. The composition of any preceding embodiment, wherein Poloxamer 407 is at least 80% by weight of the poloxamer.
521. The composition of any preceding embodiment, wherein Poloxamer 407 is at least 90% by weight of the poloxamer.
522. The composition of any preceding embodiment, wherein the poloxamer is Poloxamer 407.
523. The composition of any preceding embodiment, wherein the one or more otic therapeutic agents does not include CHIR99021 or a pharmaceutically acceptable salt thereof
524. The composition of any preceding embodiment, wherein the one or more otic therapeutic agents does not include valproic acid or a pharmaceutically acceptable salt thereof
525. The composition of any preceding embodiment, wherein the composition does not comprise a bulking agent
526. The composition of any preceding embodiment, wherein the composition does not comprise an antioxidant
527. The composition of any preceding embodiment, wherein the composition does not comprise a cryoprotectant
528. The composition of any preceding embodiment, wherein the solubility of CHIR99021 or a pharmaceutically acceptable salt thereof in the composition is enhanced by a solubilizer.
529. The composition of embodiment 528, wherein the solubilizer is DMSO.
530. The composition of embodiment 528, wherein the solubilizer is a polyoxyethylene ester of 12-hydroxy stearic acid.
531. The composition of embodiment 528, wherein the solubilizer is tocofersolan.
532. The composition of embodiment 528, wherein the solubilizer is a bile salt 533. The composition of embodiment 528, wherein the bile salt is sodium deoxycholate.
534. The composition of embodiment 528, wherein the solubilizer is a polyoxyethylene castor oil derivative.
535. The composition of embodiment 534, wherein the polyoxyethylene castor oil derivative is PEG-35 castor oil.
536. The composition of embodiment 528, wherein the solubilizer is a medium chain triglyceride.
537. The composition of embodiment 536, wherein the medium chain triglyceride is derived from caproic acid.
538. The composition of embodiment 536, wherein the medium chain triglyceride is derived from lauric acid.
539. The composition of any preceding embodiment, wherein the pharmaceutical composition is a lyophilized pharmaceutical composition
540. The composition of any preceding embodiment, wherein the concentration of poloxamer is about 2% to about 50% w/v.
541. The composition of any preceding embodiment, wherein the concentration of poloxamer is about 2% to about 40% w/v.
542. The composition of any preceding embodiment, wherein the concentration of poloxamer is about 2% to about 30% w/v.
543. The composition of any preceding embodiment, wherein the concentration of poloxamer is about 2% to about 20% w/v.
544. The composition of any preceding embodiment, wherein the concentration of poloxamer is about 10% to about 20% w/v.
545. The composition of any preceding embodiment, wherein the concentration of poloxamer is about 12.5% to about 17.5% w/v.
546. The composition of any preceding embodiment, wherein the concentration of poloxamer is about 13% to about 17.5% w/v.
547. The composition of any preceding embodiment, wherein the concentration of poloxamer is about 13% to about 17% w/v.
548. The composition of any preceding embodiment, wherein the concentration of poloxamer is about 13.5% to about 17% w/v.
549. The composition of any preceding embodiment, wherein the concentration of poloxamer is about 13.5% to about 16.5% w/v.
550. The composition of any preceding embodiment, wherein the concentration of poloxamer is about 14% to about 16.5% w/v.
551. The composition of any preceding embodiment, wherein the concentration of poloxamer is about 14% to about 16% w/v.
552. The composition of any preceding embodiment, wherein the concentration of poloxamer is about 15% to about 17.5% w/v.
553. The composition or method of any preceding embodiment, wherein in the composition the poloxamer distribution has a number average molecular weight of about 10,800 to about 11,200 Da.
554. The composition or method of any preceding embodiment, wherein the poloxamer has a number average molecular weight of about 10,600 to about 11,400 Da.
555. The composition of any preceding embodiment, wherein in the composition the poloxamer distribution has a weight average molecular weight of about 11,500 to about 11,700 Da.
556. The composition or method of any preceding embodiment, wherein in the composition the poloxamer distribution is from 0 to about 16,600 Da.
557. The composition or method of any preceding embodiment, wherein in the poloxamer has a weight average molecular weight of about 11,300 to about 11,900 Da.
558. The composition of any preceding embodiment, wherein the poloxamer has a polydispersity index of about less than 1.07.
559. The composition of any preceding embodiment, wherein the poloxamer is from 0 to about 16,600 Da.
560. The composition of any preceding embodiment, wherein the poloxamer comprises purified poloxamer.
561. The composition of any preceding embodiment, wherein the poloxamer is purified poloxamer.
562. The composition of any preceding embodiment, wherein the purified poloxamer is prepared by liquid-liquid extraction.
563. The composition of any preceding embodiment, wherein the purified poloxamer is prepared by size exclusion chromatography.
564. The composition of any preceding embodiment, wherein the composition further comprises dimethyl sulfoxide (DMSO).
565. The composition of any preceding embodiment, wherein the concentration of DMSO is less than about 25% by weight
566. The composition of any preceding embodiment, wherein the concentration of DMSO is about 15 to about 25% by weight
567. The composition of any preceding embodiment, wherein the concentration of DMSO is less than about 20% by weight
568. The composition of any preceding embodiment, wherein the concentration of DMSO is about 10 to about 20% by weight
569. The composition of any preceding embodiment, wherein the concentration of DMSO is less than about 15% by weight
570. The composition of any preceding embodiment, wherein the concentration of DMSO is about 5 to about 15% by weight
571. The composition of any preceding embodiment, the concentration of DMSO is less than about 10% by weight
572. The composition of any preceding embodiment, wherein the concentration of DMSO is about 5 to about 10% by weight
573. The composition of any preceding embodiment, wherein the pharmaceutically acceptable salt of valproic acid is sodium valproate.
574. The composition of any preceding embodiment, wherein the pharmaceutically acceptable salt of valproic acid is potassium valproate.
575. The composition of any preceding embodiment, wherein the pharmaceutically acceptable salt of valproic acid is lithium valproate.
576. The composition of any preceding embodiment, wherein the pharmaceutically acceptable salt of valproic acid is calcium valproate.
577. The composition of any preceding embodiment, wherein the pharmaceutically acceptable salt of valproic acid is magnesium valproate.
578. The composition or method of any preceding embodiment, wherein the poloxamer is a thermoreversible gel.
579. The composition or method of any preceding embodiment, wherein the poloxamer is a gel at about 20° C. to about 40° C.
580. The composition or method of any preceding embodiment, wherein the poloxamer is a gel at about 25° C. to about 40° C.
581. The composition or method of any preceding embodiment, wherein the poloxamer is a gel at about 30° C. to about 40° C.
582. The composition or method of any preceding embodiment, wherein the poloxamer forms a gel at about 35° C. to about 40° C.
583. The composition or method of any preceding embodiment, wherein the poloxamer is a gel at about 37° C.
584. The composition or method of any preceding embodiment, wherein the poloxamer is a gel at about body temperature.
585. The composition or method of any preceding embodiment, wherein the poloxamer is an immobile gel at about body temperature.
586. The composition or method of any preceding embodiment, wherein the poloxamer has a viscosity greater than about 1×10⁶ mPa·s.
587. The composition or method of any preceding embodiment, wherein the poloxamer has a viscosity of about 1.0 to about 5.0×10⁶ mPa·s.
588. The composition or method of any preceding embodiment, wherein the poloxamer has a viscosity of about 2.0 to about 5.0×10⁶ mPa·s.
589. The composition or method of any preceding embodiment, wherein the poloxamer has a viscosity of about 2.0 to about 4.0×10⁶ mPa·s.
590. The composition or method of any preceding embodiment, wherein the poloxamer has a viscosity of about 3.0 to about 4.0×10⁶ mPa·s.
591. The lyophilized composition of any preceding embodiment, comprising about 0.01 to about 2% by weight CHIR99021.
592. The lyophilized composition of any preceding embodiments, comprising at least about 30% by weight valproic acid or a pharmaceutically acceptable salt thereof
593. The lyophilized composition of any preceding embodiments, comprising about 1 to about 2% by weight CHIR99021.
594. The lyophilized composition of any preceding embodiment, wherein the composition comprises at least about 40% by weight valproic acid or a pharmaceutically acceptable salt thereof
595. The lyophilized composition of any preceding embodiment, wherein the composition comprises at least about 50% by weight valproic acid or a pharmaceutically acceptable salt thereof
596. The lyophilized composition of any preceding embodiments, wherein the composition comprises about 30 to about 50% by weight valproic acid or a pharmaceutically acceptable salt thereof
597. The lyophilized composition of any preceding embodiment, wherein the composition comprises at least about 50% by weight poloxamer.
598. The lyophilized composition of any preceding embodiment, wherein the composition comprises at least about 60% by weight poloxamer.
599. The lyophilized composition of any preceding embodiment, wherein the composition comprises about 50 to about 70% by weight poloxamer.
600. The lyophilized composition of any preceding embodiment, wherein the lyophilized pharmaceutical composition comprises about 50 to about 500 mg of poloxamer and about 50 to about 500 mg of a compound of formula (I), for example valproic acid or a pharmaceutically acceptable salt thereof
601. The lyophilized composition of any preceding embodiment, wherein the lyophilized pharmaceutical composition comprises about 50 to about 500 mg of poloxamer and about 50 to about 500 mg of valproic acid or a pharmaceutically acceptable salt thereof
602. The lyophilized composition of any preceding embodiment, comprising about 50 to about 300 mg of valproic acid or a pharmaceutically acceptable salt thereof
603. The lyophilized composition of any preceding embodiment, comprising about 50 to about 200 mg of valproic acid or a pharmaceutically acceptable salt thereof
604. The lyophilized composition of any preceding embodiment, comprising about 50 to about 150 mg of valproic acid or a pharmaceutically acceptable salt thereof
605. The lyophilized composition of any preceding embodiment, comprising about 100 to about 500 mg of valproic acid or a pharmaceutically acceptable salt thereof
606. The lyophilized composition of any preceding embodiment, comprising about 100 to about 300 mg of valproic acid or a pharmaceutically acceptable salt thereof
607. The lyophilized composition of any preceding embodiment, comprising about 100 to about 200 mg of valproic acid or a pharmaceutically acceptable salt thereof
608. The lyophilized composition of any preceding embodiment, comprising about 100 to about 150 mg of valproic acid or a pharmaceutically acceptable salt thereof
609. The lyophilized composition of any preceding embodiment, comprising about 50 to about 300 mg of poloxamer.
610. The lyophilized composition of any preceding embodiment, comprising about 50 to about 200 mg of poloxamer.
611. The lyophilized composition of any preceding embodiment, comprising about 50 to about 150 mg of poloxamer.
612. The lyophilized composition of any preceding embodiment, comprising about 100 to about 500 mg of poloxamer.
613. The lyophilized composition of any preceding embodiment, comprising about 100 to about 300 mg of poloxamer.
614. The lyophilized composition of any preceding embodiment, comprising about 100 to about 200 mg of poloxamer.
615. The lyophilized composition of any preceding embodiment, comprising about 100 to about 150 mg of poloxamer.
616. The lyophilized composition of any preceding embodiment, wherein the composition comprises about 0.01 to about 1.5 to about 2% by weight CHIR99021, about 42.5 to about 47.5% by weight sodium valproate, and the remaining weight percent is Poloxamer 407.
617. The lyophilized composition of any preceding embodiment, wherein the poloxamer comprises poloxamer 407.
618. The lyophilized composition of any preceding embodiment, wherein the poloxamer is poloxamer 407.
619. The lyophilized composition of any preceding embodiment, wherein the poloxamer is purified poloxamer.
620. The lyophilized composition of any preceding embodiments, wherein the composition is substantially free from water and/or DMSO.
621. The lyophilized composition of any preceding embodiments, wherein the composition contains less than about 5% by weight of water and/or DMSO.
622. The lyophilized composition of any preceding embodiments, wherein the composition contains less than about 4% by weight of water and/or DMSO.
623. The lyophilized composition of any preceding embodiments, wherein the composition contains less than about 3% by weight of water and/or DMSO.
624. The lyophilized composition of any preceding embodiments, wherein the composition contains less than about 2% by weight of water and/or DMSO.
625. The lyophilized composition of any preceding embodiments, wherein the composition contains less than about 1% by weight of water and/or DMSO.
626. A lyophilized pharmaceutical composition comprising one or more otic therapeutic agents and a gelling agent 627. The lyophilized pharmaceutical composition of any preceding embodiment, wherein the one or more otic therapeutic agents are one or more hearing loss treatment agents.
628. The lyophilized pharmaceutical composition of any preceding embodiment, wherein the one or more otic therapeutic agents are modulators of one or more biological pathways and biological targets associated with hearing loss.
629. The lyophilized pharmaceutical composition of any one of the preceding embodiments, wherein the one or more otic therapeutic agents are selected from the group consisting of Wnt pathway agonists, histone deacetylase (HDAC) inhibitors, Dkk1 inhibitors, Axin inhibitors, SFRP1 inhibitors, bone morphogenetic protein (BMP) inhibitors, beta-catenin agonists, CyclinD1 activators, REST corepressor 1 (CoREST) inhibitors, NOTCH agonists, TGF-beta inhibitors, cAMP response element binding protein (CREB) activators, cyclin-dependent kinase (CDK) activators, CDK inhibitors, PI3K-AKT activators, PI3K-AKT inhibitors, PTEN inhibitors, ATOH1 agonists, ATOH1 antagonists, POU4F3 agonists, POU4F3 antagonists, GFI1 agonists, GFI1 antagonists, ERK/MAPK agonists, ERK/MAPK antagonists, FGF agonists, FGF antagonists, γ-ammobutynic acids (GABAs), voltage-gated Na+ channel antagonists, inositol, PKC agonists, PKC antagonists, FOXO inhibitors, FOXO agonists, Kv3 channel antagonists, p27Kip1 inhibitors, IL-1β, N-Methyl-D-aspartate (NMDA) receptor antagonists, NADPH quinone oxidoreductase 1, gamma secretase inhibitors, gamma secretase activators, NK1 receptor antagonist, NK1 receptor agonist, AMPA receptor agonist, AMPA receptor antagonist, Toll-Like Receptor (TLR) agonist, Toll-Like Receptor (TLR) antagonist, histamine H4 receptor agonist, H4 receptor antagonist, 5-HT3 receptor agonist, 5-HT3 receptor antagonist, Oct4 activators, Sox2 activators, Sox17 inducers, Klf4 inducers, cMyc activators, Sonic Hedgehog agonists, Sonic Hedgehog antagonists, Epidermal Growth Factor (EGF), Insulin Like Growth Factor (IGF), vascular endothelial growth factor (VEGF), endothelial nitric oxide synthase (eNOS), prostaglandin E (PGE), Brain-derived neurotrophic factor (BDNF), SMAD inhibitors, Sall4 inducers, Gata4 inducers, Gata6 inducers, proteasome inhibitors, retinoic acid receptor agonists, mTOR inhibitors, mTOR activators, Ascorbic acid, 2-phospho-1-ascorbic acid, KDM inhibitors, TTNPB, neurotrophin 3, DNA-modifying enzymes, LSD-1 inhibitors, Nicotinomide, Sirtuin, Histone methyl transferase inhibitors, Histone demethylase inhibitors, Histone Lysine Methyltransferase inhibitors, DNMT inhibitors, p53 inhibitors, p21 inhibitors, AMPK activators, Hippo activators, Hippo inhibitors, YAP/TAZ inhibitors, Mst1/2 inhibitors, CK1 activators, CK1 inhibitors, Noggin, R-spondin 1, BET activators, Sirt1 activators, Sirt1 inhibitors, Sirt2 activators, Sirt2 inhibitors, Sirt3 activators, Sirt3 inhibitors, JMJD3 inhibitors, DMNT inhibitors, Stat3 inhibitors, LSD1 inhibitors, active prostaglandins, cAMP activators, Oxidative phosphorylation uncouplers, arginine methyltransferase inhibitors, ALK4 inhibitors, Peroxisome proliferator-activated receptor gamma activators, EGFR inhibitors, SHH inhibitors, VitD activators, DOT1L inhibitors, Thyroid hormones, E box-dependent transcriptional activators, and protein degradation inhibitors.
630. The lyophilized pharmaceutical composition of any one of the preceding embodiments, wherein the one or more otic therapeutic agents are selected from GSK3-β pathway agonists.
631. The lyophilized pharmaceutical composition of any one of the preceding embodiments, wherein the one or more otic therapeutic agents are selected from of Wnt pathway agonists, histone deacetylase (HDAC) inhibitors.
632. The lyophilized pharmaceutical composition of any one of the preceding embodiments, wherein the one or more otic therapeutic agents are hair cell regeneration agents and/or octoprotective agents.
633. The lyophilized pharmaceutical composition of any one of the preceding embodiments, wherein the one or more otic therapeutic agents are selected from the group consisting of the agents described in Tables 1-13, and pharmaceutical salts thereof
634. The lyophilized pharmaceutical composition of any one of the preceding embodiments, wherein the one or more otic therapeutic agents are selected from the group consisting of the agents described in Tables 14-25, and pharmaceutical salts thereof
635. The lyophilized pharmaceutical composition of any one of the preceding embodiments, wherein the one or more otic therapeutic agents are CHIR99021 or a pharmaceutical acceptable salt thereof, and valproic acid or a pharmaceutical acceptable salt thereof
636. The lyophilized pharmaceutical composition of any one of the preceding embodiments, wherein the one or more otic therapeutic agents are LY2090314 or a pharmaceutical acceptable salt thereof, and valproic acid or a pharmaceutical acceptable salt thereof
637. The lyophilized pharmaceutical composition of any preceding embodiment, wherein the pharmaceutically acceptable salt of valproic acid is a sodium salt; optionally, the pharmaceutically acceptable salt of valproic acid is sodium valproate.
638. The lyophilized pharmaceutical composition of any preceding embodiment, wherein the pharmaceutically acceptable salt of valproic acid is a sodium salt
639. The lyophilized pharmaceutical composition of any preceding embodiment, wherein the pharmaceutically acceptable salt of valproic acid is sodium valproate.
640. The lyophilized pharmaceutical composition of any one of the preceding embodiments, wherein the gelling agent is a thermoreversible gelling agent
641. The lyophilized pharmaceutical composition of embodiment 640, wherein the thermoreversible gelling agent comprises a poloxamer.
642. The lyophilized pharmaceutical composition of embodiment 642, wherein the poloxamer is selected from the group consisting of Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, and Poloxamer 407;
   optionally, the poloxamer is Poloxamer 188 or Poloxamer 407; and optionally, the poloxamer is Poloxamer 407.
643. The composition of embodiment 642, wherein the poloxamer is Poloxamer 407.
644. The composition of embodiment 642, wherein the poloxamer is Poloxamer 188.
645. The composition of any one of the preceding embodiments, wherein the Poloxamer 407 has an average molecular weight of about 9 kDa or greater.
646. The composition of any one of the preceding embodiments, wherein the Poloxamer 407 has an average molecular weight of about 9.2 kDa or greater.
647. The composition of any one of the preceding embodiments, wherein the Poloxamer 407 has an average molecular weight of about 9.4 kDa or greater.
648. The composition of any one of the preceding embodiments, wherein the Poloxamer 407 has an average molecular weight of about 9.6 kDa or greater.
649. The composition of any one of the preceding embodiments, wherein the Poloxamer 407 has an average molecular weight of about 9.8 kDa or greater/650.
650. The composition of any one of the preceding embodiments, wherein the Poloxamer 407 has an average molecular weight of about 10 kDa or greater.
651. The composition of any one of the preceding embodiments, wherein the Poloxamer 407 has an average molecular weight of about 10.2 kDa or greater.
652. The composition of any one of the preceding embodiments, wherein the Poloxamer 407 has an average molecular weight of about 10.4 kDa or greater.
653. The composition of any one of the preceding embodiments, wherein the Poloxamer 407 has an average molecular weight of about 10.6 kDa or greater.
654. The composition of any one of the preceding embodiments, wherein the Poloxamer 407 has an average molecular weight of about 10.8 kDa or greater.
655. The composition of any one of the preceding embodiments, wherein the Poloxamer 407 has an average molecular weight of about 11 kDa or greater.
656. The composition of any one of the preceding embodiments, wherein the Poloxamer 407 has an average molecular weight of about 11.2 kDa or greater.
657. The composition of any one of the preceding embodiments, wherein the Poloxamer 407 has an average molecular weight of about 11.4 kDa or greater.
658. The composition of any one of the preceding embodiments, wherein the Poloxamer 407 has an average molecular weight of about 11.6 kDa or greater.
659. The composition of any one of the preceding embodiments, wherein the Poloxamer 407 has an average molecular weight of about 11.8 kDa or greater.
660. The composition of any one of the preceding embodiments, wherein the Poloxamer 407 has an average molecular weight of about 12 kDa or greater.
661. The composition of any one of the preceding embodiments, wherein the Poloxamer 407 has an average molecular weight of about 12.1 kDa or greater.
662. The lyophilized pharmaceutical composition of any one of the preceding embodiments, wherein the poloxamer is a purified poloxamer; optionally, the poloxamer is purified Poloxamer 407.
663. The composition of any one of the preceding embodiments, wherein the poloxamer is purified Poloxamer 407.
664. The composition of any one of the preceding embodiments, wherein the poloxamer is purified Poloxamer 188.
665. The lyophilized pharmaceutical composition of any one of the preceding embodiments, wherein the purified Poloxamer 407 has an average molecular weight of about 9 kDa or greater, about 9.2 kDa or greater, about 9.4 kDa or greater, about 9.6 kDa or greater, about 9.8 kDa or greater, about 10 kDa or greater, about 10.2 kDa or greater, about 10.4 kDa or greater, about 10.6 kDa or greater, about 10.8 kDa or greater, about 11 kDa or greater, about 11.2 kDa or greater, about 11.4 kDa or greater, about 11.6 kDa or greater, about 11.8 kDa or greater, about 12 kDa or greater, or about 12.1 kDa or greater.
666. The lyophilized pharmaceutical composition of any one of the preceding embodiments, wherein the purified Poloxamer 407 has an average molecular weight of about 9 kDa or greater.
667. The lyophilized pharmaceutical composition of any one of the preceding embodiments, wherein the purified Poloxamer 407 has an average molecular weight of about 9.2 kDa or greater.
668. The lyophilized pharmaceutical composition of any one of the preceding embodiments, wherein the purified Poloxamer 407 has an average molecular weight of about 9.4 kDa or greater.
669. The lyophilized pharmaceutical composition of any one of the preceding embodiments, wherein the purified Poloxamer 407 has an average molecular weight of about 9.6 kDa or greater.
670. The lyophilized pharmaceutical composition of any one of the preceding embodiments, wherein the purified Poloxamer 407 has an average molecular weight of about 9.8 kDa or greater/671.

671 The lyophilized pharmaceutical composition of any one of the preceding embodiments, wherein the purified Poloxamer 407 has an average molecular weight of about 10 kDa or greater.
672. The lyophilized pharmaceutical composition of any one of the preceding embodiments, wherein the purified Poloxamer 407 has an average molecular weight of about 10.2 kDa or greater.
673. The lyophilized pharmaceutical composition of any one of the preceding embodiments, wherein the purified Poloxamer 407 has an average molecular weight of about 10.4 kDa or greater.
674. The lyophilized pharmaceutical composition of any one of the preceding embodiments, wherein the purified Poloxamer 407 has an average molecular weight of about 10.6 kDa or greater.
675. The lyophilized pharmaceutical composition of any one of the preceding embodiments, wherein the purified Poloxamer 407 has an average molecular weight of about 10.8 kDa or greater.
676. The lyophilized pharmaceutical composition of any one of the preceding embodiments, wherein the purified Poloxamer 407 has an average molecular weight of about 11 kDa or greater.
677. The lyophilized pharmaceutical composition of any one of the preceding embodiments, wherein the purified Poloxamer 407 has an average molecular weight of about 11.2 kDa or greater.
678. The lyophilized pharmaceutical composition of any one of the preceding embodiments, wherein the purified Poloxamer 407 has an average molecular weight of about 11.4 kDa or greater.
679. The lyophilized pharmaceutical composition of any one of the preceding embodiments, wherein the purified Poloxamer 407 has an average molecular weight of about 11.6 kDa or greater.
680. The lyophilized pharmaceutical composition of any one of the preceding embodiments, wherein the purified Poloxamer 407 has an average molecular weight of about 11.8 kDa or greater.
681. The lyophilized pharmaceutical composition of any one of the preceding embodiments, wherein the purified Poloxamer 407 has an average molecular weight of about 12 kDa or greater.
682. The lyophilized pharmaceutical composition of any one of the preceding embodiments, wherein the purified Poloxamer 407 has an average molecular weight of about 12.1 kDa or greater.
683. The lyophilized pharmaceutical composition of any one of the preceding embodiments, wherein the purified Poloxamer 407 is prepared by liquid-liquid extraction or size exclusion chromatography.
684. The lyophilized pharmaceutical composition of any one of the preceding embodiments, wherein the purified Poloxamer 407 is prepared by liquid-liquid extraction.
685. The lyophilized pharmaceutical composition of any one of the preceding embodiments, wherein the purified Poloxamer 407 is prepared by size exclusion chromatography.
686. The lyophilized pharmaceutical composition of any one of the preceding embodiments, wherein about 10% or more, about 20% or more, about 30% or more, about 40% or more, about 50% or more, about 60% or more, about 70% or more, about 80% or more, about 90% or more, about 95% or more, about 98% or more, or about 99% or more of the one or more impurities having molecular weights below 9 kDa are removed from the Poloxamer 407 during the purification
687. The lyophilized pharmaceutical composition of any one of the preceding embodiments, wherein about 10% or more of the one or more impurities having molecular weights below 9 kDa are removed from the Poloxamer 407 during the purification
688. The lyophilized pharmaceutical composition of any one of the preceding embodiments, wherein about 20% or more of the one or more impurities having molecular weights below 9 kDa are removed from the Poloxamer 407 during the purification
689. The lyophilized pharmaceutical composition of any one of the preceding embodiments, wherein about 30% or more of the one or more impurities having molecular weights below 9 kDa are removed from the Poloxamer 407 during the purification
690. The lyophilized pharmaceutical composition of any one of the preceding embodiments, wherein about 40% or more of the one or more impurities having molecular weights below 9 kDa are removed from the Poloxamer 407 during the purification
691. The lyophilized pharmaceutical composition of any one of the preceding embodiments, wherein about 50% or more of the one or more impurities having molecular weights below 9 kDa are removed from the Poloxamer 407 during the purification
692. The lyophilized pharmaceutical composition of any one of the preceding embodiments, wherein about 60% or more of the one or more impurities having molecular weights below 9 kDa are removed from the Poloxamer 407 during the purification
693. The lyophilized pharmaceutical composition of any one of the preceding embodiments, wherein about 70% or more of the one or more impurities having molecular weights below 9 kDa are removed from the Poloxamer 407 during the purification
694. The lyophilized pharmaceutical composition of any one of the preceding embodiments, wherein about 80% or more of the one or more impurities having molecular weights below 9 kDa are removed from the Poloxamer 407 during the purification
695. The lyophilized pharmaceutical composition of any one of the preceding embodiments, wherein about 90% or more of the one or more impurities having molecular weights below 9 kDa are removed from the Poloxamer 407 during the purification
696. The lyophilized pharmaceutical composition of any one of the preceding embodiments, wherein about 95% or more of the one or more impurities having molecular weights below 9 kDa are removed from the Poloxamer 407 during the purification
697. The lyophilized pharmaceutical composition of any one of the preceding embodiments, wherein about 99% or more of the one or more impurities having molecular weights below 9 kDa are removed from the Poloxamer 407 during the purification
698. The lyophilized pharmaceutical composition of any one of the preceding embodiments, being in the form of a lyophilized cake.
699. The lyophilized pharmaceutical composition of any one of the preceding embodiments, having a higher stability to oxygen and/or light as compared to a comparable pharmaceutical composition comprising one or more solvents.
700. The lyophilized pharmaceutical composition of any one of the preceding embodiments, wherein the level of an impurity presented in the lyophilized pharmaceutical composition is less than about 10000 parts per million (ppm), less than about 1000 ppm, less than about 100 ppm, less than about 10 ppm, less than about 1 ppm, or less than about 0.1 ppm.
701. The lyophilized pharmaceutical composition of any one of the preceding embodiments, wherein the level of an impurity presented in the lyophilized pharmaceutical composition is less than about 10000 parts per million (ppm).
702. The lyophilized pharmaceutical composition of any one of the preceding embodiments, wherein the level of an impurity presented in the lyophilized pharmaceutical composition is less than about 1000 ppm.
703. The lyophilized pharmaceutical composition of any one of the preceding embodiments, wherein the level of an impurity presented in the lyophilized pharmaceutical composition is less than about 100 ppm.
704. The lyophilized pharmaceutical composition of any one of the preceding embodiments, wherein the level of an impurity presented in the lyophilized pharmaceutical composition is less than about 10 ppm.
705. The lyophilized pharmaceutical composition of any one of the preceding embodiments, wherein the level of an impurity presented in the lyophilized pharmaceutical composition is less than about 1 ppm.
706. The lyophilized pharmaceutical composition of any one of the preceding embodiments, wherein the level of an impurity presented in the lyophilized pharmaceutical composition is less than about 0.1 ppm.
707. The lyophilized pharmaceutical composition of any one of the preceding embodiments, wherein impurity is a residual solvent
708. The lyophilized pharmaceutical composition of any one of the preceding embodiments, wherein impurity is selected from the group consisting of 1-acetate-2-formate-1,2-propanediol, acetic acid, formic acid, formaldehyde, acetaldehyde, and propionaldehyde.
709. The lyophilized pharmaceutical composition of any preceding embodiment, wherein the concentration of aldehydes is less than about 1, about 2, about 3, about 4, about 5 or about 10 ppm (μg/g).
710. The lyophilized pharmaceutical composition of any preceding embodiment, wherein the concentration of aldehydes is less than about 10 ppm (μg/g).
711. The lyophilized pharmaceutical composition of any preceding embodiment, wherein the concentration of aldehydes is less than about 5 ppm (μg/g).
712. The lyophilized pharmaceutical composition of any preceding embodiment, wherein the concentration of aldehydes is less than about 4 ppm (μg/g).
713. The lyophilized pharmaceutical composition of any preceding embodiment, wherein the concentration of aldehydes is less than about 3 ppm (μg/g).
714. The lyophilized pharmaceutical composition of any preceding embodiment, wherein the concentration of aldehydes is less than about 2 ppm (μg/g).
715. The lyophilized pharmaceutical composition of any preceding embodiment, wherein the concentration of aldehydes is less than about 1 ppm (μg/g).
716. The lyophilized pharmaceutical composition of any preceding embodiment, wherein the aldehydes are volatile aldehydes.
717. The composition of any preceding embodiment, wherein the aldehydes are selected from the group of formaldehyde, acetaldehyde, and/or propionaldehyde.
718. The lyophilized pharmaceutical composition of any preceding embodiment, wherein the aldehydes comprise formaldehyde, acetaldehyde, and/or propionaldehyde.
719. The lyophilized pharmaceutical composition of any preceding embodiment, wherein the aldehyde comprises formaldehyde.
720. The lyophilized pharmaceutical composition of any preceding embodiment, wherein the aldehyde comprises acetaldehyde.
721. The lyophilized pharmaceutical composition of any preceding embodiment, wherein the aldehyde comprises propionaldehyde
722. The lyophilized pharmaceutical composition of any one of the preceding embodiments, wherein the level of polyethylene oxide presented in the lyophilized pharmaceutical composition is below about 3%, below about 2%, below about 1%, below about 0.5%, or below about 0.1%, as measured by high-performance liquid chromatography (HPLC).
723. The lyophilized pharmaceutical composition of any one of the preceding embodiments, wherein the level of polyethylene oxide presented in the lyophilized pharmaceutical composition is below about 3%, as measured by high-performance liquid chromatography (HPLC).
724. The lyophilized pharmaceutical composition of any one of the preceding embodiments, wherein the level of polyethylene oxide presented in the lyophilized pharmaceutical composition is below about 2%, as measured by high-performance liquid chromatography (HPLC).
725. The lyophilized pharmaceutical composition of any one of the preceding embodiments, wherein the level of polyethylene oxide presented in the lyophilized pharmaceutical composition is below about 1%, as measured by high-performance liquid chromatography (HPLC).
726. The lyophilized pharmaceutical composition of any one of the preceding embodiments, wherein the level of polyethylene oxide presented in the lyophilized pharmaceutical composition is below about 0.5%, as measured by high-performance liquid chromatography (HPLC).
727. The lyophilized pharmaceutical composition of any one of the preceding embodiments, wherein the level of polyethylene oxide presented in the lyophilized pharmaceutical composition is below about 0.1%, as measured by high-performance liquid chromatography (HPLC).
728. The lyophilized pharmaceutical composition of any one of the preceding embodiments, wherein the total level of one or more impurities with c Log P of about 1 or less presented in the lyophilized pharmaceutical composition is from about 30% to about 35%, from about 25% to about 29%, from about 20% to about 25%, from about 15% to about 19%, from about 10% to about 14%, from about 5% to about 9%, or from about 0% to about 4%, as measured by high-performance liquid chromatography (HPLC).
729. The lyophilized pharmaceutical composition of any one of the preceding embodiments, wherein the total level of one or more impurities with c Log P of about 1 or less presented in the lyophilized pharmaceutical composition is from about 30% to about 35% as measured by high-performance liquid chromatography (HPLC).
730. The lyophilized pharmaceutical composition of any one of the preceding embodiments, wherein the total level of one or more impurities with c Log P of about 1 or less presented in the lyophilized pharmaceutical composition is from about 25% to about 29% as measured by high-performance liquid chromatography (HPLC).
731. The lyophilized pharmaceutical composition of any one of the preceding embodiments, wherein the total level of one or more impurities with c Log P of about 1 or less presented in the lyophilized pharmaceutical composition is from about 20% to about 25% as measured by high-performance liquid chromatography (HPLC).
732. The lyophilized pharmaceutical composition of any one of the preceding embodiments, wherein the total level of one or more impurities with c Log P of about 1 or less presented in the lyophilized pharmaceutical composition is from about 15% to about 19% as measured by high-performance liquid chromatography (HPLC).
733. The lyophilized pharmaceutical composition of any one of the preceding embodiments, wherein the total level of one or more impurities with c Log P of about 1 or less presented in the lyophilized pharmaceutical composition is from about 10% to about 14% as measured by high-performance liquid chromatography (HPLC).
734. The lyophilized pharmaceutical composition of any one of the preceding embodiments, wherein the total level of one or more impurities with c Log P of about 1 or less presented in the lyophilized pharmaceutical composition is from about 5% to about 9% as measured by high-performance liquid chromatography (HPLC).
735. The lyophilized pharmaceutical composition of any one of the preceding embodiments, wherein the total level of one or more impurities with c Log P of about 1 or less presented in the lyophilized pharmaceutical composition is from about 0% to about 4%, as measured by high-performance liquid chromatography (HPLC).
736. The lyophilized pharmaceutical composition of any one of the preceding embodiments, wherein the total level of one or more impurities having a boiling of about 220° C. or less presented in the lyophilized pharmaceutical composition is from about 35% to about 40%, from about 30% to about 34%, from about 25% to about 29%, from about 20% to about 25%, from about 15% to about 19%, from about 10% to about 14%, from about 5% to about 9%, or from about 0% to about 4%, as measured by high-performance liquid chromatography (HPLC)
737. The lyophilized pharmaceutical composition of any one of the preceding embodiments, wherein the total level of one or more impurities having a boiling of about 220° C. or less presented in the lyophilized pharmaceutical composition is from about 35% to about 40%, as measured by high-performance liquid chromatography (HPLC).
738. The lyophilized pharmaceutical composition of any one of the preceding embodiments, wherein the total level of one or more impurities having a boiling of about 220° C. or less presented in the lyophilized pharmaceutical composition is from about 30% to about 34%, as measured by high-performance liquid chromatography (HPLC).
739. The lyophilized pharmaceutical composition of any one of the preceding embodiments, wherein the total level of one or more impurities having a boiling of about 220° C. or less presented in the lyophilized pharmaceutical composition is from about 25% to about 29%, as measured by high-performance liquid chromatography (HPLC).
740. The lyophilized pharmaceutical composition of any one of the preceding embodiments, wherein the total level of one or more impurities having a boiling of about 220° C. or less presented in the lyophilized pharmaceutical composition is from about 20% to about 25%, as measured by high-performance liquid chromatography (HPLC).
741. The lyophilized pharmaceutical composition of any one of the preceding embodiments, wherein the total level of one or more impurities having a boiling of about 220° C. or less presented in the lyophilized pharmaceutical composition is from about 15% to about 19%, as measured by high-performance liquid chromatography (HPLC).
742. The lyophilized pharmaceutical composition of any one of the preceding embodiments, wherein the total level of one or more impurities having a boiling of about 220° C. or less presented in the lyophilized pharmaceutical composition is from about 10% to about 14%, as measured by high-performance liquid chromatography (HPLC).
743. The lyophilized pharmaceutical composition of any one of the preceding embodiments, wherein the total level of one or more impurities having a boiling of about 220° C. or less presented in the lyophilized pharmaceutical composition is from about 5% to about 9%, as measured by high-performance liquid chromatography (HPLC).
744. The lyophilized pharmaceutical composition of any one of the preceding embodiments, wherein the total level of one or more impurities having a boiling of about 220° C. or less presented in the lyophilized pharmaceutical composition is from about 0% to about 4%, as measured by high-performance liquid chromatography (HPLC).
745. The lyophilized pharmaceutical composition of any one of the preceding embodiments, wherein the lyophilized pharmaceutical composition comprises purified Poloxamer 407, and wherein the level of the one or more otic therapeutic agents presented in the lyophilized pharmaceutical composition is about 1.5 fold or higher, about 1.8 fold or higher, about 2 fold or higher, about 2.5 fold or higher, about 3 fold or higher, about 5 fold or higher, or about 10 fold or higher as compared to a comparable lyophilized pharmaceutical composition without purified Poloxamer 407;
   optionally, the comparable lyophilized pharmaceutical composition comprises unpurified Poloxamer 407.
746. The lyophilized pharmaceutical composition of any one of the preceding embodiments, wherein the lyophilized pharmaceutical composition comprises purified Poloxamer 407, and wherein the level of the one or more otic therapeutic agents presented in the lyophilized pharmaceutical composition is about 1.5 fold or higher as compared to a comparable lyophilized pharmaceutical composition without purified Poloxamer 407;
   optionally, the comparable lyophilized pharmaceutical composition comprises unpurified Poloxamer 407.
747. The lyophilized pharmaceutical composition of any one of the preceding embodiments, wherein the lyophilized pharmaceutical composition comprises purified Poloxamer 407, and wherein the level of the one or more otic therapeutic agents presented in the lyophilized pharmaceutical composition is about 1.8 fold or higher as compared to a comparable lyophilized pharmaceutical composition without purified Poloxamer 407;
   optionally, the comparable lyophilized pharmaceutical composition comprises unpurified Poloxamer 407.
748. The lyophilized pharmaceutical composition of any one of the preceding embodiments, wherein the lyophilized pharmaceutical composition comprises purified Poloxamer 407, and wherein the level of the one or more otic therapeutic agents presented in the lyophilized pharmaceutical composition is about 2 fold or higher as compared to a comparable lyophilized pharmaceutical composition without purified Poloxamer 407;
   optionally, the comparable lyophilized pharmaceutical composition comprises unpurified Poloxamer 407.
749. The lyophilized pharmaceutical composition of any one of the preceding embodiments, wherein the lyophilized pharmaceutical composition comprises purified Poloxamer 407, and wherein the level of the one or more otic therapeutic agents presented in the lyophilized pharmaceutical composition is about 2.5 fold or higher as compared to a comparable lyophilized pharmaceutical composition without purified Poloxamer 407;
   optionally, the comparable lyophilized pharmaceutical composition comprises unpurified Poloxamer 407.
750. The lyophilized pharmaceutical composition of any one of the preceding embodiments, wherein the lyophilized pharmaceutical composition comprises purified Poloxamer 407, and wherein the level of the one or more otic therapeutic agents presented in the lyophilized pharmaceutical composition is about 3 fold or higher as compared to a comparable lyophilized pharmaceutical composition without purified Poloxamer 407;
   optionally, the comparable lyophilized pharmaceutical composition comprises unpurified Poloxamer 407.
751. The lyophilized pharmaceutical composition of any one of the preceding embodiments, wherein the lyophilized pharmaceutical composition comprises purified Poloxamer 407, and wherein the level of the one or more otic therapeutic agents presented in the lyophilized pharmaceutical composition is about 5 fold or higher as compared to a comparable lyophilized pharmaceutical composition without purified Poloxamer 407;
   optionally, the comparable lyophilized pharmaceutical composition comprises unpurified Poloxamer 407.
752. The lyophilized pharmaceutical composition of any one of the preceding embodiments, wherein the lyophilized pharmaceutical composition comprises purified Poloxamer 407, and wherein the level of the one or more otic therapeutic agents presented in the lyophilized pharmaceutical composition is about 10 fold or higher as compared to a comparable lyophilized pharmaceutical composition without purified Poloxamer 407;
   optionally, the comparable lyophilized pharmaceutical composition comprises unpurified Poloxamer 407.
753. The lyophilized pharmaceutical composition of any one of the preceding embodiments, wherein the comparable lyophilized pharmaceutical composition comprises unpurified Poloxamer 407.
754. The lyophilized pharmaceutical composition of any one of the preceding embodiments, wherein the lyophilized pharmaceutical composition comprises purified Poloxamer 407, and wherein the lyophilized pharmaceutical composition has lower batch-to-batch variability of one or more gelation properties (e.g., gelation temperature, viscosity, and/or stability) as compared to a comparable lyophilized pharmaceutical composition without purified Poloxamer 407;
   optionally, the comparable lyophilized pharmaceutical composition comprises unpurified Poloxamer 407.
755. The lyophilized pharmaceutical composition of embodiment 97, wherein the comparable lyophilized pharmaceutical composition comprises unpurified Poloxamer 407.
756. The lyophilized pharmaceutical composition of any one of the preceding embodiments, wherein the lyophilized pharmaceutical composition comprises purified Poloxamer 407, and wherein the lyophilized pharmaceutical composition has a lower gelation temperature as compared to a comparable lyophilized pharmaceutical composition without purified Poloxamer 407.
757. The lyophilized pharmaceutical composition of any one of the preceding embodiments, wherein the lyophilized pharmaceutical composition comprises purified Poloxamer 407, and wherein the lyophilized pharmaceutical composition has a higher viscosity as compared to a comparable lyophilized pharmaceutical composition without purified Poloxamer 407.
758. The lyophilized pharmaceutical composition of any one of the preceding embodiments, wherein the lyophilized pharmaceutical composition comprises purified Poloxamer 407, and wherein the lyophilized pharmaceutical composition has a lower gelation temperature, a narrower gelation temperature range, and/or a higher viscosity as compared to a comparable lyophilized pharmaceutical composition without purified Poloxamer 407;
   optionally, the comparable lyophilized pharmaceutical composition comprises unpurified Poloxamer 407.
759. The lyophilized pharmaceutical composition of embodiment 758, wherein the comparable lyophilized pharmaceutical composition comprises unpurified Poloxamer 407.
760. The lyophilized pharmaceutical composition of any one of the preceding embodiments, wherein the lyophilized pharmaceutical composition comprises purified Poloxamer 407, and wherein the lyophilized pharmaceutical composition has a reduced degradation rate as compared to a comparable lyophilized pharmaceutical composition without purified Poloxamer 407;
   optionally, the comparable lyophilized pharmaceutical composition comprises unpurified Poloxamer 407.
761. A method for reconstituting lyophilized pharmaceutical composition comprising dissolving the lyophilized pharmaceutical composition of any preceding claim in a diluent
762. The method of embodiment 761, wherein the composition dissolves in the diluent in less than about an hour.
763. The method of embodiment 761 or 762, wherein the composition dissolves in the diluent in less than about 30 minutes.
764. The method of embodiments 761 to 763, wherein the diluent comprises water.
765. A reconstituted pharmaceutical composition obtained by the method of embodiments 761 to 764.
766. A reconstituted pharmaceutical composition comprising a lyophilized pharmaceutical composition of any preceding embodiment and a diluent
767. The composition of embodiment 766, wherein the lyophilized pharmaceutical composition is dissolved in the diluent
768. The lyophilized pharmaceutical composition of any one of the preceding embodiments, being suitable for preparing a reconstituted solution by a reconstitution process.
769. The lyophilized pharmaceutical composition of any one of the preceding embodiments, wherein the reconstitution process is of less than about 1 hour.
770. The lyophilized pharmaceutical composition of any one of the preceding embodiments, wherein the reconstitution process is of less than about 45 minutes.
771. The lyophilized pharmaceutical composition of any one of the preceding embodiments, wherein the reconstitution process is of less than about 30 minutes.
772. The lyophilized pharmaceutical composition of any one of the preceding embodiments, wherein the reconstitution process is of less than about 15 minutes.
773. The lyophilized pharmaceutical composition of any one of the preceding embodiments, wherein the reconstitution process is of less than about 10 minutes.
774. The lyophilized pharmaceutical composition of any one of the preceding embodiments, wherein the reconstituted solution is suitable for injection;
   optionally, the reconstituted solution is suitable for intratympanic injection
775. The lyophilized pharmaceutical composition of embodiment 774, wherein the reconstituted composition is suitable for injection
776. The lyophilized pharmaceutical composition of embodiment 774 or 775, wherein the reconstituted composition is suitable for intratympanic injection
777. The lyophilized pharmaceutical composition of any one of the preceding embodiments, wherein the reconstituted solution maintains one or more rheometric properties of a pre-lyophilized solution which is used for preparing the lyophilized pharmaceutical composition
778. The lyophilized pharmaceutical composition of any one of the preceding embodiments, wherein the reconstituted solution has a reduced degradation rate as compared to a reconstituted solution prepared from a comparable lyophilized pharmaceutical composition without purified Poloxamer 407;
   optionally, the comparable lyophilized pharmaceutical composition comprises unpurified Poloxamer 407.
779. The lyophilized pharmaceutical composition of embodiment 778, wherein the comparable lyophilized pharmaceutical composition comprises unpurified Poloxamer 407.
780. The lyophilized pharmaceutical composition of any one of the preceding embodiments for treating hearing loss m a subject in need thereof
781. Use of the lyophilized pharmaceutical composition of any one of the preceding embodiments in the preparation of a reconstituted solution for treating hearing loss in a subject in need thereof
782. A method of treating hearing loss, comprising administering to a subject in need thereof a pharmaceutically acceptable amount of a reconstituted solution, wherein the reconstituted solution is prepared by a reconstitution process using the lyophilized pharmaceutical composition of any one of the preceding embodiments.
783. A method for reconstituting a lyophilized pharmaceutical composition, the method comprising:
   (a) providing the lyophilized pharmaceutical composition of any preceding embodiment
   (b) reconstituting the lyophilized pharmaceutical composition with a pharmaceutically acceptable diluent; and
   (c) obtaining a reconstituted pharmaceutical composition
784. The method of embodiment 783, wherein reconstituting the lyophilized pharmaceutical composition comprises dissolving the lyophilized pharmaceutical composition in the pharmaceutically acceptable diluent
785. The method of embodiment 783 or 784, wherein dissolving the lyophilized pharmaceutical composition in the pharmaceutically acceptable diluent takes less than about 1 hour.
786. The method of any preceding embodiment, wherein dissolving the lyophilized pharmaceutical composition in the pharmaceutically acceptable diluent takes less than about 45 minutes.
787. The method of any preceding embodiment, wherein dissolving the lyophilized pharmaceutical composition in the pharmaceutically acceptable diluent takes less than about 30 minutes.
788. The method of any preceding embodiment, wherein dissolving the lyophilized pharmaceutical composition in the pharmaceutically acceptable diluent takes less than about 15 minutes.
789. The method of any preceding embodiment, wherein dissolving the lyophilized pharmaceutical composition in the pharmaceutically acceptable diluent takes less than about 10 minutes.
790. A reconstituted pharmaceutical composition obtained by the method of embodiment 783.
791. A reconstituted pharmaceutical composition comprising the lyophilized composition of any preceding embodiment and a diluent
792. The reconstituted pharmaceutical composition of embodiment 791, wherein the composition reconstitutes in less about 1 hour.
793. The reconstituted pharmaceutical composition of embodiment 791 or 792, wherein the composition reconstitutes in less than about 45 minutes.
794. The reconstituted pharmaceutical composition of any preceding embodiment, wherein the composition reconstitutes in less than about 30 minutes.
795. The reconstituted pharmaceutical composition of any preceding embodiment, wherein the composition reconstitutes in less than about 15 minutes.
796. The reconstituted pharmaceutical composition of any preceding embodiment, wherein the composition reconstitutes in less than about 10 minutes.
797. A pharmaceutical composition, comprising: CHIR99021 or a pharmaceutically acceptable salt thereof being present at a concentration ranging from 0.025 mg/ml to about 25 mg/ml.
798. The pharmaceutical composition of embodiment 797, further comprising:
valproic acid or a pharmaceutically acceptable salt thereof being present at a concentration ranging from 0.5 mg/ml to about 500 mg/ml.
799. The pharmaceutical composition of embodiment 797 or 798, further comprising:
poloxamer 407 being present at a concentration ranging from 1 wt % to about 25 wt %.
800. The pharmaceutical composition of any preceding embodiment, further comprising:
dimethyl sulfoxide (DMSO) being present at a concentration below 20 wt %.
801. A pharmaceutical composition, comprising:
   i) CHIR99021 or a pharmaceutically acceptable salt thereof being present at a concentration ranging from 0.025 mg/ml to about 25 mg/ml;
   ii) valproic acid or a pharmaceutically acceptable salt thereof being present at a concentration ranging from 0.5 mg/ml to about 500 mg/ml;
   iii) poloxamer 407 being present at a concentration ranging from 1 wt % to about 25 wt %; and iv) dimethyl sulfoxide (DMSO) being present at a concentration below 25 wt %.
802. The pharmaceutical composition of embodiment 801, wherein the pharmaceutically acceptable salt of valproic acid is a sodium salt;
   optionally, wherein the pharmaceutically acceptable salt of valproic acid is sodium valproate.
803. The pharmaceutical composition of embodiment 802, wherein the pharmaceutically acceptable salt of valproic acid is sodium valproate.
804. The pharmaceutical composition of any one of the preceding embodiments, wherein the concentration of CHIR99021 or the pharmaceutically acceptable salt thereof ranges from about 0.05 mg/ml to about 5 mg/ml, from about 0.25 mg/ml to about 2.5 mg/ml, from about 0.5 mg/ml to about 1.75 mg/ml, or from about 1.45 mg/ml to about 1.65 mg/ml;
   optionally, the concentration of CHIR99021 or the pharmaceutically acceptable salt thereof is about 1.55 mg/ml.
805. The pharmaceutical composition of any one of the preceding embodiments, wherein the concentration of CHIR99021 or the pharmaceutically acceptable salt thereof ranges from about 0.05 mg/ml to about 5 mg/ml.
806. The pharmaceutical composition of any one of the preceding embodiments, wherein the concentration of CHIR99021 or the pharmaceutically acceptable salt thereof ranges from about 0.25 mg/ml to about 2.5 mg/ml.
807. The pharmaceutical composition of any one of the preceding embodiments, wherein the concentration of CHIR99021 or the pharmaceutically acceptable salt thereof ranges from about 0.5 mg/ml to about 1.75 mg/ml.
808. The pharmaceutical composition of any one of the preceding embodiments, wherein the concentration of CHIR99021 or the pharmaceutically acceptable salt thereof ranges from about 1.45 mg/ml to about 1.65 mg/ml.
809. The pharmaceutical composition of any one of the preceding embodiments, wherein the concentration of CHIR99021 or the pharmaceutically acceptable salt thereof is about 1.55 mg/ml.
810. The pharmaceutical composition of any one of the preceding embodiments, wherein the concentration of valproic acid or the pharmaceutically acceptable salt thereof ranges from about 2.5 mg/ml to about 200 mg/ml, from about 5 mg/ml to about 100 mg/ml, from about 15 mg/ml to about 50 mg/ml, or from about 43 mg/ml to about 46 mg/ml;
   optionally, the concentration of valproic acid or the pharmaceutically acceptable salt thereof is about 44.5 mg/ml.
811. The pharmaceutical composition of any one of the preceding embodiments, wherein the concentration of valproic acid or the pharmaceutically acceptable salt thereof ranges from about 2.5 mg/ml to about 200 mg/ml.
812. The pharmaceutical composition of any one of the preceding embodiments, wherein the concentration of valproic acid or the pharmaceutically acceptable salt thereof ranges from about 5 mg/ml to about 100 mg/ml.
813. The pharmaceutical composition of any one of the preceding embodiments, wherein the concentration of valproic acid or the pharmaceutically acceptable salt thereof ranges from about 15 mg/ml to about 50 mg/ml.
814. The pharmaceutical composition of any one of the preceding embodiments, wherein the concentration of valproic acid or the pharmaceutically acceptable salt thereof ranges from about 43 mg/ml to about 46 mg/ml.
815. The pharmaceutical composition of any one of the preceding embodiments, wherein the concentration of valproic acid or the pharmaceutically acceptable salt thereof is about 44.5 mg/ml.
816. The composition of any one of the preceding embodiments, wherein the concentration of valproic acid or the pharmaceutically acceptable salt thereof is at least about 100 mg/mL.
817. The composition of any one of the preceding embodiments, wherein the concentration of valproic acid or the pharmaceutically acceptable salt thereof is at least about 110 mg/mL.
818. The composition of any one of the preceding embodiments, wherein the concentration of valproic acid or the pharmaceutically acceptable salt thereof is at least about 120 mg/mL.
819. The composition of any one of the preceding embodiments, wherein the concentration of valproic acid or the pharmaceutically acceptable salt thereof is at least about 125 mg/mL.
820. The composition of any one of the preceding embodiments, wherein the concentration of valproic acid or the pharmaceutically acceptable salt thereof is at least about 130 mg/mL.
821. The composition of any one of the preceding embodiments, wherein the concentration of valproic acid or the pharmaceutically acceptable salt thereof ranges from about 100 to about 500 mg/mL.
822. The composition of any one of the preceding embodiments, wherein the concentration of valproic acid or the pharmaceutically acceptable salt thereof ranges from about 100 to about 350 mg/mL.
823. The composition of any one of the preceding embodiments, wherein the concentration of valproic acid or the pharmaceutically acceptable salt thereof ranges from about 110 to about 140 mg/mL.
824. The composition of any one of the preceding embodiments, wherein the concentration of valproic acid or the pharmaceutically acceptable salt thereof ranges from about 120 to about 150 mg/mL.
825. The composition of any one of the preceding embodiments, wherein the concentration of valproic acid or the pharmaceutically acceptable salt thereof ranges from about 120 to about 140 mg/mL.
826. The composition of any one of the preceding embodiments, wherein the concentration of valproic acid or the pharmaceutically acceptable salt thereof ranges from about 125 to about 150 mg/mL.
827. The composition of any one of the preceding embodiments, wherein the concentration of valproic acid or the pharmaceutically acceptable salt thereof ranges from about 125 to about 140 mg/mL.
828. The composition of any one of the preceding embodiments, wherein the concentration of valproic acid or the pharmaceutically acceptable salt thereof ranges from about 125 to about 135 mg/mL.
829. The composition of any one of the preceding embodiments, wherein the concentration of valproic acid or the pharmaceutically acceptable salt thereof is at least about 70 mg/mL.
830. The composition of any one of the preceding embodiments, wherein the concentration of valproic acid or the pharmaceutically acceptable salt thereof is at least about 75 mg/mL.
831. The composition of any one of the preceding embodiments, wherein the concentration of valproic acid or the pharmaceutically acceptable salt thereof is at least about 80 mg/mL.
832. The composition of any one of the preceding embodiments, wherein the concentration of valproic acid or the pharmaceutically acceptable salt thereof ranges from about 70 mg/mL to about 90 mg/mL.
833. The composition of any one of the preceding embodiments, wherein the concentration of valproic acid or the pharmaceutically acceptable salt thereof ranges from about 75 mg/mL to about 95 mg/mL.
834. The composition of any one of the preceding embodiments, wherein the concentration of valproic acid or the pharmaceutically acceptable salt thereof ranges from about 75 mg/mL to about 85 mg/mL.
835. The pharmaceutical composition of any one of the preceding embodiments, wherein the concentration of poloxamer 407 ranges from about 2.5 wt % to about 12.5 wt %, from about 5 wt % to about 11 wt %, from about 6 wt % to about 10 wt %, or from about 7 wt % to about 8.5 wt %;
   optionally, the concentration of poloxamer 407 is about 8 wt %.
836. The pharmaceutical composition of any one of the preceding embodiments, wherein the concentration of poloxamer 407 ranges from about 2.5 wt % to about 12.5 wt %.
837. The pharmaceutical composition of any one of the preceding embodiments, wherein the concentration of poloxamer 407 ranges from about 5 wt % to about 11 wt %.
838. The pharmaceutical composition of any one of the preceding embodiments, wherein the concentration of poloxamer 407 ranges from about 6 wt % to about 10 wt %.
839. The pharmaceutical composition of any one of the preceding embodiments, wherein the concentration of poloxamer 407 ranges from about 7 wt % to about 8.5 wt %.
840. The pharmaceutical composition of any one of the preceding embodiments, wherein the concentration of poloxamer 407 is about 8 wt %.
841. The pharmaceutical composition of any one of the preceding embodiments, wherein the concentration of DMSO ranges from about 0.5 wt % to about 5 wt %, from about 1 wt % to about 4 wt %, from about 1.5 wt % to about 3.5 wt %, or from about 2 wt % to about 3 wt %;
   optionally, the concentration of DMSO is about 2.5 wt %.
842. The pharmaceutical composition of any one of the preceding embodiments, wherein the concentration of DMSO ranges from about 0.5 wt % to about 25 wt %.
843. The pharmaceutical composition of any one of the preceding embodiments, wherein the concentration of DMSO ranges from about 0.5 wt % to about 20 wt %.
844. The pharmaceutical composition of any one of the preceding embodiments, wherein the concentration of DMSO ranges from about 0.5 wt % to about 15 wt %.
845. The pharmaceutical composition of any one of the preceding embodiments, wherein the concentration of DMSO ranges from about 0.5 wt % to about 10 wt %.
846. The pharmaceutical composition of any one of the preceding embodiments, wherein the concentration of DMSO ranges from about 0.5 wt % to about 5 wt %.
847. The pharmaceutical composition of any one of the preceding embodiments, wherein the concentration of DMSO ranges from about 1 wt % to about 4 wt %.
848. The pharmaceutical composition of any one of the preceding embodiments, wherein the concentration of DMSO ranges from about 1.5 wt % to about 3.5 wt %.
849. The pharmaceutical composition of any one of the preceding embodiments, wherein the concentration of DMSO ranges or from about 2 wt % to about 3 wt %.
850. The pharmaceutical composition of any one of the preceding embodiments, wherein the concentration of DMSO is about 2.5 wt %.
851. The pharmaceutical composition of any one of the preceding embodiments, wherein the weight ratio between CHIR99021 or the pharmaceutically acceptable salt thereof and valproic acid or the pharmaceutically acceptable salt thereof ranges from about 1:5 to about 1:10, from about 1:10 to about 1:50, from about 1:20 to about 1:35, from about 1:25 to about 1:31, or from about 1:27 to about 1:29.
852. The pharmaceutical composition of any one of the preceding embodiments, wherein the weight ratio between CHIR99021 or the pharmaceutically acceptable salt thereof and valproic acid or the pharmaceutically acceptable salt thereof ranges from about 1:5 to about 1:10.
853. pharmaceutical composition of any one of the preceding embodiments, wherein the weight ratio between CHIR99021 or the pharmaceutically acceptable salt thereof and valproic acid or the pharmaceutically acceptable salt thereof ranges from about 1:10 to about 1:50.
854. pharmaceutical composition of any one of the preceding embodiments, wherein the weight ratio between CHIR99021 or the pharmaceutically acceptable salt thereof and valproic acid or the pharmaceutically acceptable salt thereof ranges from about 1:20 to about 1:35.
855. pharmaceutical composition of any one of the preceding embodiments, wherein the weight ratio between CHIR99021 or the pharmaceutically acceptable salt thereof and valproic acid or the pharmaceutically acceptable salt thereof ranges from about 1:25 to about 1:31.
856. pharmaceutical composition of any one of the preceding embodiments, wherein the weight ratio between CHIR99021 or the pharmaceutically acceptable salt thereof and valproic acid or the pharmaceutically acceptable salt thereof ranges or from about 1:27 to about 1:29.
857. The pharmaceutical composition of any one of the preceding embodiments, wherein the weight ratio between poloxamer 407 and the DMSO ranges from about 1:5 to about 40:1, from about 1:2 to about 15:1, from about 1:1 to about 8:1, from about 2:1 to about 4:1, or from about 2.5:1 to about 3.5:1; optionally, the weight ratio between poloxamer 407 and the DMSO is about 3:1.
858. The pharmaceutical composition of any one of the preceding embodiments, wherein the weight ratio between poloxamer 407 and the DMSO ranges from about 1:5 to about 40:1.
859. The pharmaceutical composition of any one of the preceding embodiments, wherein the weight ratio between poloxamer 407 and the DMSO ranges from about 1:2 to about 15:1.
860. The pharmaceutical composition of any one of the preceding embodiments, wherein the weight ratio between poloxamer 407 and the DMSO ranges from about 1:1 to about 8:1.
861. The pharmaceutical composition of any one of the preceding embodiments, wherein the weight ratio between poloxamer 407 and the DMSO ranges from about 2:1 to about 4:1.
862. The pharmaceutical composition of any one of the preceding embodiments, wherein the weight ratio between poloxamer 407 and the DMSO ranges or from about 2.5:1 to about 3.5:1.
863. The pharmaceutical composition of any one of the preceding embodiments, wherein the weight ratio between poloxamer 407 and the DMSO is about 3:1.
864. The pharmaceutical composition of any one of the preceding embodiments, wherein:
   the weight ratio between CHIR99021 and poloxamer 407 is about 0.02:1;
   the weight ratio between CHIR99021 and the DMSO is about 0.06:1;
   the weight ratio between valproic acid sodium salt and poloxamer 407 is about 0.54:1; and/or
   the weight ratio between valproic acid sodium salt and the DMSO is about 3.2:1.
865. The pharmaceutical composition of any one of the preceding embodiments, wherein the weight ratio between CHIR99021 and poloxamer 407 is about 0.02:1.
866. The pharmaceutical composition of any one of the preceding embodiments, wherein the weight ratio between CHIR99021 and the DMSO is about 0.06:1.
867. The pharmaceutical composition of any one of the preceding embodiments, wherein the weight ratio between valproic acid sodium salt and poloxamer 407 is about 0.54:1.
868. The pharmaceutical composition of any one of the preceding embodiments, wherein the weight ratio between valproic acid sodium salt and the DMSO is about 3.2:1.
869. The pharmaceutical composition of any one of the preceding embodiments, wherein:
   the concentration of CHIR99021 or the pharmaceutically acceptable salt thereof ranges from about 1.45 mg/ml to about 1.65 mg/ml;
   the concentration of valproic acid or the pharmaceutically acceptable salt thereof ranges from about 43 mg/ml to about 46 mg/ml;
   the concentration of poloxamer 407 ranges from about 7 wt % to about 8.5 wt %; and
   the concentration of DMSO ranges from about 2 wt % to about 3 wt %.
870. The pharmaceutical composition of any one of the preceding embodiments, wherein:
   the concentration of CHIR99021 or the pharmaceutically acceptable salt thereof ranges from about 1.45 mg/ml to about 1.65 mg/ml.
871. The pharmaceutical composition of any one of the preceding embodiments, wherein the concentration of valproic acid or the pharmaceutically acceptable salt thereof ranges from about 43 mg/ml to about 46 mg/ml.
872. The pharmaceutical composition of any one of the preceding embodiments, wherein the concentration of poloxamer 407 ranges from about 7 wt % to about 8.5 wt %.
873. The pharmaceutical composition of any one of the preceding embodiments, wherein the concentration of DMSO ranges from about 2 wt % to about 3 wt %.
874. The pharmaceutical composition of any one of the preceding embodiments, wherein:
   the concentration of CHIR99021 or the pharmaceutically acceptable salt thereof is about 1.55 mg/ml;
   the concentration of valproic acid or the pharmaceutically acceptable salt thereof is about 44.5 mg/ml;
   the concentration of poloxamer 407 is about 8 wt %; and
   the concentration of DMSO is about 2.5 wt %.
875. The pharmaceutical composition of any one of the preceding embodiments, wherein
   the concentration of CHIR99021 or the pharmaceutically acceptable salt thereof is about 1.55 mg/ml.
876. The pharmaceutical composition of any one of the preceding embodiments, wherein the concentration of valproic acid or the pharmaceutically acceptable salt thereof is about 44.5 mg/ml.
877. The pharmaceutical composition of any one of the preceding embodiments, wherein the concentration of poloxamer 407 is about 8 wt %.
878. The pharmaceutical composition of any one of the preceding embodiments, wherein the concentration of DMSO is about 2.5 wt %.
879. A pharmaceutical composition, comprising:
   i) CHIR99021 or a pharmaceutically acceptable salt thereof being present at a concentration ranging from 0.025 mg/ml to about 25 mg/ml;
   ii) valproic acid or a pharmaceutically acceptable salt thereof being present at a concentration ranging from 1 mg/ml to about 500 mg/ml;
   iii) poloxamer 407 being present at a concentration ranging from 1 wt % to about 25 wt %; and
   iv) dimethyl sulfoxide (DMSO) being present at a concentration below 7.5 wt %.
880. The pharmaceutical composition of any preceding embodiment, comprising valproic acid or a pharmaceutically acceptable salt thereof being present at a concentration ranging from 1 mg/ml to about 500 mg/ml.
881. The pharmaceutical composition of embodiment 879 or 880, wherein the pharmaceutically acceptable salt of valproic acid is a sodium salt;
   optionally, the pharmaceutically acceptable salt of valproic acid is sodium valproate.
882. The pharmaceutical composition of embodiment 881, wherein the pharmaceutically acceptable salt of valproic acid is sodium valproate.
883. The pharmaceutical composition of any one of the preceding embodiments, wherein the concentration of CHIR99021 or the pharmaceutically acceptable salt thereof ranges from about 0.05 mg/ml to about 5 mg/ml, from about 0.25 mg/ml to about 2.5 mg/ml, from about 0.5 mg/ml to about 1.75 mg/ml, or from about 1.45 mg/ml to about 1.65 mg/ml;
   optionally, the concentration of CHIR99021 or the pharmaceutically acceptable salt thereof is about 1.55 mg/ml.
884. The pharmaceutical composition of any one of the preceding embodiments, wherein the concentration of CHIR99021 or the pharmaceutically acceptable salt thereof ranges from about 0.05 mg/ml to about 10 mg/ml.
885. The pharmaceutical composition of any one of the preceding embodiments, wherein the concentration of CHIR99021 or the pharmaceutically acceptable salt thereof ranges from about 0.25 mg/ml to about 2.5 mg/ml.
886. The pharmaceutical composition of any one of the preceding embodiments, wherein the concentration of CHIR99021 or the pharmaceutically acceptable salt thereof ranges from about 0.5 mg/ml to about 1.75 mg/ml.
887. The pharmaceutical composition of any one of the preceding embodiments, wherein the concentration of CHIR99021 or the pharmaceutically acceptable salt thereof ranges from about 0.85 mg/ml to about 1.15 mg/ml; 888. The pharmaceutical composition of any one of the preceding embodiments, wherein the concentration of CHIR99021 or the pharmaceutically acceptable salt thereof is about 1.05 mg/ml.
889. The pharmaceutical composition of any one of the preceding embodiments, wherein the concentration of valproic acid or the pharmaceutically acceptable salt thereof ranges from about 2.5 mg/ml to about 200 mg/ml, from about 5 mg/ml to about 100 mg/ml, from about 15 mg/ml to about 50 mg/ml, from about 28 mg/ml to about 31 mg/ml;
   optionally, the concentration of valproic acid or the pharmaceutically acceptable salt thereof is about 29.5 mg/ml.
890. The pharmaceutical composition of any one of the preceding embodiments, wherein the concentration of valproic acid or the pharmaceutically acceptable salt thereof ranges from about 2.5 mg/ml to about 200 mg/ml.
891. The pharmaceutical composition of any one of the preceding embodiments, wherein the concentration of valproic acid or the pharmaceutically acceptable salt thereof ranges from about 5 mg/ml to about 100 mg/ml.
892. The pharmaceutical composition of any one of the preceding embodiments, wherein the concentration of valproic acid or the pharmaceutically acceptable salt thereof ranges from about 15 mg/ml to about 50 mg/ml, from about 28 mg/ml to about 31 mg/ml.
893. The pharmaceutical composition of any one of the preceding embodiments, wherein the concentration of valproic acid or the pharmaceutically acceptable salt thereof is about 29.5 mg/ml.
894. The pharmaceutical composition of any one of the preceding embodiments, wherein the concentration of poloxamer 407 ranges from about 2.5 wt % to about 12.5 wt %, from about 5 wt % to about 11 wt %, from about 11 wt % to about 10 wt %, from about 7 wt % to about 8.5 wt %;
   optionally, the concentration of poloxamer 407 is about 7.5 wt %.
895. The pharmaceutical composition of any one of the preceding embodiments, wherein the concentration of poloxamer 407 ranges from about 2.5 wt % to about 12.5 wt %.
896. The pharmaceutical composition of any one of the preceding embodiments, wherein the concentration of poloxamer 407 ranges from about 5 wt % to about 11 wt %.
897. The pharmaceutical composition of any one of the preceding embodiments, wherein the concentration of poloxamer 407 ranges from about 11 wt % to about 10 wt %.
898. The pharmaceutical composition of any one of the preceding embodiments, wherein the concentration of poloxamer 407 ranges from about 7 wt % to about 8.5 wt %
899. The pharmaceutical composition of any one of the preceding embodiments, wherein the concentration of poloxamer 407 is about 7.5 wt %.
900. The pharmaceutical composition of any one of the preceding embodiments, wherein the concentration of DMSO ranges from about 0.5 wt % to about 5 wt %, from about 1 wt % to about 4 wt %, from about 1.5 wt % to about 3.5 wt %, from about 2 wt % to about 3 wt %;
   optionally, the concentration of DMSO is about 2.5 wt %.
901. The pharmaceutical composition of any one of the preceding embodiments, wherein the concentration of DMSO ranges from about 0.5 wt % to about 5 wt %.
902. The pharmaceutical composition of any one of the preceding embodiments, wherein the concentration of DMSO ranges from about 1 wt % to about 4 wt %.
903. The pharmaceutical composition of any one of the preceding embodiments, wherein the concentration of DMSO ranges from about 1.5 wt % to about 3.5 wt %.
904. The pharmaceutical composition of any one of the preceding embodiments, wherein the concentration of DMSO ranges from about 2 wt % to about 3 wt %; 905. The pharmaceutical composition of any one of the preceding embodiments, wherein the concentration of DMSO is about 2.5 wt %.
906. The pharmaceutical composition of any one of the preceding embodiments, wherein the weight ratio between CHIR99021 or the pharmaceutically acceptable salt thereof and valproic acid or the pharmaceutically acceptable salt thereof ranges from about 1:5 to about 1:10, from about 1:10 to about 1:50, from about 1:20 to about 1:35, from about 1:25 to about 1:31, or from about 1:27 to about 1:29.
907. The pharmaceutical composition of any one of the preceding embodiments, wherein the weight ratio between CHIR99021 or the pharmaceutically acceptable salt thereof and valproic acid or the pharmaceutically acceptable salt thereof ranges from about 1:5 to about 1:10.
908. The pharmaceutical composition of any one of the preceding embodiments, wherein the weight ratio between CHIR99021 or the pharmaceutically acceptable salt thereof and valproic acid or the pharmaceutically acceptable salt thereof ranges from about 1:10 to about 1:50
909. The pharmaceutical composition of any one of the preceding embodiments, wherein the weight ratio between CHIR99021 or the pharmaceutically acceptable salt thereof and valproic acid or the pharmaceutically acceptable salt thereof ranges from about 1:20 to about 1:35.
910. The pharmaceutical composition of any one of the preceding embodiments, wherein the weight ratio between CHIR99021 or the pharmaceutically acceptable salt thereof and valproic acid or the pharmaceutically acceptable salt thereof ranges from about 1:25 to about 1:31.
911. The pharmaceutical composition of any one of the preceding embodiments, wherein the weight ratio between CHIR99021 or the pharmaceutically acceptable salt thereof and valproic acid or the pharmaceutically acceptable salt thereof ranges from about 1:27 to about 1:29.
912. The pharmaceutical composition of any one of the preceding embodiments, wherein the weight ratio between poloxamer 407 and the DMSO ranges from about 1:5 to about 40:1, from about 1:2 to about 15:1, from about 1:1 to about 8:1, from about 2:1 to about 4:1, or from about 2.5:1 to about 3.5:1;
   optionally, the weight ratio between poloxamer 407 and the DMSO is about 3:1.
913. The pharmaceutical composition of any one of the preceding embodiments, wherein the weight ratio between poloxamer 407 and the DMSO ranges from about 1:5 to about 40:1.
914. The pharmaceutical composition of any one of the preceding embodiments, wherein the weight ratio between poloxamer 407 and the DMSO ranges from about 1:2 to about 15:1.
915. The pharmaceutical composition of any one of the preceding embodiments, wherein the weight ratio between poloxamer 407 and the DMSO ranges from about 1:1 to about 8:1.
916. The pharmaceutical composition of any one of the preceding embodiments, wherein the weight ratio between poloxamer 407 and the DMSO ranges from about 2:1 to about 4:1, from about 2.5:1 to about 3.5:1.
917. The pharmaceutical composition of any one of the preceding embodiments, wherein the weight ratio between poloxamer 407 and the DMSO is about 3:1.
918. The pharmaceutical composition of any one of the preceding embodiments, wherein:
   the weight ratio between CHIR99021 and poloxamer 407 is about 0.016:1;
   the weight ratio between the CHIR99021 and the DMSO is about 0.06:1;
   the weight ratio between valproic acid sodium salt and poloxamer 407 is about 0.42:1; and/or the weight ratio between valproic acid sodium salt and the DMSO is about 1.5:1.
919. The pharmaceutical composition of any one of the preceding embodiments, wherein the weight ratio between CHIR99021 and poloxamer 407 is about 0.016:1
920. The pharmaceutical composition of any one of the preceding embodiments, wherein the weight ratio between the CHIR99021 and the DMSO is about 0.06:1.
921. The pharmaceutical composition of any one of the preceding embodiments, wherein the weight ratio between valproic acid sodium salt and poloxamer 407 is about 0.42:1.
922. The pharmaceutical composition of any one of the preceding embodiments, wherein the weight ratio between valproic acid sodium salt and the DMSO is about 1.5:1.
923. The pharmaceutical composition of any one of the preceding embodiments, wherein:
   the concentration of CHIR99021 or the pharmaceutically acceptable salt thereof ranges from about 0.95 mg/ml to about 1.15 mg/ml;
   the concentration of valproic acid or the pharmaceutically acceptable salt thereof ranges from about 28 mg/ml to about 31 mg/ml;
   the concentration of poloxamer 407 ranges from about 7 wt % to about 8.5 wt %; and
   the concentration of DMSO ranges from about 2 wt % to about 3 wt %.
924. The pharmaceutical composition of any one of the preceding embodiments, wherein the concentration of CHIR99021 or the pharmaceutically acceptable salt thereof ranges from about 0.95 mg/ml to about 1.15 mg/ml.
925. The pharmaceutical composition of any one of the preceding embodiments, wherein the concentration of valproic acid or the pharmaceutically acceptable salt thereof ranges from about 28 mg/ml to about 31 mg/ml.
926. The pharmaceutical composition of any one of the preceding embodiments, wherein the concentration of poloxamer 407 ranges from about 7 wt % to about 8.5 wt %;
927. The pharmaceutical composition of any one of the preceding embodiments, wherein the concentration of DMSO ranges from about 2 wt % to about 3 wt %.
928. The pharmaceutical composition of any one of the preceding embodiments, wherein:
   the concentration of CHIR99021 or the pharmaceutically acceptable salt thereof is about 1.05 mg/ml;
   the concentration of valproic acid or the pharmaceutically acceptable salt thereof is about 29.5 mg/ml;
   the concentration of poloxamer 407 is about 7.5 wt %; and
   the concentration of DMSO is about 2.5 wt %.
929. The pharmaceutical composition of any one of the preceding embodiments, wherein the concentration of CHIR99021 or the pharmaceutically acceptable salt thereof is about 1.05 mg/ml.
930. The pharmaceutical composition of any one of the preceding embodiments, wherein the concentration of valproic acid or the pharmaceutically acceptable salt thereof is about 29.5 mg/ml.
931. The pharmaceutical composition of any one of the preceding embodiments, wherein the concentration of poloxamer 407 is about 7.5 wt %.
932. The pharmaceutical composition of any one of the preceding embodiments, wherein the concentration of DMSO is about 2.5 wt %.
933. A pharmaceutical composition, comprising:
   i) CHIR99021 or a pharmaceutically acceptable salt thereof being present at a concentration ranging from 0.025 mg/ml to about 25 mg/ml;
   ii) valproic acid or a pharmaceutically acceptable salt thereof being present at a concentration ranging from 0.5 mg/ml to about 500 mg/ml;
   iii) poloxamer 407 being present at a concentration ranging from 1 wt % to about 25 wt %; and
   iv) dimethyl sulfoxide (DMSO) being present at a concentration below 7.5 wt %.
934. The pharmaceutical composition of embodiment 933, wherein the pharmaceutically acceptable salt of valproic acid is a sodium salt;
   optionally, the pharmaceutically acceptable salt of valproic acid is sodium valproate.
935. The pharmaceutical composition of embodiment 934, wherein the pharmaceutically acceptable salt of valproic acid is sodium valproate.
936. The pharmaceutical composition of any one of the preceding embodiments, wherein the concentration of CHIR99021 or the pharmaceutically acceptable salt thereof ranges from about 0.05 mg/ml to about 5 mg/ml, from about 0.25 mg/ml to about 2.5 mg/ml, from about 0.5 mg/ml to about 1.75 mg/ml, or from about 0.6 mg/ml to about 0.75 mg/ml;
   optionally, the concentration of CHIR99021 or the pharmaceutically acceptable salt thereof ranges is about 0.7 mg/ml.
937. The pharmaceutical composition of any one of the preceding embodiments, wherein the concentration of CHIR99021 or the pharmaceutically acceptable salt thereof ranges from about 0.05 mg/ml to about 5 mg/ml.
938. The pharmaceutical composition of any one of the preceding embodiments, wherein the concentration of CHIR99021 or the pharmaceutically acceptable salt thereof ranges from about 0.25 mg/ml to about 2.5 mg/ml.
939. The pharmaceutical composition of any one of the preceding embodiments, wherein the concentration of CHIR99021 or the pharmaceutically acceptable salt thereof ranges from about 0.5 mg/ml to about 1.75 mg/ml.
940. The pharmaceutical composition of any one of the preceding embodiments, wherein the concentration of CHIR99021 or the pharmaceutically acceptable salt thereof ranges or from about 0.6 mg/ml to about 0.75 mg/ml.
941. The pharmaceutical composition of any one of the preceding embodiments, wherein the concentration of CHIR99021 or the pharmaceutically acceptable salt thereof ranges is about 0.7 mg/ml.
942. The pharmaceutical composition of any one of the preceding embodiments, wherein the concentration of valproic acid or the pharmaceutically acceptable salt thereof ranges from about 2.5 mg/ml to about 200 mg/ml, from about 5 mg/ml to about 100 mg/ml, from about 15 mg/ml to about 50 mg/ml, or from about 18 mg/ml to about 21 mg/ml;
   optionally, the concentration of valproic acid or the pharmaceutically acceptable salt thereof is about 19.5 mg/ml.
943. The pharmaceutical composition of any one of the preceding embodiments, wherein the concentration of valproic acid or the pharmaceutically acceptable salt thereof ranges from about 2.5 mg/ml to about 200 mg/ml.
944. The pharmaceutical composition of any one of the preceding embodiments, wherein the concentration of valproic acid or the pharmaceutically acceptable salt thereof ranges from about 5 mg/ml to about 100 mg/ml.
945. The pharmaceutical composition of any one of the preceding embodiments, wherein the concentration of valproic acid or the pharmaceutically acceptable salt thereof ranges from about 15 mg/ml to about 50 mg/ml.
946. The pharmaceutical composition of any one of the preceding embodiments, wherein the concentration of valproic acid or the pharmaceutically acceptable salt thereof ranges from about 18 mg/ml to about 21 mg/ml.
947. The pharmaceutical composition of any one of the preceding embodiments, wherein the concentration of valproic acid or the pharmaceutically acceptable salt thereof is about 19.5 mg/ml.
948. The pharmaceutical composition of any one of the preceding embodiments, wherein the concentration of poloxamer 407 ranges from about 2.5 wt % to about 12.5 wt %, from about 5 wt % to about 11 wt %, from about 6 wt % to about 10 wt %, or from about 7 wt % to about 8.5 wt %;
   optionally, the concentration of poloxamer 407 is about 7.5 wt %.
949. The pharmaceutical composition of any one of the preceding embodiments, wherein the concentration of poloxamer 407 ranges from about 2.5 wt % to about 12.5 wt %.
950. The pharmaceutical composition of any one of the preceding embodiments, wherein the concentration of poloxamer 407 ranges from about 5 wt % to about 11 wt %.
951. The pharmaceutical composition of any one of the preceding embodiments, wherein the concentration of poloxamer 407 ranges from about 6 wt % to about 10 wt %.
952. The pharmaceutical composition of any one of the preceding embodiments, wherein the concentration of poloxamer 407 ranges from about 7 wt % to about 8.5 wt %.
953. The pharmaceutical composition of any one of the preceding embodiments, wherein the concentration of poloxamer 407 is about 7.5 wt %.
954. The pharmaceutical composition of any one of the preceding embodiments, wherein the concentration of DMSO ranges from about 0.5 wt % to about 5 wt %, from about 1 wt % to about 4 wt %, from about 1.5 wt % to about 3.5 wt %, or from about 2 wt % to about 3 wt %;
   optionally, the concentration of DMSO is about 5 wt %.
955. The pharmaceutical composition of any one of the preceding embodiments, wherein the concentration of DMSO ranges from about 0.5 wt % to about 5 wt %.
956. The pharmaceutical composition of any one of the preceding embodiments, wherein the concentration of DMSO ranges from about 1 wt % to about 4 wt %.
957. The pharmaceutical composition of any one of the preceding embodiments, wherein the concentration of DMSO ranges from about 1.5 wt % to about 3.5 wt %.
958. The pharmaceutical composition of any one of the preceding embodiments, wherein the concentration of DMSO ranges from about 2 wt % to about 3 wt %.
959. The pharmaceutical composition of any one of the preceding embodiments, wherein the concentration of DMSO is about 5 wt %.
960. The pharmaceutical composition of any one of the preceding embodiments, wherein the weight ratio between CHIR99021 or the pharmaceutically acceptable salt thereof and valproic acid or the pharmaceutically acceptable salt thereof ranges from about 1:5 to about 1:10, from about 1:10 to about 1:50, from about 1:20 to about 1:35, from about 1:25 to about 1:31, or from about 1:27 to about 1:29.
961. The pharmaceutical composition of any one of the preceding embodiments, wherein the weight ratio between CHIR99021 or the pharmaceutically acceptable salt thereof and valproic acid or the pharmaceutically acceptable salt thereof ranges from about 1:5 to about 1:10.
962. The pharmaceutical composition of any one of the preceding embodiments, wherein the weight ratio between CHIR99021 or the pharmaceutically acceptable salt thereof and valproic acid or the pharmaceutically acceptable salt thereof ranges from about 1:10 to about 1:50.
963. The pharmaceutical composition of any one of the preceding embodiments, wherein the weight ratio between CHIR99021 or the pharmaceutically acceptable salt thereof and valproic acid or the pharmaceutically acceptable salt thereof ranges from about 1:20 to about 1:35.
964. The pharmaceutical composition of any one of the preceding embodiments, wherein the weight ratio between CHIR99021 or the pharmaceutically acceptable salt thereof and valproic acid or the pharmaceutically acceptable salt thereof ranges from about 1:25 to about 1:31.
965. The pharmaceutical composition of any one of the preceding embodiments, wherein the weight ratio between CHIR99021 or the pharmaceutically acceptable salt thereof and valproic acid or the pharmaceutically acceptable salt thereof ranges from about 1:27 to about 1:29.
966. The pharmaceutical composition of any one of the preceding embodiments, wherein the weight ratio between poloxamer 407 and the DMSO ranges from about 1:5 to about 40:1, from about 1:2 to about 15:1, from about 1:1 to about 8:1, from about 2:1 to about 4:1, from about 2.5:1 to about 3.5:1.
967. The pharmaceutical composition of any one of the preceding embodiments, wherein the weight ratio between poloxamer 407 and the DMSO ranges from about 1:5 to about 40:1.
968. The pharmaceutical composition of any one of the preceding embodiments, wherein the weight ratio between poloxamer 407 and the DMSO ranges from about 1:2 to about 15:1.
969. The pharmaceutical composition of any one of the preceding embodiments, wherein the weight ratio between poloxamer 407 and the DMSO ranges from about 1:1 to about 8:1.
970. The pharmaceutical composition of any one of the preceding embodiments, wherein the weight ratio between poloxamer 407 and the DMSO ranges from about 2:1 to about 4:1.
971. The pharmaceutical composition of any one of the preceding embodiments, wherein the weight ratio between poloxamer 407 and the DMSO ranges from about 2.5:1 to about 3.5:1.
972. The pharmaceutical composition of any one of the preceding embodiments, wherein:
   the weight ratio between poloxamer 407 and the DMSO is about 3:1;
   the weight ratio between the CHIR99021 and poloxamer 407 is about 0.013:1;
   the weight ratio between CHIR99021 and the DMSO is about 0.06:1;
   the weight ratio between valproic acid sodium salt and poloxamer 407 is about 0.23:1; and/or
   the weight ratio between valproic acid sodium salt and the DMSO is about 1.8:1.
973. The pharmaceutical composition of any one of the preceding embodiments, wherein the weight ratio between poloxamer 407 and the DMSO is about 3:1.
974. The pharmaceutical composition of any one of the preceding embodiments, wherein the weight ratio between the CHIR99021 and poloxamer 407 is about 0.013:1.
975. The pharmaceutical composition of any one of the preceding embodiments, wherein the weight ratio between CHIR99021 and the DMSO is about 0.06:1.
976. The pharmaceutical composition of any one of the preceding embodiments, wherein the weight ratio between valproic acid sodium salt and poloxamer 407 is about 0.23:1.
977. The pharmaceutical composition of any one of the preceding embodiments, wherein the weight ratio between valproic acid sodium salt and the DMSO is about 1.8:1.
978. The pharmaceutical composition of any one of the preceding embodiments, wherein:
   the concentration of CHIR99021 or the pharmaceutically acceptable salt thereof ranges from about 0.6 mg/ml to about 0.75 mg/ml;
   the concentration of valproic acid or the pharmaceutically acceptable salt thereof ranges from about 18 mg/ml to about 21 mg/ml;
   the concentration of poloxamer 407 ranges from about 7 wt % to about 8.5 wt %; and the concentration of DMSO ranges from about 2 wt % to about 3 wt %.
979. The pharmaceutical composition of any one of the preceding embodiments, wherein the concentration of CHIR99021 or the pharmaceutically acceptable salt thereof ranges from about 0.6 mg/ml to about 0.75 mg/ml.
980. The pharmaceutical composition of any one of the preceding embodiments, wherein the concentration of valproic acid or the pharmaceutically acceptable salt thereof ranges from about 18 mg/ml to about 21 mg/ml.
981. The pharmaceutical composition of any one of the preceding embodiments, wherein the concentration of poloxamer 407 ranges from about 7 wt % to about 8.5 wt %.
982. The pharmaceutical composition of any one of the preceding embodiments, wherein the concentration of DMSO ranges from about 2 wt % to about 3 wt %.
983. The pharmaceutical composition of any one of the preceding embodiments, wherein:
   the concentration of CHIR99021 or the pharmaceutically acceptable salt thereof is about 0.7 mg/ml;
   the concentration of valproic acid or the pharmaceutically acceptable salt thereof is about 19.5 mg/ml;
   the concentration of poloxamer 407 is about 7.5 wt %; and
   the concentration of DMSO is about 2.5 wt %.
984. The pharmaceutical composition of any one of the preceding embodiments, wherein the concentration of CHIR99021 or the pharmaceutically acceptable salt thereof is about 0.7 mg/ml.
985. The pharmaceutical composition of any one of the preceding embodiments, wherein the concentration of valproic acid or the pharmaceutically acceptable salt thereof is about 19.5 mg/ml.
986. The pharmaceutical composition of any one of the preceding embodiments, wherein the concentration of poloxamer 407 is about 7.5 wt %.
987. The pharmaceutical composition of any one of the preceding embodiments, wherein the concentration of DMSO is about 2.5 wt %.
988. The pharmaceutical composition of any one of the preceding embodiments, comprising one or more of:
   water or a buffering agent;
   a bulking agent;
   a stabilizing agent;
   a tonicity-adjusting agent; and
   a soothing agent
989. The pharmaceutical composition of any one of the preceding embodiments, comprising water or a buffering agent
990. The pharmaceutical composition of any one of the preceding embodiments, comprising a bulking agent
991. The pharmaceutical composition of any one of the preceding embodiments, comprising a stabilizing agent
992. The pharmaceutical composition of any one of the preceding embodiments, comprising a tonicity-adjusting agent
993. The pharmaceutical composition of any one of the preceding embodiments, comprising a soothing agent
994. A lyophilized pharmaceutical composition comprising the pharmaceutical composition of any preceding embodiment
995. A method for lyophilizing a pharmaceutical composition, the method comprising:
   (a) Providing a pharmaceutical composition;
   (b) Lyophilizing the composition; and
   (c) obtaining a lyophilized pharmaceutical composition
996. The method of embodiment 995, wherein step (b) further comprises:
   (i) reducing the temperature in the lyophilizer to less than about −15° C.
997. The method of embodiment 996 or 997, wherein step (b) further comprises:
   (i) reducing the temperature in the lyophilizer to less than about −30° C.
998. The method of any preceding embodiment, wherein step (b) further comprises:
   (i) reducing the temperature in the lyophilizer to less than −45° C.
999. The method of any preceding embodiment, wherein step (b)(i) further comprises reducing the temperature at a rate of less than about 2° C. per minute.
1000. The method of any preceding embodiment, wherein step (b)(i) further comprises reducing the temperature at a rate of less than about 1° C. per minute.
1001. The method of any preceding embodiment, wherein step (b)(i) further comprises reducing the temperature at a rate of less than about 0.5° C. per minute.
1002. The method of any preceding embodiment, wherein step (b)(i) further additionally comprises maintaining the temperature for less than 5 hours.
1003. The method of any preceding embodiment, wherein step (b)(i) further additionally comprises maintaining the temperature for less than 4 hours.
1004. The method of any preceding embodiment, wherein step (b)(i) further additionally comprises maintaining the temperature for less than 3 hours.
1005. The method of any preceding embodiment, wherein step (b) comprises:
   (i) reducing the temperature in the lyophilizer to less than about −40° C. at a rate of less than about 0.75° C. per minute, and then holding it at less than about −40° C. for less than about 3.5 hours.
1006. The method of any preceding embodiment, wherein step (b) comprises:
   (i) reducing the temperature in the lyophilizer to about −45° C. at a rate of about 0.5° C. per minute, and then holding it at about −45° C. for about 3 hours.
1007. The method of any preceding embodiment, wherein step (b) further comprises:
   (ii) applying a vacuum of less than 150 mTorr.
1008. The method of any preceding embodiment, wherein step (b) further comprises:
   (ii) applying a vacuum of less than 100 mTorr.
1009. The method of any preceding embodiment, wherein step (b) further comprises:
   (ii) applying a vacuum of less than 90 mTorr.
1010. The method of any preceding embodiment, wherein step (b) further comprises:
   (ii) applying a vacuum of about 80 mTorr.
1011. The method of any preceding embodiment, wherein step (b) further comprises:
   (iii) increasing the temperature to less than about −10° C.
1012. The method of any preceding embodiment, wherein step (b) further comprises:
   (iii) increasing the temperature to less than about −20° C.
1013. The method of any preceding embodiment, wherein step (b) further comprises:
   (iii) increasing the temperature to less than −30° C.
1014. The method of any preceding embodiment, wherein step (b)(iii) further comprises reducing the temperature at a rate of less than about 2° C. per minute.
1015. The method of any preceding embodiment, wherein step (b)(iii) further comprises reducing the temperature at a rate of less than about 1° C. per minute.
1016. The method of any preceding embodiment, wherein step (b)(iii) further comprises reducing the temperature at a rate of less than about 0.5° C. per minute.
1017. The method of any preceding embodiment, wherein step (b)(iii) further comprises maintaining the temperature for less than 25 hours.
1018. The method of any preceding embodiment, wherein step (b)(iii) further comprises maintaining the temperature for less than 20 hours.
1019. The method of any preceding embodiment, wherein step (b)(iii) further comprises maintaining the temperature for less than 15 hours.
1020. The method of any preceding embodiment, wherein step (b)(iii) further comprises maintaining the vacuum of less than about 200 mTorr.
1021. The method of any preceding embodiment, wherein step (b)(iii) further comprises maintaining the vacuum of less than about 150 mTorr.
1022. The method of any preceding embodiment, wherein step (b)(iii) further comprises maintaining the vacuum of less than about 100 mTorr.
1023. The method of any preceding embodiment, wherein step (b) comprises:
   (iii) increasing the temperature to less than about −25° C. at a rate of less than about 0.75° C. per minute, and holding it at less than about −25° C. for less than about 17.5 hours under a vacuum of less than 100 mTorr.
1024. The method of any preceding embodiment, wherein step (b) comprises:
   (iii) increasing the temperature to about −30° C. at a rate of about 0.5° C. per minute, and holding it at about −30° C. for about 15 hours under a vacuum of about 80 mTorr.
1025. The method of any preceding embodiment, wherein step (b) comprises:
   (iv) increasing the temperature to greater than about 15° C.
1026. The method of any preceding embodiment, wherein step (b) comprises:
   (iv) increasing the temperature to greater than about 10° C.
1027. The method of any preceding embodiment, wherein step (b) comprises:
   (iv) increasing the temperature to greater than about 5° C.
1028. The method of any preceding embodiment, wherein step (b)(iv) further comprises increasing the temperature at a rate of less than about 2° C. per minute.
1029. The method of any preceding embodiment, wherein step (b)(iv) further comprises increasing the temperature at a rate of less than about 1.5° C. per minute.
1030. The method of any preceding embodiment, wherein step (b)(iv) further comprises increasing the temperature at a rate of less than about 1.0° C. per minute.
1031. The method of any preceding embodiment, wherein step (b)(iv) further comprises increasing the temperature at a rate of less than about 0.5° C. per minute.
1032. The method of any preceding embodiment, wherein step (b) comprises:
   (v) holding the temperature at greater than about 15° C.
1033. The method of any preceding embodiment, wherein step (b) comprises:
   (v) holding the temperature at greater than about 10° C.
1034. The method of any preceding embodiment, wherein step (b) comprises:
   (v) holding the temperature at greater than about 5° C.
1035. The method of any preceding embodiment, wherein step (b)(v) further comprises maintaining the temperature for more than about 5 hours.
1036. The method of any preceding embodiment, wherein step (b)(v) further comprises maintaining the temperature for more than about 10 hours.
1037. The method of any preceding embodiment, wherein step (b)(v) further comprises maintaining the temperature for more than about 15 hours.
1038. The method of any preceding embodiment, wherein step (b)(v) further comprises maintaining the temperature for more than about 20 hours.
1039. The method of any preceding embodiment, wherein step (b)(v) further comprises maintaining the vacuum of less than about 200 mTorr.
1040. The method of any preceding embodiment, wherein step (b)(v) further comprises maintaining the vacuum of less than about 150 mTorr.
1041. The method of any preceding embodiment, wherein step (b)(v) further comprises maintaining the vacuum of less than about 100 mTorr.
1042. The method of any preceding embodiment, wherein step (b)(v) further comprises maintaining the vacuum of about 80 mTorr.
1043. The method of any preceding embodiment, further comprising an annealing step.
1044. The method of any preceding embodiment, wherein the method comprises:
   (a) Providing a pharmaceutical composition;
   (b) Lyophilizing the composition by:
   (i) reducing the temperature in the lyophilizer to −45° C. at a rate of 0.5° C. per minute, and then holding it at −45° C. for 3 hours;
   (ii) applying a vacuum of 80 mTorr;
   (iii) increasing the temperature to −30° C. (at a rate of 0.5° C. per minute) and holding it at −30° C. for 15 hours under a vacuum of 80 mTorr;
   (iv) increasing the temperature to 15° C. (at a rate of 0.5° C. per minute); and/or
   (v) holding the temperature at 15° C. for 20 hours under a vacuum of 80 mTorr; and
   (c) obtaining a lyophilized pharmaceutical composition
1045. The method of any preceding embodiment, wherein the composition is subjected to a temperature of at least −50° C. prior to lyophilization.
1046. A method of processing the pharmaceutical composition of any preceding embodiment to form a lyophilized pharmaceutical composition
1047. The method of embodiment 1046, comprising the steps of:
   i) cooling the pharmaceutical composition at a first temperature below 0° C. for a first period of time;
   ii) removing one or more solvents from the resulting mixture of step (i) at a second temperature below 0° C., and at a reduced pressure below 760 Torr, for a second period of time.
1048. The method of embodiment 1046 or embodiment 1047, comprising one or more steps selected from:
   0a) dispensing the pharmaceutical composition in a sterile vial;
   ia) cooling the pharmaceutical composition at a rate ranging from about 0.1° C. per minute to about 5° C. per minute to the first temperature ranging from about −20° C. to about −80° C.;
   ib) holding the pharmaceutical composition at the first temperature for the first period of time ranging from about 1 hour to about 6 hours;
   iia) subjecting the pharmaceutical composition to the reduced pressure ranging from about 1 mTorr to 1000 mTorr and warming the pharmaceutical composition at a rate ranging from about 0.1° C. per minute to about 5° C. per minute to the second temperature ranging from about −10° C. to −50° C.;
   iib) holding the pharmaceutical composition at the second temperature and under the reduced pressure or the second period of time ranging from about 10 hours to about 30 hours;
   iiia) filling the sterile vial with nitrogen; and iiib) capping and crimping the sterile vial.
1049. The method of any preceding embodiment, comprising dispensing the pharmaceutical composition in a sterile vial.
1050. The method of any preceding embodiment, comprising cooling the pharmaceutical composition at a rate ranging from about 0.1° C. per minute to about 5° C. per minute to the first temperature ranging from about −20° C. to about −80° C.
1051. The method of any preceding embodiment, comprising holding the pharmaceutical composition at the first temperature for the first period of time ranging from about 1 hour to about 6 hours
1052. The method of any preceding embodiment, comprising subjecting the pharmaceutical composition to the reduced pressure ranging from about 1 mTorr to 1000 mTorr and warming the pharmaceutical composition at a rate ranging from about 0.1° C. per minute to about 5° C. per minute to the second temperature ranging from about −10° C. to −50° C.
1053. The method of any preceding embodiment, comprising holding the pharmaceutical composition at the second temperature and under the reduced pressure or the second period of time ranging from about 10 hours to about 30 hours.
1054. The method of any preceding embodiment, comprising filling the sterile vial with nitrogen.
1055. The method of any preceding embodiment, comprising capping and crimping the sterile vial.
1056. The method of any any preceding embodiment, wherein the pharmaceutical composition comprises the one or more otic therapeutic agents and the poloxamer;
   optionally, the pharmaceutical composition comprises the one or more otic therapeutic agents and poloxamer 407; and
   optionally, the pharmaceutical composition comprises the one or more otic therapeutic agents and purified poloxamer 407.
   The method of any any preceding embodiment, wherein the pharmaceutical composition comprises the one or more otic therapeutic agents and the poloxamer;
1057. The method of any preceding embodiment, wherein the pharmaceutical composition comprises the one or more otic therapeutic agents and poloxamer 407.
1058. The method of any preceding embodiment, wherein the pharmaceutical composition comprises the one or more otic therapeutic agents and purified poloxamer 407.
1059. The method of any preceding embodiment, wherein the pharmaceutical composition comprises CHIR99021, valproic acid sodium salt, the poloxamer, DMSO, and water;
   optionally, the pharmaceutical composition comprises CHIR99021, valproic acid sodium salt, poloxamer 407, DMSO, and water;
   optionally, the pharmaceutical composition comprises CHIR99021, valproic acid sodium salt, purified poloxamer 407, DMSO, and water.
1060. The method of any preceding embodiment, wherein the pharmaceutical composition comprises CHIR99021, valproic acid sodium salt, the poloxamer, DMSO, and water.
1061. The method of any any preceding embodiment, wherein the pharmaceutical composition comprises CHIR99021, valproic acid sodium salt, poloxamer 407, DMSO, and water.
1062. The method of any preceding embodiment, wherein the pharmaceutical composition comprises CHIR99021, valproic acid sodium salt, purified poloxamer 407, DMSO, and water.
1063. The method of any preceding embodiment, comprising one or more steps selected from:
   0a) dispensing the pharmaceutical composition in a sterile vial;
   ia) cooling the pharmaceutical composition at a rate of about 0.5° C. per minute to the first temperature of about −45° C.;
   ib) holding the pharmaceutical composition at the first temperature for the first period of time of about 3 hours;
   iia) subjecting the pharmaceutical composition to the reduced pressure of about 80 mTorr to 1000 mTorr and warming the pharmaceutical composition at a rate of about 0.5° C. per minute to the second temperature of about −30° C.;
   iib) holding the pharmaceutical composition at the second temperature and under the reduced pressure for the second period of time ranging from about 10 hours to about 15 hours;
   iic) warming the pharmaceutical composition at a rate of about 0.5° C. per minute to 20° C.;
   iid) holding the pharmaceutical composition at 20° C. and under the reduced pressure for 20 hours,
   iiia) filling the sterile vial with nitrogen; and
   iiib) capping and crimping the sterile vial.
1064. The method of any preceding embodiment, comprising
1065. The method of any preceding embodiment, comprising dispensing the pharmaceutical composition in a sterile vial.
1066. The method of any preceding embodiment, comprising cooling the pharmaceutical composition at a rate of about 0.5° C. per minute to the first temperature of about −45° C.
1067. The method of any preceding embodiment, comprising holding the pharmaceutical composition at the first temperature for the first period of time of about 3 hours.
1068. The method of any preceding embodiment, comprising subjecting the pharmaceutical composition to the reduced pressure of about 80 mTorr to 1000 mTorr and warming the pharmaceutical composition at a rate of about 0.5° C. per minute to the second temperature of about −30° C.
1069. The method of any preceding embodiment, comprising holding the pharmaceutical composition at the second temperature and under the reduced pressure for the second period of time ranging from about 10 hours to about 15 hours.
1070. The method of any preceding embodiment, comprising warming the pharmaceutical composition at a rate of about 0.5° C. per minute to 20° C.
1071. The method of any preceding embodiment, comprising holding the pharmaceutical composition at 20° C. and under the reduced pressure for 20 hours.
1072. The method of any preceding embodiment, comprising filling the sterile vial with nitrogen.
1073. The method of any preceding embodiment, comprising capping and crimping the sterile vial.
1074. A lyophilized pharmaceutical composition being prepared by lyophilizing the pharmaceutical composition of any preceding embodiment
1075. A lyophilized pharmaceutical composition being prepared by the method of any preceding embodiment
1076. A reconstituted solution being prepared by adding a diluent to the lyophilized pharmaceutical composition of any preceding embodiment
1077. A reconstituted solution being prepared by adding a diluent to a lyophilized pharmaceutical composition which is prepared by lyophilizing the pharmaceutical composition of any preceding embodiment
1078. A reconstituted solution being prepared by adding a diluent to a lyophilized pharmaceutical composition which is prepared by the method of any preceding embodiment
1079. A reconstituted solution being prepared by adding a diluent to a lyophilized pharmaceutical composition, comprising one or more otic therapeutic agents and a gelling agent
1080. The reconstituted solution of any one of the preceding embodiments, wherein the one or more otic therapeutic agents are one or more hearing loss treatment agents.
1081. The reconstituted solution of any one of the preceding embodiments, wherein the one or more otic therapeutic agents are CHR99021 or a pharmaceutical acceptable salt thereof, and valproic acid or a pharmaceutical acceptable salt thereof
1082. The reconstituted solution of embodiment 1081, wherein the pharmaceutical acceptable salt of valproic acid is a sodium salt;
   optionally, the pharmaceutically acceptable salt of valproic acid is sodium valproate.
1083. The reconstituted solution of embodiment 1082, wherein the pharmaceutical acceptable salt of valproic acid is a sodium salt
1084. The reconstituted solution of embodiment 1082, wherein the pharmaceutically acceptable salt of valproic acid is sodium valproate.
1085. The reconstituted solution of any one of the preceding embodiments, wherein the gelling agent is a thermoreversible gelling agent
1086. The reconstituted solution of embodiment 1085, wherein the thermoreversible gelling agent comprises a poloxamer.
1087. The reconstituted solution of embodiment 1086, wherein the poloxamer is selected from the group consisting of Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, and Poloxamer 407;
   optionally, the poloxamer is Poloxamer 188 or Poloxamer 407; and
   optionally, the poloxamer is Poloxamer 407.
1088. The reconstituted solution of embodiment 1087, wherein the poloxamer is
1089. The reconstituted solution of embodiment 1087, wherein the poloxamer is Poloxamer 101.
1090. The reconstituted solution of embodiment 1087, wherein the poloxamer is Poloxamer 105.
1091. The reconstituted solution of embodiment 1087, wherein the poloxamer is Poloxamer 108.
1092. The reconstituted solution of embodiment 1087, wherein the poloxamer is Poloxamer 122.
1093. The reconstituted solution of embodiment 1087, wherein the poloxamer is Poloxamer 123.
1094. The reconstituted solution of embodiment 1087, wherein the poloxamer is Poloxamer 124.
1095. The reconstituted solution of embodiment 1087, wherein the poloxamer is Poloxamer 181.
1096. The reconstituted solution of embodiment 1087, wherein the poloxamer is Poloxamer 182.
1097. The reconstituted solution of embodiment 1087, wherein the poloxamer is Poloxamer 183.
1098. The reconstituted solution of embodiment 1087, wherein the poloxamer is Poloxamer 184.
1099. The reconstituted solution of embodiment 1087, wherein the poloxamer is Poloxamer 185.
1100. The reconstituted solution of embodiment 1087, wherein the poloxamer is Poloxamer 188.
1101. The reconstituted solution of embodiment 1087, wherein the poloxamer is Poloxamer 212.
1102. The reconstituted solution of embodiment 1087, wherein the poloxamer is Poloxamer 215.
1103. The reconstituted solution of embodiment 1087, wherein the poloxamer is Poloxamer 217.
1104. The reconstituted solution of embodiment 1087, wherein the poloxamer is Poloxamer 231.
1105. The reconstituted solution of embodiment 1087, wherein the poloxamer is Poloxamer 234.
1106. The reconstituted solution of embodiment 1087, wherein the poloxamer is Poloxamer 235.
1107. The reconstituted solution of embodiment 1087, wherein the poloxamer is Poloxamer 237.
1108. The reconstituted solution of embodiment 1087, wherein the poloxamer is Poloxamer 238.
1109. The reconstituted solution of embodiment 1087, wherein the poloxamer is Poloxamer 282.
1110. The reconstituted solution of embodiment 1087, wherein the poloxamer is Poloxamer 284.
1111. The reconstituted solution of embodiment 1087, wherein the poloxamer is Poloxamer 288.
1112. The reconstituted solution of embodiment 1087, wherein the poloxamer is Poloxamer 331.
1113. The reconstituted solution of embodiment 1087, wherein the poloxamer is Poloxamer 333.
1114. The reconstituted solution of embodiment 1087, wherein the poloxamer is Poloxamer 334.
1115. The reconstituted solution of embodiment 1087, wherein the poloxamer is Poloxamer 335.
1116. The reconstituted solution of embodiment 1087, wherein the poloxamer is Poloxamer 338.
1117. The reconstituted solution of embodiment 1087, wherein the poloxamer is Poloxamer 401.
1118. The reconstituted solution of embodiment 1087, wherein the poloxamer is Poloxamer 402.
1119. The reconstituted solution of embodiment 1087, wherein the poloxamer is Poloxamer 403.
1120. The reconstituted solution of embodiment 1087, wherein the poloxamer is Poloxamer 407;
1121. The reconstituted solution of any one of the preceding embodiments, wherein the poloxamer is a purified poloxamer;
   optionally, the poloxamer is purified Poloxamer 407.
1122. The reconstituted solution of any one of the preceding embodiments, wherein the poloxamer is a purified poloxamer.
1123. The reconstituted solution of any one of the preceding embodiments, wherein the poloxamer is purified Poloxamer 407.
1124. The reconstituted solution of any one of the preceding embodiments, wherein the purified Poloxamer 407 has an average molecular weight of about 9 kDa or greater, about 9.2 kDa or greater, about 9.4 kDa or greater, about 9.6 kDa or greater, about 9.8 kDa or greater, about 10 kDa or greater, about 10.2 kDa or greater, about
10.4 kDa or greater, about 10.6 kDa or greater, about 10.8 kDa or greater, about 11 kDa or greater, about 11.2 kDa or greater, about 11.4 kDa or greater, about 11.6 kDa or greater, about 11.8 kDa or greater, about 12 kDa or greater, or about 12.1 kDa or greater.
1125. The reconstituted solution of any one of the preceding embodiments, wherein the purified Poloxamer 407 has an average molecular weight of about 9 kDa or greater.
1126. The reconstituted solution of any one of the preceding embodiments, wherein the purified Poloxamer 407 has an average molecular weight of about 9.2 kDa or greater.
1127. The reconstituted solution of any one of the preceding embodiments, wherein the purified Poloxamer 407 has an average molecular weight of about 9.4 kDa or greater.
1128. The reconstituted solution of any one of the preceding embodiments, wherein the purified Poloxamer 407 has an average molecular weight of about 9.6 kDa or greater.
1129. The reconstituted solution of any one of the preceding embodiments, wherein the purified Poloxamer 407 has an average molecular weight of about 9.8 kDa or greater.
1130. The reconstituted solution of any one of the preceding embodiments, wherein the purified Poloxamer 407 has an average molecular weight of about 10 kDa or greater.
1131. The reconstituted solution of any one of the preceding embodiments, wherein the purified Poloxamer 407 has an average molecular weight of about 10.2 kDa or greater.
1132. The reconstituted solution of any one of the preceding embodiments, wherein the purified Poloxamer 407 has an average molecular weight of about 10.4 kDa or greater.
1133. The reconstituted solution of any one of the preceding embodiments, wherein the purified Poloxamer 407 has an average molecular weight of about 10.6 kDa or greater.
1134. The reconstituted solution of any one of the preceding embodiments, wherein the purified Poloxamer 407 has an average molecular weight of about 10.8 kDa or greater.
1135. The reconstituted solution of any one of the preceding embodiments, wherein the purified Poloxamer 407 has an average molecular weight of about 11 kDa or greater.
1136. The reconstituted solution of any one of the preceding embodiments, wherein the purified Poloxamer 407 has an average molecular weight of about 11.2 kDa or greater.
1137. The reconstituted solution of any one of the preceding embodiments, wherein the purified Poloxamer 407 has an average molecular weight of about 11.4 kDa or greater.
1138. The reconstituted solution of any one of the preceding embodiments, wherein the purified Poloxamer 407 has an average molecular weight of about 11.6 kDa or greater.
1139. The reconstituted solution of any one of the preceding embodiments, wherein the purified Poloxamer 407 has an average molecular weight of about 11.8 kDa or greater.
1140. The reconstituted solution of any one of the preceding embodiments, wherein the purified Poloxamer 407 has an average molecular weight of about 12 kDa or greater.
1141. The reconstituted solution of any one of the preceding embodiments, wherein the purified Poloxamer 407 has an average molecular weight of about 12.1 kDa or greater.
1142. The reconstituted solution of any one of the preceding embodiments, wherein the purified Poloxamer 407 is prepared by liquid-liquid extraction or size exclusion chromatography.
1143. The reconstituted solution of any one of the preceding embodiments, wherein the purified Poloxamer 407 is prepared by liquid-liquid extraction
1144. The reconstituted solution of any one of the preceding embodiments, wherein the purified Poloxamer 407 is prepared by size exclusion chromatography.
1145. The reconstituted solution of any one of the preceding embodiments, wherein about 10% or more, about 20% or more, about 30% or more, about 40% or more, about 50% or more, about 60% or more, about 70% or more, about 80% or more, about 90% or more, about 95% or more, about 98% or more, or about 99% or more of the one or more impurities having molecular weights below 9 kDa are removed from the Poloxamer 407 during the purification
1146. The reconstituted solution of any one of the preceding embodiments, wherein about 10% or more of the one or more impurities having molecular weights below 9 kDa are removed from the Poloxamer 407 during the purification.
1147. The reconstituted solution of any one of the preceding embodiments, wherein about 20% or more of the one or more impurities having molecular weights below 9 kDa are removed from the Poloxamer 407 during the purification.
1148. The reconstituted solution of any one of the preceding embodiments, wherein about 30% or more of the one or more impurities having molecular weights below 9 kDa are removed from the Poloxamer 407 during the purification.
1149. The reconstituted solution of any one of the preceding embodiments, wherein about 40% or more of the one or more impurities having molecular weights below 9 kDa are removed from the Poloxamer 407 during the purification.
1150. The reconstituted solution of any one of the preceding embodiments, wherein about 50% or more of the one or more impurities having molecular weights below 9 kDa are removed from the Poloxamer 407 during the purification
1151. The reconstituted solution of any one of the preceding embodiments, wherein about 60% or more of the one or more impurities having molecular weights below 9 kDa are removed from the Poloxamer 407 during the purification
1152. The reconstituted solution of any one of the preceding embodiments, wherein about 70% or more of the one or more impurities having molecular weights below 9 kDa are removed from the Poloxamer 407 during the purification.
1153. The reconstituted solution of any one of the preceding embodiments, wherein about 80% or more of the one or more impurities having molecular weights below 9 kDa are removed from the Poloxamer 407 during the purification.
1154. The reconstituted solution of any one of the preceding embodiments, wherein about 90% or more of the one or more impurities having molecular weights below 9 kDa are removed from the Poloxamer 407 during the purification.
1155. The reconstituted solution of any one of the preceding embodiments, wherein about 95% or more of the one or more impurities having molecular weights below 9 kDa are removed from the Poloxamer 407 during the purification.
1156. The reconstituted solution of any one of the preceding embodiments, wherein about 98% or more of the one or more impurities having molecular weights below 9 kDa are removed from the Poloxamer 407 during the purification.
1157. The reconstituted solution of any one of the preceding embodiments, wherein about 99% or more of the one or more impurities having molecular weights below 9 kDa are removed from the Poloxamer 407 during the purification.
1158. The reconstituted solution of any one of the preceding embodiments, wherein the diluent comprises water and dimethyl sulfoxide (DMSO).
1159. The reconstituted solution of any one of the preceding embodiments, wherein the diluent comprises water.
1160. The reconstituted solution of any one of the preceding embodiments, wherein the diluent further comprises dimethyl sulfoxide (DMSO).
1161. The reconstituted solution of any one of the preceding embodiments, wherein the concentration of DMSO in the diluent ranges from about 1% w/w to about 15% w/w, from about 2% w/w to about 12% w/w, from about 3% w/w to about 10% w/w, from about 4% w/w to about 9% w/w, from about 5% w/w to about 8% w/w, from about 5.5% w/w to about 7.5% w/w, from about 5.8% w/w to about 7% w/w, from about 6% w/w to about 6.8% w/w, or from about 6.2% w/w to about 6.6% w/w;
   optionally, the concentration of DMSO in the diluent is about 6.4% w/w.
1162. The reconstituted solution of any one of the preceding embodiments, wherein the concentration of DMSO in the diluent ranges from about 1% w/w to about 15% w/w.
1163. The reconstituted solution of any one of the preceding embodiments, wherein the concentration of DMSO in the diluent ranges from about 2% w/w to about 12% w/w.
1164. The reconstituted solution of any one of the preceding embodiments, wherein the concentration of DMSO in the diluent ranges from about 3% w/w to about 10% w/w.
1165. The reconstituted solution of any one of the preceding embodiments, wherein the concentration of DMSO in the diluent ranges from about 4% w/w to about 9% w/w.
1166. The reconstituted solution of any one of the preceding embodiments, wherein the concentration of DMSO in the diluent ranges from about 5% w/w to about 8% w/w.
1167. The reconstituted solution of any one of the preceding embodiments, wherein the concentration of DMSO in the diluent ranges from about 5.5% w/w to about 7.5% w/w.
1168. The reconstituted solution of any one of the preceding embodiments, wherein the concentration of DMSO in the diluent ranges from about 5.8% w/w to about 7% w/w.
1169. The reconstituted solution of any one of the preceding embodiments, wherein the concentration of DMSO in the diluent ranges from about 6% w/w to about 6.8% w/w.
1170. The reconstituted solution of any one of the preceding embodiments, wherein the concentration of DMSO in the diluent ranges from about 6.2% w/w to about 6.6% w/w.
1171. The reconstituted solution of any one of the preceding embodiments, wherein the concentration of DMSO in the diluent is about 6.4% w/w.
1172. The reconstituted solution of any one of the preceding embodiments, wherein the amount of the diluent added during the constitution ranges from about 1 μL to about 6 μL, from about 2 μL to about 5 μL, from about 2.5 μL to about 4.5 μL, from about 2.8 μL to about 4 μL, from about 3 μL to about 3.8 μL, or from about 3.2 μL to about 3.6 μL per mg of the lyophilized pharmaceutical composition;
   optionally, the amount of the diluent added during the constitution is about 3.4 μL per mg of the lyophilized pharmaceutical composition
1173. The reconstituted solution of any one of the preceding embodiments, wherein the amount of the diluent added during the constitution ranges from about 1 μL to about 6 μL per mg of the lyophilized pharmaceutical composition
1174. The reconstituted solution of any one of the preceding embodiments, wherein the amount of the diluent added during the constitution ranges from about 2 μL to about 5 μL per mg of the lyophilized pharmaceutical composition
1175. The reconstituted solution of any one of the preceding embodiments, wherein the amount of the diluent added during the constitution ranges from about 2.5 μL to about 4.5 μL per mg of the lyophilized pharmaceutical composition
1176. The reconstituted solution of any one of the preceding embodiments, wherein the amount of the diluent added during the constitution ranges from about 2.8 μL to about 4 μL per mg of the lyophilized pharmaceutical composition
1177. The reconstituted solution of any one of the preceding embodiments, wherein the amount of the diluent added during the constitution ranges from about 3 μL to about 3.8 μL per mg of the lyophilized pharmaceutical composition
1178. The reconstituted solution of any one of the preceding embodiments, wherein the amount of the diluent added during the constitution ranges from about 3.2 μL to about 3.6 μL per mg of the lyophilized pharmaceutical composition
1179. The reconstituted solution of any one of the preceding embodiments, wherein the amount of the diluent added during the constitution is about 3.4 μL per mg of the lyophilized pharmaceutical composition
1180. The reconstituted solution of any one of the preceding embodiments, comprising:
   CHIR99021 or a pharmaceutically acceptable salt thereof being present at a concentration ranging from 0.05 mg/ml to about 50 mg/ml.
1181. The reconstituted solution of any one of the preceding embodiments, further comprising:
   valproic acid or a pharmaceutically acceptable salt thereof being present at a concentration ranging from 1 mg/ml to about 1000 mg/ml.
1182. The reconstituted solution of any one of the preceding embodiments, further comprising:
   poloxamer 407 being present at a concentration ranging from 2 wt % to about 50 wt %.
1183. The reconstituted solution of any one of the preceding embodiments, comprising:
   i) CHIR99021 or a pharmaceutically acceptable salt thereof being present at a concentration ranging from 0.05 mg/ml to about 50 mg/ml;
   ii) valproic acid or a pharmaceutically acceptable salt thereof being present at a concentration ranging from 1 mg/ml to about 1000 mg/ml;
   iii) poloxamer 407 being present at a concentration ranging from 2 wt % to about 50 wt %; and
   iv) dimethyl sulfoxide (DMSO) being present at a concentration below 15 wt %.
1184. The reconstituted solution of embodiment 1183, wherein the pharmaceutically acceptable salt of valproic acid is a sodium salt;
   optionally, the pharmaceutically acceptable salt of valproic acid is sodium valproate.
1185. The reconstituted solution of embodiment 1183, wherein the pharmaceutically acceptable salt of valproic acid is sodium valproate.
1186. The reconstituted solution of any one of the preceding embodiments, wherein the concentration of CHIR99021 or the pharmaceutically acceptable salt thereof ranges from about 0.1 mg/ml to about 10 mg/ml, from about 0.5 mg/ml to about 5 mg/ml, from about 1 mg/ml to about 3.5 mg/ml, or from about 2.9 mg/ml to about 3.3 mg/ml;
   optionally, the concentration of CHIR99021 or the pharmaceutically acceptable salt thereof is about 3.1 mg/ml.
1187. The reconstituted solution of any one of the preceding embodiments, wherein the concentration of CHIR99021 or the pharmaceutically acceptable salt thereof ranges from about 0.1 mg/ml to about 10 mg/ml.
1188. The reconstituted solution of any one of the preceding embodiments, wherein the concentration of CHIR99021 or the pharmaceutically acceptable salt thereof ranges from about 0.5 mg/ml to about 5 mg/ml.
1189. The reconstituted solution of any one of the preceding embodiments, wherein the concentration of CHIR99021 or the pharmaceutically acceptable salt thereof ranges from about 1 mg/ml to about 3.5 mg/ml.
1190. The reconstituted solution of any one of the preceding embodiments, wherein the concentration of CHIR99021 or the pharmaceutically acceptable salt thereof ranges from about 2.9 mg/ml to about 3.3 mg/ml.
1191. The reconstituted solution of any one of the preceding embodiments, wherein the concentration of CHIR99021 or the pharmaceutically acceptable salt thereof is about 3.1 mg/ml.
1192. The reconstituted solution of any one of the preceding embodiments, wherein the concentration of valproic acid or the pharmaceutically acceptable salt thereof ranges from about 5 mg/ml to about 400 mg/ml, from about 10 mg/ml to about 200 mg/ml, from about 30 mg/ml to about 100 mg/ml, or from about 86 mg/ml to about 92 mg/ml;
   optionally, the concentration of valproic acid or the pharmaceutically acceptable salt thereof is about 89 mg/ml.
1193. The reconstituted solution of any one of the preceding embodiments, wherein the concentration of valproic acid or the pharmaceutically acceptable salt thereof ranges from about 5 mg/ml to about 400 mg/ml.
1194. The reconstituted solution of any one of the preceding embodiments, wherein the concentration of valproic acid or the pharmaceutically acceptable salt thereof ranges from about 10 mg/ml to about 200 mg/ml.
1195. The reconstituted solution of any one of the preceding embodiments, wherein the concentration of valproic acid or the pharmaceutically acceptable salt thereof ranges from about 30 mg/ml to about 100 mg/ml.
1196. The reconstituted solution of any one of the preceding embodiments, wherein the concentration of valproic acid or the pharmaceutically acceptable salt thereof ranges from about 86 mg/ml to about 92 mg/ml.
1197. The reconstituted solution of any one of the preceding embodiments, wherein the concentration of valproic acid or the pharmaceutically acceptable salt thereof ranges the concentration of valproic acid or the pharmaceutically acceptable salt thereof is about 89 mg/ml.
1198. The reconstituted solution of any one of the preceding embodiments, wherein the concentration of poloxamer 407 ranges from about 5 wt % to about 25 wt %, from about 10 wt % to about 22 wt %, from about 12 wt % to about 20 wt %, or from about 14 wt % to about 17 wt %;
   optionally, the concentration of poloxamer 407 is about 16 wt %.
1199. The reconstituted solution of any one of the preceding embodiments, wherein the concentration of poloxamer 407 ranges from about 5 wt % to about 25 wt %.
1200. The reconstituted solution of any one of the preceding embodiments, wherein the concentration of poloxamer 407 ranges from about 10 wt % to about 22 wt %.
1201. The reconstituted solution of any one of the preceding embodiments, wherein the concentration of poloxamer 407 ranges from about 12 wt % to about 20 wt %.
1202. The reconstituted solution of any one of the preceding embodiments, wherein the concentration of poloxamer 407 ranges from about 14 wt % to about 17 wt %.
1203. The reconstituted solution of any one of the preceding embodiments, wherein the concentration of poloxamer 407 is about 16 wt %.
1204. The reconstituted solution of any one of the preceding embodiments, wherein the concentration of DMSO ranges from about 1 wt % to about 10 wt %, from about 2 wt % to about 8 wt %, from about 3 wt % to about 7 wt %, or from about 4 wt % to about 6 wt %;
   optionally, the concentration of DMSO is about 5 wt %.
1205. The reconstituted solution of any one of the preceding embodiments, wherein the concentration of DMSO ranges from about 1 wt % to about 10 wt %.
1206. The reconstituted solution of any one of the preceding embodiments, wherein the concentration of DMSO ranges from about 2 wt % to about 8 wt %.
1207. The reconstituted solution of any one of the preceding embodiments, wherein the concentration of DMSO ranges from about 3 wt % to about 7 wt %.
1208. The reconstituted solution of any one of the preceding embodiments, wherein the concentration of DMSO ranges from about 4 wt % to about 6 wt %.
1209. The reconstituted solution of any one of the preceding embodiments, wherein the concentration of DMSO is about 5 wt %.
1210. The reconstituted solution of any one of the preceding embodiments, wherein the weight ratio between CHIR99021 or the pharmaceutically acceptable salt thereof and valproic acid or the pharmaceutically acceptable salt thereof ranges from about 1:5 to about 1:10, from about 1:10 to about 1:50, from about 1:20 to about 1:35, from about 1:25 to about 1:31, or from about 1:27 to about 1:29.
1211. The reconstituted solution of any one of the preceding embodiments, wherein the weight ratio between CHIR99021 or the pharmaceutically acceptable salt thereof and valproic acid or the pharmaceutically acceptable salt thereof ranges from about 1:5 to about 1:10.
1212. The reconstituted solution of any one of the preceding embodiments, wherein the weight ratio between CHIR99021 or the pharmaceutically acceptable salt thereof and valproic acid or the pharmaceutically acceptable salt thereof ranges from about 1:10 to about 1:50.
1213. The reconstituted solution of any one of the preceding embodiments, wherein the weight ratio between CHIR99021 or the pharmaceutically acceptable salt thereof and valproic acid or the pharmaceutically acceptable salt thereof ranges from about 1:20 to about 1:35.
1214. The reconstituted solution of any one of the preceding embodiments, wherein the weight ratio between CHIR99021 or the pharmaceutically acceptable salt thereof and valproic acid or the pharmaceutically acceptable salt thereof ranges from about 1:25 to about 1:31.
1215. The reconstituted solution of any one of the preceding embodiments, wherein the weight ratio between CHIR99021 or the pharmaceutically acceptable salt thereof and valproic acid or the pharmaceutically acceptable salt thereof ranges from about 1:27 to about 1:29.
1216. The reconstituted solution of any one of the preceding embodiments, wherein the weight ratio between poloxamer 407 and the DMSO ranges from about 1:5 to about 40:1, from about 1:2 to about 15:1, from about 1:1 to about 8:1, from about 2:1 to about 4:1, or from about 2.5:1 to about 3.5:1; optionally, the weight ratio between poloxamer 407 and the DMSO is about 3:1.
1217. The reconstituted solution of any one of the preceding embodiments, wherein the weight ratio between poloxamer 407 and the DMSO ranges from about 1:5 to about 40:1.
1218. The reconstituted solution of any one of the preceding embodiments, wherein the weight ratio between poloxamer 407 and the DMSO ranges from about 1:2 to about 15:1.
1219. The reconstituted solution of any one of the preceding embodiments, wherein the weight ratio between poloxamer 407 and the DMSO ranges from about 1:1 to about 8:1.
1220. The reconstituted solution of any one of the preceding embodiments, wherein the weight ratio between poloxamer 407 and the DMSO ranges from about 2:1 to about 4:1.
1221. The reconstituted solution of any one of the preceding embodiments, wherein the weight ratio between poloxamer 407 and the DMSO ranges from about 2.5:1 to about 3.5:1.
1222. The reconstituted solution of any one of the preceding embodiments, wherein the weight ratio between poloxamer 407 and the DMSO is about 3:1.
1223. The reconstituted solution of any one of the preceding embodiments, wherein:
   the weight ratio between CHIR99021 and poloxamer 407 is about 0.02:1;
   the weight ratio between CHIR99021 and the DMSO is about 0.06:1;
   the weight ratio between valproic acid sodium salt and poloxamer 407 is about 0.54:1; and/or
   the weight ratio between valproic acid sodium salt and the DMSO is about 3.2:1.
1224. The reconstituted solution of any one of the preceding embodiments, wherein the weight ratio between CHIR99021 and poloxamer 407 is about 0.02:1.
1225. The reconstituted solution of any one of the preceding embodiments, wherein the weight ratio between CHIR99021 and the DMSO is about 0.06:1.
1226. The reconstituted solution of any one of the preceding embodiments, wherein the weight ratio between valproic acid sodium salt and poloxamer 407 is about 0.54:1.
1227. The reconstituted solution of any one of the preceding embodiments, wherein the weight ratio between valproic acid sodium salt and the DMSO is about 3.2:1.
1228. The reconstituted solution of any one of the preceding embodiments, wherein:
   the concentration of CHIR99021 or the pharmaceutically acceptable salt thereof ranges from about 2.9 mg/ml to about 3.3 mg/ml;
   the concentration of valproic acid or the pharmaceutically acceptable salt thereof ranges from about 86 mg/ml to about 92 mg/ml;
   the concentration of poloxamer 407 ranges from about 14 wt % to about 17 wt %; and
   the concentration of DMSO ranges from about 4 wt % to about 6 wt %.
1229. The reconstituted solution of any one of the preceding embodiments, wherein
   the concentration of CHIR99021 or the pharmaceutically acceptable salt thereof ranges from about 2.9 mg/ml to about 3.3 mg/ml.
1230. The reconstituted solution of any one of the preceding embodiments, wherein the concentration of valproic acid or the pharmaceutically acceptable salt thereof ranges from about 86 mg/ml to about 92 mg/ml.
1231. The reconstituted solution of any one of the preceding embodiments, wherein the concentration of poloxamer 407 ranges from about 14 wt % to about 17 wt %.
1232. The reconstituted solution of any one of the preceding embodiments, wherein the concentration of DMSO ranges from about 4 wt % to about 6 wt %.
1233. The reconstituted solution of any one of the preceding embodiments, wherein:
   the concentration of CHIR99021 or the pharmaceutically acceptable salt thereof is about 3.1 mg/ml;
   the concentration of valproic acid or the pharmaceutically acceptable salt thereof is about 89 mg/ml;
   the concentration of poloxamer 407 is about 16 wt %; and
   the concentration of DMSO is about 5 wt %.
1234. The reconstituted solution of any one of the preceding embodiments, wherein the concentration of CHIR99021 or the pharmaceutically acceptable salt thereof is about 3.1 mg/ml.
1235. The reconstituted solution of any one of the preceding embodiments, wherein the concentration of valproic acid or the pharmaceutically acceptable salt thereof is about 89 mg/ml.
1236. The reconstituted solution of any one of the preceding embodiments, wherein the concentration of poloxamer 407 is about 16 wt %.
1237. The reconstituted solution of any one of the preceding embodiments, wherein the concentration of DMSO is about 5 wt %.
1238. The reconstituted solution of any one of the preceding embodiments, comprising:
   i) CHIR99021 or a pharmaceutically acceptable salt thereof being present at a concentration ranging from 0.05 mg/ml to about 50 mg/ml;
   ii) valproic acid or a pharmaceutically acceptable salt thereof being present at a concentration ranging from 1 mg/ml to about 1000 mg/ml;
   iii) poloxamer 407 being present at a concentration ranging from 2 wt % to about 50 wt %; and
   iv) dimethyl sulfoxide (DMSO) being present at a concentration below 15 wt %.
1239. The reconstituted solution of any one of the preceding embodiments, comprising
   CHIR99021 or a pharmaceutically acceptable salt thereof being present at a concentration ranging from 0.05 mg/ml to about 50 mg/ml.
1240. The reconstituted solution of any one of the preceding embodiments, comprising valproic acid or a pharmaceutically acceptable salt thereof being present at a concentration ranging from 1 mg/ml to about 1000 mg/ml.
1241. The reconstituted solution of any one of the preceding embodiments, comprising poloxamer 407 being present at a concentration ranging from 2 wt % to about 50 wt %.
1242. The reconstituted solution of any one of the preceding embodiments, comprising dimethyl sulfoxide (DMSO) being present at a concentration below 15 wt %.
1243. The reconstituted solution of embodiment 1242, wherein the pharmaceutically acceptable salt of valproic acid is a sodium salt;
   optionally, the pharmaceutically acceptable salt of valproic acid is sodium valproate.
1244. The reconstituted solution of embodiment 1242, wherein the pharmaceutically acceptable salt of valproic acid is sodium valproate.
1245. The reconstituted solution of any one of the preceding embodiments, wherein the concentration of CHIR99021 or the pharmaceutically acceptable salt thereof ranges from about 0.1 mg/ml to about 10 mg/ml, from about 0.5 mg/ml to about 5 mg/ml, from about 1 mg/ml to about 3.5 mg/ml, from about 1.9 mg/ml to about 2.3 mg/ml;
   optionally, the concentration of CHIR99021 or the pharmaceutically acceptable salt thereof is about 2.1 mg/ml.
1246. The reconstituted solution of any one of the preceding embodiments, wherein the concentration of CHIR99021 or the pharmaceutically acceptable salt thereof ranges from about 0.1 mg/ml to about 10 mg/ml.
1247. The reconstituted solution of any one of the preceding embodiments, wherein the concentration of CHIR99021 or the pharmaceutically acceptable salt thereof ranges from about 0.5 mg/ml to about 5 mg/ml.
1248. The reconstituted solution of any one of the preceding embodiments, wherein the concentration of CHIR99021 or the pharmaceutically acceptable salt thereof ranges from about 1 mg/ml to about 3.5 mg/ml.
1249. The reconstituted solution of any one of the preceding embodiments, wherein the concentration of CHIR99021 or the pharmaceutically acceptable salt thereof ranges from about 1.9 mg/ml to about 2.3 mg/ml.
1250. The reconstituted solution of any one of the preceding embodiments, wherein the concentration of CHIR99021 or the pharmaceutically acceptable salt thereof is about 2.1 mg/ml.
1251. The reconstituted solution of any one of the preceding embodiments, wherein the concentration of valproic acid or the pharmaceutically acceptable salt thereof ranges from about 5 mg/ml to about 400 mg/ml, from about 10 mg/ml to about 200 mg/ml, from about 30 mg/ml to about 100 mg/ml, from about 56 mg/ml to about 62 mg/ml;
   optionally, the concentration of valproic acid or the pharmaceutically acceptable salt thereof is about 59 mg/ml.
1252. The reconstituted solution of any one of the preceding embodiments, wherein the concentration of valproic acid or the pharmaceutically acceptable salt thereof ranges from about 5 mg/ml to about 400 mg/ml.
1253. The reconstituted solution of any one of the preceding embodiments, wherein the concentration of valproic acid or the pharmaceutically acceptable salt thereof ranges from about 10 mg/ml to about 200 mg/ml.
1254. The reconstituted solution of any one of the preceding embodiments, wherein the concentration of valproic acid or the pharmaceutically acceptable salt thereof ranges from about 30 mg/ml to about 100 mg/ml.
1255. The reconstituted solution of any one of the preceding embodiments, wherein the concentration of valproic acid or the pharmaceutically acceptable salt thereof ranges from about 56 mg/ml to about 62 mg/ml.
1256. The reconstituted solution of any one of the preceding embodiments, wherein the concentration of valproic acid or the pharmaceutically acceptable salt thereof is about 59 mg/ml.
1257. The reconstituted solution of any one of the preceding embodiments, wherein the concentration of poloxamer 407 ranges from about 5 wt % to about 25 wt %, from about 10 wt % to about 22 wt %, from about 12 wt % to about 20 wt %, from about 14 wt % to about 17 wt %;
   optionally, the concentration of poloxamer 407 is about 15 wt %.
1258. The reconstituted solution of any one of the preceding embodiments, wherein the concentration of poloxamer 407 ranges from about 5 wt % to about 25 wt %.
1259. The reconstituted solution of any one of the preceding embodiments, wherein the concentration of poloxamer 407 ranges from about 10 wt % to about 22 wt %.
1260. The reconstituted solution of any one of the preceding embodiments, wherein the concentration of poloxamer 407 ranges from about 12 wt % to about 20 wt %.
1261. The reconstituted solution of any one of the preceding embodiments, wherein the concentration of poloxamer 407 ranges from about 14 wt % to about 17 wt %.
1262. The reconstituted solution of any one of the preceding embodiments, wherein the concentration of poloxamer 407 is about 15 wt %.
1263. The reconstituted solution of any one of the preceding embodiments, wherein the concentration of DMSO ranges from about 1 wt % to about 10 wt %, from about 2 wt % to about 8 wt %, from about 3 wt % to about 7 wt %, from about 4 wt % to about 6 wt %;
   optionally, the concentration of DMSO is about 5 wt %.
1264. The reconstituted solution of any one of the preceding embodiments, wherein the concentration of DMSO ranges from about 1 wt % to about 10 wt %.
1265. The reconstituted solution of any one of the preceding embodiments, wherein the concentration of DMSO ranges from about 2 wt % to about 8 wt %.
1266. The reconstituted solution of any one of the preceding embodiments, wherein the concentration of DMSO ranges from about 3 wt % to about 7 wt %.
1267. The reconstituted solution of any one of the preceding embodiments, wherein the concentration of DMSO ranges from about 4 wt % to about 6 wt %.
1268. The reconstituted solution of any one of the preceding embodiments, wherein the concentration of DMSO is about 5 wt %.
1269. The reconstituted solution of any one of the preceding embodiments, wherein the weight ratio between CHIR99021 or the pharmaceutically acceptable salt thereof and valproic acid or the pharmaceutically acceptable salt thereof ranges from about 1:5 to about 1:10, from about 1:10 to about 1:50, from about 1:20 to about 1:35, from about 1:25 to about 1:31, or from about 1:27 to about 1:29.
1270. The reconstituted solution of any one of the preceding embodiments, wherein the weight ratio between CHIR99021 or the pharmaceutically acceptable salt thereof and valproic acid or the pharmaceutically acceptable salt thereof ranges from about 1:5 to about 1:10.
1271. The reconstituted solution of any one of the preceding embodiments, wherein the weight ratio between CHIR99021 or the pharmaceutically acceptable salt thereof and valproic acid or the pharmaceutically acceptable salt thereof ranges from about 1:10 to about 1:50.
1272. The reconstituted solution of any one of the preceding embodiments, wherein the weight ratio between CHIR99021 or the pharmaceutically acceptable salt thereof and valproic acid or the pharmaceutically acceptable salt thereof ranges from about 1:20 to about 1:35.
1273. The reconstituted solution of any one of the preceding embodiments, wherein the weight ratio between CHIR99021 or the pharmaceutically acceptable salt thereof and valproic acid or the pharmaceutically acceptable salt thereof ranges from about 1:25 to about 1:31.
1274. The reconstituted solution of any one of the preceding embodiments, wherein the weight ratio between CHIR99021 or the pharmaceutically acceptable salt thereof and valproic acid or the pharmaceutically acceptable salt thereof ranges from about 1:27 to about 1:29.
1275. The reconstituted solution of any one of the preceding embodiments, wherein the weight ratio between poloxamer 407 and the DMSO ranges from about 1:5 to about 40:1, from about 1:2 to about 15:1, from about 1:1 to about 8:1, from about 2:1 to about 4:1, from about 2.5:1 to about 3.5:1;
   optionally, the weight ratio between poloxamer 407 and the DMSO is about 3:1.
1276. The reconstituted solution of any one of the preceding embodiments, wherein the weight ratio between poloxamer 407 and the DMSO ranges from about 1:5 to about 40:1.
1277. The reconstituted solution of any one of the preceding embodiments, wherein the weight ratio between poloxamer 407 and the DMSO ranges from about 1:2 to about 15:1.
1278. The reconstituted solution of any one of the preceding embodiments, wherein the weight ratio between poloxamer 407 and the DMSO ranges from about 1:1 to about 8:1.
1279. The reconstituted solution of any one of the preceding embodiments, wherein the weight ratio between poloxamer 407 and the DMSO ranges from about 2:1 to about 4:1.
1280. The reconstituted solution of any one of the preceding embodiments, wherein the weight ratio between poloxamer 407 and the DMSO ranges from about 2.5:1 to about 3.5:1.
1281. The reconstituted solution of any one of the preceding embodiments, wherein the weight ratio between poloxamer 407 and the DMSO is about 3:1.
1282. The reconstituted solution of any one of the preceding embodiments, wherein:
   the weight ratio between CHIR99021 and poloxamer 407 is about 0.016:1;
   the weight ratio between the CHIR99021 and the DMSO is about 0.06:1;
   the weight ratio between valproic acid sodium salt and poloxamer 407 is about 0.42:1; and/or
   the weight ratio between valproic acid sodium salt and the DMSO is about 1.5:1.
1283. The reconstituted solution of any one of the preceding embodiments, wherein the weight ratio between CHIR99021 and poloxamer 407 is about 0.016:1.
1284. The reconstituted solution of any one of the preceding embodiments, wherein the weight ratio between the CHIR99021 and the DMSO is about 0.06:1.
1285. The reconstituted solution of any one of the preceding embodiments, wherein the weight ratio between valproic acid sodium salt and poloxamer 407 is about 0.42:1.
1286. The reconstituted solution of any one of the preceding embodiments, wherein the weight ratio between valproic acid sodium salt and the DMSO is about 1.5:1.
1287. The reconstituted solution of any one of the preceding embodiments, wherein:
   the concentration of CHIR99021 or the pharmaceutically acceptable salt thereof ranges from about 1.9 mg/ml to about 2.3 mg/ml;
   the concentration of valproic acid or the pharmaceutically acceptable salt thereof ranges from about 56 mg/ml to about 62 mg/ml;
   the concentration of poloxamer 407 ranges from about 14 wt % to about 17 wt %; and
   the concentration of DMSO ranges from about 4 wt % to about 6 wt %.
1288. The reconstituted solution of any one of the preceding embodiments, wherein the concentration of CHIR99021 or the pharmaceutically acceptable salt thereof ranges from about 1.9 mg/ml to about 2.3 mg/ml.
1289. The reconstituted solution of any one of the preceding embodiments, wherein the concentration of valproic acid or the pharmaceutically acceptable salt thereof ranges from about 56 mg/ml to about 62 mg/ml.
1290. The reconstituted solution of any one of the preceding embodiments, wherein the concentration of poloxamer 407 ranges from about 14 wt % to about 17 wt %.
1291. The reconstituted solution of any one of the preceding embodiments, wherein the concentration of DMSO ranges from about 4 wt % to about 6 wt %.
1292. The reconstituted solution of any one of the preceding embodiments, wherein:
   the concentration of CHIR99021 or the pharmaceutically acceptable salt thereof is about 2.1 mg/ml;
   the concentration of valproic acid or the pharmaceutically acceptable salt thereof is about 59 mg/ml;
   the concentration of poloxamer 407 is about 15 wt %; and
   the concentration of DMSO is about 5 wt %.
1293. The reconstituted solution of any one of the preceding embodiments, wherein the concentration of CHIR99021 or the pharmaceutically acceptable salt thereof is about 2.1 mg/ml.
1294. The reconstituted solution of any one of the preceding embodiments, wherein the concentration of valproic acid or the pharmaceutically acceptable salt thereof is about 59 mg/ml.
1295. The reconstituted solution of any one of the preceding embodiments, wherein the concentration of poloxamer 407 is about 15 wt %.
1296. The reconstituted solution of any one of the preceding embodiments, wherein the concentration of DMSO is about 5 wt %.
1297. The reconstituted solution of any one of the preceding embodiments, comprising:
   i) CHIR99021 or a pharmaceutically acceptable salt thereof being present at a concentration ranging from 0.05 mg/ml to about 50 mg/ml;
   ii) valproic acid or a pharmaceutically acceptable salt thereof being present at a concentration ranging from 1 mg/ml to about 1000 mg/ml;
   iii) poloxamer 407 being present at a concentration ranging from 2 wt % to about 50 wt %; and
   iv) dimethyl sulfoxide (DMSO) being present at a concentration below 15 wt %.
1298. The reconstituted solution of embodiment 1297, wherein the pharmaceutically acceptable salt of valproic acid is a sodium salt;
   optionally, the pharmaceutically acceptable salt of valproic acid is sodium valproate.
1299. The reconstituted solution of embodiment 1297, wherein the pharmaceutically acceptable salt of valproic acid is sodium valproate.
1300. The reconstituted solution of any one of the preceding embodiments, wherein the concentration of CHIR99021 or the pharmaceutically acceptable salt thereof ranges from about 0.1 mg/ml to about 10 mg/ml, from about 0.5 mg/ml to about 5 mg/ml, from about 1 mg/ml to about 3.5 mg/ml, or from about 1.2 mg/ml to about 1.5 mg/ml;
   optionally, the concentration of CHIR99021 or the pharmaceutically acceptable salt thereof ranges is about 1.4 mg/ml.
1301. The reconstituted solution of any one of the preceding embodiments, wherein the concentration of CHIR99021 or the pharmaceutically acceptable salt thereof ranges from about 0.1 mg/ml to about 10 mg/ml.
1302. The reconstituted solution of any one of the preceding embodiments, wherein the concentration of CHIR99021 or the pharmaceutically acceptable salt thereof ranges from about 0.5 mg/ml to about 5 mg/ml.
1303. The reconstituted solution of any one of the preceding embodiments, wherein the concentration of CHIR99021 or the pharmaceutically acceptable salt thereof ranges from about 1 mg/ml to about 3.5 mg/ml.
1304. The reconstituted solution of any one of the preceding embodiments, wherein the concentration of CHIR99021 or the pharmaceutically acceptable salt thereof ranges from about 1.2 mg/ml to about 1.5 mg/ml.
1305. The reconstituted solution of any one of the preceding embodiments, wherein the concentration of CHIR99021 or the pharmaceutically acceptable salt thereof ranges is about 1.4 mg/ml.
1306. The reconstituted solution of any one of the preceding embodiments, wherein the concentration of valproic acid or the pharmaceutically acceptable salt thereof ranges from about 5 mg/ml to about 400 mg/ml, from about 10 mg/ml to about 200 mg/ml, from about 30 mg/ml to about 100 mg/ml, or from about 36 mg/ml to about 42 mg/ml;
   optionally, the concentration of valproic acid or the pharmaceutically acceptable salt thereof is about 39 mg/ml.
1307. The reconstituted solution of any one of the preceding embodiments, wherein the concentration of valproic acid or the pharmaceutically acceptable salt thereof ranges from about 5 mg/ml to about 400 mg/ml.
1308. The reconstituted solution of any one of the preceding embodiments, wherein the concentration of valproic acid or the pharmaceutically acceptable salt thereof ranges from about from about 10 mg/ml to about 200 mg/ml.
1309. The reconstituted solution of any one of the preceding embodiments, wherein the concentration of valproic acid or the pharmaceutically acceptable salt thereof ranges from about from about 100 mg/ml to about 200 mg/ml.
1310. The reconstituted solution of any one of the preceding embodiments, wherein the concentration of valproic acid or the pharmaceutically acceptable salt thereof ranges from about from about 120 mg/ml to about 140 mg/ml.
1311. The reconstituted solution of any one of the preceding embodiments, wherein the concentration of valproic acid or the pharmaceutically acceptable salt thereof ranges from about from about 30 mg/ml to about 100 mg/ml.
1312. The reconstituted solution of any one of the preceding embodiments, wherein the concentration of valproic acid or the pharmaceutically acceptable salt thereof ranges from about from about 36 mg/ml to about 42 mg/ml.
1313. The reconstituted solution of any one of the preceding embodiments, wherein the concentration of valproic acid or the pharmaceutically acceptable salt thereof is about 39 mg/ml.
1314. The reconstituted solution of any one of the preceding embodiments, wherein the concentration of poloxamer 407 ranges from about 5 wt % to about 25 wt %, from about 10 wt % to about 22 wt %, from about 12 wt % to about 20 wt %, or from about 14 wt % to about 17 wt %;
   optionally, the concentration of poloxamer 407 is about 15 wt %.
1315. The reconstituted solution of any one of the preceding embodiments, wherein the concentration of poloxamer 407 ranges from about 5 wt % to about 25 wt %.
1316. The reconstituted solution of any one of the preceding embodiments, wherein the concentration of poloxamer 407 ranges from about 10 wt % to about 22 wt %.
1317. The reconstituted solution of any one of the preceding embodiments, wherein the concentration of poloxamer 407 ranges from about 12 wt % to about 20 wt %.
1318. The reconstituted solution of any one of the preceding embodiments, wherein the concentration of poloxamer 407 ranges from about 14 wt % to about 17 wt %.
1319. The reconstituted solution of any one of the preceding embodiments, wherein the concentration of poloxamer 407 is about 15 wt %.
1320. The reconstituted solution of any one of the preceding embodiments, wherein the concentration of DMSO ranges from about 1 wt % to about 10 wt %, from about 2 wt % to about 8 wt %, from about 3 wt % to about 7 wt %, or from about 4 wt % to about 6 wt %;
   optionally, the concentration of DMSO is about 5 wt %.
1321. The reconstituted solution of any one of the preceding embodiments, wherein the concentration of DMSO ranges from about 1 wt % to about 10 wt %.
1322. The reconstituted solution of any one of the preceding embodiments, wherein the concentration of DMSO ranges from about 2 wt % to about 8 wt %.
1323. The reconstituted solution of any one of the preceding embodiments, wherein the concentration of DMSO ranges from about 3 wt % to about 7 wt %.
1324. The reconstituted solution of any one of the preceding embodiments, wherein the concentration of DMSO ranges from about 4 wt % to about 6 wt %.
1325. The reconstituted solution of any one of the preceding embodiments, wherein the concentration of DMSO is about 5 wt %.
1326. The reconstituted solution of any one of the preceding embodiments, wherein the weight ratio between CHIR99021 or the pharmaceutically acceptable salt thereof and valproic acid or the pharmaceutically acceptable salt thereof ranges from about 1:5 to about 1:10, from about 1:10 to about 1:50, from about 1:20 to about 1:35, from about 1:25 to about 1:31, or from about 1:27 to about 1:29.
1327. The reconstituted solution of any one of the preceding embodiments, wherein the weight ratio between CHIR99021 or the pharmaceutically acceptable salt thereof and valproic acid or the pharmaceutically acceptable salt thereof ranges from about 1:5 to about 1:10.
1328. The reconstituted solution of any one of the preceding embodiments, wherein the weight ratio between CHIR99021 or the pharmaceutically acceptable salt thereof and valproic acid or the pharmaceutically acceptable salt thereof ranges from about 1:10 to about 1:50.
1329. The reconstituted solution of any one of the preceding embodiments, wherein the weight ratio between CHIR99021 or the pharmaceutically acceptable salt thereof and valproic acid or the pharmaceutically acceptable salt thereof ranges from about 1:20 to about 1:35.
1330. The reconstituted solution of any one of the preceding embodiments, wherein the weight ratio between CHIR99021 or the pharmaceutically acceptable salt thereof and valproic acid or the pharmaceutically acceptable salt thereof ranges from about 1:25 to about 1:31.
1331. The reconstituted solution of any one of the preceding embodiments, wherein the weight ratio between CHIR99021 or the pharmaceutically acceptable salt thereof and valproic acid or the pharmaceutically acceptable salt thereof ranges from about 1:27 to about 1:29.
1332. The reconstituted solution of any one of the preceding embodiments, wherein the weight ratio between poloxamer 407 and the DMSO ranges from about 1:5 to about 40:1, from about 1:2 to about 15:1, from about 1:1 to about 8:1, from about 2:1 to about 4:1, from about 2.5:1 to about 3.5:1.
1333. The reconstituted solution of any one of the preceding embodiments, wherein the weight ratio between poloxamer 407 and the DMSO ranges from about 1:5 to about 40:1.
1334. The reconstituted solution of any one of the preceding embodiments, wherein the weight ratio between poloxamer 407 and the DMSO ranges from about 1:2 to about 15:1.
1335. The reconstituted solution of any one of the preceding embodiments, wherein the weight ratio between poloxamer 407 and the DMSO ranges from about 1:1 to about 8:1.
1336. The reconstituted solution of any one of the preceding embodiments, wherein the weight ratio between poloxamer 407 and the DMSO ranges from about 2:1 to about 4:1.
1337. The reconstituted solution of any one of the preceding embodiments, wherein the weight ratio between poloxamer 407 and the DMSO ranges from about 2.5:1 to about 3.5:1.
1338. The reconstituted solution of any one of the preceding embodiments, wherein:
   the weight ratio between poloxamer 407 and the DMSO is about 3:1;
   the weight ratio between the CHIR99021 and poloxamer 407 is about 0.013:1;
   the weight ratio between CHIR99021 and the DMSO is about 0.06:1;
   the weight ratio between valproic acid sodium salt and poloxamer 407 is about 0.23:1; and/or
   the weight ratio between valproic acid sodium salt and the DMSO is about 1.8:1.
1339. The reconstituted solution of any one of the preceding embodiments, wherein the weight ratio between poloxamer 407 and the DMSO is about 3:1.
1340. The reconstituted solution of any one of the preceding embodiments, wherein the weight ratio between the CHIR99021 and poloxamer 407 is about 0.013:1.
1341. The reconstituted solution of any one of the preceding embodiments, wherein the weight ratio between CHIR99021 and the DMSO is about 0.06:1.
1342. The reconstituted solution of any one of the preceding embodiments, wherein the weight ratio between valproic acid sodium salt and poloxamer 407 is about 0.23:1.
1343. The reconstituted solution of any one of the preceding embodiments, wherein the weight ratio between valproic acid sodium salt and the DMSO is about 1.8:1.
1344. The reconstituted solution of any one of the preceding embodiments, wherein:
   the concentration of CHIR99021 or the pharmaceutically acceptable salt thereof ranges from about 1.2 mg/ml to about 1.5 mg/ml;
   the concentration of valproic acid or the pharmaceutically acceptable salt thereof ranges from about 36 mg/ml to about 42 mg/ml;
   the concentration of poloxamer 407 ranges from about 14 wt % to about 17 wt %; and
   the concentration of DMSO ranges from about 4 wt % to about 6 wt %.
1345. The reconstituted solution of any one of the preceding embodiments, wherein the concentration of CHIR99021 or the pharmaceutically acceptable salt thereof ranges from about 1.2 mg/ml to about 1.5 mg/ml.
1346. The reconstituted solution of any one of the preceding embodiments, wherein the concentration of valproic acid or the pharmaceutically acceptable salt thereof ranges from about 36 mg/ml to about 42 mg/ml.
1347. The reconstituted solution of any one of the preceding embodiments, wherein the concentration of poloxamer 407 ranges from about 14 wt % to about 17 wt %.
1348. The reconstituted solution of any one of the preceding embodiments, wherein the concentration of DMSO ranges from about 4 wt % to about 6 wt %.
1349. The reconstituted solution of any one of the preceding embodiments, wherein:
   the concentration of CHIR99021 or the pharmaceutically acceptable salt thereof is about 1.4 mg/ml;
   the concentration of valproic acid or the pharmaceutically acceptable salt thereof is about 39 mg/ml;
   the concentration of poloxamer 407 is about 15 wt %; and
   the concentration of DMSO is about 5 wt %.
1350. The reconstituted solution of any one of the preceding embodiments, wherein the concentration of CHIR99021 or the pharmaceutically acceptable salt thereof is about 1.4 mg/ml.
1351. The reconstituted solution of any one of the preceding embodiments, wherein the concentration of valproic acid or the pharmaceutically acceptable salt thereof is about 39 mg/ml.
1352. The reconstituted solution of any one of the preceding embodiments, wherein the concentration of poloxamer 407 is about 15 wt %.
1353. The reconstituted solution of any one of the preceding embodiments, wherein the concentration of DMSO is about 5 wt %.
1354. The reconstituted solution of any one of the preceding embodiments, comprising one or more of:
   water or a buffering agent;
   a bulking agent;
   a stabilizing agent;
   a tonicity-adjusting agent; and
   a soothing agent
1355. The reconstituted solution of any one of the preceding embodiments, comprising water.
1356. The reconstituted solution of any one of the preceding embodiments, comprising a buffering agent
1357. The reconstituted solution of any one of the preceding embodiments, comprising a bulking agent
1358. The reconstituted solution of any one of the preceding embodiments, comprising a stabilizing agent
1359. The reconstituted solution of any one of the preceding embodiments, comprising a tonicity-adjusting agent
1360. The reconstituted solution of any one of the preceding embodiments, comprising a soothing agent
1361. The reconstituted solution of any one of the preceding embodiments, wherein the reconstituted solution comprises purified Poloxamer 407, and wherein the reconstituted solution has a higher stability to oxygen and/or light as compared to a comparable reconstituted solution without purified Poloxamer 407; optionally, the comparable reconstituted solution comprises unpurified Poloxamer 407.
1362. The reconstituted solution of any one of the preceding embodiments, wherein the level of an impurity presented in the reconstituted solution is less than about 10000 parts per million (ppm), less than about 1000 ppm, less than about 100 ppm, less than about 10 ppm, less than about 1 ppm, or less than about 0.1 ppm.
1363. The reconstituted solution of any one of the preceding embodiments, wherein the level of an impurity presented in the reconstituted solution is less than about 10000 parts per million (ppm).
1364. The reconstituted solution of any one of the preceding embodiments, wherein the level of an impurity presented in the reconstituted solution is less than about 1000 ppm.
1365. The reconstituted solution of any one of the preceding embodiments, wherein the level of an impurity presented in the reconstituted solution is less than about 100 ppm.
1366. The reconstituted solution of any one of the preceding embodiments, wherein the level of an impurity presented in the reconstituted solution is less than about 10 ppm.
1367. The reconstituted solution of any one of the preceding embodiments, wherein the level of an impurity presented in the reconstituted solution is less than about 1 ppm, or less than about 0.1 ppm.
1368. The reconstituted solution of any one of the preceding embodiments, wherein impurity is selected from the group consisting of 1-acetate-2-formate-1,2-propanediol, acetic acid, formic acid, formaldehyde, acetaldehyde, and propionaldehyde.
1369. The reconstituted solution of any one of the preceding embodiments, wherein impurity is 1-acetate-2-formate-1,2-propanediol.
1370. The reconstituted solution of any one of the preceding embodiments, wherein impurity is acetic acid.
1371. The reconstituted solution of any one of the preceding embodiments, wherein impurity is formic acid.
1372. The reconstituted solution of any one of the preceding embodiments, wherein impurity is formaldehyde.
1373. The reconstituted solution of any one of the preceding embodiments, wherein impurity is acetaldehyde.
1374. The reconstituted solution of any one of the preceding embodiments, wherein impurity is and propionaldehyde.
1375. The reconstituted solution of any one of the preceding embodiments, wherein the level of polyethylene oxide presented in the reconstituted solution is below about 3%, below about 2%, below about 1%, below about 0.5%, or below about 0.1%, as measured by high-performance liquid chromatography (HPLC).
1376. The reconstituted solution of any one of the preceding embodiments, wherein the level of polyethylene oxide presented in the reconstituted solution is below about 3%, as measured by high-performance liquid chromatography (HPLC).
1377. The reconstituted solution of any one of the preceding embodiments, wherein the level of polyethylene oxide presented in the reconstituted solution is below about 2%, as measured by high-performance liquid chromatography (HPLC).
1378. The reconstituted solution of any one of the preceding embodiments, wherein the level of polyethylene oxide presented in the reconstituted solution is below about 1%, as measured by high-performance liquid chromatography (HPLC).
1379. The reconstituted solution of any one of the preceding embodiments, wherein the level of polyethylene oxide presented in the reconstituted solution is below about 0.5%, as measured by high-performance liquid chromatography (HPLC).
1380. The reconstituted solution of any one of the preceding embodiments, wherein the level of polyethylene oxide presented in the reconstituted solution is or below about 0.1%, as measured by high-performance liquid chromatography (HPLC).
1381. The reconstituted solution of any one of the preceding embodiments, wherein the total level of one or more impurities with c Log P of about 1 or less presented in the reconstituted solution is from about 30% to about 35%, from about 25% to about 29%, from about 20% to about 25%, from about 15% to about 19%, from about 10% to about 14%, from about 5% to about 9%, or from about 0% to about 4%, as measured by high-performance liquid chromatography (HPLC).
1382. The reconstituted solution of any one of the preceding embodiments, wherein the total level of one or more impurities with c Log P of about 1 or less presented in the reconstituted solution is from about 30% to about 35%, as measured by high-performance liquid chromatography (HPLC).
1383. The reconstituted solution of any one of the preceding embodiments, wherein the total level of one or more impurities with c Log P of about 1 or less presented in the reconstituted solution is from about 25% to about 29%, as measured by high-performance liquid chromatography (HPLC).
1384. The reconstituted solution of any one of the preceding embodiments, wherein the total level of one or more impurities with c Log P of about 1 or less presented in the reconstituted solution is from about 20% to about 25%, as measured by high-performance liquid chromatography (HPLC).
1385. The reconstituted solution of any one of the preceding embodiments, wherein the total level of one or more impurities with c Log P of about 1 or less presented in the reconstituted solution is from about 15% to about 19%, as measured by high-performance liquid chromatography (HPLC).
1386. The reconstituted solution of any one of the preceding embodiments, wherein the total level of one or more impurities with c Log P of about 1 or less presented in the reconstituted solution is from about 10% to about 14%, as measured by high-performance liquid chromatography (HPLC).
1387. The reconstituted solution of any one of the preceding embodiments, wherein the total level of one or more impurities with c Log P of about 1 or less presented in the reconstituted solution is from about 5% to about 9%, as measured by high-performance liquid chromatography (HPLC).
1388. The reconstituted solution of any one of the preceding embodiments, wherein the total level of one or more impurities with c Log P of about 1 or less presented in the reconstituted solution is from about 0% to about 4%, as measured by high-performance liquid chromatography (HPLC).
1389. The reconstituted solution of any one of the preceding embodiments, wherein the total level of one or more impurities having a boiling of about 220° C. or less presented in the reconstituted solution is from about 35% to about 40%, from about 30% to about 34%, from about 25% to about 29%, from about 20% to about 25%, from about 15% to about 19%, from about 10% to about 14%, from about 5% to about 9%, or from about 0% to about 4%, as measured by high-performance liquid chromatography (HPLC).
1390. The reconstituted solution of any one of the preceding embodiments, wherein the total level of one or more impurities having a boiling of about 220° C. or less presented in the reconstituted solution is from about 35% to about 40%, as measured by high-performance liquid chromatography (HPLC).
1391. The reconstituted solution of any one of the preceding embodiments, wherein the total level of one or more impurities having a boiling of about 220° C. or less presented in the reconstituted solution is from about 30% to about 34%, as measured by high-performance liquid chromatography (HPLC).
1392. The reconstituted solution of any one of the preceding embodiments, wherein the total level of one or more impurities having a boiling of about 220° C. or less presented in the reconstituted solution is from about 25% to about 29%, as measured by high-performance liquid chromatography (HPLC).
1393. The reconstituted solution of any one of the preceding embodiments, wherein the total level of one or more impurities having a boiling of about 220° C. or less presented in the reconstituted solution is from about 20% to about 25%, as measured by high-performance liquid chromatography (HPLC).
1394. The reconstituted solution of any one of the preceding embodiments, wherein the total level of one or more impurities having a boiling of about 220° C. or less presented in the reconstituted solution is from about 15% to about 19%, as measured by high-performance liquid chromatography (HPLC).
1395. The reconstituted solution of any one of the preceding embodiments, wherein the total level of one or more impurities having a boiling of about 220° C. or less presented in the reconstituted solution is from about 10% to about 14%, as measured by high-performance liquid chromatography (HPLC).
1396. The reconstituted solution of any one of the preceding embodiments, wherein the total level of one or more impurities having a boiling of about 220° C. or less presented in the reconstituted solution is from about 5% to about 9%, as measured by high-performance liquid chromatography (HPLC).
1397. The reconstituted solution of any one of the preceding embodiments, wherein the total level of one or more impurities having a boiling of about 220° C. or less presented in the reconstituted solution is from about 0% to about 4%, as measured by high-performance liquid chromatography (HPLC).
1398. The reconstituted solution of any one of the preceding embodiments, wherein the reconstituted solution comprises purified Poloxamer 407, and wherein the level of the one or more otic therapeutic agents presented in the reconstituted solution is about 1.5 fold or higher, about 1.8 fold or higher, about 2 fold or higher, about 2.5 fold or higher, about 3 fold or higher, about 5 fold or higher, or about 10 fold or higher as compared to a comparable reconstituted solution without purified Poloxamer 407;
   optionally, the comparable reconstituted solution comprises unpurified Poloxamer 407.
1399. The reconstituted solution of any one of the preceding embodiments, wherein the reconstituted solution comprises purified Poloxamer 407, and wherein the level of the one or more otic therapeutic agents presented in the reconstituted solution is about 1.5 fold or higher as compared to a comparable reconstituted solution without purified Poloxamer 407.
1400. The reconstituted solution of any one of the preceding embodiments, wherein the reconstituted solution comprises purified Poloxamer 407, and wherein the level of the one or more otic therapeutic agents presented in the reconstituted solution is about 1.8 fold or higher as compared to a comparable reconstituted solution without purified Poloxamer 407.
1401. The reconstituted solution of any one of the preceding embodiments, wherein the reconstituted solution comprises purified Poloxamer 407, and wherein the level of the one or more otic therapeutic agents presented in the reconstituted solution is about 2 fold or higher as compared to a comparable reconstituted solution without purified Poloxamer 407.
1402. The reconstituted solution of any one of the preceding embodiments, wherein the reconstituted solution comprises purified Poloxamer 407, and wherein the level of the one or more otic therapeutic agents presented in the reconstituted solution is about 2.5 fold or higher as compared to a comparable reconstituted solution without purified Poloxamer 407.
1403. The reconstituted solution of any one of the preceding embodiments, wherein the reconstituted solution comprises purified Poloxamer 407, and wherein the level of the one or more otic therapeutic agents presented in the reconstituted solution is about 3 fold or higher as compared to a comparable reconstituted solution without purified Poloxamer 407.
1404. The reconstituted solution of any one of the preceding embodiments, wherein the reconstituted solution comprises purified Poloxamer 407, and wherein the level of the one or more otic therapeutic agents presented in the reconstituted solution is about 5 fold or higher as compared to a comparable reconstituted solution without purified Poloxamer 407.
1405. The reconstituted solution of any one of the preceding embodiments, wherein the reconstituted solution comprises purified Poloxamer 407, and wherein the level of the one or more otic therapeutic agents presented in the reconstituted solution is about 10 fold or higher as compared to a comparable reconstituted solution without purified Poloxamer 407.
1406. The reconstituted solution of any one of the preceding embodiments, wherein the comparable reconstituted solution comprises unpurified Poloxamer 407.
1407. The reconstituted solution of any one of the preceding embodiments, wherein the reconstituted solution comprises purified Poloxamer 407, and wherein the reconstituted solution has lower batch-to-batch variability of one or more gelation properties (e.g., gelation temperature, viscosity, and/or stability) as compared to a comparable reconstituted solution without purified Poloxamer 407;
   optionally, the comparable reconstituted solution comprises unpurified Poloxamer 407.
1408. The reconstituted solution of any one of the preceding embodiments, wherein the reconstituted solution comprises purified Poloxamer 407, and wherein the reconstituted solution has a lower gelation temperature, a narrower gelation temperature range, a more sustained release of the hearing loss treatment agent, and/or a higher viscosity as compared to a reconstituted solution without purified Poloxamer 407; optionally, the comparable reconstituted solution comprises unpurified Poloxamer 407.
1409. The reconstituted solution of any one of the preceding embodiments, wherein the reconstituted solution comprises purified Poloxamer 407, and wherein the reconstituted solution has a reduced degradation rate as compared to a comparable reconstituted solution without purified Poloxamer 407;
   optionally, the comparable reconstituted solution comprises unpurified Poloxamer 407.
1410. The reconstituted solution of any one of the preceding embodiments, being suitable for injection;
   optionally, the reconstituted solution is suitable for intratympanic injection
1411. The reconstituted solution of any one of the preceding embodiments, wherein the reconstituted solution maintains one or more rheometric properties of a pharmaceutical composition which is used for preparing the lyophilized pharmaceutical composition
1412. The reconstituted solution of any one of the preceding embodiments, wherein the reconstituted solution has a reduced degradation rate as compared to a reconstituted solution prepared from a comparable lyophilized pharmaceutical composition without purified Poloxamer 407;
   optionally, the comparable lyophilized pharmaceutical composition comprises unpurified Poloxamer 407.
1413. The reconstituted solution of any one of the preceding embodiments, comprising one or more o£
   water or a buffering agent;
   a bulking agent;
   a stabilizing agent;
   a tonicity-adjusting agent; and a soothing agent
1414. The reconstituted solution of any one of the preceding embodiments, comprising water or a buffering agent
1415. The reconstituted solution of any one of the preceding embodiments, comprising a bulking agent
1416. The reconstituted solution of any one of the preceding embodiments, comprising a stabilizing agent
1417. The reconstituted solution of any one of the preceding embodiments, comprising a tonicity-adjusting agent
1418. The reconstituted solution of any one of the preceding embodiments, comprising a soothing agent
1419. A method of facilitating the generation of a tissue and/or a cell, comprising delivering a pharmaceutically effective amount of the lyophilized pharmaceutical composition, pharmaceutical composition, or reconstituted composition of any preceding embodiment to the tissue and/or the cell.
1420. A method of treating a subject who has, or is at risk of developing, a disease associated with absence or a lack of a tissue and/or a cell, comprising administering to the subject a pharmaceutically effective amount of the lyophilized pharmaceutical, pharmaceutical composition, or reconstituted composition of any preceding embodiment
1421. A method of increasing a population of vestibular cells in a vestibular tissue, comprising delivering a pharmaceutically effective amount of the lyophilized pharmaceutical composition, pharmaceutical composition, or reconstituted composition of any preceding embodiment
1422. A method of treating a subject who has, or is at risk of developing a vestibular condition, comprising administering to the subject a pharmaceutically effective amount of the lyophilized pharmaceutical, pharmaceutical composition, or reconstituted composition of any preceding embodiment
1423. A method of increasing a population of cochlear cells in a cochlear tissue, comprising delivering a pharmaceutically effective amount of the lyophilized pharmaceutical composition, pharmaceutical composition, or reconstituted composition of any preceding embodiment
1424. A method of treating a subject who has, or is at risk of developing a cochlear condition, comprising administering to the subject a pharmaceutically effective amount of the lyophilized pharmaceutical, pharmaceutical composition, or reconstituted composition of any preceding embodiment
1425. A method of increasing a population of cells found in the Organ of Corti, comprising delivering a pharmaceutically effective amount of the lyophilized pharmaceutical composition, pharmaceutical composition, or reconstituted composition of any preceding embodiment
1426. A method of increasing a population of hair cells found in the Organ of Corti, comprising delivering a pharmaceutically effective amount of the lyophilized pharmaceutical composition, pharmaceutical composition, or reconstituted composition of any preceding embodiment
1427. A method of increasing a population of inner hair cells found in the Organ of Corti, comprising delivering a pharmaceutically effective amount of the lyophilized pharmaceutical composition, pharmaceutical composition, or reconstituted composition of any preceding embodiment
1428. A method of increasing a population of outer hair cells found in the Organ of Corti, comprising delivering a pharmaceutically effective amount of the lyophilized pharmaceutical composition, pharmaceutical composition, or reconstituted composition of any preceding embodiment
1429. A method of increasing a population of neuronal cells found in the Organ of Corti, comprising delivering a pharmaceutically effective amount of the lyophilized pharmaceutical composition, pharmaceutical composition, or reconstituted composition of any preceding embodiment
1430. A method of treating a subject who has, or is at risk of developing a hearing condition, comprising administering to the subject a pharmaceutically effective amount of the lyophilized pharmaceutical composition, pharmaceutical composition, or reconstituted composition of any preceding embodiment
1431. The method of embodiment 1430, wherein the hearing condition is sensorineural hearing loss.
1432. A lyophilized composition, pharmaceutical composition, or reconstituted composition of any preceding embodiment for use in therapy.
1433. The lyophilized pharmaceutical composition, pharmaceutical composition, or reconstituted composition of any preceding embodiment for use in facilitating the generation of a tissue and/or a cell.
1434. The lyophilized pharmaceutical composition, pharmaceutical composition, or reconstituted composition of any preceding embodiment for use in in treating a subject who has, or is at risk of developing, a disease associated with absence or a lack of a tissue and/or a cell.
1435. The lyophilized pharmaceutical composition, pharmaceutical composition, or reconstituted composition of any preceding embodiment for use in increasing a population of vestibular cells in a vestibular tissue.
1436. The lyophilized pharmaceutical composition, pharmaceutical composition, or reconstituted composition of any preceding embodiment for use in treating a subject who has, or is at risk of developing a vestibular condition
1437. The lyophilized pharmaceutical composition, pharmaceutical composition, or reconstituted composition of any preceding embodiment for use in increasing a population of cochlear cells in a cochlear tissue.
1438. The lyophilized pharmaceutical composition, pharmaceutical composition, or reconstituted composition of any preceding embodiment for use in treating a subject who has, or is at risk of developing a cochlear condition
1439. The lyophilized pharmaceutical composition, pharmaceutical composition, or reconstituted composition of any preceding embodiment for use in increasing a population of cells found in the Organ of Corti.
1440. The lyophilized pharmaceutical composition, pharmaceutical composition, or reconstituted composition of any preceding embodiment for use in increasing a population of hair cells found in the Organ of Corti.
1441. The lyophilized pharmaceutical composition, pharmaceutical composition, or reconstituted composition of any preceding embodiment for use in increasing a population of inner hair cells found in the Organ of Corti.
1442. The lyophilized pharmaceutical composition, pharmaceutical composition, or reconstituted composition of any preceding embodiment for use in increasing a population of outer hair cells found in the Organ of Corti.
1443. The lyophilized pharmaceutical composition, pharmaceutical composition, or reconstituted composition of any preceding embodiment for use in increasing a population of neuronal cells found in the Organ of Corti.
1444. The lyophilized pharmaceutical composition, pharmaceutical composition, or reconstituted composition of any preceding embodiment for use in treating a subject who has, or is at risk of developing a hearing condition
1445. The composition for use according to embodiment 1444, wherein the hearing condition is sensorineural hearing loss.
1446. Use of the lyophilized pharmaceutical composition, pharmaceutical composition, or reconstituted composition of any preceding embodiment in the manufacture of a medicament for facilitating the generation of a tissue and/or a cell.
1447. Use of the lyophilized pharmaceutical composition, pharmaceutical composition, or reconstituted composition of any preceding embodiment in the manufacture of a medicament for in treating a subject who has, or is at risk of developing, a disease associated with absence or a lack of a tissue and/or a cell.
1448. Use of the lyophilized pharmaceutical composition, pharmaceutical composition, or reconstituted composition of any preceding embodiment in the manufacture of a medicament for increasing a population of vestibular cells in a vestibular tissue.
1449. Use of the lyophilized pharmaceutical composition, pharmaceutical composition, or reconstituted composition of any preceding embodiment in the manufacture of a medicament for treating a subject who has, or is at risk of developing a vestibular condition
1450. Use of the lyophilized pharmaceutical composition, pharmaceutical composition, or reconstituted composition of any preceding embodiment in the manufacture of a medicament for increasing a population of cochlear cells in a cochlear tissue.
1451. Use of the lyophilized pharmaceutical composition, pharmaceutical composition, or reconstituted composition of any preceding embodiment in the manufacture of a medicament for treating a subject who has, or is at risk of developing a cochlear condition
1452. Use of the lyophilized pharmaceutical composition, pharmaceutical composition, or reconstituted composition of any preceding embodiment in the manufacture of a medicament for increasing a population of cells found in the Organ of Corti.
1453. Use of the lyophilized pharmaceutical composition, pharmaceutical composition, or reconstituted composition of any preceding embodiment in the manufacture of a medicament for increasing a population of hair cells found in the Organ of Corti.
1454. Use of the lyophilized pharmaceutical composition, pharmaceutical composition, or reconstituted composition of any preceding embodiment, in the manufacture of a medicament for increasing a population of inner hair cells found in the Organ of Corti.
1455. Use of the lyophilized pharmaceutical composition, pharmaceutical composition, or reconstituted composition of any preceding embodiment in the manufacture of a medicament for increasing a population of outer hair cells found in the Organ of Corti.
1456. Use of the lyophilized pharmaceutical, pharmaceutical composition, or reconstituted composition of any preceding embodiment in the manufacture of a medicament for increasing a population of neuronal cells found in the Organ of Corti.
1457. Use of the lyophilized pharmaceutical composition, pharmaceutical composition, or reconstituted composition of any preceding embodiment in the manufacture of a medicament for treating a subject who has, or is at risk of developing a hearing condition
1458. The use of embodiment 1457, wherein the hearing condition is sensorineural hearing loss.

1.-88. (canceled)

89. A lyophilized pharmaceutical composition comprising a poloxamer, 30% to 50% by weight of valproic acid or a pharmaceutically acceptable salt thereof, and 1% to 2% by weight of CHIR99021 or a pharmaceutically acceptable salt thereof.

90. The lyophilized pharmaceutical composition of claim 89, wherein the poloxamer has a polydispersity index of less than 1.07.

91. The lyophilized pharmaceutical composition of claim 89, wherein the concentration of aldehydes is less than 10 ppm (μg/g).

92. The lyophilized pharmaceutical composition of claim 91, wherein the aldehydes are selected from formaldehyde, acetaldehyde, and/or propionaldehyde.

93. The lyophilized pharmaceutical composition of claim 91, wherein the aldehydes are selected from acetaldehyde and/or propionaldehyde.

94. The lyophilized pharmaceutical composition of claim 89, wherein the composition comprises 50% to 70% by weight of poloxamer.

95. The lyophilized pharmaceutical composition of claim 89, wherein the poloxamer comprises a polyethylene oxide-polypropylene oxide-polyethylene oxide triblock copolymer.

96. The lyophilized pharmaceutical composition of claim 89, wherein the poloxamer comprise: 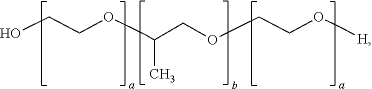
wherein b is 56+/−10%, and each a is independently 101+/−10%.

97. The lyophilized pharmaceutical composition of claim 89, wherein the poloxamer is poloxamer 407.

98. The lyophilized pharmaceutical composition of claim 89, comprising 50 to 200 mg of valproic acid or a pharmaceutically acceptable salt thereof; and/or comprising 100 to 200 mg of the poloxamer.

99. The lyophilized pharmaceutical composition of claim 89, wherein the pharmaceutically acceptable salt of valproic acid is sodium valproate.

100. The lyophilized pharmaceutical composition of claim 89, for preparing a reconstituted solution by a reconstitution process wherein the poloxamer is a thermoreversible gel.

101. A method of treating a subject comprising administering to the subject a lyophilized pharmaceutical composition of claim 89, wherein the subject has, or is at risk of developing a hearing condition.

102. The method of claim 101, wherein the hearing condition is sensorineural hearing loss.

103. A reconstituted pharmaceutical composition obtainable by a method comprising adding a diluent to the lyophilized pharmaceutical composition of claim 89.

104. The reconstituted pharmaceutical composition of claim 103, wherein the diluent comprises water.

105. The reconstituted pharmaceutical composition of claim 103, wherein the concentration of valproic acid or a pharmaceutically acceptable salt thereof is greater than 100 mg/ml; and/or
the concentration of CHIR99021 or a pharmaceutically acceptable salt thereof is less than 7.5 mg/mL.

106. A method of treating a subject comprising administering to the subject a reconstituted pharmaceutical composition of claim 105, wherein the subject has, or is at risk of developing a hearing condition.

107. The method of claim 106, wherein the hearing condition is sensorineural hearing loss.
